SEQUENCE LISTING (31 sequences)

SEQ ID NO: 1 - protein, 33 aa, Artificial Sequence
  Synthetic peptide - Truncated amino acid sequence of human ALCAM protein corresponding to residues 551 to 583 (corresponding to the cytosolic tail of ALCAM)
MKKSKTASKHVNKDLGNMEENKKLEENNHKTEA

SEQ ID NO: 2 - protein, 61 aa, Artificial Sequence
  Synthetic peptide - Truncated amino acid sequence of human BCAM protein corresponding to residues 569 to 628 (corresponding to the cytosolic tail of BCAM) with the addition of an initiating Methionine residue
MYCVRRKGGPCCRQRREKGAPPPGEPGLSHSGSEQPEQTGLLMGGASGGARGGSGGFGDEC

SEQ ID NO: 3 - protein, 55 aa, Artificial Sequence
  Synthetic peptide - Truncated amino acid sequence of human MCAM protein corresponding to residues 584 to 637 (corresponding to the cytosolic tail of MCAM) with the addition of an initiating methionine residue
MKKGKLPCRRSGKQEITLPPSRKSELVVEVKSDKLPEEMGLLQGSSGDKRAPGDQ

SEQ ID NO: 4 - protein, 27 aa, Artificial Sequence
  Synthetic peptide - Truncated amino acid sequence of human EpCAM protein corresponding to residues 289 to 314 (corresponding to the cytosolic tail of EpCAM) with the addition of an initiating methionine residue
MSRKKRMAKYEKAEIKEMGEMHRELNA

SEQ ID NO: 5 - protein, 42 aa, Artificial Sequence
  Synthetic peptide - Truncated amino acid sequence of human CADM4 protein corresponding to residues 346 to 388 (corresponding to the cytosolic tail of CADM4) with the addition of an initiating methionine residue
MSVRQKGSYLTHEASGLDEQGEAREAFLNGSDGHKRKEEFFI

SEQ ID NO: 6 - protein, 23 aa, Artificial Sequence
  Synthetic peptide - Truncated amino acid sequence of human ALCAM protein corresponding to residues 559 to 580 of human ALCAM protein with the addition of an initiating methionine residue
MKHVNKDLGNMEENKKLEENNHK

SEQ ID NO: 7 - protein, 22 aa, Artificial Sequence
  Synthetic peptide - Truncated amino acid sequence of human RAGE protein corresponding to residues 370 to 390 of human RAGE protein with the addition of an initiating methionine residue
MGEERKAPENQEEEEERAELNQ

SEQ ID NO: 8 - protein, 44 aa, Artificial Sequence
  Synthetic peptide - Truncated amino acid sequence of human RAGE protein corresponding to residues 362 to 404 of human RAGE protein with a mutation of Serine 391 to Alanine plus an initiating methionine residue
MLWQRRQRRGEERKAPENQEEEEERAELNQAEEPEAGESSTGGP

SEQ ID NO: 9 - protein, 583 aa, Homo sapiens
MESKGASSCRLLFCLLISATVFRPGLGWYTVNSAYGDTIIIPCRLDVPQNLMFGKWKYEKPDGSPVFIAFRSSTKKSVQYDDVPEYKDRLNLSENYTLSISNARISDEKRFVCMLVTEDNVFEAPTIVKVFKQPSKPEIVSKALFLETEQLKKLGDCISEDSYPDGNITWYRNGKVLHPLEGAVVIIFKKEMDPVTQLYTMTSTLEYKTTKADIQMPFTCSVTYYGPSGQKTIHSEQAVFDIYYPTEQVTIQVLPPKNAIKEGDNITLKCLGNGNPPPEEFLFYLPGQPEGIRSSNTYTLMDVRRNATGDYKCSLIDKKSMIASTAITVHYLDLSLNPSGEVTRQIGDALPVSCTISASRNATVVWMKDNIRLRSSPSFSSLHYQDAGNYVCETALQEVEGLKKRESLTLIVEGKPQIKMTKKTDPSGLSKTIICHVEGFPKPAIQWTITGSGSVINQTEESPYINGRYYSKIIISPEENVTLTCTAENQLERTVNSLNVSAISIPEHDEADEISDENREKVNDQAKLIVGIVVGLLLAALVAGVVYWLYMKKSKTASKHVNKDLGNMEENKKLEENNHKTEA

SEQ ID NO: 10 - protein, 628 aa, Homo sapiens
MEPPDAPAQARGAPRLLLLAVLLAAHPDAQAEVRLSVPPLVEVMRGKSVILDCTPTGTHDHYMLEWFLTDRSGARPRLASAEMQGSELQVTMHDTRGRSPPYQLDSQGRLVLAEAQVGDERDYVCVVRAGAAGTAEATARLNVFAKPEATEVSPNKGTLSVMEDSAQEIATCNSRNGNPAPKITWYRNGQRLEVPVEMNPEGYMTSRTVREASGLLSLTSTLYLRLRKDDRDASFHCAAHYSLPEGRHGRLDSPTFHLTLHYPTEHVQFWVGSPSTPAGWVREGDTVQLLCRGDGSPSPEYTLFRLQDEQEEVLNVNLEGNLTLEGVTRGQSGTYGCRVEDYDAADDVQLSKTLELRVAYLDPLELSEGKVLSLPLNSSAVVNCSVHGLPTPALRWTKDSTPLGDGPMLSLSSITFDSNGTYVCEASLPTVPVLSRTQNFTLLVQGSPELKTAEIEPKADGSWREGDEVTLICSARGHPDPKLSWSQLGGSPAEPIPGRQGWVSSSLTLKVTSALSRDGISCEASNPHGNKRHVFHFGTVSPQTSQAGVAVMAVAVSVGLLLLVVAVFYCVRRKGGPCCRQRREKGAPPPGEPGLSHSGSEQPEQTGLLMGGASGGARGGSGGFGDEC

SEQ ID NO: 11 - protein, 646 aa, Homo sapiens
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQGLDLDTMISLLSEPQELLVNYVSDVRVSPAAPERQEGSSLTLTCEAESSQDLEFQWLREETGQVLERGPVLQLHDLKREAGGGYRCVASVPSIPGLNRTQLVNVAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLPEPESRGVVIVAVIVCILVLAVLGAVLYFLYKKGKLPCRRSGKQEITLPPSRKSELVVEVKSDKLPEEMGLLQGSSGDKRAPGDQGEKYIDLRH

SEQ ID NO: 12 - protein, 314 aa, Homo sapiens
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSMCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAGVIAVIVVVVIAVVAGIVVLVISRKKRMAKYEKAEIKEMGEMHRELNA

SEQ ID NO: 13 - protein, 388 aa, Homo sapiens
MGRARRFQWPLLLLWAAAAGPGAGQEVQTENVTVAEGGVAEITCRLHQYDGSIVVIQNPARQTLFFNGTRALKDERFQLEEFSPRRVRIRLSDARLEDEGGYFCQLYTEDTHHQIATLTVLVAPENPVVEVREQAVEGGEVELSCLVPRSRPAATLRWYRDRKELKGVSSSQENGKVWSVASTVRFRVDRKDDGGIIICEAQNQALPSGHSKQTQYVLDVQYSPTARIHASQAVVREGDTLVLTCAVTGNPRPNQIRWNRGNESLPERAEAVGETLTLPGLVSADNGTYTCEASNKHGHARALYVLVVYDPGAVVEAQTSVPYAIVGGILALLVFLIICVLVGMVWCSVRQKGSYLTHEASGLDEQGEAREAFLNGSDGHKRKEEFFI

SEQ ID NO: 14 - protein, 404 aa, Homo sapiens
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRGEERKAPENQEEEEERAELNQSEEPEAGESSTGGP

SEQ ID NO: 15 - protein, 359 aa, Homo sapiens [sequence omitted]

SEQ ID NO: 16 - protein, 583 aa, Mus musculus
MASKVSPSCRLVFCLLISAAVLRPGLGWYTVNSAYGDTIVMPCRLDVPQNLMFGKWKYEKPDGSPVFIAFRSSTKKSVQYDDVPEYKDRLSLSENYTLSIANAKISDEKRFVCMLVTEDNVFEAPTLVKVFKQPSKPEIVNKAPFLETDQLKKLGDCISRDSYPDGNITWYRNGKVLQPVEGEVAILFKKEIDPGTQLYTVTSSLEYKTTRSDIQMPFTCSVTYYGPSGQKTIYSEQEIFDIYYPTEQVTIQVLPPKNAIKEGDNITLQCLGNGNPPPEEFMFYLPGQPEGIRSSNTYTLTDVRRNATGDYKCSLIDKRNMAASTTITVHYLDLSLNPSGEVTKQIGDTLPVSCTISASRNATVVWMKDNIRLRSSPSFSSLHYQDAGNYVCETALQEVEGLKKRESLTLIVEGKPQIKMTKKTDPSGLSKTIICHVEGFPKPAIHWTITGSGSVINQTEESPYINGRYYSKIIISPEENVTLTCTAENQLERTVNSLNVSAISIPEHDEADDISDENREKVNDQAKLIVGIVVGLLLAALVAGVVYWLYMKKSKTASKHVNKDLGNMEENKKLEENNHKTEA

SEQ ID NO: 17 - protein, 306 aa, Gallus gallus
MELLRGAALLLLLCAAACAQDSCTCTKNKRVTNCKLIDNVCHCNSIGSSVSVNCEILTSKCLLMKAEMANTKSGRREKPKDALQDTDGLYDPECGNNGLFKAKQCNGTTCWCVNTAGVRRTDKHDTDLKCNQLVRTTWIIIEMRHAERKTPLNAESLIRYLKDTITSRYMLDGRYISGVVYENPTITIDLKQNSSDKTPGDVDITDVAYYFEKDVKDDSIFLNNKLNMNIDNEELKFDNMMVYYVDEVPPEFSMKSLTAGVIAVIVIVVLAIVAGIIGLVLSRRRKGKYVKAEMKEMNEMHRGLNA

SEQ ID NO: 18 - protein, 350 aa, Homo sapiens
MNSFNYTTPDYGHYDDKDTLDLNTPVDKTSNTLRVPDILALVIFAVVFLVGVLGNALVVWVTAFEAKRTINAIWFLNLAVADFLSCLALPILFTSIVQHHHWPFGGAACSILPSLILLNMYASILLLATISADRFLLVFKPIWCQNFRGAGLAWIACAVAWGLALLLTIPSFLYRVVREEYFPPKVLCGVDYSHDKRRERAVAIVRLVLGFLWPLLTLTICYTFILLRTWSRRATRSTKTLKVVVAVVASFFIFWLPYQVTGIMMSFLEPSSPTFLLLNKLDSLCVSFAYINCCINPIIYVVAGQGFQGRLRKSLPSLLRNVLTEESVVRESKSFTRSTVDTMAQKTQAV

SEQ ID NO: 19 - protein, 24 aa, Artificial Sequence
  Synthetic peptide - Truncated amino acid sequence corresponding to residues 338 to 361 of human RAGE protein (corresponding to the transmembrane domain and juxta- membrane residues of RAGE)
LGTLALALGILGGLGTAALLIGVI

SEQ ID NO: 20 - protein, 11 aa, Artificial Sequence
  Synthetic peptide - Amino acid sequence of residues 47-57 of HIV-1 tat protein (corresponding to the cell penetrating element of the cationic domain)
YGRKKRRQRRR

SEQ ID NO: 21 - protein, 12 aa, Artificial Sequence
  Synthetic peptide - Truncated amino acid sequence corresponding to residues 379 to 390 of human RAGE protein
QEEEEERAELNQ

SEQ ID NO: 22 - protein, 13 aa, Artificial Sequence
  Synthetic peptide - Truncated amino acid sequence corresponding to residues 379 to 390 of human RAGE protein with the addition of an initiating methionine residue
MQEEEEERAELNQ

SEQ ID NO: 23 - protein, 30 aa, Artificial Sequence
  Synthetic peptide - Truncated amino acid sequence corresponding to residues 362 to 391 of human RAGE protein with mutation of Serine 391 to Alanine
LWQRRQRRGEERKAPENQEEEEERAELNQA

SEQ ID NO: 24 - protein, 29 aa, Artificial Sequence
  Synthetic peptide - Truncated amino acid sequence corresponding to residues 362 to 390 of human RAGE protein
LWQRRQRRGEERKAPENQEEEEERAELNQ

SEQ ID NO: 25 - protein, 12 aa, Artificial Sequence
  Synthetic peptide - Truncated amino acid sequence corresponding to residues 379 to 390 of human RAGE protein with a mutation of Glutamine 390 to Arginine
QEEEEERAELNR

SEQ ID NO: 26 - protein, 12 aa, Artificial Sequence
  Synthetic peptide - Truncated amino acid sequence corresponding to residues 379 to 390 of human RAGE protein with a mutation of Glutamine 390 to Lysine
QEEEEERAELNK

SEQ ID NO: 27 - protein, 12 aa, Artificial Sequence
  Synthetic peptide - Truncated amino acid sequence corresponding to residues 379 to 390 of human RAGE protein with a mutation of Glutamine 379 to Lysine
KEEEEERAELNQ

SEQ ID NO: 28 - protein, 12 aa, Artificial Sequence
  Synthetic peptide - Truncated amino acid sequence corresponding to residues 379 to 390 of human RAGE protein with a mutation of Glutamine 379 to Lysine and Glutamine 390 to Lysine
KEEEEERAELNK

SEQ ID NO: 29 - protein, 12 aa, Artificial Sequence
  Synthetic peptide - Truncated amino acid sequence corresponding to residues 379 to 390 of human RAGE protein with a mutation of Glutamine 379 to Lysine and Glutamine 390 to Arginine
KEEEEERAELNR

SEQ ID NO: 30 - protein, 19 aa, Artificial Sequence
  Synthetic peptide - Truncated amino acid sequence corresponding to residues 343 to 361 of human RAGE protein (corresponding to the transmembrane domain)
LALGILGGLGTAALLIGVI

SEQ ID NO: 31 - protein, 43 aa, Artificial Sequence
  Synthetic peptide - Truncated amino acid sequence corresponding to residues 362 to 404 of human RAGE protein (corresponding to the cytosolic tail of RAGE)
LWQRRQRRGEERKAPENQEEEEERAELNQSEEPEAGESSTGGP

(19) United States
(12) Patent Application Publication
(10) Pub. No.: US 2022/0169693 A1
(43) Pub. Date: Jun. 2, 2022

(54) Screening Assays, Modulators and Modulation of Intracellular Signalling Mediated by Immunoglobulin Superfamily Cell Adhesion Molecules

(71) Applicants: Monash University, Clayton (AU); The University of Western Australia, Nedlands (AU)

(72) Inventors: Kevin Donald George PFLEGER, Nedlands (AU); Merlin Christopher THOMAS, Ashburton (AU); Raelene Jane PICKERING, Cheltenham (AU); Carlos ROSADO, Sunshine (AU); Elizabeth Katherine Mary JOHNSTONE, Duncraig (AU)

(21) Appl. No.: 17/312,317

(22) PCT Filed: Dec. 10, 2019
(86) PCT No.: PCT/AU2019/051358

§ 371 Date: Jun. 9, 2021

(30) Foreign Application Priority Data
Dec. 10, 2018 (AU) 2018904691

(51) Int. Cl. C07K 14/705; C12N 15/85

(52) U.S. Cl. CPC C07K 14/705; A61K 38/1774; C12N 15/85

(57) ABSTRACT

The invention relates to modulators of activation of Immunoglobulin Superfamily Cell Adhesion Molecules (IgSF CAMs) and modulators of activation of Receptor for Advanced Glycation End Products (RAGE) as well as screening assays for identifying modulators of activation of molecules associated with certain diseases and/or conditions in which IgSF CAMs and/or RAGE are implicated, and to medicaments and methods of treatment comprising administration of such modulators.

### CROSS-REFERENCE TO RELATED APPLICATIONS

This application is a National Stage Entry of International Patent Application No. PCT/AU2019/051358, filed on Dec. 10, 2019, entitled “Screening Assays, Modulators and Modulation of Intracellular Signalling Mediated by Immunoglobulin Superfamily Cell Adhesion Molecules”, which claims priority to Australian Patent Application No. 2018904691, filed on Dec. 10, 2018, entitled “Screening Assays, Modulators and Modulation of Intracellular Signalling Mediated by Immunoglobulin Superfamily Cell Adhesion Molecules”. The disclosures of all of the above applications are hereby incorporated herein by reference in their entirety.

### REFERENCE TO A SEQUENCE LISTING SUBMITTED IN A COMPUTER READABLE FORMAT

The application includes an electronic sequence listing in a file named 288446_CAM_Sequence_Listing_ST25.txt, created on Jan. 6, 2022, and containing 50,445 bytes, which is here by incorporated by reference in its entirety for all purposes.

### FIELD OF THE INVENTION

This invention relates generally to screening assays for identifying modulators of activation of molecules associated with certain diseases and/or conditions, to such modulators, and to methods of treatment comprising administration of such modulators. More specifically, the invention relates to modulators of activation of Immunoglobulin Superfamily Cell Adhesion Molecules (IgSF CAMs), including but not limited to Activated Leukocyte Cell Adhesion Molecule (ALCAM, also known as cluster of differentiation 166 [CD166]), Melanoma Cell Adhesion Molecule (MCAM, also known as CD146/MUC18), Basal Cell Adhesion Molecule (BCAM, also known as the Lutheran blood group glycoprotein), Epithelial Cell Adhesion Molecule (EpCAM, also known as TACSTD1 (tumor-associated calcium signal transducer 1), CD326 (cluster of differentiation 326), or the 17-1A antigen), Cell Adhesion Molecule 4 (CADM4, also known as TSLL2, IGSF4C, SynCAM4, or NecI-4) via IgSF CAM ligand-independent mechanisms by certain co-located, activated G Protein-Coupled Receptors (GPCRs), including activated type 1 angiotensin receptor (AT1R), with or without also modulating activation of IgSF CAMs by IgSF CAM ligands. This invention also relates to screening assays for identifying such modulators, and to methods of treatment of IgSF CAM-related disorders using said modulators. This invention also relates to modulators of activation of the Receptor for Advanced Glycation End-products (RAGE) via RAGE ligand-independent mechanisms by certain co-located, activated G Protein-Coupled Receptors (GPCRs), including activated type 1 angiotensin receptor (AT1R) and activated complement receptor C5a receptor 1, with or without also modulating activation of RAGE by RAGE ligands, where the modulators are analogues, fragments or derivatives of members of the IgSF CAM superfamily, including ALCAM₅₅₉₋₅₈₀. This invention also relates to screening assays for identifying such modulators, and to methods of treatment of RAGE-related disorders using said modulators.

### BACKGROUND OF THE INVENTION

Cell adhesion molecules (CAMs) facilitate interactions between cells and their external environment, and include cadherins, integrins, selectins, and IgSF CAMs. The Immunoglobulin Superfamily is characterised by an extracellular domain (which contains one or more Ig-like domains), a single transmembrane domain, and a cytoplasmic tail. IgSF CAMs are cell adhesion molecules (CAMs) that belong to the Immunoglobulin Superfamily (IgSF). They mediate adhesion through their N-terminal Ig-like ectodomains, which commonly bind other Ig-like domains of the same structure on an opposing cell surface (homophilic adhesion) but may also interact with integrins and carbohydrates (heterophilic adhesion).

Some IgSF CAMs can also act as pattern recognition receptors, and become activated by diverse ligands, triggering intracellular signalling pathways, mediated by the C-terminal intracellular domains of IgSF CAM members which interact with cytoskeletal or adaptor proteins involved in the propagation of signalling events mediated by ligand binding.

Activated Leukocyte Cell Adhesion Molecule (ALCAM) is a 105-kDa type I transmembrane protein and member of the IgSF CAM superfamily. ALCAM contains a multi-ligand binding extracellular immunoglobulin-like ectodomain, comprising two N-terminal, membrane-distal variable-(V)-type and three membrane-proximal constant-(C2)-type Ig folds (VVC2C2C2) ectodomain, a single-span transmembrane domain and a short (32 amino acid) cytosolic domain.

ALCAM is primarily implicated in cell adhesion between adjacent cells, mediated by homophilic trans interactions (ALCAM-ALCAM) or heterophilic interaction (ALCAM-CD6) specifically via its NH₂-terminal V-type immunoglobulin folds (Patel et al., 1995, Swart, 2002, van Kempen et al., 2001, Zimmerman et al., 2006) while the proximal C-type immunoglobulin folds mediate oligomerization in cis.

The ectodomain of ALCAM also functions as a pattern recognition multi-ligand receptor with diverse ligands including S100 proteins that trigger activation of NFKB dependent pathways (von Bauer, Oikonomou et al. 2013) accompanied by activation of the small GTPases RhoA, Rac1 and Cdc42.

ALCAM is shed upon activation by MMPs/ADAMs, releasing a soluble isoform (Hebron, Li et al. 2018). ALCAM is partly regulated by alternative splicing that modulates the rate of shedding. It is known that NFKB activation directly enhances ALCAM expression by binding to the ALCAM promoter (Wang, Gu et al. 2011).

ALCAM is also a nerve-derived growth factor (NGF) and brain-derived neurotrophic factor (BDNF) co-receptor, and is involved in neurite outgrowth and neuron survival in cooperation with fibroblast growth factor signalling (Wade, Thomas et al. 2012). Notably the extracellular (ligand binding) ectodomain of ALCAM is required for potentiation of NGF-dependent neurite outgrowth and constructs without the intracellular cytoplasmic domain retain this function.

ALCAM expression was first identified on leucocytes, but is broadly detectable in a wide variety of cell types, including epithelial cells, fibroblasts, neuronal cells, hepatocytes, podocytes and bone marrow cells, although typically restricted to subsets of cells involved in dynamic growth and migration.

ALCAM is involved in several important physiological processes such as maturation of hematopoietic stem cells in blood forming tissues, angiogenesis, neural development, axon fasciculation, the immune response, and osteogenesis.

Dynamic alteration of cell adhesion is an integral step to cancer progression and ALCAM has been associated with the progression of diverse types of cancer. ALCAM has been implicated in many aspects of tumour biology including growth, migration and invasion of tumour cells. ALCAM's participation in malignant progression has been recognized in numerous studies for many common cancers including but not limited to: pancreatic cancer (Hong, Michalski et al. 2010), melanoma (Penna, Orso et al. 2013), prostate cancer (Hansen, Arnold et al. 2014) breast cancer (Piao, Jiang et al. 2012), liver cancer/hepatoma (Yu, Wang et al. 2014), mesothelioma (Inaguma, Lasota et al. 2018), gastric cancer (Ye, Du et al. 2015), bladder cancer (Arnold Egloff, Du et al. 2017), brain tumours (Atukeren, Turk et al. 2017) and colon cancer (Kozovska, Gabrisova et al. 2014), in which elevated ALCAM shedding directly relates to poor patient outcome and a more invasive tumour pattern. ALCAM is thought to be directly involved in cell migration, invasion, spread and metastasis. Soluble ALCAM (sALCAM) or ALCAM-IgG-Fc chimeras containing the ectodomain are able to inhibit cell-cell adhesion and modulate cell migration.

ALCAM has also been implicated in a range of immunological disorders including but not limited to asthma (Kim, Hong et al. 2018), delayed-type hypersensitivity (von Bauer, Oikonomou et al. 2013), and food allergy (Kim, Kim et al. 2018). ALCAM has been identified as an important costimulatory molecule on antigen-presenting cells (APCs) contributing to the antigen specific induction of T cell activation and proliferation relevant to immunological disorders, including allergy and autoimmunity.

ALCAM has also been implicated in a range of brain disorders, in particular those neuro-inflammatory disorders in which leukocyte migration across the blood-brain barrier is implicated including multiple sclerosis (Lecuyer, Saint-Laurent et al. 2017), encephalomyelitis (Lecuyer, Saint-Laurent et al. 2017) and retinal vascular disease (Smith, Chipps et al. 2012).

ALCAM has also been implicated in a range of chronic inflammatory diseases including but not limited to chronic kidney disease (Smith, Chipps et al. 2012) diabetic nephropathy (Sulaj, Kopf et al. 2017), atherosclerosis (Rauch, Rosenkranz et al. 2011), stroke (Smedbakken, Jensen et al. 2011), and aortic valve sclerosis (Guerraty, Grant et al. 2011).

Although the ligand-binding actions of the ectodomain of ALCAM are well known, the functions of the short (32 amino acid) cytoplasmic domain of ALCAM are poorly understood. It is thought that the cytosolic tail of ALCAM possibly regulates adhesion through links with the cytoskeleton. The cytoplasmic tail of ALCAM contains a positive-charge-rich domain at the membrane proximal site and a KTEA peptide motif at the C-terminus, facilitating interactions with adaptor proteins, ezrin and syntenin-1 respectively (Weidle, Eggle et al. 2010, Te Riet, Helenius et al. 2014). ALCAM also binds IQ-GAP1 following homotypic interactions (ALCAM-ALCAM) of ectodomains. PKCα also plays a role in the modulation of ALCAM-dependent adhesion (Zimmerman, Nelissen et al. 2004). However, the cytoplasmic domain does not contain conserved PKC-phosphorylation motifs and despite there being two serines and two threonines present in the cytoplasmic domain of ALCAM, these are dispensable for ALCAM-mediated adhesion (Zimmerman, Nelissen et al. 2004).

Prior art teaches away from the cytoplasmic domain of ALCAM being significantly involved in ALCAM-mediated adhesion. Mutant ALCAM constructs in which the cytoplasmic domain has been deleted retain the actions of full-length ALCAM on cell proliferation, while constructs lacking the extracellular N-terminal V-domain are non-functional with respect to adhesion, ligand binding and proliferation.

Melanoma cell adhesion molecule (MCAM) (also known as M-CAM, CD146 or cell MUC18) is a 113 kDa cell adhesion molecule of the immunoglobulin superfamily of cell adhesion molecules (IgSF CAM) with 22.7% identity and 41.7% similarity to ALCAM. Like ALCAM, it contains a large multi-ligand binding extracellular immunoglobulin-like ectodomain, comprising two N-terminal, membrane-distal variable-(V)-type and three membrane-proximal constant-(C2)-type Ig folds (VVC2C2C2) ectodomain, a single-span transmembrane domain and a short (63 amino acid) cytosolic domain.

MCAM is primarily implicated in cell adhesion between adjacent cells, mediated by homophilic trans interactions (MCAM-MCAM) or heterophilic interaction (MCAM-laminin4) specifically via its NH₂-terminal V-type immunoglobulin folds while the proximal C-type immunoglobulin folds mediate oligomerization in cis (Wang and Yan 2013).

MCAM also functions as a pattern recognition multi-ligand receptor with diverse ligands including S100 proteins that trigger activation of NFKB dependent pathways (Ruma, Putranto et al. 2016).

The interaction of MCAM with VEGFR-2 on the endothelial cell surface has been shown to activate AKT and p38 signaling and increase cell migration (Jouve, Bachelier et al. 2015). Interaction with Laminin-4 facilitates Th17 cell entry into the central nervous system. Binding of Netrin-1 to CD146/MCAM was reported to activate an array of downstream signaling and increase endothelial cell proliferation, migration, and angiogenesis (Tu, Zhang et al. 2015). Recently, CD146 was reported to interact with galectin-1 and galectin-3 (Colomb, Wang et al. 2017). Notably, the extracellular (ligand binding) ectodomain of MCAM appears to be critical for these functions.

MCAM is actively involved in many normal cellular processes including vascular development, signal transduction, cell migration, mesenchymal stem cell differentiation, angiogenesis and immune response (Shih 1999).

MCAM is highly expressed by endothelial cells and has been used for the identification of endothelial progenitors in the circulation. MCAM is also expressed on other vascular cells including smooth muscle and pericytes. Soluble MCAM thought to be a marker of endothelial damage. MCAM is also a differentiation marker of intermediary placental trophoblast, and is expressed in mammary lobular and ductal epithelium (Guezguez et al. 2006).

MCAM is also known to be highly expressed by melanoma cells where it is associated with melanoma metastasis (Johnson 1999). CD146 is also overly expressed on a large variety of carcinomas in addition to melanoma (Wang and Yan 2013). It is thought that CD146 promotes tumor growth, angiogenesis, and metastasis, and CD146 is regarded as a promising target for tumor therapy (Wang and Yan 2013).

Although the ligand-binding actions of the ectodomain of MCAM are known, the functions of the cytoplasmic domain of MCAM are poorly understood. It is thought that the cytosolic tail of MCAM possibly regulates adhesion through links with the cytoskeleton. The cytosolic tail of MCAM contains two potential recognition sites for protein kinase C (PKC), an ERM (protein complex of ezrin, radixin and moesin) binding site, a motif with microvilli extension and a double leucine motif for baso-lateral targeting in epithelia. Although the cytosolic tail is not required for ligand binding and adhesion, the mutant MCAM in which the cytosolic tail has been deleted is unable to activate NFKB after activation with S100A8/A9 (Ruma, Putranto et al. 2016).

Basal cell adhesion molecule (BCAM), also known as the Lutheran blood group glycoprotein, is a 78-85 kDa cell adhesion molecule of the immunoglobulin superfamily of cell adhesion molecules (IgSF CAM) similar to ALCAM. Like ALCAM, BCAM contains a large multi-ligand binding extracellular immunoglobulin-like ectodomain, comprising two N-terminal, membrane-distal variable-(V)-type and three membrane-proximal constant-(C2)-type Ig folds (VVC2C2C2) ectodomain, a single-span transmembrane domain and a short cytosolic domain. The 78 kDa isoform exhibits the same N-terminal amino acid sequence as the 85 kDa but lacks the last 40 C-terminal amino acids of the cytoplasmic tail.

BCAM is primarily implicated in cell adhesion between adjacent cells, mediated by homophilic (trans) interactions (BCAM-BCAM) or heterophilic interaction (BCAM-laminina5 or BCAM-integrin α4β1) specifically via its NH₂-terminal V-type immunoglobulin folds while the proximal C-type immunoglobulin folds mediate oligomerization in cis. The extracellular domains of BCAM represent high affinity laminin receptors (El Nemer, Gane et al. 1998). Furthermore, the long-tail (85 kDa) or the short-tail (78 kDa) BCAM confer to transfected cells the same laminin binding capacity.

BCAM was originally identified in the Lutheran blood group system and is the major laminin-binding protein in sickle red cells (Zen, Batchvarova et al. 2004), myeloproliferative neoplasms (Novitzky-Basso, Spring et al. 2018), and polycythemia rubra vera (De Grandis, Cassinat et al. 2015), where it mediates endothelial cell adhesion.

BCAM may also have a role in leukocyte recruitment in inflamed tissue, including crescentic glomerulonephritis, where BCAM deficiency was sufficient to prevent severe glomerular damage and renal failure in mice (Huang, Filipe et al. 2014).

BCAM is implicated in the development and progression of a range of cancers. BCAM has been shown to be upregulated in skin, brain, and endometrial-ovarian tumors, in hepatocellular carcinoma, and in breast cancer, where it represents an independent marker of response to neoadjuvant chemotherapy (Bartolini, Cardaci et al. 2016). Data suggest that BCAM-targeted agents might have broad application in different tumor types (Bartolini, Cardaci et al. 2016).

Although the ligand-binding actions of the ectodomain of BCAM are known, the functions of the cytoplasmic domain of BCAM are poorly understood. The predominant 78 kDa isoform has a tail of only 20 amino acids, while the 85 kDa isoform's tail is 60 amino acids long. It is thought that the cytosolic tail of BCAM possibly regulates adhesion through links with the cytoskeleton. The Arg573Lys574 motif in the shared cytoplasmic tail of BCAM attaches to the spectrin cytoskeleton and regulates cell adhesive activity and actin organization in epithelial cells. The cytoplasmic tail carries an SH3 binding motif, a di-leucine motif, and potential phosphorylation sites. Protein kinase A phosphorylates Ser621 in the cytoplasmic tail and stimulates adhesion of sickled red blood cells to laminin under flow conditions. A constitutively active JAK2 promotes Lu-mediated red cell adhesion through the Rap1/Akt pathway. The abnormal adhesion of red blood cells to laminin α5 is due to the Ser621 phosphorylation of Lu/BCAM by the JAK2/Rap1/Akt pathway (Kikkawa, Ogawa et al. 2013).

Epithelial cell adhesion molecule (EpCAM) is a 30- to 40-kDa type I membrane glycoprotein. EpCAM is known to play a role in cell adhesion through homotypic interactions as well as cell signaling, migration, proliferation, and differentiation. Like other IgSF CAMs it contains an immunoglobulin-like extracellular domain, a single transmembrane domain and a short (26 amino acids) intracellular domain, sometimes referred to as EpICD. The intracellular domain of EpCAM (EpICD) is required for EpCAM to mediate intercellular adhesion due to its ability to interact with the intracellular actin cytoskeleton via alpha-actinin.

EpCAM is highly expressed in epithelial cancers, including colon carcinoma where it is thought to play a role in oncogenicity, tumorigenesis and metastasis. EpCAM expression has been considered to be a prognostic marker as well as a potential target for immunotherapeutic strategies.

EpCAM can be cleaved from the cell surface, releasing the extracellular domain into the area surrounding the cell, and EpICD is released into the cytoplasm, where it forms a complex with the proteins FHL2, β-catenin, and Lef that binds to DNA and promotes the transcription of genes that promote tumor growth. Nuclear localisation of EpICD is a poor prognostic feature of epithelial cancers.

Cell Adhesion Molecule 4 (CADM4) is a type I membrane glycoprotein known to play a role in cell adhesion through homotypic interactions as well as cell signaling, migration, proliferation, and differentiation. Like other IgSF CAMs it contains an immunoglobulin-like extracellular domain, a single transmembrane domain and a short (43 amino acid) intracellular domain. The loss of or reduced expression of CADM4 is associated with tumor progression in some cancers.

CADM4 is also a member of the nectin-like (Ned) adhesion proteins also known as SynCAMs, NecI-proteins play an important role in nerve myelination and neurodevelopment, partly through regulation of axon-glia interactions.

The renin-angiotensin aldosterone system (RAAS) is a key homeostatic pathway that is also implicated in the development and progression of many common diseases and disease processes. Inhibition of the renin-angiotensin aldosterone system (RAAS) with angiotensin-converting enzyme (ACE) inhibitors, or angiotensin II receptor type 1 (AT₁R) blockers (inhibitors) is widely used for the management of many diseases and/or conditions including hypertension, cardiovascular disease (CVD), heart failure, chronic kidney disease (CKD), and diabetic complications. RAAS inhibition has also been shown to have benefits in preventing diabetes (Tikellis et al., 2004), in neuroprotection (Thoene-Reineke et al., 2011), modifying the growth of certain cancers (Shen et al., 2016) and even in ageing, with genetic deletion of AT₁R conferring longevity in mice (Benigni et al., 2009).

These actions of RAAS blockers are additional to and independent of blood pressure lowering conferred by RAAS blockers, as comparable lowering of the blood pressure with other agents does not confer the same benefits (Lee et al., 1993). Specifically, activation of the AT₁R by angiotensin II (Ang II) triggers induction of oxidative stress, activation of Nuclear Factor κB (NFκB) and inflammation through pathways that are distinct from those that cause vasoconstriction.

Activation of the renin-angiotensin aldosterone system (RAAS) is known to be an important mediator of atherosclerosis (Lee et al., 1993; and Jacoby et al., 2003). Atherogenesis is increased following an infusion of angiotensin (Ang) II and in experimental models is associated with physiological RAAS activation, including a low salt diet (Tikellis et al., 2012), diabetes (Goldin et al., 2006; and Soro-Paavonen et al., 2008) and genetic deletion of angiotensin converting enzyme 2 (Ace2) (Thomas et al., 2010), independent of its effects on blood pressure homeostasis. Similarly, inhibition of the RAAS has anti-atherosclerotic actions that are additional to and independent of lowering systemic blood pressure (Candido et al., 2002; Candido et al., 2004; and Knowles et al., 2000). Ang II has a number of direct pro-atherosclerotic effects (Daugherty et al., 2000; Ferrario et al., 2006; and Ekholm et al., 2009), including the induction of oxidative stress (Rajagopalan et al., 1996), vascular adhesion (Grafe et al., 1997) and inflammation (Marvar et al., 2010).

These pro-atherosclerotic actions are thought to be primarily mediated by activation of the type 1 angiotensin receptor (AT₁R) and subsequent induction of reactive oxygen species (ROS) and activation of NFκB signalling (Li et al., 2008). However, the signalling mechanisms that underlie these actions are poorly understood, including their relative independence from conventional vasoconstrictor signalling via the AT₁R.

It is against this background that the inventors describe the selective interactions between certain activated GPCRs, such as AT₁R, and the cytosolic tail of certain IgSF CAMs, independently of any IgSF CAM ligand, or the transmembrane domain or ectodomain of said IgSF CAMs, initiating downstream signalling leading to activation of NFκB, a key transcription factor implicated in inflammation, oxidative stress, fibrogenesis, cellular proliferation and cellular survival.

The inventors have shown that selective modulation, such as inhibition, of IgSF CAM ligand-independent activation (transactivation) of the cytosolic tail of certain IgSF CAMs by certain activated GPCRs, can be achieved by targeting this pathway using common signalling elements shared by these IgSF CAMs, and the inventors' assays and modulators identified therefrom, act upon this transactivation (ligand-independent activation of an IgSF CAM) process.

The inventors show that an analogue, fragment or derivative of an IgSF CAM can modulate signalling mediated by the cytosolic tails of certain IgSF CAMs.

The inventors further show that an analogue, fragment or derivative of the Receptor for Advanced Glycation End-Products (RAGE) can also modulate signalling mediated by the cytosolic tails of certain IgSF CAMs.

The inventors further show that an analogue, fragment or derivative of certain IgSF CAMs can modulate RAGE ligand-independent activation of RAGE by certain activated co-located GPCRs and resultant signalling mediated by the cytosolic tail of RAGE.

### SUMMARY OF THE INVENTION

The inventors have shown that key elements in the cytosolic tail of RAGE can modulate activation of IgSF CAMs, specifically ALCAM, BCAM, EpCAM, CADM4 and MCAM.

The inventors have further shown that key elements in the cytosolic tail of RAGE can modulate IgSF CAM ligand-independent activation of IgSF CAMs, specifically ALCAM, BCAM, EpCAM, CADM4 and MCAM, by certain activated co-located GPCRs, specifically the AT1 receptor by Angiotensin II.

The inventors have shown that following activation of certain co-located GPCRs, such as AT₁R by Ang II, the cytosolic tail of IgSF CAMs, and specifically ALCAM, BCAM, EpCAM, CADM4 and MCAM, can be activated, independently of any cognate ligand or the ectodomain of said IgSF CAMs, initiating downstream signalling leading to activation of NFκB, a key transcription factor implicated in inflammation, oxidative stress, fibrogenesis, cellular proliferation and cellular survival.

In one form of the invention, the human ALCAM ectodomain (500 amino acids) corresponds to residues 28-527.

In one form of the invention, the human MCAM ectodomain (536 amino acids) corresponds to residues 24-559.

In one form of the invention, the human BCAM ectodomain (516 amino acids) corresponds to residues 32-547.

In one form of the invention, the human EpCAM ectodomain (242 amino acids) corresponds to residues 24-265.

In one form of the invention, the human CADM4 ectodomain (304 amino acids) corresponds to residues 21-324.

In one form of the invention, the human ALCAM cytosolic tail (cytosolic domain; 34 amino acids) corresponds to residues 550-583.

In one form of the invention, the human ALCAM cytosolic tail (cytosolic domain; 33 amino acids) corresponds to residues 551-583.

In one form of the invention, the human MCAM cytosolic tail (cytosolic domain; 63 amino acids) corresponds to residues 584-646.

In one form of the invention, the MCAM cytosolic tail (cytosolic domain; 54 amino acids) corresponds to residues 584-637.

In one form of the invention, the human BCAM cytosolic tail (cytosolic domain; 60 amino acids) corresponds to residues 569-628.

In one form of the invention, the human EpCAM cytosolic tail (cytosolic domain 26 amino acids) corresponds to residues 289-314.

In one form of the invention, the human CADM4 cytosolic tail (cytosolic domain 43 amino acids) corresponds to residues 346-388.

The inventors have further shown that key elements in the cytosolic tail of an IgSF CAM, specifically ALCAM, can also modulate RAGE ligand-independent activation of RAGE by certain activated co-located GPCRs, specifically activation of the AT1 receptor by Angiotensin II.

Prior art does not suggest or disclose any evidence for a functional interaction between the cytosolic tail of an IgSF CAM and a GPCR, such as an angiotensin receptor, such as AT₁R. Nor does it anticipate that activation of a GPCR by that GPCR's cognate ligand, such as an angiotensin receptor by Ang II, would directly result in activation of an IgSF CAM, in particular the cytosolic tail, nor the subsequent induction of signalling via an IgSF CAM, in the absence of any ligand for the IgSF CAM or indeed without requiring the presence of the ligand-binding ectodomain of these proteins, which is considered necessary for signalling and teaches away from these findings. Consequently, it could not be anticipated that modulation of ligand-independent activation of the cytosolic tail of an IgSF CAM would involve modulation of signalling induced following activation of a certain co-located GPCR, such as by binding of Ang II to the AT₁R.

In a preferred form of the present invention, the IgSF CAM is ALCAM.

In another preferred form of the present invention, the IgSF CAM is BCAM.

In another preferred form of the present invention, the IgSF CAM is MCAM.

In another preferred form of the present invention, the IgSF CAM is EpCAM.

In another preferred form of the present invention, the IgSF CAM is CADM4.

In a particularly preferred form of the present invention, the IgSF CAM is ALCAM or BCAM.

In another particularly preferred form of the present invention, the IgSF CAM is ALCAM or MCAM.

In another particularly preferred form of the present invention, the IgSF CAM is ALCAM or EpCAM.

In another particularly preferred form of the present invention, the IgSF CAM is ALCAM or CADM4.

In another particularly preferred form of the present invention, the IgSF CAM is BCAM or MCAM.

In another particularly preferred form of the present invention, the IgSF CAM is BCAM or EpCAM.

In another particularly preferred form of the present invention, the IgSF CAM is BCAM or CADM4.

In another particularly preferred form of the present invention, the IgSF CAM is MCAM or EpCAM.

In another particularly preferred form of the present invention, the IgSF CAM is MCAM or CADM4.

In another particularly preferred form of the present invention, the IgSF CAM is EpCAM or CADM4.

In another particularly preferred form of the present invention, the IgSF CAM is ALCAM or BCAM or MCAM.

In another particularly preferred form of the present invention, the IgSF CAM is ALCAM or BCAM or EpCAM.

In another particularly preferred form of the present invention, the IgSF CAM is ALCAM or BCAM or CADM4.

In another particularly preferred form of the present invention, the IgSF CAM is ALCAM or MCAM or EpCAM.

In another particularly preferred form of the present invention, the IgSF CAM is ALCAM or MCAM or CADM4.

In another particularly preferred form of the present invention, the IgSF CAM is ALCAM or EpCAM or CADM4.

In another particularly preferred form of the present invention, the IgSF CAM is BCAM or MCAM or EpCAM.

In another particularly preferred form of the present invention, the IgSF CAM is BCAM or MCAM or CADM4.

In another particularly preferred form of the present invention, the IgSF CAM is MCAM or EpCAM or CADM4.

In another particularly preferred form of the present invention, the IgSF CAM is ALCAM or BCAM or MCAM or EpCAM.

In another particularly preferred form of the present invention, the IgSF CAM is ALCAM or BCAM or MCAM or CADM4.

In another particularly preferred form of the present invention, the IgSF CAM is BCAM or MCAM or EpCAM or CADM4.

In another particularly preferred form of the present invention, the IgSF CAM is ALCAM or MCAM or EpCAM or CADM4.

In another particularly preferred form of the present invention, the IgSF CAM is ALCAM or BCAM or EpCAM or CADM4.

In another particularly preferred form of the present invention, the IgSF CAM is ALCAM or BCAM or MCAM or EpCAM or CADM4.

In one form of the present invention, the IgSF CAM superfamily (IgSF CAMs) comprises: ALCAM (also known as CD166 or MEMD), BCAM (also known as CD239), BOC (also known as CDON2), CADM1 (also known as NECL2, ST17, BL2, SYNCAM, IGSF4A, NecI-2, SYNCAM1 or RA175), CADM2 (also known as NECL3, NecI-3 or SynCAM2), CADM3 (also known as BlgR, FLJ10698, TSLL1, NECL1, SynCAM3 or NecI-1), CADM4 (also known as TSLL2, NecI-4 or SynCAM4), CD2, CD244 (also known as 2B4, NAIL, NKR2B4, Nmrk or SLAMF4), CD28, CD47 (also known as IAP or OA3), CD58, CD84 (also known as SLAMF5, hCD84 or mCD84), CD96 (also known as TACTILE), CHL1 (also known as CALL, L1CAM2, FLJ44930 or MGC132578), CNTN1 (also known as F3 or GP135), CNTN2 (also known as TAG-1 or TAXI), CNTN3 (also known as BIG-1), CNTN4 (also known as BIG-2), CNTN5 (also known as NB-2 or hNB-2), CNTN6 (also known as NB-3), CRTAM (also known as CD355), DSCAM (also known as CHD2-42 or CHD2-52), DSCAML1 (also known as KIAA1132), F11R (also known as PAM-1, JCAM, JAM-1, JAM-A, JAMA or CD321), FGFRL1, GP6 (also known as GPVI), HEPACAM (also known as FLJ25530, hepaCAM or GLIALCAM), ICAM1 (also known as BB2 or CD54), ICAM2 (also known as CD102), ICAM3 (also known as CDW50, ICAM-R or CD50), ICAM4 (also known as CD242), ICAM5 (also known as TLN), IGSF5 (also known as JAM4), IZUMO1 (also known as IZUMO, MGC34799 or OBF), IZUMO1R (also known as Folbp3 or JUNO), JAM2 (also known as VE-JAM, JAM-B, JAMB or CD322), JAM3 (also known as JAM-C or JAMC), JAML (also known as Gm638 or AMICA), L1CAM (also known as CD171), LILRB2 (also known as LIR-2, ILT4, MIR-10, LIR2, CD85d or MIR10), LRFN1 (also known as KIAA1484 or SALM2), LRFN2 (also known as FIGLER2), LRFN3 (also known as MGC2656, SALM4 or FIGLER1), LRFN4 (also known as MGC3103, SALM3. or FIGLER6), LRFN5 (also known as FIGLER8, SALM5), LRRC4 (also known as NAG14), LRRC4B (also known as DKFZp761A179 or HSM), LRRC4C (also known as KIAA1580 or NGL-1), MADCAM1 (also known as MACAM1), MCAM (also known as MUC18, CD146, MeICAM, METCAM or HEMCAM), MPZ (also known as HMSNIB, CMT2I or CMT2J), MPZL2 (also known as EVA), NCAM1 (also known as NCAM or CD56), NCAM2 (also known as NCAM21 or MGC51008), NECTIN1 (also known as PRR, PRR1, PVRR1, SK-12, HIgR, CLPED1, CD111 or OFC7), NECTIN2 (also known as PVRR2, PRR2 or CD112), NECTIN3 (also known as nectin-3, PPR3, PVRR3, DKFZP566B0846, CDw113 or CD113), NECTIN4 (also known as nectin-4, PRR4 or LNIR), NEO1 (also known as NGN, HsT17534, IGDCC2 or NTN1R2), NFASC (also known as NRCAML, KIAA0756, FLJ46866 or NF), NRCAM (also known as KIAA0343 or Bravo), PECAM1 (also known as CD31), PTPRM (also known as RPTPU or hR-PTPu), PVR (also known as CD155, HVED, NecI-5, NECL5 or Tage4), ROBO1 (also known as DUTT1, FLJ21882 or SAX3), ROBO2 (also known as KIAA1568), SDK1 (also known as FLJ31425), SIGLECL1 (also known as FLJ40235), SIRPA (also known as SHPS1, SIRP, MYD-1, BIT, P84, SHPS-1, SIRPalpha, CD172a, SIRPalpha2, MFR or SIRP-ALPHA-1), SIRPG (also known as bA77C3.1, SIRP-B2, SIRPgamma or CD172g), SLAMF1 (also known as CD150), SLAMF6 (also known as KALI, NTBA, KALIb, Ly108, SF2000, NTB-A or CD352), THY1 (also known as CD90), UNC5A (also known as KIAA1976 or UNC5H1), UNC5B (also known as UNC5H2 or p53RDL1), UNC5D (also known as KIAA1777 or Unc5h4), VCAM1 (also known as CD106), CLMP (also known as ASAM, FLJ22415 or ACAM), CXADR (also known as CAR), ESAM (also known as W117m), GPA33 (also known as A33), IGSF11 (also known as BT-IgSF, MGC35227, Igsf13, VSIG3 or CT119), VSIG1 (also known as MGC44287), VSIG2 (also known as CTXL, CTH), VSIG8, OPCML (also known as OPCM, OBCAM or IGLON1), NTM (also known as HNT, NTRI, IGLON2 or CEPU-1), LSAMP (also known as LAMP or IGLON3), NEGR1 (also known as KILON, MGC46680, Ntra or IGLON4), IGLON5 (also known as LOC402665), SIGLEC1 (also known as SIGLEC-1, CD169, FLJ00051, FLJ00055, FLJ00073, FLJ32150, dJ1009E24.1 or sialoadhesin), SIGLEC10 (also known as SIGLEC-10, SLG2, PRO940 or MGC126774), SIGLEC11, SIGLEC12 (also known as SLG, S2V, Siglec-XII, Siglec-12 or Siglec-L1), SIGLEC14, SIGLEC15 (also known as HsT1361), SIGLEC16 (also known as Siglec-P16), SIGLEC17P, SIGLEC18P, CD22 (also known as SIGLEC-2 or SIGLEC2), SIGLEC20P, SIGLEC21P, SIGLEC22P, SIGLEC24P, SIGLEC25P, SIGLEC26P, SIGLEC27P, SIGLEC28P, SIGLEC29P, CD33 (also known as SIGLEC3, SIGLEC-3, p67 or FLJ00391), SIGLEC30P, SIGLEC31P, MAG (also known as SIGLEC4A, SIGLEC-4A or S-MAG), SIGLEC5 (also known as OB-BP2, SIGLEC-5 or CD170), SIGLEC6 (also known as OB-BP1, SIGLEC-6 or CD327), SIGLEC7 (also known as SIGLEC-7, p75/AIRM1, QA79 or CD328), SIGLEC8 (also known as SIGLEC-8, SAF2, SIGLEC8L or MGC59785), and SIGLEC9 (also known as CD329).

In one form of the present invention, the IgSF CAM superfamily (IgSF CAMs) comprises: ALCAM (also known as CD166 or MEMD), BCAM (also known as CD239), BOC (also known as CDON2), CADM1 (also known as NECL2, ST17, BL2, SYNCAM, IGSF4A, NecI-2, SYNCAM1 or RA175), CADM2 (also known as NECL3, NecI-3 or SynCAM2), CADM3 (also known as BlgR, FLJ10698, TSLL1, NECL1, SynCAM3 or NecI-1), CADM4 (also known as TSLL2, NecI-4 or SynCAM4), CD2, CD244 (also known as 2B4, NAIL, NKR2B4, Nmrk or SLAMF4), CD28, CD47 (also known as IAP or OA3), CD58, CD84 (also known as SLAMF5, hCD84 or mCD84), CD96 (also known as TACTILE), CHL1 (also known as CALL, L1CAM2, FLJ44930 or MGC132578), CNTN1 (also known as F3 or GP135), CNTN2 (also known as TAG-1 or TAXI), CNTN3 (also known as BIG-1), CNTN4 (also known as BIG-2), CNTN5 (also known as NB-2 or hNB-2), CNTN6 (also known as NB-3), CRTAM (also known as CD355), DSCAM (also known as CHD2-42 or CHD2-52), DSCAML1 (also known as KIAA1132), F11R (also known as PAM-1, JCAM, JAM-1, JAM-A, JAMA or CD321), FGFRL1, GP6 (also known as GPVI), HEPACAM (also known as FLJ25530, hepaCAM or GLIALCAM), ICAM1 (also known as BB2 or CD54), ICAM2 (also known as CD102), ICAM3 (also known as CDW50, ICAM-R or CD50), ICAM4 (also known as CD242), ICAM5 (also known as TLN), IGSF5 (also known as JAM4), IZUMO1 (also known as IZUMO, MGC34799 or OBF), IZUMO1R (also known as Folbp3 or JUNO), JAM2 (also known as VE-JAM, JAM-B, JAMB or CD322), JAM3 (also known as JAM-C or JAMC), JAML (also known as Gm638 or AMICA), L1CAM (also known as CD171), LILRB2 (also known as LIR-2, ILT4, MIR-10, LIR2, CD85d or MIR10), LRFN1 (also known as KIAA1484 or SALM2), LRFN2 (also known as FIGLER2), LRFN3 (also known as MGC2656, SALM4 or FIGLER1), LRFN4 (also known as MGC3103, SALM3. or FIGLER6), LRFN5 (also known as FIGLER8, SALM5), LRRC4 (also known as NAG14), LRRC4B (also known as DKFZp761A179 or HSM), LRRC4C (also known as KIAA1580 or NGL-1), MADCAM1 (also known as MACAM1), MCAM (also known as MUC18, CD146, MeICAM, METCAM or HEMCAM), MPZ (also known as HMSNIB, CMT2I or CMT2J), MPZL2 (also known as EVA), NCAM1 (also known as NCAM or CD56), NCAM2 (also known as NCAM21 or MGC51008), NECTIN1 (also known as PRR, PRR1, PVRR1, SK-12, HIgR, CLPED1, CD111 or OFC7), NECTIN2 (also known as PVRR2, PRR2 or CD112), NECTIN3 (also known as nectin-3, PPR3, PVRR3, DKFZP566B0846, CDw113 or CD113), NECTIN4 (also known as nectin-4, PRR4 or LNIR), NEO1 (also known as NGN, HsT17534, IGDCC2 or NTN1R2), NFASC (also known as NRCAML, KIAA0756, FLJ46866 or NF), NRCAM (also known as KIAA0343 or Bravo), PECAM1 (also known as CD31), PTPRM (also known as RPTPU or hR-PTPu), PVR (also known as CD155, HVED, NecI-5, NECL5 or Tage4), ROBO1 (also known as DUTT1, FLJ21882 or SAX3), ROBO2 (also known as KIAA1568), SDK1 (also known as FLJ31425), SIGLECL1 (also known as FLJ40235), SIRPA (also known as SHPS1, SIRP, MYD-1, BIT, P84, SHPS-1, SIRPalpha, CD172a, SIRPalpha2, MFR or SIRP-ALPHA-1), SIRPG (also known as bA77C3.1, SIRP-B2, SIRPgamma or CD172g), SLAMF1 (also known as CD150), SLAMF6 (also known as KALI, NTBA, KALIb, Ly108, SF2000, NTB-A or CD352), THY1 (also known as CD90), UNC5A (also known as KIAA1976 or UNC5H1), UNC5B (also known as UNC5H2 or p53RDL1), UNC5D (also known as KIAA1777 or Unc5h4), VCAM1 (also known as CD106), CLMP (also known as ASAM, FLJ22415 or ACAM), CXADR (also known as CAR), ESAM (also known as W117m), GPA33 (also known as A33), IGSF11 (also known as BT-IgSF, MGC35227, Igsf13, VSIG3 or CT119), VSIG1 (also known as MGC44287), VSIG2 (also known as CTXL, CTH), VSIG8, OPCML (also known as OPCM, OBCAM or IGLON1), NTM (also known as HNT, NTRI, IGLON2 or CEPU-1), LSAMP (also known as LAMP or IGLON3), NEGR1 (also known as KILON, MGC46680, Ntra or IGLON4) and IGLON5 (also known as LOC402665).

In one form of the present invention, the IgSF CAM superfamily (IgSF CAMs) comprises: ALCAM (also known as CD166 or MEMD), BCAM (also known as CD239), BOC (also known as CDON2), CADM1 (also known as NECL2, ST17, BL2, SYNCAM, IGSF4A, NecI-2, SYNCAM1 or RA175), CADM2 (also known as NECL3, NecI-3 or SynCAM2), CADM3 (also known as BlgR, FLJ10698, TSLL1, NECL1, SynCAM3 or NecI-1), CADM4 (also known as TSLL2, NecI-4 or SynCAM4), CD2, CD244 (also known as 2B4, NAIL, NKR2B4, Nmrk or SLAMF4), CD28, CD47 (also known as IAP or OA3), CD58, CD84 (also known as SLAMF5, hCD84 or mCD84), CD96 (also known as TACTILE), CHL1 (also known as CALL, L1CAM2, FLJ44930 or MGC132578), CNTN1 (also known as F3 or GP135), CNTN2 (also known as TAG-1 or TAXI), CNTN3 (also known as BIG-1), CNTN4 (also known as BIG-2), CNTN5 (also known as NB-2 or hNB-2), CNTN6 (also known as NB-3), CRTAM (also known as CD355), DSCAM (also known as CHD2-42 or CHD2-52), DSCAML1 (also known as KIAA1132), F11R (also known as PAM-1, JCAM, JAM-1, JAM-A, JAMA or CD321), FGFRL1, GP6 (also known as GPVI), HEPACAM (also known as FLJ25530, hepaCAM or GLIALCAM), ICAM1 (also known as BB2 or CD54), ICAM2 (also known as CD102), ICAM3 (also known as CDW50, ICAM-R or CD50), ICAM4 (also known as CD242), ICAM5 (also known as TLN), IGSF5 (also known as JAM4), IZUMO1 (also known as IZUMO, MGC34799 or OBF), IZUMO1R (also known as Folbp3 or JUNO), JAM2 (also known as VE-JAM, JAM-B, JAMB or CD322), JAM3 (also known as JAM-C or JAMC), JAML (also known as Gm638 or AMICA), L1CAM (also known as CD171), LILRB2 (also known as LIR-2, ILT4, MIR-10, LIR2, CD85d or MIR10), LRFN1 (also known as KIAA1484 or SALM2), LRFN2 (also known as FIGLER2), LRFN3 (also known as MGC2656, SALM4 or FIGLER1), LRFN4 (also known as MGC3103, SALM3. or FIGLER6), LRFN5 (also known as FIGLER8, SALM5), LRRC4 (also known as NAG14), LRRC4B (also known as DKFZp761A179 or HSM), LRRC4C (also known as KIAA1580 or NGL-1), MADCAM1 (also known as MACAM1), MCAM (also known as MUC18, CD146, MeICAM, METCAM or HEMCAM), MPZ (also known as HMSNIB, CMT2I or CMT2J), MPZL2 (also known as EVA), NCAM1 (also known as NCAM or CD56), NCAM2 (also known as NCAM21 or MGC51008), NECTIN1 (also known as PRR, PRR1, PVRR1, SK-12, HIgR, CLPED1, CD111 or OFC7), NECTIN2 (also known as PVRR2, PRR2 or CD112), NECTIN3 (also known as nectin-3, PPR3, PVRR3, DKFZP566B0846, CDw113 or CD113), NECTIN4 (also known as nectin-4, PRR4 or LNIR), NEO1 (also known as NGN, HsT17534, IGDCC2 or NTN1R2), NFASC (also known as NRCAML, KIAA0756, FLJ46866 or NF), NRCAM (also known as KIAA0343 or Bravo), PECAM1 (also known as CD31), PTPRM (also known as RPTPU or hR-PTPu), PVR (also known as CD155, HVED, NecI-5, NECL5 or Tage4), ROBO1 (also known as DUTT1, FLJ21882 or SAX3), ROBO2 (also known as KIAA1568), SDK1 (also known as FLJ31425), SIGLECL1 (also known as FLJ40235), SIRPA (also known as SHPS1, SIRP, MYD-1, BIT, P84, SHPS-1, SIRPalpha, CD172a, SIRPalpha2, MFR or SIRP-ALPHA-1), SIRPG (also known as bA77C3.1, SIRP-B2, SIRPgamma or CD172g), SLAMF1 (also known as CD150), SLAMF6 (also known as KALI, NTBA, KALIb, Ly108, SF2000, NTB-A or CD352), THY1 (also known as CD90), UNC5A (also known as KIAA1976 or UNC5H1), UNC5B (also known as UNC5H2 or p53RDL1), UNC5D (also known as KIAA1777 or Unc5h4), VCAM1 (also known as CD106), CLMP (also known as ASAM, FLJ22415 or ACAM), CXADR (also known as CAR), ESAM (also known as W117m), GPA33 (also known as A33), IGSF11 (also known as BT-IgSF, MGC35227, Igsf13, VSIG3 or CT119), VSIG1 (also known as MGC44287), VSIG2 (also known as CTXL, CTH), VSIG8, SIGLEC1 (also known as SIGLEC-1, CD169, FLJ00051, FLJ00055, FLJ00073, FLJ32150, dJ1009E24.1 or sialoadhesin), SIGLEC10 (also known as SIGLEC-10, SLG2, PRO940 or MGC126774), SIGLEC11, SIGLEC12 (also known as SLG, S2V, Siglec-XII, Siglec-12 or Siglec-L1), SIGLEC14, SIGLEC15 (also known as HsT1361), SIGLEC16 (also known as Siglec-P16), SIGLEC17P, SIGLEC18P, CD22 (also known as SIGLEC-2 or SIGLEC2), SIGLEC20P, SIGLEC21P, SIGLEC22P, SIGLEC24P, SIGLEC25P, SIGLEC26P, SIGLEC27P, SIGLEC28P, SIGLEC29P, CD33 (also known as SIGLEC3, SIGLEC-3, p67 or FLJ00391), SIGLEC30P, SIGLEC31P, MAG (also known as SIGLEC4A, SIGLEC-4A or S-MAG), SIGLEC5 (also known as OB-BP2, SIGLEC-5 or CD170), SIGLEC6 (also known as OB-BP1, SIGLEC-6 or CD327), SIGLEC7 (also known as SIGLEC-7, p75/AIRM1, QA79 or CD328), SIGLEC8 (also known as SIGLEC-8, SAF2, SIGLEC8L or MGC59785), and SIGLEC9 (also known as CD329).

In one form of the present invention, the IgSF CAM superfamily (IgSF CAMs) comprises: ALCAM (also known as CD166 or MEMD), BCAM (also known as CD239), BOC (also known as CDON2), CADM1 (also known as NECL2, ST17, BL2, SYNCAM, IGSF4A, NecI-2, SYNCAM1 or RA175), CADM2 (also known as NECL3, NecI-3 or SynCAM2), CADM3 (also known as BlgR, FLJ10698, TSLL1, NECL1, SynCAM3 or NecI-1), CADM4 (also known as TSLL2, NecI-4 or SynCAM4), CD2, CD244 (also known as 2B4, NAIL, NKR2B4, Nmrk or SLAMF4), CD28, CD47 (also known as IAP or OA3), CD58, CD84 (also known as SLAMF5, hCD84 or mCD84), CD96 (also known as TACTILE), CHL1 (also known as CALL, L1CAM2, FLJ44930 or MGC132578), CNTN1 (also known as F3 or GP135), CNTN2 (also known as TAG-1 or TAXI), CNTN3 (also known as BIG-1), CNTN4 (also known as BIG-2), CNTN5 (also known as NB-2 or hNB-2), CNTN6 (also known as NB-3), CRTAM (also known as CD355), DSCAM (also known as CHD2-42 or CHD2-52), DSCAML1 (also known as KIAA1132), F11R (also known as PAM-1, JCAM, JAM-1, JAM-A, JAMA or CD321), FGFRL1, GP6 (also known as GPVI), HEPACAM (also known as FLJ25530, hepaCAM or GLIALCAM), ICAM1 (also known as BB2 or CD54), ICAM2 (also known as CD102), ICAM3 (also known as CDW50, ICAM-R or CD50), ICAM4 (also known as CD242), ICAM5 (also known as TLN), IGSF5 (also known as JAM4), IZUMO1 (also known as IZUMO, MGC34799 or OBF), IZUMO1R (also known as Folbp3 or JUNO), JAM2 (also known as VE-JAM, JAM-B, JAMB or CD322), JAM3 (also known as JAM-C or JAMC), JAML (also known as Gm638 or AMICA), L1CAM (also known as CD171), LILRB2 (also known as LIR-2, ILT4, MIR-10, LIR2, CD85d or MIR10), LRFN1 (also known as KIAA1484 or SALM2), LRFN2 (also known as FIGLER2), LRFN3 (also known as MGC2656, SALM4 or FIGLER1), LRFN4 (also known as MGC3103, SALM3. or FIGLER6), LRFN5 (also known as FIGLER8, SALM5), LRRC4 (also known as NAG14), LRRC4B (also known as DKFZp761A179 or HSM), LRRC4C (also known as KIAA1580 or NGL-1), MADCAM1 (also known as MACAM1), MCAM (also known as MUC18, CD146, MeICAM, METCAM or HEMCAM), MPZ (also known as HMSNIB, CMT2I or CMT2J), MPZL2 (also known as EVA), NCAM1 (also known as NCAM or CD56), NCAM2 (also known as NCAM21 or MGC51008), NECTIN1 (also known as PRR, PRR1, PVRR1, SK-12, HIgR, CLPED1, CD111 or OFC7), NECTIN2 (also known as PVRR2, PRR2 or CD112), NECTIN3 (also known as nectin-3, PPR3, PVRR3, DKFZP566B0846, CDw113 or CD113), NECTIN4 (also known as nectin-4, PRR4 or LNIR), NEO1 (also known as NGN, HsT17534, IGDCC2 or NTN1R2), NFASC (also known as NRCAML, KIAA0756, FLJ46866 or NF), NRCAM (also known as KIAA0343 or Bravo), PECAM1 (also known as CD31), PTPRM (also known as RPTPU or hR-PTPu), PVR (also known as CD155, HVED, NecI-5, NECL5 or Tage4), ROBO1 (also known as DUTT1, FLJ21882 or SAX3), ROBO2 (also known as KIAA1568), SDK1 (also known as FLJ31425), SIGLECL1 (also known as FLJ40235), SIRPA (also known as SHPS1, SIRP, MYD-1, BIT, P84, SHPS-1, SIRPalpha, CD172a, SIRPalpha2, MFR or SIRP-ALPHA-1), SIRPG (also known as bA77C3.1, SIRP-B2, SIRPgamma or CD172g), SLAMF1 (also known as CD150), SLAMF6 (also known as KALI, NTBA, KALIb, Ly108, SF2000, NTB-A or CD352), THY1 (also known as CD90), UNC5A (also known as KIAA1976 or UNC5H1), UNC5B (also known as UNC5H2 or p53RDL1), UNC5D (also known as KIAA1777 or Unc5h4), OPCML (also known as OPCM, OBCAM or IGLON1), NTM (also known as HNT, NTRI, IGLON2 or CEPU-1), LSAMP (also known as LAMP or IGLON3), NEGR1 (also known as KILON, MGC46680, Ntra or IGLON4), IGLON5 (also known as LOC402665), SIGLEC1 (also known as SIGLEC-1, CD169, FLJ00051, FLJ00055, FLJ00073, FLJ32150, dJ1009E24.1 or sialoadhesin), SIGLEC10 (also known as SIGLEC-10, SLG2, PRO940 or MGC126774), SIGLEC11, SIGLEC12 (also known as SLG, S2V, Siglec-XII, Siglec-12 or Siglec-L1), SIGLEC14, SIGLEC15 (also known as HsT1361), SIGLEC16 (also known as Siglec-P16), SIGLEC17P, SIGLEC18P, CD22 (also known as SIGLEC-2 or SIGLEC2), SIGLEC20P, SIGLEC21P, SIGLEC22P, SIGLEC24P, SIGLEC25P, SIGLEC26P, SIGLEC27P, SIGLEC28P, SIGLEC29P, CD33 (also known as SIGLEC3, SIGLEC-3, p67 or FLJ00391), SIGLEC30P, SIGLEC31P, MAG (also known as SIGLEC4A, SIGLEC-4A or S-MAG), SIGLEC5 (also known as OB-BP2, SIGLEC-5 or CD170), SIGLEC6 (also known as OB-BP1, SIGLEC-6 or CD327), SIGLEC7 (also known as SIGLEC-7, p75/AIRM1, QA79 or CD328), SIGLEC8 (also known as SIGLEC-8, SAF2, SIGLEC8L or MGC59785), and SIGLEC9 (also known as CD329).

In one form of the present invention, the IgSF CAM superfamily (IgSF CAMs) comprises: ALCAM (also known as CD166 or MEMD), BCAM (also known as CD239), BOC (also known as CDON2), CADM1 (also known as NECL2, ST17, BL2, SYNCAM, IGSF4A, NecI-2, SYNCAM1 or RA175), CADM2 (also known as NECL3, NecI-3 or SynCAM2), CADM3 (also known as BlgR, FLJ10698, TSLL1, NECL1, SynCAM3 or NecI-1), CADM4 (also known as TSLL2, NecI-4 or SynCAM4), CD2, CD244 (also known as 2B4, NAIL, NKR2B4, Nmrk or SLAMF4), CD28, CD47 (also known as IAP or OA3), CD58, CD84 (also known as SLAMF5, hCD84 or mCD84), CD96 (also known as TACTILE), CHL1 (also known as CALL, L1CAM2, FLJ44930 or MGC132578), CNTN1 (also known as F3 or GP135), CNTN2 (also known as TAG-1 or TAXI), CNTN3 (also known as BIG-1), CNTN4 (also known as BIG-2), CNTN5 (also known as NB-2 or hNB-2), CNTN6 (also known as NB-3), CRTAM (also known as CD355), DSCAM (also known as CHD2-42 or CHD2-52), DSCAML1 (also known as KIAA1132), F11R (also known as PAM-1, JCAM, JAM-1, JAM-A, JAMA or CD321), FGFRL1, GP6 (also known as GPVI), HEPACAM (also known as FLJ25530, hepaCAM or GLIALCAM), ICAM1 (also known as BB2 or CD54), ICAM2 (also known as CD102), ICAM3 (also known as CDW50, ICAM-R or CD50), ICAM4 (also known as CD242), ICAM5 (also known as TLN), IGSF5 (also known as JAM4), IZUMO1 (also known as IZUMO, MGC34799 or OBF), IZUMO1R (also known as Folbp3 or JUNO), JAM2 (also known as VE-JAM, JAM-B, JAMB or CD322), JAM3 (also known as JAM-C or JAMC), JAML (also known as Gm638 or AMICA), L1CAM (also known as CD171), LILRB2 (also known as LIR-2, ILT4, MIR-10, LIR2, CD85d or MIR10), LRFN1 (also known as KIAA1484 or SALM2), LRFN2 (also known as FIGLER2), LRFN3 (also known as MGC2656, SALM4 or FIGLER1), LRFN4 (also known as MGC3103, SALM3. or FIGLER6), LRFN5 (also known as FIGLER8, SALM5), LRRC4 (also known as NAG14), LRRC4B (also known as DKFZp761A179 or HSM), LRRC4C (also known as KIAA1580 or NGL-1), MADCAM1 (also known as MACAM1), MCAM (also known as MUC18, CD146, MeICAM, METCAM or HEMCAM), MPZ (also known as HMSNIB, CMT2I or CMT2J), MPZL2 (also known as EVA), NCAM1 (also known as NCAM or CD56), NCAM2 (also known as NCAM21 or MGC51008), NECTIN1 (also known as PRR, PRR1, PVRR1, SK-12, HIgR, CLPED1, CD111 or OFC7), NECTIN2 (also known as PVRR2, PRR2 or CD112), NECTIN3 (also known as nectin-3, PPR3, PVRR3, DKFZP566B0846, CDw113 or CD113), NECTIN4 (also known as nectin-4, PRR4 or LNIR), NEO1 (also known as NGN, HsT17534, IGDCC2 or NTN1R2), NFASC (also known as NRCAML, KIAA0756, FLJ46866 or NF), NRCAM (also known as KIAA0343 or Bravo), PECAM1 (also known as CD31), PTPRM (also known as RPTPU or hR-PTPu), PVR (also known as CD155, HVED, NecI-5, NECL5 or Tage4), ROBO1 (also known as DUTT1, FLJ21882 or SAX3), ROBO2 (also known as KIAA1568), SDK1 (also known as FLJ31425), SIGLECL1 (also known as FLJ40235), SIRPA (also known as SHPS1, SIRP, MYD-1, BIT, P84, SHPS-1, SIRPalpha, CD172a, SIRPalpha2, MFR or SIRP-ALPHA-1), SIRPG (also known as bA77C3.1, SIRP-B2, SIRPgamma or CD172g), SLAMF1 (also known as CD150), SLAMF6 (also known as KALI, NTBA, KALIb, Ly108, SF2000, NTB-A or CD352), THY1 (also known as CD90), UNC5A (also known as KIAA1976 or UNC5H1), UNC5B (also known as UNC5H2 or p53RDL1), UNC5D (also known as KIAA1777 or Unc5h4), VCAM1 (also known as CD106), CLMP (also known as ASAM, FLJ22415 or ACAM), CXADR (also known as CAR), ESAM (also known as W117m), GPA33 (also known as A33), IGSF11 (also known as BT-IgSF, MGC35227, Igsf13, VSIG3 or CT119), VSIG1 (also known as MGC44287), VSIG2 (also known as CTXL, CTH) and VSIG8.

In one form of the present invention, the IgSF CAM superfamily (IgSF CAMs) comprises: ALCAM (also known as CD166 or MEMD), BCAM (also known as CD239), BOC (also known as CDON2), CADM1 (also known as NECL2, ST17, BL2, SYNCAM, IGSF4A, NecI-2, SYNCAM1 or RA175), CADM2 (also known as NECL3, NecI-3 or SynCAM2), CADM3 (also known as BlgR, FLJ10698, TSLL1, NECL1, SynCAM3 or NecI-1), CADM4 (also known as TSLL2, NecI-4 or SynCAM4), CD2, CD244 (also known as 2B4, NAIL, NKR2B4, Nmrk or SLAMF4), CD28, CD47 (also known as IAP or OA3), CD58, CD84 (also known as SLAMF5, hCD84 or mCD84), CD96 (also known as TACTILE), CHL1 (also known as CALL, L1CAM2, FLJ44930 or MGC132578), CNTN1 (also known as F3 or GP135), CNTN2 (also known as TAG-1 or TAXI), CNTN3 (also known as BIG-1), CNTN4 (also known as BIG-2), CNTN5 (also known as NB-2 or hNB-2), CNTN6 (also known as NB-3), CRTAM (also known as CD355), DSCAM (also known as CHD2-42 or CHD2-52), DSCAML1 (also known as KIAA1132), F11R (also known as PAM-1, JCAM, JAM-1, JAM-A, JAMA or CD321), FGFRL1, GP6 (also known as GPVI), HEPACAM (also known as FLJ25530, hepaCAM or GLIALCAM), ICAM1 (also known as BB2 or CD54), ICAM2 (also known as CD102), ICAM3 (also known as CDW50, ICAM-R or CD50), ICAM4 (also known as CD242), ICAM5 (also known as TLN), IGSF5 (also known as JAM4), IZUMO1 (also known as IZUMO, MGC34799 or OBF), IZUMO1R (also known as Folbp3 or JUNO), JAM2 (also known as VE-JAM, JAM-B, JAMB or CD322), JAM3 (also known as JAM-C or JAMC), JAML (also known as Gm638 or AMICA), L1CAM (also known as CD171), LILRB2 (also known as LIR-2, ILT4, MIR-10, LIR2, CD85d or MIR10), LRFN1 (also known as KIAA1484 or SALM2), LRFN2 (also known as FIGLER2), LRFN3 (also known as MGC2656, SALM4 or FIGLER1), LRFN4 (also known as MGC3103, SALM3. or FIGLER6), LRFN5 (also known as FIGLER8, SALM5), LRRC4 (also known as NAG14), LRRC4B (also known as DKFZp761A179 or HSM), LRRC4C (also known as KIAA1580 or NGL-1), MADCAM1 (also known as MACAM1), MCAM (also known as MUC18, CD146, MeICAM, METCAM or HEMCAM), MPZ (also known as HMSNIB, CMT2I or CMT2J), MPZL2 (also known as EVA), NCAM1 (also known as NCAM or CD56), NCAM2 (also known as NCAM21 or MGC51008), NECTIN1 (also known as PRR, PRR1, PVRR1, SK-12, HIgR, CLPED1, CD111 or OFC7), NECTIN2 (also known as PVRR2, PRR2 or CD112), NECTIN3 (also known as nectin-3, PPR3, PVRR3, DKFZP566B0846, CDw113 or CD113), NECTIN4 (also known as nectin-4, PRR4 or LNIR), NEO1 (also known as NGN, HsT17534, IGDCC2 or NTN1R2), NFASC (also known as NRCAML, KIAA0756, FLJ46866 or NF), NRCAM (also known as KIAA0343 or Bravo), PECAM1 (also known as CD31), PTPRM (also known as RPTPU or hR-PTPu), PVR (also known as CD155, HVED, NecI-5, NECL5 or Tage4), ROBO1 (also known as DUTT1, FLJ21882 or SAX3), ROBO2 (also known as KIAA1568), SDK1 (also known as FLJ31425), SIGLECL1 (also known as FLJ40235), SIRPA (also known as SHPS1, SIRP, MYD-1, BIT, P84, SHPS-1, SIRPalpha, CD172a, SIRPalpha2, MFR or SIRP-ALPHA-1), SIRPG (also known as bA77C3.1, SIRP-B2, SIRPgamma or CD172g), SLAMF1 (also known as CD150), SLAMF6 (also known as KALI, NTBA, KALIb, Ly108, SF2000, NTB-A or CD352), THY1 (also known as CD90), UNC5A (also known as KIAA1976 or UNC5H1), UNC5B (also known as UNC5H2 or p53RDL1), UNC5D (also known as KIAA1777 or Unc5h4), OPCML (also known as OPCM, OBCAM or IGLON1), NTM (also known as HNT, NTRI, IGLON2 or CEPU-1), LSAMP (also known as LAMP or IGLON3), NEGR1 (also known as KILON, MGC46680, Ntra or IGLON4) and IGLON5 (also known as LOC402665).

In one form of the present invention, the IgSF CAM superfamily (IgSF CAMs) comprises: ALCAM (also known as CD166 or MEMD), BCAM (also known as CD239), BOC (also known as CDON2), CADM1 (also known as NECL2, ST17, BL2, SYNCAM, IGSF4A, NecI-2, SYNCAM1 or RA175), CADM2 (also known as NECL3, NecI-3 or SynCAM2), CADM3 (also known as BlgR, FLJ10698, TSLL1, NECL1, SynCAM3 or NecI-1), CADM4 (also known as TSLL2, NecI-4 or SynCAM4), CD2, CD244 (also known as 2B4, NAIL, NKR2B4, Nmrk or SLAMF4), CD28, CD47 (also known as IAP or OA3), CD58, CD84 (also known as SLAMF5, hCD84 or mCD84), CD96 (also known as TACTILE), CHL1 (also known as CALL, L1CAM2, FLJ44930 or MGC132578), CNTN1 (also known as F3 or GP135), CNTN2 (also known as TAG-1 or TAXI), CNTN3 (also known as BIG-1), CNTN4 (also known as BIG-2), CNTN5 (also known as NB-2 or hNB-2), CNTN6 (also known as NB-3), CRTAM (also known as CD355), DSCAM (also known as CHD2-42 or CHD2-52), DSCAML1 (also known as KIAA1132), F11R (also known as PAM-1, JCAM, JAM-1, JAM-A, JAMA or CD321), FGFRL1, GP6 (also known as GPVI), HEPACAM (also known as FLJ25530, hepaCAM or GLIALCAM), ICAM1 (also known as BB2 or CD54), ICAM2 (also known as CD102), ICAM3 (also known as CDW50, ICAM-R or CD50), ICAM4 (also known as CD242), ICAM5 (also known as TLN), IGSF5 (also known as JAM4), IZUMO1 (also known as IZUMO, MGC34799 or OBF), IZUMO1R (also known as Folbp3 or JUNO), JAM2 (also known as VE-JAM, JAM-B, JAMB or CD322), JAM3 (also known as JAM-C or JAMC), JAML (also known as Gm638 or AMICA), L1CAM (also known as CD171), LILRB2 (also known as LIR-2, ILT4, MIR-10, LIR2, CD85d or MIR10), LRFN1 (also known as KIAA1484 or SALM2), LRFN2 (also known as FIGLER2), LRFN3 (also known as MGC2656, SALM4 or FIGLER1), LRFN4 (also known as MGC3103, SALM3. or FIGLER6), LRFN5 (also known as FIGLER8, SALM5), LRRC4 (also known as NAG14), LRRC4B (also known as DKFZp761A179 or HSM), LRRC4C (also known as KIAA1580 or NGL-1), MADCAM1 (also known as MACAM1), MCAM (also known as MUC18, CD146, MeICAM, METCAM or HEMCAM), MPZ (also known as HMSNIB, CMT2I or CMT2J), MPZL2 (also known as EVA), NCAM1 (also known as NCAM or CD56), NCAM2 (also known as NCAM21 or MGC51008), NECTIN1 (also known as PRR, PRR1, PVRR1, SK-12, HIgR, CLPED1, CD111 or OFC7), NECTIN2 (also known as PVRR2, PRR2 or CD112), NECTIN3 (also known as nectin-3, PPR3, PVRR3, DKFZP566B0846, CDw113 or CD113), NECTIN4 (also known as nectin-4, PRR4 or LNIR), NEO1 (also known as NGN, HsT17534, IGDCC2 or NTN1R2), NFASC (also known as NRCAML, KIAA0756, FLJ46866 or NF), NRCAM (also known as KIAA0343 or Bravo), PECAM1 (also known as CD31), PTPRM (also known as RPTPU or hR-PTPu), PVR (also known as CD155, HVED, NecI-5, NECL5 or Tage4), ROBO1 (also known as DUTT1, FLJ21882 or SAX3), ROBO2 (also known as KIAA1568), SDK1 (also known as FLJ31425), SIGLECL1 (also known as FLJ40235), SIRPA (also known as SHPS1, SIRP, MYD-1, BIT, P84, SHPS-1, SIRPalpha, CD172a, SIRPalpha2, MFR or SIRP-ALPHA-1), SIRPG (also known as bA77C3.1, SIRP-B2, SIRPgamma or CD172g), SLAMF1 (also known as CD150), SLAMF6 (also known as KALI, NTBA, KALIb, Ly108, SF2000, NTB-A or CD352), THY1 (also known as CD90), UNC5A (also known as KIAA1976 or UNC5H1), UNC5B (also known as UNC5H2 or p53RDL1), UNC5D (also known as KIAA1777 or Unc5h4), SIGLEC1 (also known as SIGLEC-1, CD169, FLJ00051, FLJ00055, FLJ00073, FLJ32150, dJ1009E24.1 or sialoadhesin), SIGLEC10 (also known as SIGLEC-10, SLG2, PRO940 or MGC126774), SIGLEC11, SIGLEC12 (also known as SLG, S2V, Siglec-XII, Siglec-12 or Siglec-L1), SIGLEC14, SIGLEC15 (also known as HsT1361), SIGLEC16 (also known as Siglec-P16), SIGLEC17P, SIGLEC18P, CD22 (also known as SIGLEC-2 or SIGLEC2), SIGLEC20P, SIGLEC21P, SIGLEC22P, SIGLEC24P, SIGLEC25P, SIGLEC26P, SIGLEC27P, SIGLEC28P, SIGLEC29P, CD33 (also known as SIGLEC3, SIGLEC-3, p67 or FLJ00391), SIGLEC30P, SIGLEC31P, MAG (also known as SIGLEC4A, SIGLEC-4A or S-MAG), SIGLEC5 (also known as OB-BP2, SIGLEC-5 or CD170), SIGLEC6 (also known as OB-BP1, SIGLEC-6 or CD327), SIGLEC7 (also known as SIGLEC-7, p75/AIRM1, QA79 or CD328), SIGLEC8 (also known as SIGLEC-8, SAF2, SIGLEC8L or MGC59785), and SIGLEC9 (also known as CD329).

In one form of the present invention, the IgSF CAM superfamily (IgSF CAMs) comprises: ALCAM (also known as CD166 or MEMD), BCAM (also known as CD239), BOC (also known as CDON2), CADM1 (also known as NECL2, ST17, BL2, SYNCAM, IGSF4A, NecI-2, SYNCAM1 or RA175), CADM2 (also known as NECL3, NecI-3 or SynCAM2), CADM3 (also known as BlgR, FLJ10698, TSLL1, NECL1, SynCAM3 or NecI-1), CADM4 (also known as TSLL2, NecI-4 or SynCAM4), CD2, CD244 (also known as 2B4, NAIL, NKR2B4, Nmrk or SLAMF4), CD28, CD47 (also known as IAP or OA3), CD58, CD84 (also known as SLAMF5, hCD84 or mCD84), CD96 (also known as TACTILE), CHL1 (also known as CALL, L1CAM2, FLJ44930 or MGC132578), CNTN1 (also known as F3 or GP135), CNTN2 (also known as TAG-1 or TAXI), CNTN3 (also known as BIG-1), CNTN4 (also known as BIG-2), CNTN5 (also known as NB-2 or hNB-2), CNTN6 (also known as NB-3), CRTAM (also known as CD355), DSCAM (also known as CHD2-42 or CHD2-52), DSCAML1 (also known as KIAA1132), F11R (also known as PAM-1, JCAM, JAM-1, JAM-A, JAMA or CD321), FGFRL1, GP6 (also known as GPVI), HEPACAM (also known as FLJ25530, hepaCAM or GLIALCAM), ICAM1 (also known as BB2 or CD54), ICAM2 (also known as CD102), ICAM3 (also known as CDW50, ICAM-R or CD50), ICAM4 (also known as CD242), ICAM5 (also known as TLN), IGSF5 (also known as JAM4), IZUMO1 (also known as IZUMO, MGC34799 or OBF), IZUMO1R (also known as Folbp3 or JUNO), JAM2 (also known as VE-JAM, JAM-B, JAMB or CD322), JAM3 (also known as JAM-C or JAMC), JAML (also known as Gm638 or AMICA), L1CAM (also known as CD171), LILRB2 (also known as LIR-2, ILT4, MIR-10, LIR2, CD85d or MIR10), LRFN1 (also known as KIAA1484 or SALM2), LRFN2 (also known as FIGLER2), LRFN3 (also known as MGC2656, SALM4 or FIGLER1), LRFN4 (also known as MGC3103, SALM3. or FIGLER6), LRFN5 (also known as FIGLER8, SALM5), LRRC4 (also known as NAG14), LRRC4B (also known as DKFZp761A179 or HSM), LRRC4C (also known as KIAA1580 or NGL-1), MADCAM1 (also known as MACAM1), MCAM (also known as MUC18, CD146, MeICAM, METCAM or HEMCAM), MPZ (also known as HMSNIB, CMT2I or CMT2J), MPZL2 (also known as EVA), NCAM1 (also known as NCAM or CD56), NCAM2 (also known as NCAM21 or MGC51008), NECTIN1 (also known as PRR, PRR1, PVRR1, SK-12, HIgR, CLPED1, CD111 or OFC7), NECTIN2 (also known as PVRR2, PRR2 or CD112), NECTIN3 (also known as nectin-3, PPR3, PVRR3, DKFZP566B0846, CDw113 or CD113), NECTIN4 (also known as nectin-4, PRR4 or LNIR), NEO1 (also known as NGN, HsT17534, IGDCC2 or NTN1R2), NFASC (also known as NRCAML, KIAA0756, FLJ46866 or NF), NRCAM (also known as KIAA0343 or Bravo), PECAM1 (also known as CD31), PTPRM (also known as RPTPU or hR-PTPu), PVR (also known as CD155, HVED, NecI-5, NECL5 or Tage4), ROBO1 (also known as DUTT1, FLJ21882 or SAX3), ROBO2 (also known as KIAA1568), SDK1 (also known as FLJ31425), SIGLECL1 (also known as FLJ40235), SIRPA (also known as SHPS1, SIRP, MYD-1, BIT, P84, SHPS-1, SIRPalpha, CD172a, SIRPalpha2, MFR or SIRP-ALPHA-1), SIRPG (also known as bA77C3.1, SIRP-B2, SIRPgamma or CD172g), SLAMF1 (also known as CD150), SLAMF6 (also known as KALI, NTBA, KALIb, Ly108, SF2000, NTB-A or CD352), THY1 (also known as CD90), UNC5A (also known as KIAA1976 or UNC5H1), UNC5B (also known as UNC5H2 or p53RDL1) and UNC5D (also known as KIAA1777 or Unc5h4).

In one form of the present invention, the IgSF CAM superfamily (IgSF CAMs) comprises: ALCAM (also known as CD166), BCAM, MCAM, Neural Cell Adhesion Molecules (NCAMs), Intercellular Cell Adhesion Molecules (ICAMs), Vascular Cell Adhesion Molecules (VCAMs), Platelet-endothelial Cell Adhesion Molecule (PECAMs), L1 family including L1 (protein), CHL1, Neurofascin and NrCAM, SIGLEC family including Myelin-associated glycoprotein (MAG, SIGLEC-4), CD22 and CD83, CTX family including CTX, Junctional adhesion molecule (JAM), BT-IgSF, Coxsackie virus and adenovirus receptor (CAR), VSIG, endothelial cell-selective adhesion molecule (ESAM), Nectins and related proteins, including CADM1 and other Synaptic Cell Adhesion Molecules, CD2, CD48, HEPACAM, HEPACAM2, Down syndrome cell adhesion molecule (DSCAM).

In one form of the present invention, the IgSF CAM superfamily (IgSF CAMs) comprises: ALCAM (also known as CD166), BCAM, MCAM, NCAM-1, NCAM-2, ICAM-1, ICAM-2, ICAM-3 (also known as CD50), ICAM-4, ICAM-5, VCAM-1, PECAM-1 (also known as CD31), L1 (protein), CHL1, Neurofascin, NrCAM, Myelin-associated glycoprotein (MAG, SIGLEC-4), CD22, CD83, CTX, Junctional adhesion molecule (JAM), BT-IgSF, Coxsackie virus and adenovirus receptor (CAR), VSIG, endothelial cell-selective adhesion molecule (ESAM), CADM1, CADM2, CADM3, CADM4, CD2, CD48, HEPACAM, HEPACAM2, and Down syndrome cell adhesion molecule (DSCAM).

1. Modulators of Ligand-Independent Activation of IgSF CAM by Activated Co-Located GPCRs

In one form, the present invention comprises modulators of IgSF CAM activity where such IgSF CAM activity is induced by certain active co-located GPCRs.

In one form, the present invention comprises modulators of IgSF CAM ligand-independent activation of an IgSF CAM by certain activated co-located GPCRs.

In one form, the present invention comprises modulators wherein the modulators are modulators of IgSF CAM-dependent signalling induced by certain activated co-located GPCRs.

In one form of the present invention, the modulators of IgSF CAM ligand-independent activation of the IgSF CAM by certain activated co-located GPCRs act in the absence of any IgSF CAM ligand.

In one form of the present invention, the modulators of IgSF CAM ligand-independent activation of the IgSF CAM by certain activated co-located GPCRs act in the presence of a truncated ectodomain of an IgSF CAM.

In one form of the present invention, the modulators of IgSF CAM ligand-independent activation of the IgSF CAM by certain activated co-located GPCRs act in the presence of a truncated ectodomain of an IgSF CAM which is not greater than 40, not greater than 20, not greater than 10 or not greater than 5 amino acids in length.

In one form of the present invention, the modulators of IgSF CAM ligand-independent activation of the IgSF CAM by certain activated co-located GPCRs, contain the entire ectodomain of an IgSF CAM conjugated to an analogue, fragment or derivative of the transmembrane domain of an IgSF CAM which is greater than 5, greater than 10, or greater than 20 amino acids in length.

In one form of the present invention, the modulators of IgSF CAM ligand-independent activation of the IgSF CAM by certain activated co-located GPCRs act in the absence of the IgSF CAM ligand-binding ectodomain of an IgSF CAM.

In one form of the present invention, the modulators of IgSF CAM ligand-independent activation of the IgSF CAM by certain activated co-located GPCRs do not contain the ectodomain of an IgSF CAM.

In one form of the present invention, the modulators of IgSF CAM ligand-independent activation of the IgSF CAM by certain activated co-located GPCRs do not contain an analogue, fragment or derivative of the ectodomain of an IgSF CAM.

In one form of the present invention, the modulators of IgSF CAM ligand-independent activation of the IgSF CAM by certain activated co-located GPCRs contain a fragment of the ectodomain of an IgSF CAM.

In one form of the present invention, the modulators of IgSF CAM ligand-independent activation of the IgSF CAM by certain activated co-located GPCRs inhibit or facilitate signalling that occurs through the C-terminal cytosolic tail of an IgSF CAM induced by an activated co-located GPCR.

In one form of the present invention, the modulators of IgSF CAM ligand-independent activation of the IgSF CAM by certain activated co-located GPCRs inhibit binding that occurs to the C-terminal cytosolic tail of an IgSF CAM.

In one form of the present invention, the modulators of IgSF CAM ligand-independent activation of the IgSF CAM by certain activated co-located GPCRs inhibit or facilitate the interaction between the IgSF CAM and certain GPCRs.

In one form of the present invention, the modulators of IgSF CAM ligand-independent activation of the IgSF CAM by certain activated co-located GPCRs inhibit or facilitate the capacity of an activated GPCR to modulate IgSF CAM-dependent signalling that is dependent upon proximity of an IgSF CAM and the certain GPCR.

In one form of the present invention, the modulators of IgSF CAM ligand-independent activation of the IgSF CAM by certain activated co-located GPCRs inhibit the capacity of an activated GPCR to modulate CAM-dependent signalling that is dependent upon proximity of the IgSF CAM and the certain GPCR.

In one form of the present invention, the modulators of IgSF CAM ligand-independent activation of the IgSF CAM by certain activated co-located GPCRs inhibit or facilitate the capacity of an activated GPCR to modulate IgSF CAM-dependent signalling that is dependent upon proximity of the IgSF CAM and the certain GPCR and/or inhibit or facilitate signalling that occurs through the C-terminal cytosolic tail of an IgSF CAM induced by an activated co-located GPCR.

In one form of the present invention, the modulators of IgSF CAM ligand-independent activation of the IgSF CAM by certain activated co-located GPCRs inhibit the capacity of an activated GPCR to modulate CAM-dependent signalling that is dependent upon proximity of the IgSF CAM and the certain GPCR and/or inhibit signalling that occurs through the C-terminal cytosolic tail of the IgSF CAM induced by an activated co-located GPCR.

Throughout this specification, unless the context requires otherwise, an activated GPCR means a GPCR that is in an active state that may result from the binding of an agonist, partial agonist and/or allosteric modulator, and/or as a consequence of constitutive activity that does not necessitate ligand binding.

Throughout this specification, unless the context requires otherwise, the certain activated co-located GPCRs of the invention are GPCRs that are expressed in the same cell as the IgSF CAM and for which an effect on the IgSF CAM, indicative of modulation of IgSF CAM activation and/or modulation of induction of IgSF CAM-dependent signalling, is detected upon activation by cognate ligands of the certain co-located GPCRs or when the GPCRs are constitutively active.

In one embodiment, an effect on the IgSF CAM indicative of modulation of IgSF CAM activation is a change in intracellular trafficking such as that detected by a change in proximity of luciferase-conjugated IgSF CAM (such as IgSF CAM/Rluc8) to intracellular compartment markers such as fluorophore-labelled Rabs, such as Rab1, Rab4, Rab5, Rab6, Rab7, Rab8, Rab9 and/or Rab11 (such as Venus-Rab1, Venus-Rab4, Venus-Rab5, Venus-Rab6, Venus-Rab7, Venus-Rab8, Venus-Rab9 and/or Venus-Rab11), and/or a plasma membrane marker, such as a fluorophore-conjugated fragment of K-ras (such as Venus-K-ras) using bioluminescence resonance energy transfer (BRET) upon addition of a cognate ligand for the co-located GPCR (Tiulpakov et al., 2016).

In another embodiment, an effect on the IgSF CAM is a change in IgSF CAM-dependent signalling, such as detected by a change in proximity of luciferase-conjugated IgSF CAM (such as IgSF CAM-Rluc8) to an IgSF CAM-interacting group, such as fluorophore-labelled proteins interacting with the cytosolic tail of the IgSF CAM, such as IQGAP-1, protein kinase C zeta (PKCζ), Dock7, MyD88, TIRAP, ERK1/2, (Jules et al., 2013; Ramasamy et al., 2016), olfactory receptor 2T2, ADP/ATP translocase 2, Protein phosphatase 1G, Intercellular adhesion molecule 1, Protein DJ-1 (PARK7), Calponin-3, Drebrin, Filamin B, Ras-related protein Rab-13, Radixin/Ezrin/Moesin, Proteolipid protein 2, Coronin, S100 A11, Succinyl-CoA ligase [GDP-forming] subunit alpha, Hsc70-interacting protein, Apoptosis Inhibitor 5, neuropilin, cleavage stimulation factor, growth factor receptor-bound protein 2, sec61 beta subunit, or Nck1.

In another embodiment, an effect on the IgSF CAM is a change in IgSF CAM-dependent signalling, such as detected by a change in canonical activation of NFκB upon activation of the certain co-located GPCRs by their cognate ligands as measured by one or more of the following:
Activity of IkB kinase (IKK) by monitoring in vitro phosphorylation of a substrate, such as GST-IκBα;
Detection of IkB Degradation Dynamics, including phosphorylation/ubiquitination and/or degradation of IκB and/or IκB-α;
Detection of p65(Rel-A) phosphorylation/ubiquitination, such as by using antibodies, gel-shift, EMSA, and/or mass spectroscopy;
Detection of cytosolic to nuclear shuttling/translocation of NFκB components/subunits, such as p65/phospho-p65;
Detection of NFκB subunit dimerization/complexation;
Detection of active NFκB components/subunits by binding to immobilized DNA sequence/oligonucleotide containing the NFκB response element/consensus NFκB binding motif, such as by using electrophoretic mobility shift assay or gel shift assay, SELEX, protein-binding microarray, or sequencing-based approaches;
Chromatin-immunoprecipitation (ChIP) assays to detect NFκB in situ binding to DNA to the promoters and enhancers of specific genes;
In vitro kinase assay for NFκB kinase activity;
Measurement of NFκB transcriptional activity using NFκB reporter assays via transgene expression of reporter constructs, such as LacZ Fluc, eGFP SEAP, and NF-gluc, using such approaches as plasmid transfection, reporter cell lines, mini-circles, retrovirus, or lentivirus;
Measuring changes in expression of downstream targets of NFκB, such as cytokines, growth factors, adhesion molecules and mitochondrial anti-apoptotic genes, by real-time PCR, protein, or functional assays (Note the pleiotropic nature of NFκB is reflected in its transcriptional targets that presently number approximately 500 (see http://www.bu.edu/nf-kb/gene-resources/target-genes/ as at 7 Dec. 2018); and
Measuring changes in function or structure induced by NFκB-dependent signalling, such as POLKADOTS in T-cells, adhesion in endothelial cells, activation in leucocytes, or oncogenicity.

In another embodiment, an effect on the IgSF CAM is a change in IgSF CAM dependent signalling, such as detected by a change in non-canonical activation of NFκB by measuring one or more of the following:
Detection of NIK (NFκB-Inducing Kinase);
Detecting IKKα Activation/phosphorylation;
Detection of NIK kinase activity by ability to autophosphorylate or to phosphorylate a substrate by performing a kinase assay;
Generation of p52-containing NFκB dimers, such as p52/RelB;
Detection of Phospho-NFκB2 p100(Ser866/870);
Detection of partial degradation (called processing) of the precursor p100 into p52;
Detecting p52/RelB translocation into the nucleus;
Detecting p52/RelB binding to NFκB sites;
Measurement of NFκB transcriptional activity using NFκB reporter assays via transgene expression of reporter constructs, such as LacZ Fluc, eGFP SEAP, NF-gluc, using such approaches as plasmid transfection, reporter cell lines, mini-circles, retrovirus, or lentivirus; and
Measuring changes in expression of downstream targets of non-canonical signalling of NFκB, such as CXCL12, by real-time PCR, protein expression or by functional assays.

In one form of the invention, the modulator is isolated.

In one form, the invention comprises a pharmaceutical composition comprising a modulator of IgSF CAM activity where such IgSF CAM activity is induced by certain active co-located GPCRs as described herein.

In one form the invention comprises the use of a modulator of IgSF CAM activity where such IgSF CAM activity is induced by certain active co-located GPCRs for the treatment or prevention of an ailment.

2. Modulators of Ligand-Independent Activation of Members of the IgSF CAM Superfamily by Activated Co-Located GPCRs

In one form, the present invention comprises modulators of members of the IgSF CAM superfamily activity where such members of the IgSF CAM superfamily activity is induced by certain active co-located GPCRs.

In one form, the present invention comprises modulators of ligand-independent activation of members of the IgSF CAM superfamily by certain activated co-located GPCRs.

In one form, the present invention comprises modulators wherein the modulators are modulators of members of the IgSF CAM superfamily dependent signalling induced by certain activated co-located GPCRs.

In one form of the present invention, the modulators of ligand-independent activation of members of the IgSF CAM superfamily by certain activated co-located GPCRs act in the absence of any members of the IgSF CAM superfamily ligand.

In one form of the present invention, the modulators of members of the IgSF CAM superfamily ligand-independent activation of members of the IgSF CAM superfamily by certain activated co-located GPCRs act in the presence of a truncated ectodomain of members of the IgSF CAM superfamily.

In one form of the present invention, the modulators of ligand-independent activation of members of the IgSF CAM superfamily by certain activated co-located GPCRs act in the presence of a truncated ectodomain of members of the IgSF CAM superfamily which is not greater than 40, not greater than 20, not greater than 10 or not greater than 5 amino acids in length.

In one form of the present invention, the modulators of ligand-independent activation of members of the IgSF CAM superfamily by certain activated co-located GPCRs, consist of the entire ectodomain of a member of the IgSF CAM superfamily.

In one form of the present invention, the modulators of ligand-independent activation of members of the IgSF CAM superfamily by certain activated co-located GPCRs, contain the entire ectodomain of members of the IgSF CAM superfamily conjugated to an analogue, fragment or derivative of the transmembrane domain of members of the IgSF CAM superfamily which is greater than 5, greater than 10, or greater than 20 amino acids in length.

In one form, the present invention comprises modulators of ligand-independent IgSF CAM activity where such IgSF CAM activity is induced by a co-located GPCR and where the modulators of IgSF CAM activity are analogues, fragments or derivatives of the C-terminal tail of IgSF CAM lacking serines or threonines, or with serines and threonines selectively mutated to other residues.

In one form, the present invention comprises modulators of ligand-independent IgSF CAM activity where such IgSF CAM activity is induced by a co-located GPCR and where the modulators of IgSF CAM activity are analogues, fragments or derivatives of the C-terminal tail of IgSF CAM lacking serines or threonines, or with serines and threonines mutated to other residues that are not negatively charged.

In one form of the present invention, the modulators of ligand-independent activation of members of the IgSF CAM superfamily by certain activated co-located GPCRs act in the absence of members of the IgSF CAM superfamily ligand-binding ectodomain of members of the IgSF CAM superfamily.

In one form of the present invention, the modulators of ligand-independent activation of members of the IgSF CAM superfamily by certain activated co-located GPCRs do not contain the ectodomain of members of the IgSF CAM superfamily.

In one form of the present invention, the modulators of ligand-independent activation of members of the IgSF CAM superfamily by certain activated co-located GPCRs do not contain an analogue, fragment or derivative of the ectodomain of members of the IgSF CAM superfamily.

In one form of the present invention, the modulators of ligand-independent activation of members of the IgSF CAM superfamily by certain activated co-located GPCRs contain a fragment of the ectodomain of members of the IgSF CAM superfamily.

In one form of the present invention, the modulators of ligand-independent activation of members of the IgSF CAM superfamily by certain activated co-located GPCRs inhibit or facilitate signalling that occurs through the C-terminal cytosolic tail of members of the IgSF CAM superfamily induced by an activated co-located GPCR.

In one form of the present invention, the modulators of ligand-independent activation of members of the IgSF CAM superfamily by certain activated co-located GPCRs inhibit binding that occurs to the C-terminal cytosolic tail of members of the IgSF CAM superfamily.

In one form of the present invention, the modulators of ligand-independent activation of members of the IgSF CAM superfamily by certain activated co-located GPCRs inhibit or facilitate the interaction between members of the IgSF CAM superfamily and certain GPCRs.

In one form of the present invention, the modulators of ligand-independent activation of members of the IgSF CAM superfamily by certain activated co-located GPCRs inhibit or facilitate the capacity of an activated GPCR to modulate members of the members of the IgSF CAM superfamily dependent signalling that is dependent upon proximity of members of the IgSF CAM superfamily and the certain GPCR.

In one form of the present invention, the modulators of ligand-independent activation of members of the IgSF CAM superfamily by certain activated co-located GPCRs inhibit the capacity of an activated GPCR to modulate members of the IgSF CAM superfamily-dependent signalling that is dependent upon proximity of the members of the IgSF CAM superfamily and the certain GPCR.

In one form of the present invention, the modulators of ligand-independent activation of members of the IgSF CAM superfamily by certain activated co-located GPCRs inhibit or facilitate the capacity of an activated GPCR to modulate members of the IgSF CAM superfamily-dependent signalling that is dependent upon proximity of members of the IgSF CAM superfamily and the certain GPCR and inhibit or facilitate signalling that occurs through the C-terminal cytosolic tail of members of the IgSF CAM superfamily induced by an activated co-located GPCR.

In one form of the present invention, the modulators of ligand-independent activation of members of the IgSF CAM superfamily by certain activated co-located GPCRs inhibit or facilitate the capacity of an activated GPCR to modulate members of the IgSF CAM superfamily-dependent signalling that is dependent upon proximity of members of the IgSF CAM superfamily and the certain GPCR or inhibit or facilitate signalling that occurs through the C-terminal cytosolic tail of members of the IgSF CAM superfamily induced by an activated co-located GPCR.

In one form of the present invention, the modulators of ligand-independent activation of members of the IgSF CAM superfamily by certain activated co-located GPCRs inhibit the capacity of an activated GPCR to modulate members of the IgSF CAM superfamily-dependent signalling that is dependent upon proximity of members of the IgSF CAM superfamily and the certain GPCR and/or inhibit signalling that occurs through the C-terminal cytosolic tail of members of the IgSF CAM superfamily induced by an activated co-located GPCR.

In one form of the invention, the modulator is isolated.

In one form, the invention comprises a pharmaceutical composition comprising a modulator of IgSF CAM superfamily activity where such IgSF CAM superfamily activity is induced by certain active co-located GPCRs as described herein.

In one form the invention comprises the use of a modulator of IgSF CAM superfamily activity where such IgSF CAM superfamily activity is induced by certain active co-located GPCRs for the treatment or prevention of an ailment.

### Co-Located GPCRs

In one embodiment, the certain activated co-located GPCRs of the invention are those GPCRs that are expressed in the same cell as an IgSF CAM and are associated with an IgSF CAM-related disorder.

In one embodiment, the certain activated co-located GPCRs of the invention are those GPCRs that are expressed in the same cell as an IgSF CAM, are associated with an IgSF CAM-related disorder(s), and upon their removal and/or inhibition result in reduction or alleviation of an IgSF CAM-related disorder(s).

In one embodiment, the certain activated co-located GPCRs of the invention are those GPCRs that are implicated in inflammation.

In one embodiment, the certain activated co-located GPCRs of the invention are those GPCRs that are implicated in inflammation, and upon their removal and/or inhibition result in reduction or alleviation of the inflammation.

In one embodiment, the certain activated co-located GPCRs of the invention are those GPCRs that are implicated in cell proliferation.

In one embodiment, the certain activated co-located GPCRs of the invention are those GPCRs that are implicated in cell proliferation, and upon their removal and/or inhibition result in reduction or alleviation of the cell proliferation.

Indeed there is evidence for many GPCRs being involved in inflammation to some degree, and these levels can be differentiated according to the level of evidence:
1—No evidence found to date;
2—Receptor structure, or motif within receptor is similar to known inflammatory/immunological receptor or motif involved in an inflammatory/immunological process;
3—Receptor binds a ligand that mediates an inflammatory/immunological process;
4—Receptor is associated with/involved in an inflammatory/immunological disease;
5—At least one paper describing direct involvement of receptor in inflammatory/immunological process;
6—Receptor is expressed in inflammatory/immune cells; and
7—Receptor's involvement in inflammatory/immunological processes is well characterised (as described in http://www.guidetopharmacology.org database).

Family A GPCRs (except olfactory, vomeronasal, opsins) and the current level of evidence for their involvement in inflammation (see key above):

| | | Level of | |
|---|---|---|---|
| Type | Subtype | Evidence | Reference |
| 5-Hydroxytryptamine receptors | 5-HT1A receptor | 7 | (Freire - Garabal et al., 2003) |
| 5-Hydroxytryptamine receptors | 5-HT1B receptor | 6 | (Stefulj et al., 2000) |
| 5-Hydroxytryptamine receptors | 5-HT1D receptor | 5 | (Rebeck et al., 1994) |
| 5-Hydroxytryptamine receptors | 5-HT1E receptor | 5 | (Granados-Soto et al., 2010) |
| 5-Hydroxytryptamine receptors | 5-HT1F receptor | 6 | (Stefulj et al., 2000) |
| 5-Hydroxytryptamine receptors | 5-HT2A receptor | 7 | (Okamoto et al., 2002) |
| 5-Hydroxytryptamine receptors | 5-HT2B receptor | 6 | (Stefulj et al., 2000) |
| 5-Hydroxytryptamine receptors | 5-HT2C receptor | 6 | (Marazziti et al., 2001) |
| 5-Hydroxytryptamine receptors | 5-HT4 receptor | 4 | (Kanazawa et al., 2011) |
| 5-Hydroxytryptamine receptors | 5-HT5A receptor | 6 | (Marazziti et al., 2001) |
| 5-Hydroxytryptamine receptors | 5-HT5B receptor | 1 | (Rees et al., 1994) - Not |
| | | | expressed in humans due to |
| | | | internal stop codon in gene |
| 5-Hydroxytryptamine receptors | 5-HT6 receptor | 6 | (Stefulj et al., 2000) |
| 5-Hydroxytryptamine receptors | 5-HT7 receptor | 6 | (Stefulj et al., 2000) |
| Acetylcholine receptors (muscarinic) | M1 receptor | 6 | (Sato et al., 1999) |
| Acetylcholine receptors (muscarinic) | M2 receptor | 6 | (Sato et al., 1999) |
| Acetylcholine receptors (muscarinic) | M3 receptor | 6 | (Sato et al., 1999) |
| Acetylcholine receptors (muscarinic) | M4 receptor | 6 | (Sato et al., 1999) |
| Acetylcholine receptors (muscarinic) | M5 receptor | 6 | (Sato et al., 1999) |
| Adenosine receptors | A1 receptor | 7 | (Satoh et al., 2000) |
| Adenosine receptors | A2A receptor | 7 | (McPherson et al., 2001) |
| Adenosine receptors | A2B receptor | 7 | (Németh et al., 2005) |
| Adenosine receptors | A3 receptor | 7 | (Zhong et al., 2003) |
| Adrenoceptors | α1A-adrenoceptor | 6 | (Tayebati et al., 2000) |
| Adrenoceptors | α1B-adrenoceptor | 6 | (Tayebati et al., 2000) |
| Adrenoceptors | α1D-adrenoceptor | 6 | (Tayebati et al., 2000) |
| Adrenoceptors | α2A-adrenoceptor | 5 | (Zhang et al., 2010a) |
| Adrenoceptors | α2B-adrenoceptor | 5 | (Calonge et al., 2005) |
| Adrenoceptors | α2C-adrenoceptor | 5 | (Laukova et al., 2010) |
| Adrenoceptors | β1-adrenoceptor | 5 | (Nishio et al., 1998) |
| Adrenoceptors | β2-adrenoceptor | 7 | (Izeboud et al., 2000) |
| Adrenoceptors | β3-adrenoceptor | 5 | (Lamas et al., 2003) |
| Complement peptide receptors | C3a receptor | 7 | (Hartmann et al., 1997) |
| Complement peptide receptors | C5a1 receptor | 7 | (Kupp et al., 1991) |
| Complement peptide receptors | C5a2 receptor | 7 | (Zhang et al., 2010b) |
| Angiotensin receptors | AT₁ receptor | 7 | (Jaffré et al., 2009) |
| Angiotensin receptors | AT₂ receptor | 5 | (Matavelli et al., 2011) |
| Apelin receptor | apelin receptor | 7 | (Zhou et al., 2003) |
| Bile acid receptor | GPBA receptor | 6 | (Kawamata et al., 2003) |
| Bombesin receptors | BB1 receptor | 5 | (Baroni et al., 2008) |
| Bombesin receptors | BB2 (GRP) receptor | 7 | (Czepielewski et al., 2012) |
| Bombesin receptors | BB3 receptor | 5 | (Fleischmann et al., 2000) |
| Bradykinin receptors | B1 receptor | 7 | (Ehrenfeld et al., 2006) |
| Bradykinin receptors | B2 receptor | 7 | (Souza et al., 2004) |
| Cannabinoid receptors | CB1 receptor | 6 | (Galiègue et al., 1995) |
| Cannabinoid receptors | CB2 receptor | 6 | (Galiègue et al., 1995) |
| Chemokine receptors | CCR1 | 7 | (Lazennec & Richmond, 2010) |
| Chemokine receptors | CCR2 | 7 | (Lazennec & Richmond, 2010) |
| Chemokine receptors | CCR3 | 7 | (Lazennec & Richmond, 2010) |
| Chemokine receptors | CCR4 | 7 | (Lazennec & Richmond, 2010) |
| Chemokine receptors | CCR5 | 7 | (Lazennec & Richmond, 2010) |
| Chemokine receptors | CCR6 | 7 | (Lazennec & Richmond, 2010) |
| Chemokine receptors | CCR7 | 7 | (Lazennec & Richmond, 2010) |
| Chemokine receptors | CCR8 | 7 | (Lazennec & Richmond, 2010) |
| Chemokine receptors | CCR9 | 7 | (Lazennec & Richmond, 2010) |
| Chemokine receptors | CCR10 | 7 | (Lazennec & Richmond, 2010) |
| Chemokine receptors | CXCR1 | 7 | (Lazennec & Richmond, 2010) |
| Chemokine receptors | CXCR2 | 7 | (Lazennec & Richmond, 2010) |
| Chemokine receptors | CXCR3 | 7 | (Lazennec & Richmond, 2010) |
| Chemokine receptors | CXCR4 | 7 | (Lazennec & Richmond, 2010) |
| Chemokine receptors | CXCR5 | 7 | (Lazennec & Richmond, 2010) |
| Chemokine receptors | CXCR6 | 7 | (Lazennec & Richmond, 2010) |
| Chemokine receptors | CX3CR1 | 7 | (Lazennec & Richmond, 2010) |
| Chemokine receptors | XCR1 | 7 | (Lazennec & Richmond, 2010) |
| Chemokine receptors | ACKR1 | 7 | (Lazennec & Richmond, 2010) |
| Chemokine receptors | ACKR2 | 7 | (Lazennec & Richmond, 2010) |
| Chemokine receptors | ACKR3 | 7 | (Lazennec & Richmond, 2010) |
| Chemokine receptors | ACKR4 | 7 | (Lazennec & Richmond, 2010) |
| Chemokine receptors | CCRL2 | 7 | (Lazennec & Richmond, 2010) |
| Cholecystokinin receptors | CCK1 receptor | 6 | (Schmitz et al., 2001) |
| Cholecystokinin receptors | CCK2 receptor | 6 | (Schmitz et al., 2001) |
| Dopamine receptors | D1 receptor | 6 | (Caronti et al., 1998) |
| Dopamine receptors | D2 receptor | 6 | (Levite et al., 2001) |
| Dopamine receptors | D3 receptor | 6 | (Levite et al., 2001) |
| Dopamine receptors | D4 receptor | 6 | (Sarkar et al., 2006) |
| Dopamine receptors | D5 receptor | 6 | (Caronti et al., 1998) |
| Endothelin receptors | ETA receptor | 5 | (Sampaio et al., 2004) |
| Endothelin receptors | ETB receptor | 5 | (Suzuki et al., 2004) |
| G protein-coupled estrogen receptor | GPER | 5 | (Heublein et al., 2012) |
| Formylpeptide receptors | FPR1 | 7 | (Schiffmann et al., 1975) |
| Formylpeptide receptors | FPR2/ALX | 7 | (Le et al., 1999) |
| Formylpeptide receptors | FPR3 | 7 | (Yang et al., 2002) |
| Free fatty acid receptors | FFA1 receptor | 6 | (Briscoe et al., 2003) |
| Free fatty acid receptors | FFA2 receptor | 7 | (Maslowski et al., 2009) |
| Free fatty acid receptors | FFA3 receptor | 6 | (Le Poul et al., 2003) |
| Free fatty acid receptors | FFA4 receptor | 7 | (Kazemian et al., 2012) |
| Free fatty acid receptors | GPR42 | 1 | (Brown et al., 2003) may be a |
| | | | pseudogene |
| Galanin receptors | GAL1 receptor | 5 | (Benya et al., 1998) |
| Galanin receptors | GAL2 receptor | 7 | (Jimenez-Andrade et al., 2004) |
| Galanin receptors | GAL3 receptor | 7 | (Schmidhuber et al., 2009) |
| Ghrelin receptor | ghrelin receptor | 7 | (Dixit et al., 2004) |
| Glycoprotein hormone receptors | FSH receptor | 6 | (Robinson et al., 2010) |
| Glycoprotein hormone receptors | LH receptor | 6 | (Sonoda et al., 2005) |
| Glycoprotein hormone receptors | TSH receptor | 5 | (Cuddihy et al., 1995) |
| Gonadotrophin-releasing hormone receptors | GnRH1 receptor | 6 | (Chen et al., 1999) |
| Gonadotrophin-releasing hormone receptors | GnRH2 receptor | 5 | (Stockhammer et al., 2010) |
| Histamine receptors | H1 receptor | 7 | (Sonobe et al., 2004) |
| Histamine receptors | H2 receptor | 7 | (Mitsuhashi et al., 1989) |
| Histamine receptors | H3 receptor | 5 | (Teuscher et al., 2007) |
| Histamine receptors | H4 receptor | 7 | (Ling et al., 2004) |
| Kisspeptin receptor | kisspeptin receptor | 6 | (Muir et al., 2001) |
| Leukotriene receptors | BLT1 receptor | 7 | (Arita et al., 2007) |
| Leukotriene receptors | BLT2 receptor | 7 | (Yokomizo et al., 2000) |
| Leukotriene receptors | CysLT1 receptor | 7 | (Capra et al., 2005) |
| Leukotriene receptors | CysLT2 receptor | 7 | (Pillai et al., 2004) |
| Leukotriene receptors | OXE receptor | 7 | (Powell & Rokach, 2013) |
| Leukotriene receptors | FPR2/ALX | 7 | (Krishnamoorthy et al., 2012) |
| Lysophospholipid (LPA) receptors | LPA1 receptor | 5 | (Swaney et al., 2010) |
| Lysophospholipid (LPA) receptors | LPA2 receptor | 6 | (An et al., 1998) |
| Lysophospholipid (LPA) receptors | LPA3 receptor | 5 | (Lin et al., 2007) |
| Lysophospholipid (LPA) receptors | LPA4 receptor | 5 | (Waters et al., 2007) |
| Lysophospholipid (LPA) receptors | LPA5 receptor | 7 | (Lundequist & Boyce, 2011) |
| Lysophospholipid (LPA) receptors | LPA6 receptor | 6 | (Pasternack et al., 2008) |
| Melanin-concentrating hormone receptors | MCH1 receptor | 7 | (Ziogas et al., 2013) |
| Melanin-concentrating hormone receptors | MCH2 receptor | 6 | (Hill et al., 2001) |
| Melanocortin receptors | MC1 receptor | 7 | (Hartmeyer et al., 1997) |
| Melanocortin receptors | MC2 receptor | 5 | (Grässel et al., 2009) |
| Melanocortin receptors | MC3 receptor | 6 | (Getting et al., 1999) |
| Melanocortin receptors | MC4 receptor | 5 | (Caruso et al., 2007) |
| Melanocortin receptors | MC5 receptor | 6 | (Chhajlani, 1996) |
| Melatonin receptors | MT1 receptor | 7 | (Carrillo-Vico et al., 2003) |
| Melatonin receptors | MT2 receptor | 7 | (Drazen & Nelson, 2001) |
| Motilin receptor | motilin receptor | 5 | (Ter Beek et al., 2008) |
| Neuromedin U receptors | NMU1 receptor | 7 | (Moriyama et al., 2005) |
| Neuromedin U receptors | NMU2 receptor | 3 | (Moriyama et al., 2005) |
| Neuropeptide FF/neuropeptide AF receptors | NPFF1 receptor | 5 | (Iwasa et al., 2014) |
| Neuropeptide FF/neuropeptide AF receptors | NPFF2 receptor | 5 | (Yang & ladarola, 2003) |
| Neuropeptide S receptor | NPS receptor | 5 | (D'Amato et al., 2007) |
| Neuropeptide W/neuropeptide B receptors | NPBW1 receptor | 6 | (Brezillon et al., 2003) |
| Neuropeptide W/neuropeptide B receptors | NPBW2 receptor | 6 | (Brezillon et al., 2003) |
| Neuropeptide Y receptors | Y1 receptor | 6 | (Mitić et al., 2011) |
| Neuropeptide Y receptors | Y2 receptor | 6 | (Mitić et al., 2011) |
| Neuropeptide Y receptors | Y4 receptor | 4 | (Lin et al., 2006) |
| Neuropeptide Y receptors | Y5 receptor | 6 | (Mitio et al., 2011) |
| Neuropeptide Y receptors | y6 receptor | 3 | (Zhu et al., 2016) |
| Neurotensin receptors | NTS1 receptor | 5 | (Bossard et al., 2007) |
| Neurotensin receptors | NTS2 receptor | 4 | (Lafrance et al., 2010) |
| Hydroxycarboxylic acid receptors | HCA1 receptor | 5 | (Hogue et al., 2014) |
| Hydroxycarboxylic acid receptors | HCA2 receptor | 6 | (Schaub et al., 2001) |
| Hydroxycarboxylic acid receptors | HCA3 receptor | 6 | (Irukayama-Tomobe et al., |
| | | | 2009) |
| Opioid receptors | δ, receptor | 6 | (Gaveriaux et al., 1995) |
| Opioid receptors | κ receptor | 7 | (Taub et al., 1991) |
| Opioid receptors | μ receptor | 7 | (Taub et al., 1991) |
| Opioid receptors | NOP receptor | 6 | (Peluso et al., 1998) |
| Orexin receptors | OX1 receptor | 3 or 4 - currently | (Xiong et al., 2013) |
| | | unclear which | |
| | | receptor subtype | |
| | | is mediating | |
| | | response | |
| Orexin receptors | OX2 receptor | 3 or 4 - currently | (Xiong et al., 2013) |
| | | unclear which | |
| | | receptor subtype | |
| | | is mediating | |
| | | response | |
| P2Y receptors | P2Y1 receptor | 7 | (Fujita et al., 2009) |
| P2Y receptors | P2Y2 receptor | 7 | (Chen et al., 2006) |
| P2Y receptors | P2Y4 receptor | 6 | (Moore et al., 2001) |
| P2Y receptors | P2Y6 receptor | 7 | (Warny et al., 2001) |
| P2Y receptors | P2Y11 receptor | 7 | (Vaughan et al., 2007) |
| P2Y receptors | P2Y12 receptor | 6 | (Sasaki et al., 2003) |
| P2Y receptors | P2Y13 receptor | 7 | (Gao et al., 2010) |
| P2Y receptors | P2Y14 receptor | 7 | (Lee et al., 2003) |
| QRFP receptor | QRFP receptor | 6 | (Jossart et al., 2013) |
| Platelet-activating factor receptor | PAF receptor | 7 | (Ferreira et al., 2004) |
| Prokineticin receptors | PKR1 | 7 | (Cook et al., 2010) |
| Prokineticin receptors | PKR2 | 7 | (Giannini et al., 2009) |
| Prolactin-releasing peptide receptor | PrRP receptor | 6 | (Dorsch et al., 2005) |
| Prostanoid receptors | DP1 receptor | 7 | (Wright et al., 2000) |
| Prostanoid receptors | DP2 receptor | 7 | (Gervais et al., 2001) |
| Prostanoid receptors | EP1 receptor | 7 | (Nagamachi et al., 2007) |
| Prostanoid receptors | EP2 receptor | 7 | (Poloso et al., 2013) |
| Prostanoid receptors | EP3 receptor | 7 | (Kunikata et al., 2005) |
| Prostanoid receptors | EP4 receptor | 7 | (Kabashima et al., 2002) |
| Prostanoid receptors | FP receptor | 7 | (Takayama et al., 2005) |
| Prostanoid receptors | IP receptor | 7 | (Ayer et al., 2008) |
| Prostanoid receptors | TP receptor | 7 | (Li & Tai, 2013) |
| Proteinase-activated receptors | PAR1 | 7 | (Antoniak et al., 2013) |
| Proteinase-activated receptors | PAR2 | 7 | (Davidson et al., 2013) |
| Proteinase-activated receptors | PAR3 | 7 | (Ishihara et al., 1997) |
| Proteinase-activated receptors | PAR4 | 7 | (Mao et al., 2010) |
| Relaxin family peptide receptors | RXFP1 receptor | 5 | (Horton et al., 2012) |
| Relaxin family peptide receptors | RXFP2 receptor | 6 | (Hsu et al., 2002) |
| Relaxin family peptide receptors | RXFP3 receptor | 1 | (Bathgate et al., 2013) |
| Relaxin family peptide receptors | RXFP4 receptor | 6 | (Liu et al., 2005) |
| Somatostatin receptors | sst1 receptor | 6 | (Taniyama et al., 2005) |
| Somatostatin receptors | sst2 receptor | 6 | (Taniyama et al., 2005) |
| Somatostatin receptors | sst3 receptor | 6 | (Taniyama et al., 2005) |
| Somatostatin receptors | sst4 receptor | 6 | (Taniyama et al., 2005) |
| Somatostatin receptors | sst5 receptor | 6 | (Taniyama et al., 2005) |
| Tachykinin receptors | NK1 receptor | 7 | (Saban et al., 2000) |
| Tachykinin receptors | NK2 receptor | 5 | (Laird et al., 2001) |
| Tachykinin receptors | NK3 receptor | 7 | (Improta et al., 2003) |
| Thyrotropin-releasing hormone receptors | TRH1 receptor | 6 | (Mellado et al., 1999) |
| Thyrotropin-releasing hormone receptors | TRH2 receptor | 1 | (Alexander et al., 2011) - not |
| | | | found in humans |
| Trace amine receptor | TA1 receptor | 6 | (D'Andrea et al., 2003) |
| Urotensin receptor | UT receptor | 5 | (Johns et al., 2004) |
| Vasopressin and oxytocin receptors | V1A receptor | 5 | (Bucher et al., 2002) |
| Vasopressin and oxytocin receptors | V1B receptor | 3 | (Sugimoto et al., 1994) |
| Vasopressin and oxytocin receptors | V2 receptor | 5 | (Boyd et al., 2008) |
| Vasopressin and oxytocin receptors | OT receptor | 5 | (İşeri et al., 2005) |
| GPR18, GPR55 and GPR119 | GPR18 | 7 | (Takenouchi et al., 2012) |
| GPR18, GPR55 and GPR119 | GPR55 | 7 | (Cantarella et al., 2011) |
| GPR18, GPR55 and GPR119 | GPR119 | 4 | (Sakamoto et al., 2006) |
| Lysophospholipid (S1P) receptors | S1P1 receptor | 7 | (Matloubian et al., 2004) |
| Lysophospholipid (S1P) receptors | S1P2 receptor | 7 | (McQuiston et al., 2011) |
| Lysophospholipid (S1P) receptors | S1P3 receptor | 7 | (Awojoodu et al., 2013) |
| Lysophospholipid (S1P) receptors | S1P4 receptor | 7 | (Allende et al., 2011) |
| Lysophospholipid (S1P) receptors | S1P5 receptor | 7 | (Jenne et al., 2009) |
| Chemerin receptor | chemerin receptor | 7 | (Haworth et al., 2011) |
| Succinate receptor | succinate receptor | 7 | (Rubic et al., 2008) |
| Oxoglutarate receptor | oxoglutarate | 6 | (Inbe et al., 2004) |
| | receptor | | |
| Taste 2 receptors | TAS2R1 | 6 | (Malki et al., 2015) |
| Taste 2 receptors | TAS2R3 | 6 | (Malki et al., 2015) |
| Taste 2 receptors | TAS2R4 | 6 | (Malki et al., 2015) |
| Taste 2 receptors | TAS2R5 | 6 | (Malki et al., 2015) |
| Taste 2 receptors | TAS2R7 | 6 | (Malki et al., 2015) |
| Taste 2 receptors | TAS2R8 | 6 | (Malki et al., 2015) |
| Taste 2 receptors | TAS2R9 | 6 | (Malki et al., 2015) |
| Taste 2 receptors | TAS2R10 | 6 | (Malki et al., 2015) |
| Taste 2 receptors | TAS2R13 | 6 | (Malki et al., 2015) |
| Taste 2 receptors | TAS2R14 | 6 | (Malki et al., 2015) |
| Taste 2 receptors | TAS2R16 | 6 | (Malki et al., 2015) |
| Taste 2 receptors | TAS2R19 | 6 | (Malki et al., 2015) |
| Taste 2 receptors | TAS2R20 | 6 | (Malki et al., 2015) |
| Taste 2 receptors | TAS2R30 | 6 | (Malki et al., 2015) |
| Taste 2 receptors | TAS2R31 | 6 | (Malki et al., 2015) |
| Taste 2 receptors | TA52R38 | 6 | (Malki et al., 2015) |
| Taste 2 receptors | TA52R39 | 6 | (Malki et al., 2015) |
| Taste 2 receptors | TAS2R40 | 6 | (Malki et al., 2015) |
| Taste 2 receptors | TAS2R41 | 6 | (Malki et al., 2015) |
| Taste 2 receptors | TA52R42 | 6 | (Malki et al., 2015) |
| Taste 2 receptors | TA52R43 | 6 | (Malki et al., 2015) |
| Taste 2 receptors | TA52R45 | 6 | (Malki et al., 2015) |
| Taste 2 receptors | TA52R46 | 6 | (Malki et al., 2015) |
| Taste 2 receptors | TAS2R50 | 6 | (Malki et al., 2015) |
| Taste 2 receptors | TAS2R60 | 6 | (Malki et al., 2015) |
| Class A Orphans | GPR1 | 6 | (Farzan et al., 1997) |
| Class A Orphans | GPR3 | 6 | (Uhlén et al., 2015) |
| Class A Orphans | GPR4 | 7 | (Chen et al., 2011) |
| Class A Orphans | GPR42 | 1 | (Brown et al., 2003) - RT-PCR |
| | | | detected no signal for GPR42 |
| | | | mRNA in samples of normal |
| | | | human tissues |
| Class A Orphans | GPR6 | 6 | (Taquet et al., 2012) |
| Class A Orphans | GPR12 | 6 | (Fomari et al., 2011) |
| Class A Orphans | GPR15 | 7 | (Kim et al., 2013) |
| Class A Orphans | GPR17 | 6 | (Maekawa et al., 2009) |
| Class A Orphans | GPR18 | 6 | (Gantz et al., 1997) |
| Class A Orphans | GPR19 | 4 | (Gazel et al., 2006) |
| Class A Orphans | GPR20 | 6 | (Taquet et al., 2012) |
| Class A Orphans | GPR21 | 7 | (Osborn et al., 2012) |
| Class A Orphans | GPR22 | 6 | (Matteucci et al., 2010) |
| Class A Orphans | GPR25 | 4 | (Consortium, 2013) |
| Class A Orphans | GPR26 | 6 | (Matteucci et al., 2010) |
| Class A Orphans | GPR27 | 6 | (Matsumoto et al., 2000) |
| Class A Orphans | GPR31 | 7 | (Schaub et al., 2001) |
| Class A Orphans | GPR32 | 7 | (Krishnamoorthy et al., 2010) |
| Class A Orphans | GPR33 | 6 | (Rompler et al., 2005) |
| Class A Orphans | GPR34 | 7 | (Sugo et al., 2006) |
| Class A Orphans | GPR35 | 6 | (Wang et al., 2006) |
| Class A Orphans | GPR37 | 4 | (Consortium, 2013) |
| Class A Orphans | GPR37L1 | 4 | (Mas et al., 2011) |
| Class A Orphans | GPR39 | 5 | (Sunuwar et al., 2016) |
| Class A Orphans | GPR45 | 5 | (Fujita et al., 2011) |
| Class A Orphans | GPR50 | 4 | (Elliott et al., 2016) |
| Class A Orphans | GPR52 | 1 | |
| Class A Orphans | GPR55 | 7 | (Schuelert & McDougall, 2011) |
| Class A Orphans | GPR61 | 6 | (Matsumura et al., 2010) |
| Class A Orphans | GPR62 | 4 | (Kwon et al., 2014) |
| Class A Orphans | GPR63 | 3 | (Niedernberg et al., 2003) |
| Class A Orphans | GPR65 | 7 | (Kottyan et al., 2009) |
| Class A Orphans | GPR68 | 7 | (Ichimonji et al., 2010) |
| Class A Orphans | GPR75 | 3 | (Ignatov et al., 2006) |
| Class A Orphans | GPR78 | 6 | (Lu et al., 2010) |
| Class A Orphans | GPR79 | 1 | |
| Class A Orphans | GPR82 | 6 | (Engel et al., 2011) |
| Class A Orphans | GPR83 | 6 | (Hansen et al., 2010) |
| Class A Orphans | GPR84 | 6 | (Venkataraman & Kuo, 2005) |
| Class A Orphans | GPR85 | 6 | (Lattin et al., 2008) |
| Class A Orphans | GPR87 | 6 | (Martinez et al., 2006) |
| Class A Orphans | GPR88 | 5 | (Jurisic et al., 2010) |
| Class A Orphans | GPR101 | 4 | (Watanabe et al., 2013) |
| Class A Orphans | GPR119 | 6 | (Parker et al., 2009) |
| Class A Orphans | GPR132 | 7 | (Frasch et al., 2008) |
| Class A Orphans | GPR135 | 4 | (Kwon et al., 2014) |
| Class A Orphans | GPR139 | 5 | (Tichelaar et al., 2007) |
| Class A Orphans | GPR141 | 4 | (Hong et al., 2015) |
| Class A Orphans | GPR142 | 6 | (Taquet et al., 2012) |
| Class A Orphans | GPR146 | 6 | (Lattin et al., 2008) |
| Class A Orphans | GPR148 | 6 | (Taquet et al., 2012) |
| Class A Orphans | GPR149 | 4 | (Sohn et al., 2009) |
| Class A Orphans | GPR150 | 4 | (Yin et al., 2014) |
| Class A Orphans | GPR151 | 4 | (Keermann et al., 2015) |
| Class A Orphans | GPR152 | 4 | (Ahmad et al., 2016) |
| Class A Orphans | GPR153 | 6 | (Shen et al., 2015) |
| Class A Orphans | GPR160 | 6 | (Lee et al., 2011) |
| Class A Orphans | GPR161 | 5 | (Swan et al., 2013) |
| Class A Orphans | GPR162 | 6 | (Lattin et al., 2008) |
| Class A Orphans | GPR171 | 5 | (Rossi et al., 2013) |
| Class A Orphans | GPR173 | 6 | (Fomari et al., 2011) |
| Class A Orphans | GPR174 | 6 | (Shen et al., 2015) |
| Class A Orphans | GPR176 | 6 | (Wensman et al., 2012) |
| Class A Orphans | GPR182 | 6 | (Matteucci et al., 2010) |
| Class A Orphans | GPR183 | 7 | (Gatto et al., 2011) |
| Class A Orphans | LGR4 | 6 | (Liu et al., 2013) |
| Class A Orphans | LGR5 | 4 | (Quigley et al., 2009) |
| Class A Orphans | LGR6 | 6 | (Aho et al., 2013) |
| Class A Orphans | MAS1 | 7 | (da Silveira et al., 2010) |
| Class A Orphans | MASL | 6 | (Foster et al., 2016) |
| Class A Orphans | MRGPRD | 5 | (Qu et al., 2014) |
| Class A Orphans | MRGPRE | 4 | (Kwon et al., 2014) |
| Class A Orphans | MRGPRF | 4 | (Liang et al., 2016) |
| Class A Orphans | MRGPRG | 6 | (Othman et al., 2015) |
| Class A Orphans | MRGPRX1 | 5 | (Solinski et al., 2013) |
| Class A Orphans | MRGPRX2 | 7 | (Subramanian et al., 2011) |
| Class A Orphans | MRGPRX3 | 5 | (Yi et al., 2012) |
| Class A Orphans | MRGPRX4 | 1 | (Bader et al., 2014) |
| Class A Orphans | OPN3 | 6 | (White et al., 2008) |
| Class A Orphans | OPN4 | 4 | (Wang et al., 2010) |
| Class A Orphans | OPN5 | 3 | (Ohshima et al., 2002) |
| Class A Orphans | P2RY8 | 6 | (Cantagrel et al., 2004) |
| Class A Orphans | P2RY10 | 6 | (Rao et al., 1999) |
| Class A Orphans | TAAR2 | 6 | (Babusyte et al., 2013) |
| Class A Orphans | TAAR3 | 4 | (D'Andrea et al., 2012) |
| Class A Orphans | TAAR4P | 1 | |
| Class A Orphans | TAAR5 | 6 | (Taquet et al., 2012) |
| Class A Orphans | TAAR6 | 6 | (D'Andrea et al., 2012) |
| Class A Orphans | TAAR8 | 6 | (D'Andrea et al., 2012) |
| Class A Orphans | TAAR9 | 6 | (Taquet et al., 2012) |

Family A olfactory GPCRs and the current level of evidence for their involvement in inflammation (see key above):

| Family | Sub | | Level of | |
|---|---|---|---|---|
| ID | Family | Symbol | Evidence | Reference |
| 1 | C | OR1C1 | 1 | |
| 1 | F | OR1F12 | 1 | |
| 1 | J | OR1J1 | 1 | |
| 1 | J | OR1J2 | 1 | |
| 1 | J | OR1J4 | 1 | |
| 1 | N | OR1N1 | 1 | |
| 1 | N | OR1N2 | 1 | |
| 1 | L | OR1L8 | 1 | |
| 1 | Q | OR1Q1 | 1 | |
| 1 | B | OR1B1 | 1 | |
| 1 | L | OR1L1 | 4 | (Garcia-Vivas et al., 2016) |
| 1 | L | OR1L3 | 1 | |
| 1 | L | OR1L4 | 1 | |
| 1 | L | OR1L6 | 1 | |
| 1 | K | OR1K1 | 1 | |
| 1 | S | OR1S2 | 4 | (Lee et al., 2011) |
| 1 | S | OR1S1 | 4 | (Lee et al., 2011) |
| 1 | F | OR1F1 | 1 | |
| 1 | D | OR1D5 | 1 | |
| 1 | D | OR1D2 | 5 | (Kalbe et al., 2016) |
| 1 | G | OR1G1 | 1 | |
| 1 | A | OR1A2 | 4 | (Garcia-Vivas et al., 2016) |
| 1 | A | OR1A1 | 1 | |
| 1 | D | OR1D4 | 1 | |
| 1 | E | OR1E1 | 1 | |
| 1 | E | OR1E2 | 1 | |
| 1 | M | OR1M1 | 1 | |
| 1 | I | OR1I1 | 1 | |
| 2 | B | OR2B11 | 6 | (Flegel et al., 2013) |
| 2 | W | OR2W5 | 1 | |
| 2 | C | OR2C3 | 6 | (Flegel et al., 2013) |
| 2 | G | OR2G2 | 1 | |
| 2 | G | OR2G3 | 1 | |
| 2 | W | OR2W3 | 6 | (Flegel et al., 2013) |
| 2 | T | OR2T8 | 1 | |
| 2 | AJ | OR2AJ1 | 1 | |
| 2 | L | OR2L8 | 1 | |
| 2 | AK | OR2AK2 | 4 | (Garcia-Vivas et al., 2016) |
| 2 | L | OR2L5 | 1 | |
| 2 | L | OR2L2 | 1 | |
| 2 | L | OR2L3 | 1 | |
| 2 | L | OR2L13 | 6 | (Flegel et al., 2013) |
| 2 | M | OR2M5 | 1 | |
| 2 | M | OR2M2 | 1 | |
| 2 | M | OR2M3 | 1 | |
| 2 | M | OR2M4 | 1 | |
| 2 | T | OR2T33 | 1 | |
| 2 | T | OR2T12 | 1 | |
| 2 | M | OR2M7 | 1 | |
| 2 | T | OR2T4 | 1 | |
| 2 | T | OR2T6 | 1 | |
| 2 | T | OR2T1 | 1 | |
| 2 | T | OR2T7 | 1 | |
| 2 | T | OR2T2 | 1 | |
| 2 | T | OR2T3 | 1 | |
| 2 | I | OR2T5 | 1 | |
| 2 | G | OR2G6 | 1 | |
| 2 | T | OR2T29 | 1 | |
| 2 | T | OR2T34 | 6 | (Flegel et al., 2013) |
| 2 | T | OR2T10 | 1 | |
| 2 | T | OR2T11 | 6 | (Flegel et al., 2013) |
| 2 | T | OR2T35 | 1 | |
| 2 | T | OR2T27 | 1 | |
| 2 | Y | OR2Y1 | 1 | |
| 2 | V | OR2V1 | 1 | |
| 2 | V | OR2V2 | 1 | |
| 2 | B | OR2B2 | 1 | |
| 2 | B | OR2B6 | 6 | (Flegel et al., 2013) |
| 2 | W | OR2W1 | 1 | |
| 2 | B | OR2B3 | 1 | |
| 2 | J | OR2J3 | 6 | (Zhao et al., 2013) |
| 2 | J | OR2J2 | 1 | |
| 2 | H | OR2H1 | 1 | |
| 2 | H | OR2H2 | 1 | |
| 2 | A | OR2A4 | 6 | (Flegel et al., 2013) |
| 2 | AE | OR2AE1 | 1 | |
| 2 | F | OR2F2 | 1 | |
| 2 | F | OR2F1 | 1 | |
| 2 | A | OR2A5 | 1 | |
| 2 | A | OR2A25 | 1 | |
| 2 | A | OR2A12 | 1 | |
| 2 | A | OR2A2 | 6 | (Flegel et al., 2013) |
| 2 | A | OR2A14 | 1 | |
| 2 | A | OR2A42 | 6 | (Flegel et al., 2013) |
| 2 | A | OR2A7 | 6 | (Flegel et al., 2013) |
| 2 | A | OR2A1 | 6 | (Flegel et al., 2013) |
| 2 | S | OR2S2 | 1 | |
| 2 | K | OR2K2 | 1 | |
| 2 | AG | OR2AG2 | 1 | |
| 2 | AG | OR2AG1 | 5 | (Kalbe et al., 2016) |
| 2 | D | OR2D2 | 4 | (Lee et al., 2011) |
| 2 | D | OR2D3 | 4 | (Lee et al., 2011) |
| 2 | AT | OR2AT4 | 1 | |
| 2 | AP | OR2AP1 | 1 | |
| 2 | C | OR2C1 | 6 | (Flegel et al., 2013) |
| 2 | Z | OR2Z1 | 1 | |
| 3 | A | OR3A2 | 1 | |
| 3 | A | OR3A1 | 1 | |
| 3 | A | OR3A4 | 1 | |
| 3 | A | OR3A3 | 6 | (Flegel et al., 2013) |
| 4 | F | OR4F5 | 1 | |
| 4 | F | OR4F29 | 1 | |
| 4 | F | OR4F16 | 1 | |
| 4 | F | OR4F3 | 1 | |
| 4 | F | OR4F21 | 1 | |
| 4 | B | OR4B1 | 1 | |
| 4 | X | OR4X2 | 1 | |
| 4 | X | OR4X1 | 1 | |
| 4 | S | OR4S1 | 1 | |
| 4 | C | OR4C3 | 1 | |
| 4 | C | OR4C5 | 1 | |
| 4 | A | OR4A47 | 1 | |
| 4 | C | OR4C13 | 4 | (Lee et al., 2011) |
| 4 | C | OR4C12 | 4 | (Garcia-Vivas et al., 2016) |
| 4 | A | OR4A5 | 1 | |
| 4 | C | OR4C46 | 1 | |
| 4 | A | OR4A16 | 1 | |
| 4 | A | OR4A15 | 4 | (Garcia-Vivas et al., 2016) |
| 4 | C | OR4C15 | 4 | (Lee et al., 2011) |
| 4 | C | OR4C16 | 4 | (Lee et al., 2011) |
| 4 | C | OR4C11 | 4 | (Lee et al., 2011) |
| 4 | P | OR4P4 | 1 | |
| 4 | S | OR4S2 | 1 | |
| 4 | C | OR4C6 | 1 | |
| 4 | D | OR4D6 | 1 | |
| 4 | D | OR4D10 | 6 | (Zhao et al., 2013) |
| 4 | D | OR4D11 | 1 | |
| 4 | D | OR4D9 | 1 | |
| 4 | D | OR4D5 | 1 | |
| 4 | Q | OR4Q3 | 6 | (Zhao et al., 2013) |
| 4 | M | OR4M1 | 6 | (Zhao et al., 2013) |
| 4 | N | OR4N2 | 1 | |
| 4 | K | OR4K2 | 1 | |
| 4 | K | OR4K5 | 1 | |
| 4 | K | OR4K1 | 1 | |
| 4 | K | OR4K15 | 4 | (Lee et al., 2011) |
| 4 | K | OR4K14 | 4 | (Lee et al., 2011) |
| 4 | K | OR4K13 | 4 | (Garcia-Vivas et al., 2016) |
| 4 | L | OR4L1 | 1 | |
| 4 | K | OR4K17 | 4 | (Garcia-Vivas et al., 2016) |
| 4 | N | OR4N5 | 4 | (Lee et al., 2011) |
| 4 | E | OR4E2 | 1 | |
| 4 | M | OR4M2 | 1 | |
| 4 | N | OR4N4 | 1 | |
| 4 | F | OR4F6 | 1 | |
| 4 | F | OR4F15 | 1 | |
| 4 | F | OR4F4 | 1 | |
| 4 | D | OR4D1 | 1 | |
| 4 | D | OR4D2 | 1 | |
| 4 | F | OR4F17 | 1 | |
| 4 | C | OR4C45 | 1 | |
| 5 | AC | OR5AC2 | 4 | (Lee et al., 2011) |
| 5 | H | OR5H1 | 1 | |
| 5 | H | OR5H14 | 1 | |
| 5 | H | OR5H15 | 1 | |
| 5 | H | OR5H6 | 1 | |
| 5 | H | OR5H2 | 1 | |
| 5 | K | OR5K4 | 1 | |
| 5 | K | OR5K3 | 4 | (Garcia-Vivas et al., 2016) |
| 5 | K | OR5K1 | 1 | |
| 5 | K | OR5K2 | 1 | |
| 5 | V | OR5V1 | 1 | |
| 5 | C | OR5C1 | 1 | |
| 5 | P | OR5P2 | 1 | |
| 5 | P | OR5P3 | 1 | |
| 5 | D | OR5D13 | 4 | (Lee et al., 2011) |
| 5 | D | OR5D14 | 4 | (Lee et al., 2011) |
| 5 | L | OR5L1 | 4 | (Lee et al., 2011) |
| 5 | D | OR5D18 | 4 | (Lee et al., 2011) |
| 5 | L | OR5L2 | 4 | (Lee et al., 2011) |
| 5 | D | OR5D16 | 4 | (Lee et al., 2011) |
| 5 | W | OR5W2 | 4 | (Lee et al., 2011) |
| 5 | I | OR5I1 | 4 | (Garcia-Vivas et al., 2016) |
| 5 | F | OR5F1 | 4 | (Lee et al., 2011) |
| 5 | AS | OR5AS1 | 4 | (Lee et al., 2011) |
| 5 | J | OR5J2 | 4 | (Lee et al., 2011) |
| 5 | T | OR5T2 | 4 | (Garcia-Vivas et al., 2016) |
| 5 | T | OR5T3 | 4 | (Garcia-Vivas et al., 2016) |
| 5 | T | OR5T1 | 4 | (Lee et al., 2011) |
| 5 | R | OR5R1 | 4 | (Lee et al., 2011) |
| 5 | M | OR5M9 | 4 | (Lee et al., 2011) |
| 5 | M | OR5M3 | 4 | (Lee et al., 2011) |
| 5 | M | OR5M8 | 4 | (Lee et al., 2011) |
| 5 | M | OR5M11 | 4 | (Lee et al., 2011) |
| 5 | M | OR5M10 | 4 | (Lee et al., 2011) |
| 5 | M | OR5M1 | 4 | (Lee et al., 2011) |
| 5 | AP | OR5AP2 | 4 | (Lee et al., 2011) |
| 5 | AR | OR5AR1 | 4 | (Lee et al., 2011) |
| 5 | AK | OR5AK2 | 4 | (Garcia-Vivas et al., 2016) |
| 5 | B | OR5B17 | 4 | (Garcia-Vivas et al., 2016) |
| 5 | B | OR5B3 | 4 | (Garcia-Vivas et al., 2016) |
| 5 | B | OR5B2 | 4 | (Lee et al., 2011) |
| 5 | B | OR5B12 | 4 | (Lee et al., 2011) |
| 5 | B | OR5B21 | 4 | (Lee et al., 2011) |
| 5 | AN | OR5AN1 | 1 | |
| 5 | A | OR5A2 | 1 | |
| 5 | A | OR5A1 | 1 | |
| 5 | AU | OR5AU1 | 1 | |
| 6 | Y | OR6Y1 | 1 | |
| 6 | P | OR6P1 | 1 | |
| 6 | K | OR6K2 | 1 | |
| 6 | K | OR6K3 | 1 | |
| 6 | K | OR6K6 | 4 | (Garcia-Vivas et al., 2016) |
| 6 | N | OR6N1 | 1 | |
| 6 | N | OR6N2 | 1 | |
| 6 | F | OR6F1 | 1 | |
| 6 | B | OR6B2 | 1 | |
| 6 | B | OR6B3 | 1 | |
| 6 | V | OR6V1 | 6 | (Feingold et al., 1999) |
| 6 | B | OR6B1 | 1 | |
| 6 | A | OR6A2 | 1 | |
| 6 | Q | OR6Q1 | 4 | (Lee et al., 2011) |
| 6 | X | OR6X1 | 4 | (Lee et al., 2011) |
| 6 | M | OR6M1 | 4 | (Lee et al., 2011) |
| 6 | T | OR6T1 | 1 | |
| 6 | C | OR6C74 | 4 | (Garcia-Vivas et al., 2016) |
| 6 | C | OR6C6 | 1 | |
| 6 | C | OR6C1 | 1 | |
| 6 | C | OR6C3 | 1 | |
| 6 | C | OR6C75 | 1 | |
| 6 | C | OR6C65 | 1 | |
| 6 | C | OR6C76 | 1 | |
| 6 | C | OR6C2 | 1 | |
| 6 | C | OR6C70 | 1 | |
| 6 | C | OR6C68 | 1 | |
| 6 | C | OR6C4 | 1 | |
| 6 | S | OR6S1 | 1 | |
| 6 | J | OR6J1 | 1 | |
| 7 | G | OR7G2 | 1 | |
| 7 | G | OR7G1 | 1 | |
| 7 | G | OR7G3 | 1 | |
| 7 | D | OR7D2 | 6 | (Flegel et al., 2013) |
| 7 | D | OR7D4 | 1 | |
| 7 | E | OR7E24 | 1 | |
| 7 | C | OR7C1 | 1 | |
| 7 | A | OR7A5 | 1 | |
| 7 | A | OR7A10 | 1 | |
| 7 | A | OR7A17 | 1 | |
| 7 | C | OR7C2 | 1 | |
| 8 | I | OR8I2 | 1 | |
| 8 | H | OR8H2 | 4 | (Lee et al., 2011) |
| 8 | H | OR8H3 | 4 | (Lee et al., 2011) |
| 8 | J | OR8J3 | 4 | (Lee et al., 2011) |
| 8 | K | OR8K5 | 4 | (Lee et al., 2011) |
| 8 | H | OR8H1 | 4 | (Lee et al., 2011) |
| 8 | K | OR8K3 | 4 | (Lee et al., 2011) |
| 8 | K | OR8K1 | 4 | (Lee et al., 2011) |
| 8 | J | OR8J1 | 4 | (Lee et al., 2011) |
| 8 | U | OR8U1 | 4 | (Lee et al., 2011) |
| 8 | D | OR8D4 | 1 | |
| 8 | G | OR8G1 | 1 | |
| 8 | G | OR8G5 | 1 | |
| 8 | D | OR8D1 | 1 | |
| 8 | D | OR8D2 | 1 | |
| 8 | B | OR8B2 | 1 | |
| 8 | B | OR8B3 | 1 | |
| 8 | B | OR8B4 | 1 | |
| 8 | B | OR8B8 | 1 | |
| 8 | B | OR8B12 | 1 | |
| 8 | A | OR8A1 | 1 | |
| 8 | S | OR8S1 | 1 | |
| 8 | U | OR8U8 | 4 | (Lee et al., 2011) |
| 8 | U | OR8U9 | 1 | |
| 9 | A | OR9A4 | 4 | (Lee et al., 2011) |
| 9 | A | OR9A2 | 6 | (Malki et al., 2015) |
| 9 | G | OR9G1 | 4 | (Lee et al., 2011) |
| 9 | G | OR9G4 | 4 | (Lee et al., 2011) |
| 9 | I | OR9I1 | 1 | |
| 9 | Q | OR9Q1 | 1 | |
| 9 | Q | OR9Q2 | 4 | (Lee et al., 2011) |
| 9 | K | OR9K2 | 1 | |
| 9 | G | OR9G9 | 4 | (Lee et al., 2011) |
| 10 | T | OR10T2 | 1 | |
| 10 | K | OR10K2 | 1 | |
| 10 | K | OR10K1 | 1 | |
| 10 | R | OR10R2 | 1 | |
| 10 | X | OR10X1 | 1 | |
| 10 | Z | OR10Z1 | 1 | |
| 10 | J | OR10J3 | 1 | |
| 10 | J | OR10J1 | 1 | |
| 10 | J | OR10J5 | 1 | |
| 10 | C | OR10C1 | 1 | |
| 10 | A | OR10A5 | 1 | |
| 10 | A | OR10A2 | 1 | |
| 10 | A | OR10A4 | 1 | |
| 10 | A | OR10A6 | 1 | |
| 10 | A | OR10A3 | 1 | |
| 10 | AG | OR10AG1 | 4 | (Lee et al., 2011) |
| 10 | Q | OR10Q1 | 4 | (Lee et al., 2011) |
| 10 | W | OR10W1 | 4 | (Lee et al., 2011) |
| 10 | V | OR10V1 | 1 | |
| 10 | S | OR10S1 | 1 | |
| 10 | G | OR10G6 | 1 | |
| 10 | G | OR10G4 | 1 | |
| 10 | G | OR10G9 | 1 | |
| 10 | G | OR10G8 | 1 | |
| 10 | G | OR10G7 | 1 | |
| 10 | D | OR10D3 | 1 | |
| 10 | AD | OR10AD1 | 1 | |
| 10 | A | OR10A7 | 4 | (Garcia-Vivas et al., 2016) |
| 10 | P | OR10P1 | 1 | |
| 10 | G | OR10G3 | 1 | |
| 10 | G | OR10G2 | 1 | |
| 10 | H | OR10H2 | 1 | |
| 10 | H | OR10H3 | 1 | |
| 10 | H | OR10H5 | 1 | |
| 10 | H | OR10H1 | 1 | |
| 10 | H | OR10H4 | 1 | |
| 11 | L | OR11L1 | 1 | |
| 11 | A | OR11A1 | 1 | |
| 11 | H | OR11H12 | 1 | |
| 11 | H | OR11H2 | 1 | |
| 11 | G | OR11G2 | 1 | |
| 11 | H | OR11H6 | 1 | |
| 11 | H | OR11H4 | 1 | |
| 11 | H | OR11H1 | 6 | (Zhao et al., 2013) |
| 12 | D | OR12D3 | 4 | (Garcia-Vivas et al., 2016) |
| 12 | D | OR12D2 | 1 | |
| 13 | G | OR13G1 | 4 | (Garcia-Vivas et al., 2016) |
| 13 | J | OR13J1 | 1 | |
| 13 | F | OR13F1 | 4 | (Lee et al., 2011) |
| 13 | C | OR13C4 | 4 | (Garcia-Vivas et al., 2016) |
| 13 | C | OR13C3 | 4 | (Lee et al., 2011) |
| 13 | C | OR13C8 | 4 | (Lee et al., 2011) |
| 13 | C | OR13C5 | 4 | (Lee et al., 2011) |
| 13 | C | OR13C2 | 4 | (Lee et al., 2011) |
| 13 | C | OR13C9 | 1 | |
| 13 | D | OR13D1 | 1 | |
| 13 | A | OR13A1 | 1 | |
| 13 | H | OR13H1 | 1 | |
| 14 | A | OR14A2 | 1 | |
| 14 | K | OR14K1 | 1 | |
| 14 | A | OR14A16 | 1 | |
| 14 | C | OR14C36 | 1 | |
| 14 | I | OR14I1 | 1 | |
| 14 | J | OR14J1 | 1 | |
| 51 | D | OR51D1 | 6 | (Malki et al., 2015) |
| 51 | E | OR51E1 | 6 | (Malki et al., 2015) |
| 51 | E | OR51E2 | 6 | (Malki et al., 2015) |
| 51 | F | OR51F1 | 6 | (Malki et al., 2015) |
| 51 | F | OR51F2 | 6 | (Malki et al., 2015) |
| 51 | S | OR51S1 | 6 | (Malki et al., 2015) |
| 51 | T | OR51T1 | 6 | (Malki et al., 2015) |
| 51 | A | OR51A7 | 6 | (Malki et al., 2015) |
| 51 | G | OR51G2 | 6 | (Malki et al., 2015) |
| 51 | G | OR51G1 | 6 | (Malki et al., 2015) |
| 51 | A | OR51A4 | 6 | (Malki et al., 2015) |
| 51 | A | OR51A2 | 6 | (Malki et al., 2015) |
| 51 | L | OR51L1 | 6 | (Malki et al., 2015) |
| 51 | V | OR51V1 | 6 | (Malki et al., 2015) |
| 51 | B | OR51B4 | 6 | (Malki et al., 2015) |
| 51 | B | OR51B2 | 6 | (Malki et al., 2015) |
| 51 | B | OR51B5 | 6 | (Malki et al., 2015) |
| 51 | B | OR51B6 | 6 | (Malki et al., 2015) |
| 51 | M | OR51M1 | 6 | (Malki et al., 2015) |
| 51 | J | OR51J1 | 6 | (Malki et al., 2015) |
| 51 | Q | OR51Q1 | 6 | (Malki et al., 2015) |
| 51 | I | OR51I1 | 6 | (Malki et al., 2015) |
| 51 | 1 | OR51I2 | 6 | (Malki et al., 2015) |
| 52 | B | OR52B4 | 6 | (Malki et al., 2015) |
| 52 | K | OR52K2 | 6 | (Malki et al., 2015) |
| 52 | K | OR52K1 | 6 | (Malki et al., 2015) |
| 52 | M | OR52M1 | 6 | (Malki et al., 2015) |
| 52 | I | OR52I2 | 6 | (Malki et al., 2015) |
| 52 | I | OR52I1 | 6 | (Malki et al., 2015) |
| 52 | R | OR52R1 | 6 | (Malki et al., 2015) |
| 52 | J | OR52J3 | 6 | (Malki et al., 2015) |
| 52 | E | OR52E2 | 6 | (Malki et al., 2015) |
| 52 | A | OR52A4 | 6 | (Malki et al., 2015) |
| 52 | A | OR52A5 | 6 | (Malki et al., 2015) |
| 52 | A | OR52A1 | 6 | (Malki et al., 2015) |
| 52 | D | OR52D1 | 6 | (Malki et al., 2015) |
| 52 | H | OR52H1 | 6 | (Malki et al., 2015) |
| 52 | B | OR52B6 | 6 | (Malki et al., 2015) |
| 52 | N | OR52N4 | 6 | (Flegel et al., 2013) |
| 52 | N | OR52N5 | 6 | (Zhao et al., 2013) |
| 52 | N | OR52N1 | 6 | (Malki et al., 2015) |
| 52 | N | OR52N2 | 6 | (Malki et al., 2015) |
| 52 | E | OR52E6 | 6 | (Malki et al., 2015) |
| 52 | E | OR52E8 | 6 | (Malki et al., 2015) |
| 52 | E | OR52E4 | 6 | (Malki et al., 2015) |
| 52 | E | OR52E5 | 6 | (Malki et al., 2015) |
| 52 | L | OR52L1 | 6 | (Malki et al., 2015) |
| 52 | B | OR52B2 | 6 | (Malki et al., 2015) |
| 52 | W | OR52W1 | 6 | (Malki et al., 2015) |
| 56 | B | OR56B1 | 6 | (Malki et al., 2015) |
| 56 | A | OR56A3 | 6 | (Malki et al., 2015) |
| 56 | A | OR56A5 | 6 | (Malki et al., 2015) |
| 56 | A | OR56A4 | 6 | (Malki et al., 2015) |
| 56 | A | OR56A1 | 6 | (Malki et al., 2015) |
| 56 | B | OR56B4 | 6 | (Malki et al., 2015) |

Family A vomeronasal and opsin GPCRs and the current level of evidence for their involvement in inflammation (see key above):

| | | | Level of | |
|---|---|---|---|---|
| Type | Subtype | Symbol | Evidence | Reference |
| Vomeronasal | vomeronasal 1 receptor 1 | VN1R1 | 1 | |
| Vomeronasal | vomeronasal 1 receptor 2 | VN1R2 | 1 | |
| Vomeronasal | vomeronasal 1 receptor 3 | VN1R3 | 1 | |
| | (gene/pseudogene) | | | |
| Vomeronasal | vomeronasal 1 receptor 4 | VN1R4 | 1 | |
| Vomeronasal | vomeronasal 1 receptor 5 | VN1R5 | 1 | |
| | (gene/pseudogene) | | | |
| Opsin | opsin 1 (cone pigments) | OPN1LW | 1 | |
| Opsin | opsin 1 (cone pigments) | OPN1MW | 1 | |
| Opsin | opsin 1 (cone pigments) | OPN1MW2 | 1 | |
| Opsin | opsin 1 (cone pigments) | OPN1MW3 | 1 | |
| Opsin | opsin 1 (cone pigments) | OPN1SW | 1 | |
| Opsin | opsin 3 | OPN3 | 1 | |
| Opsin | opsin 4 | OPN4 | 4 | (Lee et al., 2011) |
| Opsin | opsin 5 | OPN5 | 1 | |
| Opsin | retinal G protein coupled | RGR | 1 | |
| | receptor | | | |
| Opsin | rhodopsin | RHO | 1 | |
| Opsin | retinal pigment epithelium- | RRH | 1 | |
| | derived rhodopsin homolog | | | |

Family B GPCRs and the current level of evidence for their involvement in inflammation (see key above):

| Type | Subtype | Level of Evidence | Reference |
|---|---|---|---|
| Calcitonin receptors | CT receptor | 6 | (Body et al., 1990) |
| Calcitonin receptors | AMY1 receptor | 3 - currently unknown | (Masters et al., 2010) |
| | | which AMY receptor | |
| | | subtype mediates this | |
| Calcitonin receptors | AMY2 receptor | 3 - currently unknown | (Masters et al., 2010) |
| | | which AMY receptor | |
| | | subtype mediates this | |
| Calcitonin receptors | AMY3 receptor | 3 - currently unknown | (Masters et al., 2010) |
| | | which AMY receptor | |
| | | subtype mediates this | |
| Calcitonin receptors | calcitonin receptor- | 6 | (Hagner et al., 2002) |
| | like receptor | | |
| Calcitonin receptors | CGRP receptor | 5 | (Salmone t al., 2001) |
| Calcitonin receptors | AM1 receptor | 3 - currently unknown | (Elsasser & Kahl, 2002) |
| | | which AM receptor | |
| | | subtype mediates this | |
| Calcitonin receptors | AM2 receptor | 3 - currently unknown | (Elsasser & Kahl, 2002) |
| | | which AM receptor | |
| | | subtype mediates this | |
| Corticotropin-releasing | CRF1 receptor | 5 | (Tsatsanis et al., 2007) |
| factor receptors | | | |
| Corticotropin-releasing | CRF2 receptor | 5 | (Tsatsanis et al., 2007) |
| factor receptors | | | |
| Glucagon receptor family | GHRH receptor | 6 | (Chen et al., 1999) |
| Glucagon receptor family | GIP receptor | 5 | (Nie et al., 2012) |
| Glucagon receptor family | GLP-1 receptor | 5 | (Kodera et al., 2011) |
| Glucagon receptor family | GLP-2 receptor | 5 | (Cani et al., 2009) |
| Glucagon receptor family | glucagon receptor | 5 | (Buler et al., 2012) |
| Glucagon receptor family | secretin receptor | 5 | (Petersen & Myren, 1974) |
| Parathyroid hormone | PTH1 receptor | 3 - currently unknown | (Jahnsen et al., 2002) |
| receptors | | which PTH receptor | |
| | | subtype mediates this | |
| Parathyroi hormone | PTH2 receptor | 3 - currently unknown | (Jahnsen et al., 2002) |
| receptors | | which PTH receptor | |
| | | subtype mediates this | |
| VIP and PACAP receptors | PAC1 receptor | 5 | (Martinez et al., 2002) |
| VIP and PACAP receptors | VPAC1 receptor | 7 | (Yadav et al., 2011) |
| VIP and PACAP receptors | VPAC2 receptor | 7 | (Voice et al., 2003) |
| Adhesion Class GPCRs | ADGRA1 | 1 | (Nijmeijer et al., 2016) |
| Adhesion Class GPCRs | ADGRA2 | 2 | (Nijmeijer et al., 2016) |
| Adhesion Class GPCRs | ADGRA3 | 1 | (Nijmeijer et al., 2016) |
| Adhesion Class GPCRs | ADGRB1 | 5 | (Billings et al., 2016) |
| Adhesion Class GPCRs | ADGRB2 | 2 | (Nijmeijer et al., 2016) |
| Adhesion Class GPCRs | ADGRB3 | 2 | (Nijmeijer et al., 2016) |
| Adhesion Class GPCRs | CELSR1 | 2 | (Nijmeijer et al., 2016) |
| Adhesion Class GPCRs | CELSR2 | 2 | (Nijmeijer et al., 2016) |
| Adhesion Class GPCRs | CELSR3 | 2 | (Nijmeijer et al., 2016) |
| Adhesion Class GPCRs | ADGRD1 | 1 | (Nijmeijer et al., 2016) |
| Adhesion Class GPCRs | ADGRD2 | 1 | (Nijmeijer et al., 2016) |
| Adhesion Class GPCRs | ADGRE1 | 7 | (Lin et al., 2005) |
| Adhesion Class GPCRs | ADGRE2 | 7 | (Chen et al., 2011) |
| Adhesion Class GPCRs | ADGRE3 | 7 | (Stacey et al., 2001) |
| Adhesion Class GPCRs | ADGRE4P | 6 | (Caminschi et al., 2006) |
| Adhesion Class GPCRs | ADGRE5 | 7 | (Galle et al., 2006) |
| Adhesion Class GPCRs | ADGRF1 | 6 | (Harvey et al., 2010) |
| Adhesion Class GPCRs | ADGRF2 | 1 | (Nijmeijer et al., 2016) |
| Adhesion Class GPCRs | ADGRF3 | 2 | (Nijmeijer et al., 2016) |
| Adhesion Class GPCRs | ADGRF4 | 1 | (Nijmeijer et al., 2016) |
| Adhesion Class GPCRs | ADGRF5 | 1 | (Nijmeijer et al., 2016) |
| Adhesion Class GPCRs | ADGRG1 | 6 | (Peng et al., 2011) |
| Adhesion Class GPCRs | ADGRG2 | 1 | (Nijmeijer et al., 2016) |
| Adhesion Class GPCRs | ADGRG3 | 6 | (Peng et al., 2011) |
| Adhesion Class GPCRs | ADGRG4 | 2 | (Nijmeijer et al., 2016) |
| Adhesion Class GPCRs | ADGRG5 | 6 | (Peng et al., 2011) |
| Adhesion Class GPCRs | ADGRG6 | 1 | (Nijmeijer et al., 2016) |
| Adhesion Class GPCRs | ADGRG7 | 1 | (Nijmeijer et al., 2016) |
| Adhesion Class GPCRs | ADGRL1 | 1 | (Nijmeijer et al., 2016) |
| Adhesion Class GPCRs | ADGRL2 | 1 | (Nijmeijer et al., 2016) |
| Adhesion Class GPCRs | ADGRL3 | 1 | (Nijmeijer et al., 2016) |
| Adhesion Class GPCRs | ADGRL4 | 1 | (Nijmeijer et al., 2016) |
| Adhesion Class GPCRs | ADGRV1 | 2 | (Nijmeijer et al., 2016) |

Family C GPCRs and the current level of evidence for their involvement in inflammation (see key above):

| | | Level of | |
|---|---|---|---|
| Type | Subtype | Evidence | Reference |
| Calcium-sensing receptors | CaS receptor | 7 | (Bandyopadhyay et al., 2007) |
| Calcium-sensing receptors | GPRC6 receptor | 6 | (Wellendorph & Bräuner-Osborne, 2004) |
| GABAB receptors | GABAB1 | 5 | (Ito et al., 2013) |
| GABAB receptors | GABAB2 | 5 | (Ito et al., 2013) |
| GABAB receptors | GABAB receptor | 5 | (Ito et al., 2013) |
| Metabotropic glutamate | mGlu1 receptor | 7 | (Bhave et al., 2001) |
| receptors | | | |
| Metabotropic glutamate | mGlu2 receptor | 5 | (Zammataro et al., 2011) |
| receptors | | | |
| Metabotropic glutamate | mGlu3 receptor | 5 | (Boxall et al., 1997) |
| receptors | | | |
| Metabotropic glutamate | mGlu4 receptor | 6 | (Fallarino et al., 2010) |
| receptors | | | |
| Metabotropic glutamate | mGlu5 receptor | 7 | (Bhave et al., 2001) |
| receptors | | | |
| Metabotropic glutamate | mGlu6 receptor | 1 | (Volpi et al., 2012) |
| receptors | | | |
| Metabotropic glutamate | mGlu7 receptor | 6 | (Fallarino et al., 2010) |
| receptors | | | |
| Metabotropic glutamate | mGlu8 receptor | 6 | (Fallarino et al., 2010) |
| receptors | | | |
| Taste 1 receptors | TAS1R1 | 6 | (Malki et al., 2015) |
| Taste 1 receptors | TAS1R2 | 6 | (Malki et al., 2015) |
| Taste 1 receptors | TAS1R3 | 6 | (Malki et al., 2015) |
| Class C Orphans | GPR156 | 5 | (Calderón-Garcidueñas et al., 2012) |
| Class C Orphans | GPR158 | 5 | (Sima et al., 2015) |
| Class C Orphans | GPR179 | 5 | (Kononikhin et al., 2016) |
| Class C Orphans | GPRC5A | 5 | (Deng et al., 2010) |
| Class C Orphans | GPRC5B | 5 | (Kim et al., 2012) |
| Class C Orphans | GPRC5C | 5 | (Chhuon et al., 2016) |
| Class C Orphans | GPRC5D | 6 | (Bräuner-Osborne et al., 2001) |

Frizzled Family GPCRs and the current level of evidence for their involvement in inflammation (see key above):

| | Level of | |
|---|---|---|
| Subtype | Evidence | Reference |
| FZD1 | 6 | (Neumann et al., 2010) |
| FZD2 | 6 | (Zhao et al., 1995) |
| FZD3 | 6 | (Lu et al., 2004) |
| FZD4 | 5 | (You et al., 2008) |
| FZD5 | 5 | (You et al., 2008) |
| FZD6 | 7 | (Wu et al., 2009) |
| FZD7 | 5 | (Wad a et al., 2013) |
| FZD8 | 5 | (Gregory et al., 2010) |
| FZD9 | 5 | (Wad a et al., 2013) |
| FZD10 | 1 | (Dijksterhuis et al., 2014) |
| SMO | 1 | (Dijksterhuis et al., 2014) |

Other 7TM proteins that have been classified as members of the GPCR superfamily and the current level of evidence for their involvement in inflammation (see key above):

| | Level of | |
|---|---|---|
| Subtype | Evidence | Reference |
| GPR107 | 5 | (Mo et al., 2013) |
| GPR137 | 4 | (Fischer et al., 2012) |
| OR51E1 | 6 | (Uhlén et al., 2015) |
| TPRA1 | 4 | (Guénard et al., 2015) |
| GPR143 | 6 | (Hohenhaus et al., 2013) |
| GPR157 | 4 | (Jia et al., 2012) |

In one embodiment, the certain activated co-located GPCRs of the invention are GPCRs selected from the group: ADGRA2, ADGRB2, ADGRB3, ADGRF3, ADGRG4, ADGRV1, CELSR1, CELSR2, CELSR3, OX1 receptor, OX2 receptor, PTH1 receptor, PTH2 receptor, AMY1 receptor, AMY2 receptor, AMY3 receptor, AM1 receptor, AM2 receptor, GPR63, GPR75, NMU2 receptor, OPN5, V1B receptor, y6 receptor, 5-HT4 receptor, GPR101, GPR119, GPR135, GPR137, GPR141, GPR149, GPR150, GPR151, GPR152, GPR157, GPR19, GPR25, GPR37, GPR37L1, GPR50, GPR62, LGR5, MRGPRE, MRGPRF, NTS2 receptor, OPN4, OPN4, OR10A7, OR10AG1, OR10Q1, OR10W1, OR12D3, OR13C2, OR13C3, OR13C4, OR13C5, OR13C8, OR13F1, OR13G1, OR1A2, OR1L1, OR1S1, OR1S2, OR2AK2, OR2D2, OR2D3, OR4A15, OR4C11, OR4C12, OR4C13, OR4C15, OR4C16, OR4K13, OR4K14, OR4K15, OR4K17, OR4N5, OR5AC2, OR5AK2, OR5AP2, OR5AR1, OR5AS1, OR5B12, OR5B17, OR5B2, OR5B21, OR5B3, OR5D13, OR5D14, OR5D16, OR5D18, OR5F1, OR51I, OR5J2, OR5K3, OR5L1, OR5L2, OR5M1, OR5M10, OR5M11, OR5M3, OR5M8, OR5M9, OR5R1, OR5T1, OR5T2, OR5T3, OR5W2, OR6C74, OR6K6, OR6M1, OR6Q1, OR6X1, OR8H1, OR8H2, OR8H3, OR8J1, OR8J3, OR8K1, OR8K3, OR8K5, OR8U1, OR8U8, OR9A4, OR9G1, OR9G4, OR9G9, OR9Q2, TAAR3, TPRA1, Y4 receptor, 5-HT1D receptor, 5-HT1E receptor, ADGRB1, AT2 receptor, BB1 receptor, BB3 receptor, CGRP receptor, CRF1 receptor, CRF2 receptor, ETA receptor, ETB receptor, FZD4, FZD5, FZD7, FZD8, FZD9, GABAB receptor, GABAB1, GABAB2, GAL1 receptor, GIP receptor, GLP-1 receptor, GLP-2 receptor, glucagon receptor, GnRH2 receptor, GPER, GPR107, GPR139, GPR156, GPR158, GPR161, GPR171, GPR179, GPR39, GPR45, GPR88, GPRC5A, GPRC5B, GPRC5C, H3 receptor, HCA1 receptor, LPA1 receptor, LPA3 receptor, LPA4 receptor, MC2 receptor, MC4 receptor, mGlu2 receptor, mGlu3 receptor, motilin receptor, MRGPRD, MRGPRX1, MRGPRX3, NK2 receptor, NPFF1 receptor, NPFF2 receptor, NPS receptor, NTS1 receptor, OR1D2, OR2AG1, OT receptor, PAC1 receptor, RXFP1 receptor, secretin receptor, TSH receptor, UT receptor, VIA receptor, V2 receptor, α2A-adrenoceptor, α2B-adrenoceptor, α2C-adrenoceptor, β1-adrenoceptor, β3-adrenoceptor, 5-HT1B receptor, 5-HT1F receptor, 5-HT2B receptor, 5-HT2C receptor, 5-HT5A receptor, 5-HT6 receptor, 5-HT7 receptor, ADGRE4P, ADGRF1, ADGRG1, ADGRG3, ADGRG5, calcitonin receptor-like receptor, CB1 receptor, CB2 receptor, CCK1 receptor, CCK2 receptor, CT receptor, D1 receptor, D2 receptor, D3 receptor, D4 receptor, D5 receptor, FFA1 receptor, FFA3 receptor, FSH receptor, FZD1, FZD2, FZD3, GHRH receptor, GnRH1 receptor, GPBA receptor, GPR1, GPR119, GPR12, GPR142, GPR143, GPR146, GPR148, GPR153, GPR160, GPR162, GPR17, GPR173, GPR174, GPR176, GPR18, GPR182, GPR20, GPR22, GPR26, GPR27, GPR3, GPR33, GPR35, GPR6, GPR61, GPR78, GPR82, GPR83, GPR84, GPR85, GPR87, GPRC5D, GPRC6 receptor, HCA2 receptor, HCA3 receptor, kisspeptin receptor, LGR4, LGR6, LH receptor, LPA2 receptor, LPA6 receptor, M1 receptor, M2 receptor, M3 receptor, M4 receptor, M5 receptor, MAS1L, MC3 receptor, MC5 receptor, MCH2 receptor, mGlu4 receptor, mGlu7 receptor, mGlu8 receptor, MRGPRG, NOP receptor, NPBW1 receptor, NPBW2 receptor, OPN3, OR11H1, OR2A1, OR2A2, OR2A4, OR2A42, OR2A7, OR2B11, OR2B6, OR2C1, OR2C3, OR2J3, OR2L13, OR2T11, OR2T34, OR2W3, OR3A3, OR4D10, OR4M1, OR4Q3, OR51A2, OR51A4, OR51A7, OR51B2, OR51B4, OR51B5, OR51B6, OR51D1, OR51E1, OR51E1, OR51E2, OR51F1, OR51F2, OR51G1, OR51G2, OR51I1, OR51I2, OR51J1, OR51L1, OR51M1, OR51Q1, OR51S1, OR51T1, OR51V1, OR52A1, OR52A4, OR52A5, OR52B2, OR52B4, OR52B6, OR52D1, OR52E2, OR52E4, OR52E5, OR52E6, OR52E8, OR52H1, OR52I1, OR52I2, OR52J3, OR52K1, OR52K2, OR52L1, OR52M1, OR52N1, OR52N2, OR52N4, OR52N5, OR52R1, OR52W1, OR56A1, OR56A3, OR56A4, OR56A5, OR56B1, OR56B4, OR6V1, OR7D2, OR9A2, oxoglutarate receptor, P2RY10, P2RY8, P2Y12 receptor, P2Y4 receptor, PrRP receptor, QRFP receptor, RXFP2 receptor, RXFP4 receptor, sst1 receptor, sst2 receptor, sst3 receptor, sst4 receptor, sst5 receptor, TA1 receptor, TAAR2, TAAR5, TAAR6, TAAR8, TAAR9, TAS1R1, TAS1R2, TAS1R3, TAS2R1, TAS2R10, TAS2R13, TAS2R14, TAS2R16, TAS2R19, TAS2R20, TAS2R3, TAS2R30, TAS2R31, TAS2R38, TAS2R39, TAS2R4, TAS2R40, TAS2R41, TAS2R42, TAS2R43, TAS2R45, TAS2R46, TAS2R5, TAS2R50, TAS2R60, TAS2R7, TAS2R8, TAS2R9, TRH1 receptor, Y1 receptor, Y2 receptor, Y5 receptor, α1A-adrenoceptor, α1B-adrenoceptor, α1D-adrenoceptor, δ receptor, 5-HT1A receptor, 5-HT2A receptor, A1 receptor, A2A receptor, A2B receptor, A3 receptor, ACKR1, ACKR2, ACKR3, ACKR4, ADGRE1, ADGRE2, ADGRE3, ADGRE5, apelin receptor, AT1 receptor, B1 receptor, B2 receptor, BB2 (GRP) receptor, BLT1 receptor, BLT2 receptor, C3a receptor, C5a1 receptor, C5a2 receptor, CaS receptor, CCR1, CCR10, CCR2, CCR3, CCR4, CCR5, CCR6, CCR7, CCR8, CCR9, CCRL2, chemerin receptor, CX3CR1, CXCR1, CXCR2, CXCR3, CXCR4, CXCR5, CXCR6, CysLT1 receptor, CysLT2 receptor, DP1 receptor, DP2 receptor, EP1 receptor, EP2 receptor, EP3 receptor, EP4 receptor, FFA2 receptor, FFA4 receptor, FP receptor, FPR1, FPR2/ALX, FPR2/ALX, FPR3, FZD6, GAL2 receptor, GAL3 receptor, ghrelin receptor, GPR132, GPR15, GPR18, GPR183, GPR21, GPR31, GPR32, GPR34, GPR4, GPR55, GPR55, GPR65, GPR68, H1 receptor, H2 receptor, H4 receptor, IP receptor, LPA5 receptor, MAS1, MC1 receptor, MCH1 receptor, mGlu1 receptor, mGlu5 receptor, MRGPRX2, MT1 receptor, MT2 receptor, NK1 receptor, NK3 receptor, NMU1 receptor, OXE receptor, P2Y1 receptor, P2Y11 receptor, P2Y13 receptor, P2Y14 receptor, P2Y2 receptor, P2Y6 receptor, PAF receptor, PAR1, PAR2, PAR3, PAR4, PKR1, PKR2, S1P1 receptor, S1P2 receptor, S1P3 receptor, S1P4 receptor, S1P5 receptor, succinate receptor, TP receptor, VPAC1 receptor, VPAC2 receptor, XCR1, β2-adrenoceptor, κ receptor, μ receptor.

In one embodiment, the certain activated co-located GPCRs of the invention are GPCRs selected from the group: OX1 receptor, OX2 receptor, PTH1 receptor, PTH2 receptor, AMY1 receptor, AMY2 receptor, AMY3 receptor, AM1 receptor, AM2 receptor, GPR63, GPR75, NMU2 receptor, OPN5, V1B receptor, y6 receptor, 5-HT4 receptor, GPR101, GPR119, GPR135, GPR137, GPR141, GPR149, GPR150, GPR151, GPR152, GPR157, GPR19, GPR25, GPR37, GPR37L1, GPR50, GPR62, LGR5, MRGPRE, MRGPRF, NTS2 receptor, OPN4, OPN4, OR10A7, OR10AG1, OR10Q1, OR10W1, OR12D3, OR13C2, OR13C3, OR13C4, OR13C5, OR13C8, OR13F1, OR13G1, OR1A2, OR1L1, OR1S1, OR1S2, OR2AK2, OR2D2, OR2D3, OR4A15, OR4C11, OR4C12, OR4C13, OR4C15, OR4C16, OR4K13, OR4K14, OR4K15, OR4K17, OR4N5, OR5AC2, OR5AK2, OR5AP2, OR5AR1, OR5AS1, OR5B12, OR5B17, OR5B2, OR5B21, OR5B3, OR5D13, OR5D14, OR5D16, OR5D18, OR5F1, OR51I, OR5J2, OR5K3, OR5L1, OR5L2, OR5M1, OR5M10, OR5M11, OR5M3, OR5M8, OR5M9, OR5R1, OR5T1, OR5T2, OR5T3, OR5W2, OR6C74, OR6K6, OR6M1, OR6Q1, OR6X1, OR8H1, OR8H2, OR8H3, OR8J1, OR8J3, OR8K1, OR8K3, OR8K5, OR8U1, OR8U8, OR9A4, OR9G1, OR9G4, OR9G9, OR9Q2, TAAR3, TPRA1, Y4 receptor, 5-HT1D receptor, 5-HT1E receptor, ADGRB1, AT2 receptor, BB1 receptor, BB3 receptor, CGRP receptor, CRF1 receptor, CRF2 receptor, ETA receptor, ETB receptor, FZD4, FZD5, FZD7, FZD8, FZD9, GABAB receptor, GABAB1, GABAB2, GAL1 receptor, GIP receptor, GLP-1 receptor, GLP-2 receptor, glucagon receptor, GnRH2 receptor, GPER, GPR107, GPR139, GPR156, GPR158, GPR161, GPR171, GPR179, GPR39, GPR45, GPR88, GPRC5A, GPRC5B, GPRC5C, H3 receptor, HCA1 receptor, LPA1 receptor, LPA3 receptor, LPA4 receptor, MC2 receptor, MC4 receptor, mGlu2 receptor, mGlu3 receptor, motilin receptor, MRGPRD, MRGPRX1, MRGPRX3, NK2 receptor, NPFF1 receptor, NPFF2 receptor, NPS receptor, NTS1 receptor, OR1D2, OR2AG1, OT receptor, PAC1 receptor, RXFP1 receptor, secretin receptor, TSH receptor, UT receptor, VIA receptor, V2 receptor, α2A-adrenoceptor, α2B-adrenoceptor, α2C-adrenoceptor, β1-adrenoceptor, β3-adrenoceptor, 5-HT1B receptor, 5-HT1F receptor, 5-HT2B receptor, 5-HT2C receptor, 5-HT5A receptor, 5-HT6 receptor, 5-HT7 receptor, ADGRE4P, ADGRF1, ADGRG1, ADGRG3, ADGRG5, calcitonin receptor-like receptor, CB1 receptor, CB2 receptor, CCK1 receptor, CCK2 receptor, CT receptor, D1 receptor, D2 receptor, D3 receptor, D4 receptor, D5 receptor, FFA1 receptor, FFA3 receptor, FSH receptor, FZD1, FZD2, FZD3, GHRH receptor, GnRH1 receptor, GPBA receptor, GPR1, GPR119, GPR12, GPR142, GPR143, GPR146, GPR148, GPR153, GPR160, GPR162, GPR17, GPR173, GPR174, GPR176, GPR18, GPR182, GPR20, GPR22, GPR26, GPR27, GPR3, GPR33, GPR35, GPR6, GPR61, GPR78, GPR82, GPR83, GPR84, GPR85, GPR87, GPRC5D, GPRC6 receptor, HCA2 receptor, HCA3 receptor, kisspeptin receptor, LGR4, LGR6, LH receptor, LPA2 receptor, LPA6 receptor, M1 receptor, M2 receptor, M3 receptor, M4 receptor, M5 receptor, MAS1L, MC3 receptor, MC5 receptor, MCH2 receptor, mGlu4 receptor, mGlu7 receptor, mGlu8 receptor, MRGPRG, NOP receptor, NPBW1 receptor, NPBW2 receptor, OPN3, OR11H1, OR2A1, OR2A2, OR2A4, OR2A42, OR2A7, OR2B11, OR2B6, OR2C1, OR2C3, OR2J3, OR2L13, OR2T11, OR2T34, OR2W3, OR3A3, OR4D10, OR4M1, OR4Q3, OR51A2, OR51A4, OR51A7, OR51B2, OR51B4, OR51B5, OR51B6, OR51D1, OR51E1, OR51E1, OR51E2, OR51F1, OR51F2, OR51G1, OR51G2, OR51I1, OR51I2, OR51J1, OR51L1, OR51M1, OR51Q1, OR51S1, OR51T1, OR51V1, OR52A1, OR52A4, OR52A5, OR52B2, OR52B4, OR52B6, OR52D1, OR52E2, OR52E4, OR52E5, OR52E6, OR52E8, OR52H1, OR52I1, OR52I2, OR52J3, OR52K1, OR52K2, OR52L1, OR52M1, OR52N1, OR52N2, OR52N4, OR52N5, OR52R1, OR52W1, OR56A1, OR56A3, OR56A4, OR56A5, OR56B1, OR56B4, OR6V1, OR7D2, OR9A2, oxoglutarate receptor, P2RY10, P2RY8, P2Y12 receptor, P2Y4 receptor, PrRP receptor, QRFP receptor, RXFP2 receptor, RXFP4 receptor, sst1 receptor, sst2 receptor, sst3 receptor, sst4 receptor, sst5 receptor, TA1 receptor, TAAR2, TAAR5, TAAR6, TAAR8, TAAR9, TAS1R1, TAS1R2, TAS1R3, TAS2R1, TAS2R10, TAS2R13, TAS2R14, TAS2R16, TAS2R19, TAS2R20, TAS2R3, TAS2R30, TAS2R31, TAS2R38, TAS2R39, TAS2R4, TAS2R40, TAS2R41, TAS2R42, TAS2R43, TAS2R45, TAS2R46, TAS2R5, TAS2R50, TAS2R60, TAS2R7, TAS2R8, TAS2R9, TRH1 receptor, Y1 receptor, Y2 receptor, Y5 receptor, α1A-adrenoceptor, α1B-adrenoceptor, α1D-adrenoceptor, δ receptor, 5-HT1A receptor, 5-HT2A receptor, A1 receptor, A2A receptor, A2B receptor, A3 receptor, ACKR1, ACKR2, ACKR3, ACKR4, ADGRE1, ADGRE2, ADGRE3, ADGRE5, apelin receptor, AT1 receptor, B1 receptor, B2 receptor, BB2 (GRP) receptor, BLT1 receptor, BLT2 receptor, C3a receptor, C5a1 receptor, C5a2 receptor, CaS receptor, CCR1, CCR10, CCR2, CCR3, CCR4, CCR5, CCR6, CCR7, CCR8, CCR9, CCRL2, chemerin receptor, CX3CR1, CXCR1, CXCR2, CXCR3, CXCR4, CXCR5, CXCR6, CysLT1 receptor, CysLT2 receptor, DP1 receptor, DP2 receptor, EP1 receptor, EP2 receptor, EP3 receptor, EP4 receptor, FFA2 receptor, FFA4 receptor, FP receptor, FPR1, FPR2/ALX, FPR2/ALX, FPR3, FZD6, GAL2 receptor, GAL3 receptor, ghrelin receptor, GPR132, GPR15, GPR18, GPR183, GPR21, GPR31, GPR32, GPR34, GPR4, GPR55, GPR55, GPR65, GPR68, H1 receptor, H2 receptor, H4 receptor, IP receptor, LPA5 receptor, MAS1, MC1 receptor, MCH1 receptor, mGlu1 receptor, mGlu5 receptor, MRGPRX2, MT1 receptor, MT2 receptor, NK1 receptor, NK3 receptor, NMU1 receptor, OXE receptor, P2Y1 receptor, P2Y11 receptor, P2Y13 receptor, P2Y14 receptor, P2Y2 receptor, P2Y6 receptor, PAF receptor, PAR1, PAR2, PAR3, PAR4, PKR1, PKR2, S1P1 receptor, S1P2 receptor, S1P3 receptor, S1P4 receptor, S1P5 receptor, succinate receptor, TP receptor, VPAC1 receptor, VPAC2 receptor, XCR1, β2-adrenoceptor, κ receptor, μ receptor.

In one embodiment, the certain activated co-located GPCRs of the invention are GPCRs selected from the group: 5-HT4 receptor, GPR101, GPR119, GPR135, GPR137, GPR141, GPR149, GPR150, GPR151, GPR152, GPR157, GPR19, GPR25, GPR37, GPR37L1, GPR50, GPR62, LGR5, MRGPRE, MRGPRF, NTS2 receptor, OPN4, OPN4, OR10A7, OR10AG1, OR10Q1, OR10W1, OR12D3, OR13C2, OR13C3, OR13C4, OR13C5, OR13C8, OR13F1, OR13G1, OR1A2, OR1L1, OR1S1, OR1S2, OR2AK2, OR2D2, OR2D3, OR4A15, OR4C11, OR4C12, OR4C13, OR4C15, OR4C16, OR4K13, OR4K14, OR4K15, OR4K17, OR4N5, OR5AC2, OR5AK2, OR5AP2, OR5AR1, OR5AS1, OR5B12, OR5B17, OR5B2, OR5B21, OR5B3, OR5D13, OR5D14, OR5D16, OR5D18, OR5F1, OR51I, OR5J2, OR5K3, OR5L1, OR5L2, OR5M1, OR5M10, OR5M11, OR5M3, OR5M8, OR5M9, OR5R1, OR5T1, OR5T2, OR5T3, OR5W2, OR6C74, OR6K6, OR6M1, OR6Q1, OR6X1, OR8H1, OR8H2, OR8H3, OR8J1, OR8J3, OR8K1, OR8K3, OR8K5, OR8U1, OR8U8, OR9A4, OR9G1, OR9G4, OR9G9, OR9Q2, TAAR3, TPRA1, Y4 receptor, 5-HT1D receptor, 5-HT1E receptor, ADGRB1, AT2 receptor, BB1 receptor, BB3 receptor, CGRP receptor, CRF1 receptor, CRF2 receptor, ETA receptor, ETB receptor, FZD4, FZD5, FZD7, FZD8, FZD9, GABAB receptor, GABAB1, GABAB2, GAL1 receptor, GIP receptor, GLP-1 receptor, GLP-2 receptor, glucagon receptor, GnRH2 receptor, GPER, GPR107, GPR139, GPR156, GPR158, GPR161, GPR171, GPR179, GPR39, GPR45, GPR88, GPRC5A, GPRC5B, GPRC5C, H3 receptor, HCA1 receptor, LPA1 receptor, LPA3 receptor, LPA4 receptor, MC2 receptor, MC4 receptor, mGlu2 receptor, mGlu3 receptor, motilin receptor, MRGPRD, MRGPRX1, MRGPRX3, NK2 receptor, NPFF1 receptor, NPFF2 receptor, NPS receptor, NTS1 receptor, OR1D2, OR2AG1, OT receptor, PAC1 receptor, RXFP1 receptor, secretin receptor, TSH receptor, UT receptor, V1A receptor, V2 receptor, α2A-adrenoceptor, α2B-adrenoceptor, α2C-adrenoceptor, β1-adrenoceptor, β3-adrenoceptor, 5-HT1B receptor, 5-HT1F receptor, 5-HT2B receptor, 5-HT2C receptor, 5-HT5A receptor, 5-HT6 receptor, 5-HT7 receptor, ADGRE4P, ADGRF1, ADGRG1, ADGRG3, ADGRG5, calcitonin receptor-like receptor, CB1 receptor, CB2 receptor, CCK1 receptor, CCK2 receptor, CT receptor, D1 receptor, D2 receptor, D3 receptor, D4 receptor, D5 receptor, FFA1 receptor, FFA3 receptor, FSH receptor, FZD1, FZD2, FZD3, GHRH receptor, GnRH1 receptor, GPBA receptor, GPR1, GPR119, GPR12, GPR142, GPR143, GPR146, GPR148, GPR153, GPR160, GPR162, GPR17, GPR173, GPR174, GPR176, GPR18, GPR182, GPR20, GPR22, GPR26, GPR27, GPR3, GPR33, GPR35, GPR6, GPR61, GPR78, GPR82, GPR83, GPR84, GPR85, GPR87, GPRC5D, GPRC6 receptor, HCA2 receptor, HCA3 receptor, kisspeptin receptor, LGR4, LGR6, LH receptor, LPA2 receptor, LPA6 receptor, M1 receptor, M2 receptor, M3 receptor, M4 receptor, M5 receptor, MAS1L, MC3 receptor, MC5 receptor, MCH2 receptor, mGlu4 receptor, mGlu7 receptor, mGlu8 receptor, MRGPRG, NOP receptor, NPBW1 receptor, NPBW2 receptor, OPN3, OR11H1, OR2A1, OR2A2, OR2A4, OR2A42, OR2A7, OR2B11, OR2B6, OR2C1, OR2C3, OR2J3, OR2L13, OR2T11, OR2T34, OR2W3, OR3A3, OR4D10, OR4M1, OR4Q3, OR51A2, OR51A4, OR51A7, OR51B2, OR51B4, OR51B5, OR51B6, OR51D1, OR51E1, OR51E1, OR51E2, OR51F1, OR51F2, OR51G1, OR51G2, OR51I1, OR51I2, OR51J1, OR51L1, OR51M1, OR51Q1, OR51S1, OR51T1, OR51V1, OR52A1, OR52A4, OR52A5, OR52B2, OR52B4, OR52B6, OR52D1, OR52E2, OR52E4, OR52E5, OR52E6, OR52E8, OR52H1, OR52I1, OR52I2, OR52J3, OR52K1, OR52K2, OR52L1, OR52M1, OR52N1, OR52N2, OR52N4, OR52N5, OR52R1, OR52W1, OR56A1, OR56A3, OR56A4, OR56A5, OR56B1, OR56B4, OR6V1, OR7D2, OR9A2, oxoglutarate receptor, P2RY10, P2RY8, P2Y12 receptor, P2Y4 receptor, PrRP receptor, QRFP receptor, RXFP2 receptor, RXFP4 receptor, sst1 receptor, sst2 receptor, sst3 receptor, sst4 receptor, sst5 receptor, TA1 receptor, TAAR2, TAAR5, TAAR6, TAAR8, TAAR9, TAS1R1, TAS1R2, TAS1R3, TAS2R1, TAS2R10, TAS2R13, TAS2R14, TAS2R16, TAS2R19, TAS2R20, TAS2R3, TAS2R30, TAS2R31, TAS2R38, TAS2R39, TAS2R4, TAS2R40, TAS2R41, TAS2R42, TAS2R43, TAS2R45, TAS2R46, TAS2R5, TAS2R50, TAS2R60, TAS2R7, TAS2R8, TAS2R9, TRH1 receptor, Y1 receptor, Y2 receptor, Y5 receptor, α1A-adrenoceptor, α1B-adrenoceptor, α1D-adrenoceptor, δ receptor, 5-HT1A receptor, 5-HT2A receptor, A1 receptor, A2A receptor, A2B receptor, A3 receptor, ACKR1, ACKR2, ACKR3, ACKR4, ADGRE1, ADGRE2, ADGRE3, ADGRE5, apelin receptor, AT1 receptor, B1 receptor, B2 receptor, BB2 (GRP) receptor, BLT1 receptor, BLT2 receptor, C3a receptor, C5a1 receptor, C5a2 receptor, CaS receptor, CCR1, CCR10, CCR2, CCR3, CCR4, CCR5, CCR6, CCR7, CCR8, CCR9, CCRL2, chemerin receptor, CX3CR1, CXCR1, CXCR2, CXCR3, CXCR4, CXCR5, CXCR6, CysLT1 receptor, CysLT2 receptor, DP1 receptor, DP2 receptor, EP1 receptor, EP2 receptor, EP3 receptor, EP4 receptor, FFA2 receptor, FFA4 receptor, FP receptor, FPR1, FPR2/ALX, FPR2/ALX, FPR3, FZD6, GAL2 receptor, GAL3 receptor, ghrelin receptor, GPR132, GPR15, GPR18, GPR183, GPR21, GPR31, GPR32, GPR34, GPR4, GPR55, GPR55, GPR65, GPR68, H1 receptor, H2 receptor, H4 receptor, IP receptor, LPA5 receptor, MAS1, MC1 receptor, MCH1 receptor, mGlu1 receptor, mGlu5 receptor, MRGPRX2, MT1 receptor, MT2 receptor, NK1 receptor, NK3 receptor, NMU1 receptor, OXE receptor, P2Y1 receptor, P2Y11 receptor, P2Y13 receptor, P2Y14 receptor, P2Y2 receptor, P2Y6 receptor, PAF receptor, PAR1, PAR2, PAR3, PAR4, PKR1, PKR2, S1P1 receptor, S1P2 receptor, S1P3 receptor, S1P4 receptor, S1P5 receptor, succinate receptor, TP receptor, VPAC1 receptor, VPAC2 receptor, XCR1, β2-adrenoceptor, κ receptor, μ receptor.

In one embodiment, the certain activated co-located GPCRs of the invention are GPCRs selected from the group: 5-HT1 D receptor, 5-HT1E receptor, ADGRB1, AT2 receptor, BB1 receptor, BB3 receptor, CGRP receptor, CRF1 receptor, CRF2 receptor, ETA receptor, ETB receptor, FZD4, FZD5, FZD7, FZD8, FZD9, GABAB receptor, GABAB1, GABAB2, GAL1 receptor, GIP receptor, GLP-1 receptor, GLP-2 receptor, glucagon receptor, GnRH2 receptor, GPER, GPR107, GPR139, GPR156, GPR158, GPR161, GPR171, GPR179, GPR39, GPR45, GPR88, GPRC5A, GPRC5B, GPRC5C, H3 receptor, HCA1 receptor, LPA1 receptor, LPA3 receptor, LPA4 receptor, MC2 receptor, MC4 receptor, mGlu2 receptor, mGlu3 receptor, motilin receptor, MRGPRD, MRGPRX1, MRGPRX3, NK2 receptor, NPFF1 receptor, NPFF2 receptor, NPS receptor, NTS1 receptor, OR1D2, OR2AG1, OT receptor, PAC1 receptor, RXFP1 receptor, secretin receptor, TSH receptor, UT receptor, VIA receptor, V2 receptor, α2A-adrenoceptor, α2B-adrenoceptor, α2C-adrenoceptor, β1-adrenoceptor, β3-adrenoceptor, 5-HT1B receptor, 5-HT1F receptor, 5-HT2B receptor, 5-HT2C receptor, 5-HT5A receptor, 5-HT6 receptor, 5-HT7 receptor, ADGRE4P, ADGRF1, ADGRG1, ADGRG3, ADGRG5, calcitonin receptor-like receptor, CB1 receptor, CB2 receptor, CCK1 receptor, CCK2 receptor, CT receptor, D1 receptor, D2 receptor, D3 receptor, D4 receptor, D5 receptor, FFA1 receptor, FFA3 receptor, FSH receptor, FZD1, FZD2, FZD3, GHRH receptor, GnRH1 receptor, GPBA receptor, GPR1, GPR119, GPR12, GPR142, GPR143, GPR146, GPR148, GPR153, GPR160, GPR162, GPR17, GPR173, GPR174, GPR176, GPR18, GPR182, GPR20, GPR22, GPR26, GPR27, GPR3, GPR33, GPR35, GPR6, GPR61, GPR78, GPR82, GPR83, GPR84, GPR85, GPR87, GPRC5D, GPRC6 receptor, HCA2 receptor, HCA3 receptor, kisspeptin receptor, LGR4, LGR6, LH receptor, LPA2 receptor, LPA6 receptor, M1 receptor, M2 receptor, M3 receptor, M4 receptor, M5 receptor, MAS1L, MC3 receptor, MC5 receptor, MCH2 receptor, mGlu4 receptor, mGlu7 receptor, mGlu8 receptor, MRGPRG, NOP receptor, NPBW1 receptor, NPBW2 receptor, OPN3, OR11H1, OR2A1, OR2A2, OR2A4, OR2A42, OR2A7, OR2B11, OR2B6, OR2C1, OR2C3, OR2J3, OR2L13, OR2T11, OR2T34, OR2W3, OR3A3, OR4D10, OR4M1, OR4Q3, OR51A2, OR51A4, OR51A7, OR51B2, OR51B4, OR51B5, OR51B6, OR51D1, OR51E1, OR51E1, OR51E2, OR51F1, OR51F2, OR51G1, OR51G2, OR51I1, OR51I2, OR51J1, OR51L1, OR51M1, OR51Q1, OR51S1, OR51T1, OR51V1, OR52A1, OR52A4, OR52A5, OR52B2, OR52B4, OR52B6, OR52D1, OR52E2, OR52E4, OR52E5, OR52E6, OR52E8, OR52H1, OR52I1, OR52I2, OR52J3, OR52K1, OR52K2, OR52L1, OR52M1, OR52N1, OR52N2, OR52N4, OR52N5, OR52R1, OR52W1, OR56A1, OR56A3, OR56A4, OR56A5, OR56B1, OR56B4, OR6V1, OR7D2, OR9A2, oxoglutarate receptor, P2RY10, P2RY8, P2Y12 receptor, P2Y4 receptor, PrRP receptor, QRFP receptor, RXFP2 receptor, RXFP4 receptor, sst1 receptor, sst2 receptor, sst3 receptor, sst4 receptor, sst5 receptor, TA1 receptor, TAAR2, TAAR5, TAAR6, TAAR8, TAAR9, TAS1R1, TAS1R2, TAS1R3, TAS2R1, TAS2R10, TAS2R13, TAS2R14, TAS2R16, TAS2R19, TAS2R20, TAS2R3, TAS2R30, TAS2R31, TAS2R38, TAS2R39, TAS2R4, TAS2R40, TAS2R41, TAS2R42, TAS2R43, TAS2R45, TAS2R46, TAS2R5, TAS2R50, TAS2R60, TAS2R7, TAS2R8, TAS2R9, TRH1 receptor, Y1 receptor, Y2 receptor, Y5 receptor, α1A-adrenoceptor, α1B-adrenoceptor, α1D-adrenoceptor, δ receptor, 5-HT1A receptor, 5-HT2A receptor, A1 receptor, A2A receptor, A2B receptor, A3 receptor, ACKR1, ACKR2, ACKR3, ACKR4, ADGRE1, ADGRE2, ADGRE3, ADGRE5, apelin receptor, AT1 receptor, B1 receptor, B2 receptor, BB2 (GRP) receptor, BLT1 receptor, BLT2 receptor, C3a receptor, C5a1 receptor, C5a2 receptor, CaS receptor, CCR1, CCR10, CCR2, CCR3, CCR4, CCR5, CCR6, CCR7, CCR8, CCR9, CCRL2, chemerin receptor, CX3CR1, CXCR1, CXCR2, CXCR3, CXCR4, CXCR5, CXCR6, CysLT1 receptor, CysLT2 receptor, DP1 receptor, DP2 receptor, EP1 receptor, EP2 receptor, EP3 receptor, EP4 receptor, FFA2 receptor, FFA4 receptor, FP receptor, FPR1, FPR2/ALX, FPR2/ALX, FPR3, FZD6, GAL2 receptor, GAL3 receptor, ghrelin receptor, GPR132, GPR15, GPR18, GPR183, GPR21, GPR31, GPR32, GPR34, GPR4, GPR55, GPR55, GPR65, GPR68, H1 receptor, H2 receptor, H4 receptor, IP receptor, LPA5 receptor, MAS1, MC1 receptor, MCH1 receptor, mGlu1 receptor, mGlu5 receptor, MRGPRX2, MT1 receptor, MT2 receptor, NK1 receptor, NK3 receptor, NMU1 receptor, OXE receptor, P2Y1 receptor, P2Y11 receptor, P2Y13 receptor, P2Y14 receptor, P2Y2 receptor, P2Y6 receptor, PAF receptor, PAR1, PAR2, PAR3, PAR4, PKR1, PKR2, S1P1 receptor, S1P2 receptor, S1P3 receptor, S1P4 receptor, S1P5 receptor, succinate receptor, TP receptor, VPAC1 receptor, VPAC2 receptor, XCR1, β2-adrenoceptor, κ receptor, μ receptor.

In one embodiment, the certain activated co-located GPCRs of the invention are GPCRs selected from the group: 5-HT1B receptor, 5-HT1F receptor, 5-HT2B receptor, 5-HT2C receptor, 5-HT5A receptor, 5-HT6 receptor, 5-HT7 receptor, ADGRE4P, ADGRF1, ADGRG1, ADGRG3, ADGRG5, calcitonin receptor-like receptor, CB1 receptor, CB2 receptor, CCK1 receptor, CCK2 receptor, CT receptor, D1 receptor, D2 receptor, D3 receptor, D4 receptor, D5 receptor, FFA1 receptor, FFA3 receptor, FSH receptor, FZD1, FZD2, FZD3, GHRH receptor, GnRH1 receptor, GPBA receptor, GPR1, GPR119, GPR12, GPR142, GPR143, GPR146, GPR148, GPR153, GPR160, GPR162, GPR17, GPR173, GPR174, GPR176, GPR18, GPR182, GPR20, GPR22, GPR26, GPR27, GPR3, GPR33, GPR35, GPR6, GPR61, GPR78, GPR82, GPR83, GPR84, GPR85, GPR87, GPRC5D, GPRC6 receptor, HCA2 receptor, HCA3 receptor, kisspeptin receptor, LGR4, LGR6, LH receptor, LPA2 receptor, LPA6 receptor, M1 receptor, M2 receptor, M3 receptor, M4 receptor, M5 receptor, MAS1L, MC3 receptor, MC5 receptor, MCH2 receptor, mGlu4 receptor, mGlu7 receptor, mGlu8 receptor, MRGPRG, NOP receptor, NPBW1 receptor, NPBW2 receptor, OPN3, OR11H1, OR2A1, OR2A2, OR2A4, OR2A42, OR2A7, OR2B11, OR2B6, OR2C1, OR2C3, OR2J3, OR2L13, OR2T11, OR2T34, OR2W3, OR3A3, OR4D10, OR4M1, OR4Q3, OR51A2, OR51A4, OR51A7, OR51B2, OR51B4, OR51B5, OR51B6, OR51D1, OR51E1, OR51E1, OR51E2, OR51F1, OR51F2, OR51G1, OR51G2, OR51I1, OR51I2, OR51J1, OR51L1, OR51M1, OR51Q1, OR51S1, OR51T1, OR51V1, OR52A1, OR52A4, OR52A5, OR52B2, OR52B4, OR52B6, OR52D1, OR52E2, OR52E4, OR52E5, OR52E6, OR52E8, OR52H1, OR52I1, OR52I2, OR52J3, OR52K1, OR52K2, OR52L1, OR52M1, OR52N1, OR52N2, OR52N4, OR52N5, OR52R1, OR52W1, OR56A1, OR56A3, OR56A4, OR56A5, OR56B1, OR56B4, OR6V1, OR7D2, OR9A2, oxoglutarate receptor, P2RY10, P2RY8, P2Y12 receptor, P2Y4 receptor, PrRP receptor, QRFP receptor, RXFP2 receptor, RXFP4 receptor, sst1 receptor, sst2 receptor, sst3 receptor, sst4 receptor, sst5 receptor, TA1 receptor, TAAR2, TAAR5, TAAR6, TAAR8, TAAR9, TAS1R1, TAS1R2, TAS1R3, TAS2R1, TAS2R10, TAS2R13, TAS2R14, TAS2R16, TAS2R19, TAS2R20, TAS2R3, TAS2R30, TAS2R31, TAS2R38, TAS2R39, TAS2R4, TAS2R40, TAS2R41, TAS2R42, TAS2R43, TAS2R45, TAS2R46, TAS2R5, TAS2R50, TAS2R60, TAS2R7, TAS2R8, TAS2R9, TRH1 receptor, Y1 receptor, Y2 receptor, Y5 receptor, α1A-adrenoceptor, al B-adrenoceptor, α1D-adrenoceptor, δ receptor, 5-HT1A receptor, 5-HT2A receptor, A1 receptor, A2A receptor, A2B receptor, A3 receptor, ACKR1, ACKR2, ACKR3, ACKR4, ADGRE1, ADGRE2, ADGRE3, ADGRE5, apelin receptor, AT1 receptor, B1 receptor, B2 receptor, BB2 (GRP) receptor, BLT1 receptor, BLT2 receptor, C3a receptor, C5a1 receptor, C5a2 receptor, CaS receptor, CCR1, CCR10, CCR2, CCR3, CCR4, CCR5, CCR6, CCR7, CCR8, CCR9, CCRL2, chemerin receptor, CX3CR1, CXCR1, CXCR2, CXCR3, CXCR4, CXCR5, CXCR6, CysLT1 receptor, CysLT2 receptor, DP1 receptor, DP2 receptor, EP1 receptor, EP2 receptor, EP3 receptor, EP4 receptor, FFA2 receptor, FFA4 receptor, FP receptor, FPR1, FPR2/ALX, FPR2/ALX, FPR3, FZD6, GAL2 receptor, GAL3 receptor, ghrelin receptor, GPR132, GPR15, GPR18, GPR183, GPR21, GPR31, GPR32, GPR34, GPR4, GPR55, GPR55, GPR65, GPR68, H1 receptor, H2 receptor, H4 receptor, IP receptor, LPA5 receptor, MAS1, MC1 receptor, MCH1 receptor, mGlu1 receptor, mGlu5 receptor, MRGPRX2, MT1 receptor, MT2 receptor, NK1 receptor, NK3 receptor, NMU1 receptor, OXE receptor, P2Y1 receptor, P2Y11 receptor, P2Y13 receptor, P2Y14 receptor, P2Y2 receptor, P2Y6 receptor, PAF receptor, PAR1, PAR2, PAR3, PAR4, PKR1, PKR2, S1P1 receptor, S1P2 receptor, S1P3 receptor, S1P4 receptor, S1P5 receptor, succinate receptor, TP receptor, VPAC1 receptor, VPAC2 receptor, XCR1, β2-adrenoceptor, κ receptor, μ receptor.

In one embodiment, the certain activated co-located GPCRs of the invention are GPCRs selected from the group: 5-HT1A receptor, 5-HT2A receptor, A1 receptor, A2A receptor, A2B receptor, A3 receptor, ACKR1, ACKR2, ACKR3, ACKR4, ADGRE1, ADGRE2, ADGRE3, ADGRE5, apelin receptor, AT1 receptor, B1 receptor, B2 receptor, BB2 (GRP) receptor, BLT1 receptor, BLT2 receptor, C3a receptor, C5a1 receptor, C5a2 receptor, CaS receptor, CCR1, CCR10, CCR2, CCR3, CCR4, CCR5, CCR6, CCR7, CCR8, CCR9, CCRL2, chemerin receptor, CX3CR1, CXCR1, CXCR2, CXCR3, CXCR4, CXCR5, CXCR6, CysLT1 receptor, CysLT2 receptor, DP1 receptor, DP2 receptor, EP1 receptor, EP2 receptor, EP3 receptor, EP4 receptor, FFA2 receptor, FFA4 receptor, FP receptor, FPR1, FPR2/ALX, FPR2/ALX, FPR3, FZD6, GAL2 receptor, GAL3 receptor, ghrelin receptor, GPR132, GPR15, GPR18, GPR183, GPR21, GPR31, GPR32, GPR34, GPR4, GPR55, GPR55, GPR65, GPR68, H1 receptor, H2 receptor, H4 receptor, IP receptor, LPA5 receptor, MAS1, MC1 receptor, MCH1 receptor, mGlu1 receptor, mGlu5 receptor, MRGPRX2, MT1 receptor, MT2 receptor, NK1 receptor, NK3 receptor, NMU1 receptor, OXE receptor, P2Y1 receptor, P2Y11 receptor, P2Y13 receptor, P2Y14 receptor, P2Y2 receptor, P2Y6 receptor, PAF receptor, PAR1, PAR2, PAR3, PAR4, PKR1, PKR2, S1P1 receptor, S1P2 receptor, S1P3 receptor, S1P4 receptor, S1P5 receptor, succinate receptor, TP receptor, VPAC1 receptor, VPAC2 receptor, XCR1, β2-adrenoceptor, κ receptor, μ receptor.

In one embodiment, the certain activated co-located GPCRs of the invention are GPCRs selected from the group: AT1 receptor, vasopressin receptor V2R, S1P1 receptor, β2-adrenoceptor, orexin receptor 2, TRH receptor 1, CCR1, CCR2, CCR6, CCR7, CXCR2, CXCR4, CXCR6, somatostatin receptor 3, C5a receptor.

In one embodiment, the certain activated co-located GPCRs of the invention are GPCRs selected from the group: AT1 receptor, vasopressin receptor V2R, S1P1 receptor, β2-adrenoceptor, orexin receptor 2, TRH receptor 1, CCR1, CCR2, CCR6, CCR7, CXCR2, CXCR6, somatostatin receptor 3, C5a receptor.

In one embodiment, the certain activated co-located GPCRs of the invention are selected from the group: AT1 receptor and C5a receptor.

In one embodiment, the certain activated co-located GPCR of the invention is AT1 receptor.

In one embodiment, certain chemokine receptors are chemokine receptors that are co-expressed in the same cell as an IgSF CAM.

In one embodiment, certain chemokine receptors are chemokine receptors that are co-expressed in the same cell as an IgSF CAM, are implicated in inflammation, and are selected from the group: CCR1, CCR2, CCR3, CCR4, CCR5, CCR6, CCR7, CCR8, CCR9, CCR10, CXCR1, CXCR2, CXCR3, CXCR4, CXCR5, CXCR6, CXCR7, CX3CR1, XCR1.

In one embodiment, certain chemokine receptors are chemokine receptors that are co-expressed in the same cell as an IgSF CAM, are implicated in inflammation, and are selected from the group: CCR1, CCR2, CCR6, CCR7, CXCR2, CXCR4, CXCR6.

In one embodiment, certain chemokine receptors are chemokine receptors that are co-expressed in the same cell as an IgSF CAM, are implicated in inflammation, and are selected from the group: CCR1, CCR2, CCR6, CCR7, CXCR2, CXCR6.

In one form of the invention, an IgSF CAM-independent certain co-located GPCR signalling pathway is the Gq signalling pathway. In one form of the invention, an IgSF CAM-independent certain co-located GPCR signalling pathway is the Gi/o signalling pathway. In one form of the invention, an IgSF CAM-independent certain co-located GPCR signalling pathway is the Gs signalling pathway. In one form of the invention, an IgSF CAM-independent certain co-located GPCR signalling pathway is the calcium signalling pathway. In one form of the invention, an IgSF CAM-independent certain co-located GPCR signalling pathway is the phospholipase C signalling pathway. In another form of the invention, the an IgSF CAM-independent certain co-located GPCR signalling pathway is 8-arrestin-mediated extracellular regulated kinase (ERK) signalling.

In a particularly preferred embodiment, where the activated co-located GPCR is activated AT₁R, modulators of the invention do not modulate, or modulate to a lesser extent, one or more an IgSF CAM independent AT₁R signalling pathways.

In a particularly preferred embodiment, where the activated co-located GPCR is activated AT₁R, modulators of the invention do not inhibit, or inhibit to a lesser extent, one or more an IgSF CAM independent AT₁R signalling pathways.

In one form of the invention, an IgSF CAM-independent AT₁R signalling pathway is the Gq signalling pathway. In another form of the invention, an IgSF CAM-independent AT₁R signalling pathway is 8-arrestin-mediated extracellular regulated kinase (ERK) signalling.

In one form of the invention, an IgSF CAM-independent AT₁R signalling pathway is the Gi/o signalling pathway. In another form of the invention, an IgSF CAM-independent CCR2 signalling pathway is 8-arrestin-mediated extracellular regulated kinase (ERK) signalling. In another form of the invention, n IgSF CAM-independent CCR2 signalling pathway is the phospholipase C signalling pathway.

3. Modulators of IgSF CAM Ligand-Dependent Activation of an IgSF CAM

In one form of the invention, an IgSF CAM ligand is a ligand that interacts with the ectodomain of an IgSF CAM to modulate activation of an IgSF CAM.

Preferably, an IgSF CAM ligand is a ligand that interacts with the ectodomain of an IgSF CAM to modulate activation of an IgSF CAM and does not interact with the transmembrane domain or cytosolic tail of an IgSF CAM or motifs contained therein.

In one form of the invention, an IgSF CAM ligand is a ligand that interacts with the extracellular V and/or C domains of an IgSF CAM ectodomain to activate an IgSF CAM. Preferably, an IgSF CAM ligand does not interact with the transmembrane domain or cytosolic tail of an IgSF CAM or motifs contained therein.

In one form, the present invention comprises modulators of IgSF CAM activity where such IgSF CAM activity is induced by its cognate ligand and where the modulators of IgSF CAM activity are analogues, fragments or derivatives of an IgSF CAM.

In one form, the present invention comprises modulators of IgSF CAM activity where such IgSF CAM activity is induced by its cognate ligand and where the modulators of IgSF CAM activity are analogues, fragments or derivatives of the C-terminal tail of an IgSF CAM.

In one form, the present invention comprises modulators of IgSF CAM activity where such IgSF CAM activity is induced by its cognate ligand and where the modulators of IgSF CAM activity are analogues, fragments or derivatives of the C-terminal tail of an IgSF CAM lacking serines or threonines, or with serines and threonines selectively mutated to other residues.

In one form, the present invention comprises modulators of IgSF CAM activity where such IgSF CAM activity is induced by its cognate ligand and where the modulators of IgSF CAM activity are analogues, fragments or derivatives of the C-terminal tail of IgSF CAM lacking serines or threonines, or with serines and threonines mutated to other residues that are not negatively charged.

In one form of the invention, a modulator that modulates an IgSF CAM ligand-independent activation of an IgSF CAM by an activated co-located GPCR, such as activated angiotensin receptor, such as AT₁R, also modulates an IgSF CAM ligand-dependent activation of an IgSF CAM.

In preferred embodiments of the invention, modulators of the invention do not modulate, or modulate differently, or modulate to a different extent, an IgSF CAM-independent signalling pathways associated with the certain activated co-located GPCR.

In a preferred embodiment, modulators of the invention do not inhibit, or inhibit to a lesser extent, one or more an IgSF CAM independent certain co-located GPCR signalling pathways.

In one form, the present invention comprises modulators of IgSF CAM activity where such IgSF CAM activity is induced by its cognate ligand and where the modulators of IgSF CAM activity are analogues, fragments or derivatives of ALCAM₅₅₉₋₅₈₀ (SEQ ID NO: 6).

In one form, the present invention comprises modulators of IgSF CAM activity where such IgSF CAM activity is induced by its cognate ligand and where the modulators of IgSF CAM activity are analogues, fragments or derivatives of ALCAM₅₅₉₋₅₈₀ (SEQ ID NO: 6) that differ by one, two, three, four, five, six, seven, eight, nine or ten amino acids.

In one form, the present invention comprises a modulator of IgSF CAM activity where such IgSF CAM activity is induced by its cognate ligand and where the modulator of IgSF CAM activity is ALCAM₅₅₉₋₅₈₀ (SEQ ID NO: 6).

In one form, the present invention comprises modulators of IgSF CAM activity where such IgSF CAM activity is induced by its cognate ligand and where the modulators of IgSF CAM activity are analogues, fragments or derivatives of RAGE.

In one form, the present invention comprises modulators of IgSF CAM activity where such IgSF CAM activity is induced by its cognate ligand and where the modulators of IgSF CAM activity are analogues, fragments or derivatives of the cytosolic tail of RAGE.

In one form, the present invention comprises modulators of IgSF CAM activity where such IgSF CAM activity is induced by its cognate ligand and where the modulators of IgSF CAM activity are analogues, fragments or derivatives of RAGE₃₇₀₋₃₉₀ (SEQ ID NO: 7).

In one form, the present invention comprises modulators of IgSF CAM activity where such IgSF CAM activity is induced by its cognate ligand and where the modulators of IgSF CAM activity are analogues, fragments or derivatives of RAGE₃₇₀₋₃₉₀ (SEQ ID NO: 7) that differ by one, two, three, four, five, six, seven, eight, nine or ten amino acids.

In one form, the present invention comprises a modulator of IgSF CAM activity where such IgSF CAM activity is induced by its cognate ligand and where the modulator of IgSF CAM activity is RAGE₃₇₀₋₃₉₀ (SEQ ID NO: 7).

In one form, the present invention comprises modulators of IgSF CAM activity where such IgSF CAM activity is induced by its cognate ligand and where the modulators of IgSF CAM activity are analogues, fragments or derivatives of S391A-RAGE₃₆₂₋₄₀₄ (SEQ ID NO: 8).

In one form, the present invention comprises modulators of IgSF CAM activity where such IgSF CAM activity is induced by its cognate ligand and where the modulators of IgSF CAM activity are analogues, fragments or derivatives of S391A-RAGE₃₆₂₋₄₀₄ (SEQ ID NO: 8) that differ by one, two, three, four, five, six, seven, eight, nine, ten, eleven, twelve, thirteen, fourteen, fifteen, sixteen, seventeen, eighteen, nineteen or twenty amino acids.

In one form, the present invention comprises a modulator of IgSF CAM activity where such IgSF CAM activity is induced by its cognate ligand and where the modulator of IgSF CAM activity is S391A-RAGE₃₆₂₋₄₀₄ (SEQ ID NO: 8).

In one form, the present invention comprises modulators of IgSF CAM ligand-dependent activation of an IgSF CAM where the modulators of IgSF CAM ligand-dependent activation of an IgSF CAM are analogues, fragments or derivatives of IgSF CAM.

In one form of the invention the modulator of IgSF CAM ligand-dependent activation of an IgSF CAM is an analogue, fragment or derivative of ALCAM₅₅₉₋₅₈₀ (SEQ ID NO: 6).

In one form of the invention the modulator of IgSF CAM ligand-dependent activation of an IgSF CAM is an analogue, fragment or derivative of ALCAM₅₅₉₋₅₈₀ (SEQ ID NO: 6) that differs by one, two, three, four, five, six, seven, eight, nine, ten, eleven, twelve, thirteen, fourteen, fifteen, sixteen, seventeen, eighteen, nineteen or twenty amino acids.

In one form of the invention the modulator of IgSF CAM ligand-dependent activation of an IgSF CAM is ALCAM₅₅₉₋₅₈₀ (SEQ ID NO: 6).

In one form, the present invention comprises modulators of IgSF CAM ligand-dependent activation of an IgSF CAM where the modulators of IgSF CAM ligand-dependent activation of an IgSF CAM are analogues, fragments or derivatives of RAGE.

In one form, the present invention comprises modulators of IgSF CAM ligand-dependent activation of an IgSF CAM where the modulators of IgSF CAM ligand-dependent activation of an IgSF CAM are analogues, fragments or derivatives of the cytosolic tail of RAGE.

In one form of the invention the modulator of IgSF CAM ligand-dependent activation of an IgSF CAM is an analogue, fragment or derivative of RAGE₃₇₀₋₃₉₀ (SEQ ID NO: 7).

In one form of the invention the modulator of IgSF CAM ligand-dependent activation of an IgSF CAM is an analogue, fragment or derivative of RAGE₃₇₀₋₃₉₀ (SEQ ID NO: 7) that differs by one, two, three, four, five, six, seven, eight, nine or ten amino acids.

In one form of the invention the modulator of IgSF CAM ligand-dependent activation of an IgSF CAM is RAGE₃₇₀₋₃₉₀ (SEQ ID NO: 7).

In one form of the invention the modulator of IgSF CAM ligand-dependent activation of an IgSF CAM is an analogue, fragment or derivative of S391A-RAGE₃₆₂_404 (SEQ ID NO: 8).

In one form of the invention the modulator of IgSF CAM ligand-dependent activation of an IgSF CAM is an analogue, fragment or derivative of S391A-RAGE₃₆₂_404 (SEQ ID NO: 8) that differs by one, two, three, four, five, six, seven, eight, nine, ten, eleven, twelve, thirteen, fourteen, fifteen, sixteen, seventeen, eighteen, nineteen or twenty amino acids.

In one form of the invention the modulator of IgSF CAM ligand-dependent activation of an IgSF CAM is S391A-RAGE₃₆₂₋₄₀₄ (SEQ ID NO: 8).

In one form, the present invention comprises modulators wherein the modulators are modulators of IgSF CAM dependent signalling induced by its cognate ligand where the modulators of IgSF CAM dependent signalling induced by its cognate ligand are analogues, fragments or derivatives of IgSF CAM.

In one form, the present invention comprises modulators wherein the modulators are modulators of IgSF CAM dependent signalling induced by its cognate ligand where the modulators of IgSF CAM dependent signalling induced by its cognate ligand are analogues, fragments or derivatives of RAGE.

In one form of the present invention, the modulators of ligand-dependent activation of an IgSF CAM by its cognate ligand, contain the entire ectodomain of an IgSF CAM conjugated to an analogue, fragment or derivative of the transmembrane domain of an IgSF CAM which is greater than 5, greater than 10, or greater than 20 amino acids in length and the modulators of ligand-dependent activation of an IgSF CAM by its cognate ligand are analogues, fragments or derivatives of IgSF CAM.

In one form of the present invention, the modulators of ligand-dependent activation of an IgSF CAM by its cognate ligand, contain the entire ectodomain of an IgSF CAM conjugated to an analogue, fragment or derivative of the transmembrane domain of an IgSF CAM which is greater than 5, greater than 10, or greater than 20 amino acids in length and the modulators of ligand-dependent activation of an IgSF CAM by its cognate ligand are analogues, fragments or derivatives of RAGE.

In one form of the present invention, the modulators of ligand-dependent activation of an IgSF CAM by its cognate ligand contain a fragment of the ectodomain of an IgSF CAM and the modulators of ligand-dependent activation of an IgSF CAM by its cognate ligand are analogues, fragments or derivatives of IgSF CAM.

In one form of the present invention, the modulators of ligand-dependent activation of an IgSF CAM by its cognate ligand contain a fragment of the ectodomain of an IgSF CAM and the modulators of ligand-dependent activation of an IgSF CAM by its cognate ligand are analogues, fragments or derivatives of RAGE.

In one form of the present invention, the modulators of ligand-dependent activation of an IgSF CAM by its cognate ligand inhibit or facilitate signalling that occurs through the C-terminal cytosolic tail of an IgSF CAM and the modulators of ligand-dependent activation of an IgSF CAM by its cognate ligand are analogues, fragments or derivatives of IgSF CAM.

In one form of the present invention, the modulators of ligand-dependent activation of an IgSF CAM by its cognate ligand inhibit or facilitate signalling that occurs through the C-terminal cytosolic tail of an IgSF CAM and the modulators of ligand-dependent activation of an IgSF CAM by its cognate ligand are analogues, fragments or derivatives of RAGE.

In one form of the present invention, the modulators of ligand-dependent activation of an IgSF CAM by its cognate ligand inhibit binding that occurs to the C-terminal cytosolic tail of an IgSF CAM and the modulators of ligand-dependent activation of an IgSF CAM by its cognate ligand are analogues, fragments or derivatives of IgSF CAM.

In one form of the present invention, the modulators of ligand-dependent activation of an IgSF CAM by its cognate ligand inhibit binding that occurs to the C-terminal cytosolic tail of an IgSF CAM and the modulators of ligand-dependent activation of an IgSF CAM by its cognate ligand are analogues, fragments or derivatives of the cytosolic tail of IgSF CAM.

In one form of the present invention, the modulators of ligand-dependent activation of an IgSF CAM by its cognate ligand inhibit binding that occurs to the C-terminal cytosolic tail of an IgSF CAM and the modulators of ligand-dependent activation of an IgSF CAM by its cognate ligand are analogues, fragments or derivatives of ALCAM₅₅₉₋₅₈₀ (SEQ ID NO: 6).

In one form of the present invention, the modulators of ligand-dependent activation of an IgSF CAM by its cognate ligand inhibit binding that occurs to the C-terminal cytosolic tail of an IgSF CAM and the modulators of ligand-dependent activation of an IgSF CAM by its cognate ligand are analogues, fragments or derivatives of ALCAM₅₅₉₋₅₈₀ (SEQ ID NO: 6) that differs by one, two, three, four, five, six, seven, eight, nine or ten amino acids.

In one form of the present invention, the modulator of ligand-dependent activation of an IgSF CAM by its cognate ligand inhibits binding that occurs to the C-terminal cytosolic tail of an IgSF CAM and the modulator of ligand-dependent activation of an IgSF CAM by its cognate ligand is ALCAM₅₅₉₋₅₈₀ (SEQ ID NO: 6).

In one form of the present invention, the modulator of ligand-dependent activation of an IgSF CAM by its cognate ligand is ALCAM₅₅₉₋₅₈₀ (SEQ ID NO: 6).

In one form of the present invention, the modulators of ligand-dependent activation of an IgSF CAM by its cognate ligand inhibit binding that occurs to the C-terminal cytosolic tail of an IgSF CAM and the modulators of ligand-dependent activation of an IgSF CAM by its cognate ligand are analogues, fragments or derivatives of RAGE.

In one form of the invention, the modulators of ligand-dependent activation of an IgSF CAM by its cognate ligand inhibit binding that occurs to the C-terminal cytosolic tail of an IgSF CAM and the modulators of ligand-dependent activation of an IgSF CAM by its cognate ligand are analogues, fragments or derivatives of the cytosolic tail of RAGE (RAGE₃₆₂₋₄₀₄) (SEQ ID NO: 31): LWQRRQRRGEERKAPENQEEEEERAELNQSEEPEAGESSTGGP.

In one form of the invention, the modulator of ligand-dependent activation of an IgSF CAM by its cognate ligand inhibit binding that occurs to the C-terminal cytosolic tail of an IgSF CAM and the modulators of ligand-dependent activation of an IgSF CAM by its cognate ligand are analogues, fragments or derivatives of RAGE₃₇₀₋₃₉₀ (SEQ ID NO: 7).

In one form of the invention, the modulator of ligand-dependent activation of an IgSF CAM by its cognate ligand inhibit binding that occurs to the C-terminal cytosolic tail of an IgSF CAM and the modulators of ligand-dependent activation of an IgSF CAM by its cognate ligand are analogues, fragments or derivatives of RAGE₃₇₀₋₃₉₀ (SEQ ID NO: 7) that differs by one, two, three, four, five, six, seven, eight, nine or ten amino acids.

In one form of the present invention, the modulator of ligand-dependent activation of an IgSF CAM by its cognate ligand inhibits binding that occurs to the C-terminal cytosolic tail of an IgSF CAM and the modulator of ligand-dependent activation of an IgSF CAM by its cognate ligand is RAGE₃₇₀₋₃₉₀ (SEQ ID NO: 7).

In one form of the present invention, the modulator of ligand-dependent activation of an IgSF CAM by its cognate ligand is RAGE₃₇₀₋₃₉₀ (SEQ ID NO: 7).

In one form of the invention, the modulator of ligand-dependent activation of an IgSF CAM by its cognate ligand inhibit binding that occurs to the C-terminal cytosolic tail of an IgSF CAM and the modulators of ligand-dependent activation of an IgSF CAM by its cognate ligand are analogues, fragments or derivatives of S391A-RAGE₃₆₂₋₄₀₄ (SEQ ID NO: 8).

In one form of the invention, the modulator of ligand-dependent activation of an IgSF CAM by its cognate ligand inhibit binding that occurs to the C-terminal cytosolic tail of an IgSF CAM and the modulators of ligand-dependent activation of an IgSF CAM by its cognate ligand are analogues, fragments or derivatives of S391A-RAGE₃₆₂₋₄₀₄ (SEQ ID NO: 8) that differs by one, two, three, four, five, six, seven, eight, nine, ten, eleven, twelve, thirteen, fourteen, fifteen, sixteen, seventeen, eighteen, nineteen or twenty amino acids.

In one form of the present invention, the modulator of ligand-dependent activation of an IgSF CAM by its cognate ligand inhibits binding that occurs to the C-terminal cytosolic tail of an IgSF CAM and the modulator of ligand-dependent activation of an IgSF CAM by its cognate ligand is S391A-RAGE₃₆₂₋₄₀₄ (SEQ ID NO: 8).

In one form of the present invention, the modulator of ligand-dependent activation of an IgSF CAM by its cognate ligand is S391A-RAGE₃₆₂₋₄₀₄ (SEQ ID NO: 8).

In one form of the invention, the modulator is isolated.

In one form, the invention comprises a pharmaceutical composition comprising a modulator as described herein.

In one form the invention comprises the use of a modulator as described herein for the treatment or prevention of an ailment.

In one form of the invention, a modulator of the invention is an activator, an inhibitor, an allosteric modulator, or a non-functional mimic of the cytosolic tail of RAGE. A non-functional substitute is a modulator that mimics the cytosolic tail of RAGE in the presence of certain co-located GPCRs, is not able to be activated by them or induce downstream RAGE-dependent signalling, and inhibits signalling that normally occurs through activation of the cytosolic tail of IgSF CAM and IgSF CAM-dependent signalling resulting therefrom.

In one form of the invention, a modulator of the invention is an activator, an inhibitor, an allosteric modulator, or a non-functional mimic of the transmembrane domain of RAGE or part thereof.

In one form of the invention, a non-functional substitute is a modulator that mimics the transmembrane domain of RAGE in the presence of certain co-located GPCRs, is not able to be activated by them or induce downstream RAGE-dependent signalling, and inhibits signalling that normally occurs through activation of the cytosolic tail of IgSF CAM and IgSF CAM-dependent signalling resulting therefrom.

In one form of the invention, the modulator comprises a transmembrane domain of RAGE or a part thereof and a fragment of the RAGE ectodomain.

In one form of the invention, the modulator comprises a transmembrane domain of RAGE or a part thereof and a fragment of the cytosolic tail of RAGE.

In one form of the invention, the modulator comprises a transmembrane domain of RAGE or part thereof and a fragment of the RAGE ectodomain and a fragment of the cytosolic tail of RAGE.

In one form of the invention, modulators of the invention contain a fragment of the ectodomain of RAGE, which is not greater than 40, not greater than 20, not greater than 10 or not greater than 5 amino acids in length.

In one form of the invention, S391A-RAGE₃₆₂₋₄₀₄ is a non-functional substitute for RAGE that in the presence of certain co-located GPCRs is not activated by them and inhibits IgSF CAM-dependent signalling. Expression of S391A-RAGE₃₆₂₋₄₀₄ inhibits IgSF CAM ligand-independent activation of IgSF CAM by activated AT₁R and IgSF CAM ligand-dependent activation of IgSF CAM. Furthermore, in one form of the invention, when S391A-RAGE₃₆₂₋₄₀₄ is fused to a cell penetrating peptide (TAT) and a marker protein (mCherry), treatment with TAT-mCherry-S391A-RAGE₃₆₂₋₄₀₄ oligopeptide inhibits IgSF CAM ligand-independent activation of IgSF CAM by activated AT₁R to attenuate Ang II-dependent pathology.

In one form of the invention, RAGE₃₃₈₋₃₆₁ inhibits IgSF CAM ligand-independent activation of IgSF CAM by activated AT₁R.

The sequence of RAGE₃₃₈₋₃₆₁ is SEQ ID NO: 19:

[LGTLALALGILGGLGTAALLIGVI]

In one form, the present invention comprises modulators of IgSF CAM ligand-independent activation of IgSF CAM by certain activated co-located GPCRs that modulate transactivation of the cytosolic tail of IgSF CAM triggered by activation of such certain activated co-located GPCRs, such as an angiotensin receptor.

In one form, the present invention comprises modulators of IgSF CAM ligand-independent activation of the cytosolic tail of IgSF CAM by certain activated co-located GPCRs that bind to Ras GTPase-activating-like protein (IQGAP1) or other IgSF CAM-associated proteins, including protein kinase C zeta (PKCζ), Dock7, MyD88, TIRAP, IRAK4, ERK1/2, olfactory receptor 2T2, ADP/ATP translocase 2, Protein phosphatase 1G, Intercellular adhesion molecule 1, Protein DJ-1 (PARK7), Calponin-3, Drebrin, Filamin B, Ras-related protein Rab-13, Radixin/Ezrin/Moesin, Proteolipid protein 2, Coronin, S100 A11, Succinyl-CoA ligase [GDP-forming] subunit alpha, Hsc70-interacting protein, Apoptosis Inhibitor 5, neuropilin, cleavage stimulation factor, growth factor receptor-bound protein 2, sec61 beta subunit, or Nck1, or disrupt the binding of these elements to IgSF CAM, in order to modulate IgSF CAM transactivation by certain activated co-located GPCRs, such as an angiotensin receptor, such as AT₁R.

In one form of the invention, the modulators of the invention bind to the cytosolic elements of the certain activated co-located GPCR, IgSF CAM and/or elements complexed with either, including IQGAP-1, PKCζ, Dock7, MyD88, TIRAP, IRAK4, ERK1/2, olfactory receptor 2T2, ADP/ATP translocase 2, Protein phosphatase 1G, Intercellular adhesion molecule 1, Protein DJ-1 (PARK7), Calponin-3, Drebrin, Filamin B, Ras-related protein Rab-13, Radixin/Ezrin/Moesin, Proteolipid protein 2, Coronin, S100 A11, Succinyl-CoA ligase [GDP-forming] subunit alpha, Hsc70-interacting protein, Apoptosis Inhibitor 5, neuropilin, cleavage stimulation factor, growth factor receptor-bound protein 2, sec61 beta subunit, or Nck1 to modulate IgSF CAM ligand-independent signalling through the cytosolic tail of IgSF CAM, by modulating these signalling elements required for IgSF CAM transactivation by certain activated co-located GPCRs, such as an angiotensin receptor, such as AT₁R.

In one form of the invention, modulators of IgSF CAM ligand-independent activation of IgSF CAM by certain activated co-located GPCRs also modulate IgSF CAM ligand-dependent activation of the cytosolic tail of IgSF CAM, by binding to cytosolic elements of IgSF CAM and/or elements that complex with IgSF CAM in the cytosol (such as IQGAP-1, PKCζ, Dock7, MyD88, IRAK4, TIRAP, ERK1/2, olfactory receptor 2T2, ADP/ATP translocase 2, Protein phosphatase 1G, Intercellular adhesion molecule 1, Protein DJ-1 (PARK7), Calponin-3, Drebrin, Filamin B, Ras-related protein Rab-13, Radixin/Ezrin/Moesin, Proteolipid protein 2, Coronin, S100 A11, Succinyl-CoA ligase [GDP-forming] subunit alpha, Hsc70-interacting protein, Apoptosis Inhibitor 5, neuropilin, cleavage stimulation factor, growth factor receptor-bound protein 2, sec61 beta subunit, or Nck1) to inhibit IgSF CAM ligand-mediated signalling through these elements.

In some embodiments, the modulator is introduced by gene delivery (such as by using a virus or artificial non-viral gene delivery such as electroporation, microinjection, gene gun, impalefection, hydrostatic pressure, continuous infusion, sonication, lipofection, liposomes, nanobubbles and polymeric gene carriers) and the peptide fragment, biologically-active analogue or derivative being generated by the cell as a consequence of transcriptional and translational processes.

In some embodiments of this aspect, the modulator has a modified capacity to form a complex with certain co-located GPCRs, such as AT₁R, or elements that complex with them. For example, the RAGE analogue or derivative may be distinguished from a wild-type RAGE polypeptide or fragment sequence by the substitution, addition, or deletion of at least one amino acid residue or addition or substitution of unusual or non-conventional amino-acids or non-amino acid residues.

In some embodiments, the modulator lacks or has a modification of serine-391 that is normally present in a wild-type human RAGE polypeptide. In illustrative examples of this type, the fragment, analogue or derivative of the cytosolic tail of RAGE lacks a serine at position 391 of the wild-type RAGE sequence (for example, the RAGE₃₇₀₋₃₉₀ construct is truncated at Glu390). Suitably, the serine at position 391 is deleted or substituted with another amino acid residue, an analogue or derivative, in order to impair or abolish signalling conferred by a serine at this site following activation of a co-located GPCR. In one embodiment, the serine at position 391 is deleted or substituted with another amino acid residue selected from the group: alanine, aspartate, phenylalanine, histidine, lysine, arginine, tyrosine, asparagine, valine, glycine, cysteine or glutamate.

In some embodiments, the modulator lacks or has an impaired ability to bind Diaphanous 1 (Diaph1) relative to human wild-type RAGE. In illustrative examples of this type, the peptide, or analogue, fragment or derivative thereof, either lacks the RAGE-Diaph1 binding site (such as RAGE₃₇₀₋₃₉₀, RAGE₃₇₄₋₃₉₀, or RAGE₃₇₉₋₃₉₀) or has an altered Diaph1 binding site (such as 366A/367A) in order to abolish or impair this site. Suitably, the residues at 366/367 are deleted or substituted with other residues (such as with alanine) in order to impair or abolish this site, and in doing so, improve affinity for binding to other targets, by reducing constraints induced by wild-type binding to Diaph1.

In one aspect of the invention, the modulator of the present invention includes isolated or purified peptides which comprise, consist, or consists essentially of an amino acid sequence represented by Formula I:

Z1MZ2 (I)

wherein:

Z1 is absent or is selected from at least one of a proteinaceous moiety comprising from about 1 to about 50 amino acid residues; and

M is the amino acid sequence as set forth in SEQ ID NO: 1, or an analogue, fragment or derivative thereof; and

Z2 is absent or is a proteinaceous moiety comprising from about 1 to about 50 amino acid residues.

In some embodiments of the invention described above, the modulator (such as a fragment of the RAGE cytosolic tail, an analogue or derivative thereof as broadly described above and elsewhere herein) is able to penetrate a cell membrane. In non-limiting examples of this type, the RAGE modulator is conjugated, fused or otherwise linked to a cell membrane penetration molecule (e.g., the HIV TAT motif, as set forth in SEQ ID NO: 20 below).

SEQ ID NO: 20:
[YGRKKRRQRRR].

In some forms of the invention, the modulator is a non-peptide molecule that shares with the peptide modulator described above the capacity to bind to and/or interfere with elements associated with IgSF CAM ligand-independent activation of IgSF CAM by certain activated co-located GPCRs. These non-peptide modulators may or may not contain structural similarities to functionally important domains contained in peptide modulators.

In a preferred form, the non-peptide modulator contains any combination of one or more structural similarities to functionally important domains contained in the peptide modulators, as defined by the pharmacophore described vide infra.

In preferred forms of the invention, the modulator is an inhibitor.

In certain forms of the invention, in addition to being an inhibitor of IgSF CAM ligand-independent activation of IgSF CAM by a certain activated co-located GPCR, the modulator is an inhibitor of the certain co-located GPCR and/or an inhibitor of the certain co-located GPCR signalling pathway.

In certain forms of the invention, in addition to being an inhibitor of IgSF CAM ligand-independent activation of IgSF CAM by a certain activated co-located GPCR, the modulator is an inhibitor of IgSF CAM ligand-dependent activation of IgSF CAM and/or an inhibitor of constitutively-active IgSF CAM and/or an inhibitor of a IgSF CAM signalling pathway.

In certain forms of the invention, where the certain co-located GPCR is AT₁R, in addition to being an inhibitor of IgSF CAM ligand-independent activation of IgSF CAM, the modulator is an AT₁R inhibitor and/or an inhibitor of an AT₁R signalling pathway.

In certain forms of the invention, in addition to being an inhibitor of IgSF CAM ligand-independent activation of IgSF CAM by activated angiotensin receptor, preferably activated AT₁R, the modulator is an inhibitor of IgSF CAM ligand-dependent activation of IgSF CAM and/or an inhibitor of constitutively-active IgSF CAM and/or an inhibitor of a IgSF CAM signalling pathway.

In certain forms of the invention, in addition to being an inhibitor of IgSF CAM ligand-independent activation of IgSF CAM by a certain activated co-located GPCR, the modulator is an inhibitor of the certain co-located GPCR and/or an inhibitor of the certain co-located GPCR signalling pathway and an inhibitor of IgSF CAM ligand-dependent activation of IgSF CAM and/or an inhibitor of constitutively-active IgSF CAM and/or an inhibitor of a IgSF CAM signalling pathway.

In certain forms of the invention, in addition to being an inhibitor of IgSF CAM ligand-independent activation of IgSF CAM by activated angiotensin receptor, preferably activated AT₁R, the modulator is an AT₁R inhibitor and/or an inhibitor of an AT₁R signalling pathway and an inhibitor of IgSF CAM ligand-dependent activation of IgSF CAM and/or an inhibitor of constitutively-active IgSF CAM and/or an inhibitor of a IgSF CAM signalling pathway.

In certain forms of the invention, the modulator is a non-functional substitute for the cytosolic tail of RAGE or a part thereof, which is not able to be activated by a co-located GPCR or facilitate downstream RAGE-dependent signalling and inhibits signalling that occurs through the cytosolic tail of IgSF CAM and IgSF CAM-dependent signalling.

In certain forms of the invention, the modulator is a non-functional substitute for the transmembrane domain of IgSF CAM or a part thereof, which is not able to be activated by a co-located GPCR or facilitate downstream IgSF CAM-dependent signalling and inhibits signalling that occurs through the cytosolic tail of IgSF CAM and IgSF CAM-dependent signalling.

In certain forms of the invention, the modulator comprises a transmembrane domain of RAGE or a part thereof and a fragment of the RAGE ectodomain. In certain forms of the invention, the modulator comprises a transmembrane domain of RAGE or a part thereof and a fragment of the cytosolic tail of RAGE.

In certain forms of the invention, the modulator comprises a transmembrane domain of RAGE or part thereof and a fragment of the RAGE ectodomain and a fragment of the cytosolic tail of RAGE.

In certain forms of the invention, the modulators of IgSF CAM ligand-independent activation of IgSF CAM by certain activated co-located GPCRs contain a fragment of the ligand-binding ectodomain of human wild-type RAGE, which is not greater than 40, not greater than 20, not greater than 10 or not greater than 5 amino acids in length.

The inventors have discovered that a peptide comprising residues 370-390 of the cytosolic tail of RAGE (see SEQ ID NO: 7) is an inhibitory peptide, inhibiting both IgSF CAM ligand-independent and IgSF CAM ligand-dependent activation of full length IgSF CAM.

A solution NMR structure exists for RAGE₃₆₃₋₄₀₄ (Rai V et al., 2012) showing that the N-terminus (residues 363-376) of this peptide is ordered. A Rosetta-derived model exists for RAGE-₃₆₂₋₄₀₄ (model4) which is consistent with the NMR structure (http://www.rcsb.org/pdb/explore/explore.do?structureId=2LMB, accessed 25 Aug. 2016)) and also suggests that the remainder of the peptide forms an alpha helix.

An initial model of RAGE₃₇₀₋₃₉₀ was constructed by truncating model4 (model4_₃₇₀₋₃₉₀). Model4 is a theoretical model of the RAGE cytosolic tail, generated by inputting the sequence into the I-Tasser web server (http://zhanglab.ccmb.med.umich.edu/I-TASSER/). See also Yang et al (2015), Roy et al (2010) and Y Zhang (2008). All five models presented by the 1-Tasser server predicted the region 370-390 to form a helix. The models and the NMR structure were aligned by the C-alpha carbons of the backbones of the peptide sequences. Model 4 was selected as the preferred model, as the predicted structure of the region corresponding to the Diaphanous 1 binding site in model4 was closest to the documented NMR structure for this region.

A 20 ns molecular dynamics simulation of model4 in water was run using GROMACS (Hess et al., 2008). The molecular dynamics simulation suggests that the alpha helix region of model4_₃₇₀₋₃₉₀ is stable. Strong interactions are observed between a number of charged side chains, suggesting that these interactions stabilise the folded structure and that any conservation of these residues might result from their role in stabilising the peptide structure.

A Blast search was used to identify homologous sequences for RAGE₃₇₀₋₃₉₀. The sequences were aligned as follows:

CLUSTAL 2.0.10 multiple sequence alignment
mode14_3₇₀₋₃₉₀.pdb ----GEERKAPENQ--EEEEERAELNQ---
gi|505855911|ref|XP_004621364. RRRRGEERKVPENQ--EEEEERAELKQSGE
gi|836716008|ref|XP_012791097. RRRRGEERKVPENQ--EEEEERAELKQSGE
gi|830242517|ref|XP_012589882. RRR-GEQRKAPENR--EEEEERAELNQSEE
gi|830242520|ref|XP_012589883. RRR-GEQRKAPENR--EEEEERAELNQSEE
gi|830242532|ref|XP_012589884. RRR-GEQRKAPENR--EEEEERAELNQSEE
gi|859958468|ref|XP_012905636. RPR-REERKAPENQ--EEEEERAELNQSEE
gi|505855913|ref|XP_004621365. RRRRGEERKVPENQ--EEEEERAELKQSGE
gi|859958474|ref|XP_012905637. RPR-REERKAPENQ--EEEEERAELNQSEE
gi|674092933|ref|XP_008819684. QHR-GEERKTPENQ--EDEEERAELNQSEE
gi|852803202|ref|XP_012890437. QHR-GEERKAPENQ--EEEEERAELNQSEE
gi|586986169|ref|XP_006931651. RRQ-GEERKAPENQEEEEEEEREELNQSGE
gi|752437365|ref|XP_011235981. RHR-REERKAPENQ--EEEEERAELNQSEE
gi|671038558|ref|XP_008710071. RHR-REERKAPENQ--EEEEERAELNQSVE
gi|859958450|ref|XP_012905633. RPR-REERKAPENQ--EEEEERAELNQSEE
gi|1040099494|gb|OBS60144.11 QPR-GEERKTPENQ--EDEEERAELNQSED
gi|674092931|ref|XP_008819683. QHR-GEERKTPENQ--EDEEERAELNQSEE
gi|641730582|ref|XP_008155542. RHR-GEERKAPENQA-EEEEERAELNQSQE
gi|641730580|ref|XP_008155541. RHR-GEERKAPENQA-EEEEERAELNQSQE
gi|946738855|ref|XP_014389946. RRR-GEERKAPENQ--EEEEERAELHQSQE
gi|940771956|ref|XP_006104444. RRR-GEERKAPENQ--EEEEERAELHQSQE
gi|355748446|gb|EHH52929.11 RRQ-GEERKASENQ--EEEEERAELNQSEE
gi|355561569|gb|EHH18201.11 RRQ-GEERKASENQ--EEEEERAELNQSEE
gi|544428837|ref|XP_005553456. RRQ-GEERKASENQ--EEEEERAELNQSEE
gi|635095937|ref|XP_007971201. RRQ-GEERKASENQ--EEEEERAELNQSEE
gi|402866556|ref|XP_003897445. RRQ-REERKASENQ--EEEEERAELNQSEE
gi|795466133|ref|XP_011890032. RRQ-GEERKASENQ--EEEEERAELNQSEE
gi|795466129|ref|XP_011890031. RRQ-GEERKASENQ--EEEEERAELNQSEE
gi|795317622|ref|XP_011824818. RRQ-GEERKASENQ--EEEEERAELNQSEE
gi|326693968|ref|NP_001192046. RRQ-GEERKASENQ--EEEEERAELNQSEE
gi|724802002|ref|XP_010376439. RRQ-GEERKAPENQ--EEEEERAELNQSEE
gi|724801999|ref|XP_010376432. RRQ-GEERKAPENQ--EEEEERAELNQSEE
gi|795178216|ref|XP_011800170. RRQ-GEERKAPENQ--EEEEERAELNQSEE
gi|312182478|gb|ADQ42279.11 RRQ-GEERKASENQ--EEEEERAELNQSEE
gi|795178211|ref|XP_011800169. RRQ-GEERKAPENQ--EEEEERAELNQSEE
gi|332800965|ref|NP_001193858. QRR-GEERKAPENQ--EEEEERAELNQSEE
gi|10835203|ref|NP_001127.11 QRR-GEERKAPENQ--EEEEERAELNQSEE
gi|332800967|ref|NP_001193861. QRR-GEERKAPENQ--EEEEERAELNQSEE
gi|823672830|gb|AK171626.11 QRR-GEERKAPENQ--EEEEERAELNQSEE
gi|1908461gb|AAA03574.11 QRR-GEERKAPENQ--EEEEERAELNQSEE
gi|194389738|dbj|BAG60385.11 QRR-GEERKAPENQ--EEEEERAELNQSEE
gi|694915715|ref|XP_009449249. QRQ-GEERKAPENQ--EEEEERAELNQSEE
gi|694915717|ref|XP_009449250. QRQ-GEERKAPENQ--EEEEERAELNQSEE
gi|694915721|ref|XP_009449252. QRQ-GEERKAPENQ--EEEEERAELNQSEE
gi|397519329|ref|XP_003829814. QRQ-GEERKAPENQ--EEEEERAELNQSEE
gi|397519323|ref|XP_003829811. QRQ-GEERKAPENQ--EEEEERAELNQSEE
gi|820970747|ref|XP_012358508. RRQ-GEERKAPENQ--EEEEERAELNQSEE
gi|820970749|ref|XP_012358509. RRQ-GEERKAPENQ--EEEEERAELNQSEE
gi|817330292|ref|XP_012292176. RRR-GEERKAPENQ--EEEEEHAELNQSEE
gi|817330294|ref|XP_012292177. RRR-GEERKAPENQ--EEEEEHAELNQSEE
gi|725608250|ref|XP_010330526. RRR-GEERKAPENQ--EEEEEHAELNQSEE
gi|725608252|ref|XP_010330527. RRR-GEERKAPENQ--EEEEEHAELNQSEE
gi|296197788|ref|XP_002746422. RRRRGEERKAPENQ--EEEEEHAELNQSEE
gi|826320184|ref|XP_012509111. RGQ-GEERKAPENQ--EEEEERAELNQSEE
gi|826320169|ref|XP_012509105. RGQ-GEERKAPENQ--EEEEERAELNQSEE
gi|826320175|ref|XP_012509107. RGQ-GEERKAPENQ--EEEEERAELNQSEE
gi|826320172|ref|XP_012509106. RGQ-GEERKAPENQ--EEEEERAELNQSEE
gi|829933710|ref|XP_012596554. RHQ-GEERKAPENQ--EEEEERAELNQSEE
gi|829933718|ref|XP_012596557. RHQ-GEERKAPENQ--EEEEERAELNQSEE
gi|829933722|ref|XP_012596558. RHQ-GEERKAPENQ--EEEEERAELNQSEE
gi|743731194|ref|XP_010959751. QRR-GEERKAPENQ-EEEEEERAELNQQEE
gi|560905029|ref|XP_006178871. QRR-GEERKAPENQ-EEEEEERAELNQQEE
gi|593759840|ref|XP_007118666. QRR-GEERKAPENQ-EEEEEERTELNQPEE
gi|560986474|ref|XP_006215428. QRR-GEERKAPENQ-EEEEEERAELNQQEE
gi|927155182|ref|XP_013833109. QRR-GQERKAPENQ-EEDEEERAELNQPED
gi|147225137|emb|CAN13265.11 QRR-GQERKAPENQ-EEDEEERAELNQPED
gi|178056480|ref|NP_001116690. QRR-GQERKAPENQ-EEDEEERAELNQPED
gi|162138238|gb|ABX82823.11 QRR-GQERKAPENQ-EEDEEERAELNQPED
gi|471418692|ref|XP_004390841. KHR-GEERKAPENQ--EEEEEHAELNQSEE
gi|471418700|ref|XP_004390845. KHR-GEERKAPENQ--EEEEEHAELNQSEE
gi|471418694|ref|XP_004390842. KHR-GEERKAPENQ--EEEEEHAELNQSEE
gi|829933714|ref|XP_012596556. RHQ-GEERKAPENQ--EEEEERAELNQSEE
gi|831224940|ref|XP_012660273. QCQ-GEERKAPENQ--EEEEERTELNQSEE
gi|984103351|ref|XP_015342983. RRQ-GEERKAPENQ--EEEEERAELNQSEE
gi|532108558|ref|XP_005339001. RRQ-GEERKAPENQ--EEEEERAELNQSEE
gi|955504646|ref|XP_014638416. QHR-REERKAPENQ--EEEEERAELNQSEE
gi|478500097|ref|XP_004424372. QHR-REERKAPENQ--EEEEERAELNQSEE
gi|955504650|ref|XP_014638417. QHR-REERKAPENQ--EEEEERAELNQSEE
gi|1048457071|ref|XP_017510394 QCR-GEERKAPENQ--EEEEERAELSQSEE
gi|589966171|ref|XP_006995615. QPR-REERKAPENQ--EDEEERAELNQSED
gi|589966173|ref|XP_006995616. QPR-REERKAPENQ--EDEEERAELNQSED
gi|532056239|ref|XP_005370828. QPR--EERKAPENE--EDEEERAELNQSED
gi|532056241|ref|XP_005370829. QPR--EERKAPENE--EDEEERAELNQSED
gi|532056245|ref|XP_005370831. QPR--EERKAPENE--EDEEERAELNQSED
gi|641730578|ref|XP_008155540. RHR-GEERKAPENQA-EEEEERAELNQSQE

This analysis identified a number of strongly conserved residues in RAGE₃₇₀₋₃₉₀ marked with as follows: * (asterisk) indicates positions which have a single, fully conserved residue. : (colon) indicates conservation between groups of strongly similar properties—scoring >0.5 in the Gonnet PAM 250 matrix. . (period) indicates conservation between groups of weakly similar properties—scoring=<0.5 in the Gonnet PAM 250 matrix:

| | | : | : | * | * | . | . | * | * | . | * | : | * | * | * | : | . | * | * | . | * |
|---|---|---|---|---|---|---|---|---|---|---|---|---|---|---|---|---|---|---|---|---|---|
| RAGE- | G | E | E | R | K | A | P | E | N | Q | E | E | E | E | E | R | A | E | L | N | Q |

Highly conserved residues are likely to play a structural role. Residues underlined are located on one face of the helix and likely represent the binding pharmacophore.

Examination of the model4_RAGE₃₇₀₋₃₉₀ structure and the molecular dynamics simulation results shows that a number of salt bridges are present in the structure. The molecular dynamics simulations show that these interactions are important structural features. Structural function is a likely reason for the conserved nature of these amino acids.

A number of strongly conserved amino acids are not involved in salt-bridge formation. These are present on one face of the RAGE₃₇₀₋₃₉₀ helix and likely represent the binding interface. These are Glu380, Glu384, Glu387 and Leu388. Another highly conserved residue, Glu377 is also present on this face of the peptide and may also be involved in binding, in addition to forming an alpha-helix-stabilising salt bridge to Lys374.

In a preferred form of the invention, the modulator of IgSF CAM ligand-independent activation of IgSF CAM by a certain activated co-located GPCR, such as an angiotensin receptor, such as AT₁R, is a peptide QEEEEERAELNQ as set forth in SEQ ID NO: 21, or a derivative thereof.

SEQ ID NO: 21: QEEEEERAELNQ.

The peptide may also have an initiating methionine and therefore have the sequence SEQ ID NO: 22: MQEEEEERAELNQ.

A pharmacophore for RAGE₃₇₉₋₃₉₀ peptide derived from the structure model4_RAGE₃₇₀₋₃₉₀ is represented below:

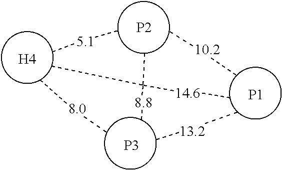

H4 is a hydrophobic residue, and P1-P3 are polar residues, and distances are shown in Angstroms. A matrix of distances between site points is as follows, where P represents a polar site point (hydrogen bonding or charged), and H represents a hydrophobic site point. Distances are in Angstroms. A tolerance should be applied to the position of each point.

| AA seq | | | | |
|---|---|---|---|---|
| # | 380 (P1) | 384 (P2) | 387 (P3) | 388 (H4) |
| 380 | 0 | | | |
| 384 | 10.2 Å | 0 | | |
| 387 | 13.2 Å | 8.8 Å | 0 | |
| 388 | 14.6 Å | 5.1 Å | 8Å | 0 |

The molecular dynamics simulations show that the interacting groups of RAGE₃₇₉₋₃₉₀ are mobile and a tolerance should be applied to the position of each group of up to ±10A provided the distances between the site points is positive in magnitude.

As would be understood by a person skilled in the art, additional, smaller pharmacophores can be generated by taking subsets of the above, and the present invention encompasses such pharmacophores, methods for using such to identify compounds, and compounds so identified.

In one form, the present invention further comprises a modulator of IgSF CAM ligand-independent activation of IgSF CAM by a certain activated co-located GPCR comprising two or more features selected from the group: a first charged or hydrogen bonding group (A), a second charged or hydrogen bonding group (B), a third charged or hydrogen bonding group (C), and a hydrophobic group (D) wherein the distances between the site points of the features are as follows, within a tolerance of up to ±10 Å, provided the distances between the site points is positive in magnitude:

| | A | B | C | D |
|---|---|---|---|---|
| A | | | | |
| B | 10.2 Å | | | |
| C | 13.2 Å | 8.8 Å | | |
| D | 14.6 Å | 5.1 Å | 8 Å | |

In a preferred form of the invention, the tolerance is up to ±5 Å, provided the distances between the site points is positive in magnitude. In a preferred form of the invention, the tolerance is up to ±2 Å, provided the distances between the site points is positive in magnitude. In a preferred form of the invention, the tolerance is up to ±1 Å, provided the distances between the site points is positive in magnitude.

In a preferred form of the invention, the modulator comprises three or more features selected from the above-specified group.

In a preferred form of the invention, the modulator comprises four features from the above-specified group.

In one form of the invention, there is provided a modulator characterised in that the modulator comprises at least two features chosen from one of the following combinations: AB, AC, AD, BC, BD, and CD.

In one form of the invention, there is provided a modulator, characterised in that the modulator comprises at least three features chosen from one of the following combinations: ABC, ABD, ACD, and BCD.

In one form of the invention, there is provided a modulator characterised in that the modulator comprises at least four features chosen from one of the following combinations: ABCD.

In one form of the invention, there is provided a modulator characterised in that the modulator comprises an additional charged or hydrogen bonding group (P1), consistent with the conserved stabilizing actions of E377 in RAGE₃₇₀₋₃₉₀, and therefore comprises two or more features selected from the group: a first charged or hydrogen bonding group (A), a second charged or hydrogen bonding group (B), a third charged or hydrogen bonding group (C), a fourth charged or hydrogen group (D), and a hydrophobic group (E) wherein the distances between the site points of the features are as follows, within a tolerance of ±10 Å:

| AA seq | 377 (P1) | 380 (P2) | 384 (P3) | 387 (P4) | 388 (H5) |
|---|---|---|---|---|---|
| # | A | B | C | D | E |
| A | | | | | |
| B | 7.4 Å | | | | |
| C | 13.9 Å | 10.2 Å | | | |
| D | 19.5 Å | 13.2 Å | 8.8 Å | | |
| E | 18.5 Å | 14.6 Å | 5.1 Å | 8 Å | |

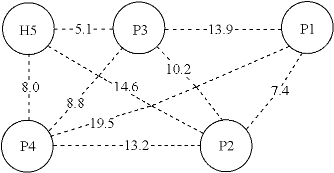

The modulator of IgSF CAM ligand-independent activation of IgSF CAM may be a peptide, or a non-peptidyl compound.

In one form of the invention, the hydrophobic group is an amino acid residue selected from the group: Ala, Val, Leu, Ile, Phe, Trp, Tyr.

In one form of the invention, the hydrophobic group is a chemical moiety selected from the group: C₁₋₈ alkyl, C₁₋₈ alkenyl, C₃₋₆ cycloalkyl, aryl, substituted aryl, alkyl aryl, heteroaryl, alkyl heteroaryl.

“Alkyl” means an aliphatic hydrocarbon group, which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain.

“Lower alkyl” means a group having about 1 to about 6 carbon atoms in the chain which may be straight or branched. The alkyl group may be optionally substituted by one or more substituents which may be the same or different, each substituent being independently selected from the group consisting of halo, alkyl, aryl, cycloalkyl, cyano, hydroxy, alkoxy, alkylthio, amino, —NH(alkyl), —NH(cycloalkyl), —N(alkyl)₂, carboxy and —C(O)O-alkyl. Non-limiting examples of suitable alkyl groups include methyl, ethyl, n-propyl, isopropyl and t-butyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkenyl chain.

“Lower alkenyl” means about 2 to about 6 carbon atoms in the chain which may be straight or branched. Non-limiting examples of suitable alkenyl groups include ethenyl, propenyl, 2-butenyl and 3-methylbutenyl. The term “substituted alkenyl” means that the alkenyl group may be substituted by one or more substituents which may be the same or different, each substituent being independently selected from the group consisting of alkyl, aryl and cycloalkyl.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain.

“Lower alkynyl” means about 2 to about 6 carbon atoms in the chain which may be straight or branched. Non-limiting examples of suitable alkynyl groups include ethynyl, propynyl, 2-butynyl and 3-methylbutynyl. The term “substituted alkynyl” means that the alkynyl group may be substituted by one or more substituents which may be the same or different, each substituent being independently selected from the group consisting of alkyl, aryl and cycloalkyl.

“Aliphatic” means and includes straight or branched chains of paraffinic, olefinic or acetylenic carbon atoms. The aliphatic group can be optionally substituted by one or more substituents which may be the same or different, each substituent being independently selected from the group consisting of H, halo, halogen, alkyl, aryl, cycloalkyl, cycloalkylamino, alkenyl, heterocyclic, alkynyl, cycloalkylaminocarbonyl, hydroxyl, thio, cyano, hydroxy, alkoxy, alkylthio, amino, —NH(alkyl), —NH(cycloalkyl), —N(alkyl)₂) carboxyl, —C(O)O-alkyl, heteroaryl, aralkyl, alkylaryl, aralkenyl, heteroaralkyl, alkylheteroaryl, heteroaralkenyl, heteroalkyl, carbonyl, hydroxyalkyl, aryloxy, aralkoxy, acyl, aroyl, nitro, amino, amido, ester, carboxylic acid aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, cycloalkenyl, heterocyclyl, heterocyclenyl, carbamate, urea, ketone, aldehyde, cyano, sulfonamide, sulfoxide, sulfone, sulfonyl urea, sulfonyl, hydrazide, hydroxamate, S(alkyl)Y1Y2N-alkyl-, Y1Y2N-alkyl-, Y1Y2NC(O)— and Y1Y2NSO₂—, wherein Y1 and Y2 can be the same or different and are independently selected from the group consisting of hydrogen, alkyl, aryl, and aralkyl.

“Heteroaliphatic” means an otherwise aliphatic group that contains at least one heteroatom (such as oxygen, nitrogen or sulfur). The term heteroaliphatic includes substituted heteroaliphatic.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroalkyl” means an alkyl as defined above, wherein one or more hydrogen atoms are substituted by a heteroatom selected from N, S, or O.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl- group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl- group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multi-cyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like, as well as partially saturated species such as, for example, indanyl, tetrahydronaphthyl and the like. “Halogen” means fluorine, chlorine, bromine, or iodine. Preferred are fluorine, chlorine and bromine.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, —C(═N—CN)—NH₂, —C(═NH)—NH₂, —C(═NH)—NH(alkyl), Y1Y2N—, Y1Y2N-alkyl-, Y1Y2NC(O)—, Y1Y2NSO₂— and —SO₂NY1Y2, wherein Y1 and Y2 can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such moieties are methylene dioxy, ethylenedioxy, —C(CH₃)₂— and the like which form moieties such as, for example:

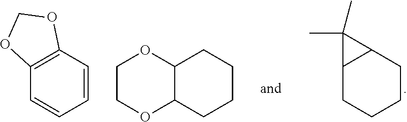

It should be noted that in hetero-atom containing ring systems of this invention, there are no hydroxyl groups on carbon atoms adjacent to a N, O or S1 as well as there are no N or S groups on carbon adjacent to another heteroatom. Thus, for example, in the ring:

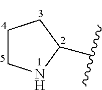 [SMILES: *C1CCCN1]

there is no —OH attached directly to carbons marked 2 and 5.

It should also be noted that tautomeric forms such as, for example, the moieties:

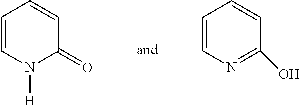 [SMILES: O=c1cccc[nH]1 ; Oc1ccccn1]

are considered equivalent in certain embodiments of this invention.

“Alkynylalkyl” means an alkynyl-alkyl- group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylmethyl.

“Heteroaralkyl” means a heteroaryl-alkyl- group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl- group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds.

By “stable compound” or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, Greene et al (1991).

When any variable (e.g., aryl, heterocycle, R2) occurs more than one time in any constituent or in the present invention, its definition on each occurrence is independent of its definition at every other occurrence.

In one form of the invention, each of the charged or hydrogen bonding groups is an amino acid residue selected, independently, from the group: Asp, Glu.

In one form of the invention, each of the charged or hydrogen bonding groups is an amino acid residue having a carboxylic acid moiety.

In one form of the invention, each of the charged or hydrogen bonding groups is a chemical moiety selected, independently, from the group: carboxylic acid, Hydroxaymic acids, phosphonic and phosphinic acids, sulfonic and sulfinic acids, sulphonamides, acylsulfonamides and sulfonylureas, 2,2,2-Trifluoroethan-1-ol and Trifluoromethylketones, tetrazoles, 5-Oxo-1,2,4-oxadiazole and 5-Oxo-1,2,4-thiadiazoles, Thiazolidinedione, Oxazolidinedione, and Oxadiazolidine-diones, 3-Hydroxyisoxazole and 3-Hydroxyisothiazoles, substituted phenols, squaric acids, 3- and 4-Hydroxyquinolin-2-ones, Tetronic and Tetramic Acids, Cyclopentane-1,3-diones and other cyclic and acyclic structures, including boronic acids, mercaptoazoles, and sulfonimidamides (Ballatore et al., 2013).

In one form, the invention provides a method for identifying a non-peptidyl modulator of IgSF CAM ligand-independent activation of IgSF CAM by a certain activated co-located GPCR, such as an angiotensin receptor, such as AT₁R, said method comprising the steps of: (1) comparing the three dimensional structure of the non-peptidyl compound with a pharmacophore comprising two or more features selected from the group: a first charged or hydrogen bonding group (A), a second charged or hydrogen bonding group (B), a third charged or hydrogen bonding group (C), and a hydrophobic group (D) wherein the distances in between the features are as follows, within a tolerance of ±10 Å:

| | A | B | C | D |
|---|---|---|---|---|
| A | | | | |
| B | 10.2 Å | | | |
| C | 13.2 Å | 8.8 Å | | |
| D | 14.6 Å | 5.1 Å | 8 Å | |

and (2) selecting a non-peptidyl compound with hydrophobic and/or charged or hydrogen bonding chemical moieties so located.

In a preferred form of the invention, the tolerance is up to ±5 Å, provided the distances between the site points is positive in magnitude. In a preferred form of the invention, the tolerance is up to ±2 Å, provided the distances between the site points is positive in magnitude. In a preferred form of the invention, the tolerance is up to ±1 Å, provided the distances between the site points is positive in magnitude.

In a preferred form of the invention, the modulator comprises three or more features selected from the above-specified group.

In a preferred form of the invention, the modulator comprises four features from the above-specified group.

In one form of the invention, comparison of the three dimensional structure of the non-peptidyl compound with the pharmacophore involves comparison of a minimum energy structure of the non-peptidyl compound with the pharmacophore.

An efficient means to select a non-peptidyl compound from a potentially large number of non-peptidyl compounds involves comparing non-peptidyl compounds against the pharmacophore of the invention using a computer program, for example Catalyst (MSI), to screen one or more computerised databases of three dimensional chemical structures of non-peptidyl compounds.

In one form of the invention, the modulator of IgSF CAM ligand-independent activation of IgSF CAM by a certain activated co-located GPCR, such as an angiotensin receptor, such as AT₁R, is a peptide that has an amino acid sequence as set forth in SEQ ID NO: 7, or an analogue, fragment or derivative thereof that contains at least residues 379-390.

In one form of the invention, the modulator of IgSF CAM ligand-independent activation of IgSF CAM by a certain activated co-located GPCR, such as an angiotensin receptor, such as AT₁R, is a peptide of the formula SEQ ID NO: 1, or an analogue, fragment or derivative thereof.

In one form of the invention, the modulator of IgSF CAM ligand-independent activation of IgSF CAM by a certain activated co-located GPCR, such as an angiotensin receptor, such as AT₁R, is a peptide of the formula SEQ ID NO: 2, or an analogue, fragment or derivative thereof.

In one form of the invention, the modulator of IgSF CAM ligand-independent activation of IgSF CAM by a certain activated co-located GPCR, such as an angiotensin receptor, such as AT₁R, is a peptide of the formula SEQ ID NO: 3, or an analogue, fragment or derivative thereof.

In one form of the invention, the modulator of IgSF CAM ligand-independent activation of IgSF CAM by a certain activated co-located GPCR, such as an angiotensin receptor, such as AT₁R, is a peptide of the formula SEQ ID NO: 4, or an analogue, fragment or derivative thereof.

In one form of the invention, the modulator of IgSF CAM ligand-independent activation of IgSF CAM by a certain activated co-located GPCR, such as an angiotensin receptor, such as AT₁R, is a peptide of the formula SEQ ID NO: 5, or an analogue, fragment or derivative thereof.

In one form of the invention, the modulator of IgSF CAM ligand-independent activation of IgSF CAM by a certain activated co-located GPCR, such as an angiotensin receptor, such as AT₁R, is a peptide of the formula SEQ ID NO: 6, or an analogue, fragment or derivative thereof.

In one form of the invention, the modulator of IgSF CAM ligand-independent activation of IgSF CAM by a certain activated co-located GPCR, such as an angiotensin receptor, such as AT₁R, is a peptide of the formula SEQ ID NO: 7, or an analogue, fragment or derivative thereof.

In one form of the invention, the modulator of IgSF CAM ligand-independent activation of IgSF CAM by a certain activated co-located GPCR, such as an angiotensin receptor, such as AT₁R, is a peptide of the formula SEQ ID NO: 8, or an analogue, fragment or derivative thereof.

In one form of the invention, the modulator of IgSF CAM ligand-independent activation of IgSF CAM by a certain activated co-located GPCR, such as an angiotensin receptor, such as AT₁R, is a peptide of the formula SEQ ID NO: 19, or an analogue, fragment or derivative thereof.

In one form of the invention, the modulator of IgSF CAM ligand-independent activation of IgSF CAM by a certain activated co-located GPCR, such as an angiotensin receptor, such as AT₁R, is a peptide of the formula SEQ ID NO: 21, or an analogue, fragment or derivative thereof.

In one form of the invention, the modulator of IgSF CAM ligand-independent activation of IgSF CAM by a certain activated co-located GPCR, such as an angiotensin receptor, such as AT₁R, is a peptide of the formula SEQ ID NO: 22, or an analogue, fragment or derivative thereof.

In one form of the invention, the modulator of IgSF CAM ligand-independent activation of IgSF CAM by a certain activated co-located GPCR, such as an angiotensin receptor, such as AT₁R, is a S391A-E392X-RAGE peptide as set forth in SEQ ID NO: 23, or an analogue or derivative thereof.

SEQ ID NO: 23: LWQRRQRRGEERKAPENQEEEEERAELNQA

In one form of the invention, the modulator of IgSF CAM ligand-independent activation of IgSF CAM by a certain activated co-located GPCR, such as an angiotensin receptor, such as AMR, is a S391X-RAGE peptide as set forth in of SEQ ID NO: 24, or an analogue or derivative thereof.

SEQ ID NO: 24: LWQRRQRRGEERKAPENQEEEEERAELNQ

Preferred specific derivatives include Q₃₇₉EEEEERAELNR₃₉₀, as set forth in SEQ ID NO: 25, Q₃₇₉EEEEERAELNK₃₉₀ as set forth in SEQ ID NO: 26, K₃₇₉EEEEERAELNQ₃₉₀ as set forth in SEQ ID NO: 27, K₃₇₉EEEERAELNK₃₉₀ as set forth in SEQ ID NO: 28, and K₃₇₉EEEEERAELNR₃₉₀ as set forth in SEQ ID NO: 29 below.

SEQ ID NO: 25: [Q379EEEEERAELNR390]
SEQ ID NO: 26: [Q379EEEEERAELNK390]
SEQ ID NO: 27: [K379EEEEERAELNQ390]
SEQ ID NO: 28: [K379EEEEERAELNK390]
SEQ ID NO: 29: [K379EEEEERAELNR390]

The term “derivative” as used herein in connection with modulators of the invention, such as SEQ ID NO: 1 to 8, 19, 21 to 31, refers to a modulator characterised in that its primary structure is taken from or owes its derivation to the C-terminal cytosolic tail of RAGE or fragment thereof, but which includes amino acid additions, substitutions, truncations, chemical and/or biochemical modifications (acetylation, carboxylation, phosphorylation, glycosylation, ubiquitination, side chain methylation), labelling with radionucleotides or halogens, unusual or artificial amino acids (such as D-amino acids, N-methylated amino acids, tetra-substitution, 8-peptides, pyroglutamic acid; 2-Aminoadipic acid; 3-Aminoadipic acid; beta-Alanine; beta-Aminopropionic acid; 2-Aminobutyric acid; 4-Aminobutyric acid; Piperidinic acid; 6-Aminocaproic acid; 2-Aminoheptanoic acid; 2-Aminoisobutyric acid; 3-Aminoisobutyric acid; 2-Aminopimelic acid; 2,4-Diaminobutyric acid; Desmosine; 2,2″-Diaminopimelic acid; 2,3-Diaminopropionic acid; N-Ethylglycine; N-Ethylasparagine; Hydroxylysine; allo-Hydroxylysine; 3-Hydroxyproline; 4-Hydroxyproline; Isodesmosine; allo-Isoleucine; N-Methylglycine; Sarcosine; N-Methylisoleucine; N-Methylvaline; Norvaline; Norleucine; Ornithine; Statine), retroinverted sequences, cyclic peptides, peptoids, or linkage to a non-peptide drug, non-peptide label, non-peptide carrier, or non-peptide resin.

In one form of the invention, the modulator is a peptide comprising residues 343-361 of wild-type RAGE (SEQ ID NO: 30) which is an inhibitory peptide, that inhibits both IgSF CAM ligand-independent and IgSF CAM ligand-dependent activation of IgSF CAM.

Substitutions encompass amino acid alterations in which an amino acid is replaced with a different naturally-occurring or a non-conventional amino acid residue. Such substitutions may be classified as “conservative”, in which case an amino acid residue contained in a polypeptide is replaced with another naturally-occurring amino acid of similar character either in relation to polarity, side chain functionality, or size, for example Ser↔Thr↔ProHyp↔Gly↔Ala, Val↔Ile↔Leu, His↔Lys↔Arg, Asn↔-Gln↔Asp↔Glu or Phe↔Trp↔Tyr. It is to be understood that some non-conventional amino acids may also be suitable replacements for the naturally occurring amino acids. For example ornithine, homoarginine and dimethyllysine are related to His, Arg and Lys.

Substitutions encompassed by the present invention may also be “non-conservative”, in which an amino acid residue which is present in a polypeptide is substituted with an amino acid having different properties, such as a naturally-occurring amino acid from a different group (e.g. substituting a charged or hydrophobic amino acid with alanine), or alternatively, in which a naturally-occurring amino acid is substituted with a non-conventional amino acid.

Amino acid substitutions are typically of single residues, but may be of multiple residues, either clustered or dispersed. Preferably, amino acid substitutions are conservative.

Additions encompass the addition of one or more naturally occurring or non-conventional amino acid residues. Deletion encompasses the deletion of one or more amino acid residues.

As stated above the present invention includes peptides in which one or more of the amino acids has undergone sidechain modifications. Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivatisation, for example, to a corresponding amide. Sulphydryl groups may be modified by methods such as carboxymethylation with iodoacetic acid or iodoacetamide; performic acid oxidation to cysteic acid; formation of mixed disulfides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH. In a preferred form of the invention, any modification of cysteine residues must not affect the ability of the peptide to form the necessary disulfide bonds. It is also possible to replace the sulphydryl groups of cysteine with selenium equivalents such that the peptide forms a di-selenium bond in place of one or more of the disulfide bonds.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate. Proline residues may be modified by, for example, hydroxylation in the 4-position.

A list of some amino acids having modified side chains and other unnatural amino acids is shown in the following table:

| Non-conventional | | Non-conventional | |
|---|---|---|---|
| amino acid | Code | amino acid | Code |
| L-α-aminobutyric acid | Abu | L-α-methylhistidine | Mhis |
| α-amino-α-methylbutyrate | Mgabu | L-α-methylisoleucine | Mile |
| aminocyclopropane- | Cpro | L-α-methylleucine | Mleu |
| carboxylate | | L-α-methylmethionine | Mmet |
| aminoisobutyric acid | Aib | L-α-methylnorvaline | Mnva |
| aminonorbornyl- | Norb | L-α-methylphenylalanine | Mphe |
| carboxylate | | L-α-methylserine | Mser |
| cyclohexylalanine | Chexa | L-α-methyltryptophan | Mtrp |
| cyclopentylalanine | Cpen | L-α-methylvaline | Mval |
| D-alanine | DAla | N-(N-(2,2 diphenylethyl) | Nnbluu |
| D-arginine | DArg | carbamylmethylglycine | |
| D-asparagine | DAsn | 1-carboxy-1-(2,2-diphenyl- | Nmbc |
| D-aspartic acid | DAsp | ethylamino)cyclopropane | |
| D-cysteine | DCys | L-N-methylalanine | Nmala |
| D-glutamine | DGln | L-N-methylarginine | Nmarg |
| D-glutamic acid | DGlu | L-N-methylaspartic acid | Nmasp |
| D-histidine | DHis | L-N-methylcysteine | Nmcys |
| D-isoleucine | DIle | L-N-methylglutamine | Nmgln |
| D-leucine | DLeu | L-N-methylglutamic acid | Nmglu |
| D-lysine | DLys | L-N-methylhistidine | Nmhis |
| D-methionine | DMet | L-N-methylisolleucine | Nmile |
| D-ornithine | DOrn | L-N-methylleucine | Nmleu |
| D-phenylalanine | DPhe | L-N-methyllysine | Nmlys |
| D-proline | DPro | L-N -methylmethionine | Nmmet |
| D-serine | DSer | L-N-methylnorleucine | Nmnle |
| D-threonine | DThr | L-N-methylnorvaline | Nmnva |
| D-tryptophan | DTrp | L-N-methylornithine | Nmorn |
| D-tyrosine | DTyr | L-N-methylphenylalanne | Nmphe |
| D-valine | DVal | L-N-methylproline | Nmpro |
| D-α-methylalanine | DMala | L-N-methylserine | Nmser |
| D-α-methylarginine | DMarg | L-N-methylthreonine | Nmthr |
| D-α-methylasparagine | DMasn | L-N-methyltryptophan | Nmtrp |
| D-α-methylaspartate | DMasp | L-N-methyltyrosine | Nmtyr |
| D-α-methylcysteine | DMcys | L-N-methylvaline | Nmval |
| D-α-methyl glutami ne | DMgln | L-N-methylethylglycine | Nmetg |
| D-α-methylhistidine | DMhis | L-N-methyl-t- | Nmtbug |
| | | butylglycine | |
| D-α-methylisoleucine | DMile | L-norleucine | Nle |
| D-α-methylleucine | DMleu | L-norvaline | Nva |
| D-α-methyllysine | DMlys | α-methyl-aminoisobutyrate | Maib |
| D-α-methylmethionine | DMmet | α-methyl-y-aminobutyrate | Mgabu |
| D-α-methylornithine | DMorn | α-methylcyclohcxylalanine | Mchexa |
| D-α-methylphenylalanine | DMphe | α-methylcyclopentylalanine | Mcpen |
| D-α-methylproline | DMpro | α-methyl-α-napthylalanine | Manap |
| D-α-methylserine | DMser | α-methylpenicillamine | Mpen |
| D-α-methylthreonine | DMthr | N-(4-aminobutyl)glycine | Nglu |
| D-α-methyltyptophan | DMtrp | N-(2-aminoethyl)glycine | Naeg |
| D-α-methyltyrosine | DMty | N-(3-aminopropyl)glycine | Norn |
| D-α-methylvaline | DMval | N-amino-α-methylbutyrate | Nmaabu |
| D-N-methylalanine | DNmala | α-napthylalanine | Anap |
| D-N-methylarginine | DNmarg | N-benzylglycine | Nphe |
| D-N-methylasparagine | DNmasn | N-(2-carbamylethyl)glycine | Ngln |
| D-N-methylaspartate | DNmasp | N-(carbamylmethyl)glycine | Nasn |
| D-N-methylcysteine | DNmcys | N-(2-carboxyethyl)glycine | Nglu |
| D-N-methylglutamine | DNmgln | N-(carboxymethyl)glycine | Nasp |
| γ-carboxyglutamate | Gla | N-cyclobutylglycine | Ncbut |
| 4-hydroxyproline | Hyp | N-cyclodecylglycine | Ncdec |
| 5-hydroxylysine | Hlys | N-cylcododecylglycine | Ncdod |
| 2-aminobenzoyl | Abz | N-cyclooctylglycine | Ncoct |
| (anthraniloyl) | | N-cyclopropylglycine | Ncpro |
| Cyclohexylalanine | Cha | N-cycloundecylglycine | Ncund |
| Phenylglycine | Phg | N-(2,2-diphenylethyl)glycine | Nbhm |
| 4-phenyl-phenylalanine | Bib | N-(3,3-diphenylpropyl) | Nbhe |
| | | glycine | |
| L-Citrulline | Cit | N-(hydroxyethyl)glycine | Nser |
| L-1,2,3,4-tetrahydroiso- | Tic | N-(imidazolylethyl)glycine | Nhis |

These types of modifications may be important to stabilise the peptide if administered to an individual or for use as a diagnostic reagent.

Conservative amino acid substitutions, as used herein, may include amino acid residues within a group which have sufficiently similar physicochemical properties, so that a substitution between members of the group will preserve the biological activity of the molecule (see for example Grantham, R., 1974). Particularly, conservative amino acid substitutions are preferably substitutions in which the amino acids originate from the same class of amino acids (e.g. basic amino acids, acidic amino acids, polar amino acids, amino acids with aliphatic side chains, amino acids with positively or negatively charged side chains, amino acids with aromatic groups in the side chains, amino acids the side chains of which can enter into hydrogen bridges, e.g. side chains which have a hydroxyl function). Conservative substitutions are in the present case for example substituting a basic amino acid residue (Lys, Arg, His) for another basic amino acid residue (Lys, Arg, His), substituting an aliphatic amino acid residue (Gly, Ala, Val, Leu, lie) for another aliphatic amino acid residue, substituting an aromatic amino acid residue (Phe, Tyr, Trp) for another aromatic amino acid residue, substituting threonine by serine or leucine by isoleucine. Further conservative amino acid exchanges will be known to the person skilled in the art. The isomer form should preferably be maintained, e.g. K is preferably substituted for R or H, while k is preferably substituted for r and h.

When considering replacement amino acids, preferred replacements of the present invention are those described as having a D of less than 100 in Grantham, R. (1974), the contents of which are incorporated by reference. Most preferred replacements are those described as having a D of less than 50.

Peptide modulators of the present invention include retro inverso isomers of, or modified or substituted variants of, SEQ ID NO: 1 to 8, 19, 21 to 31, or peptides formed by additions thereto or deletions therefrom (Li et al., 2010).

4. Modulators that are an Analogue, Fragment or Derivative of an IgSF CAM

In one form of the invention, a modulator of the invention is an analogue, fragment or derivative of IgSF CAM that is an activator, an inhibitor, an allosteric modulator, or a non-functional mimic of the cytosolic tail of IgSF CAM. In a preferred form of the invention, a non-functional substitute is a modulator that mimics the cytosolic tail of IgSF CAM in the presence of certain co-located GPCRs, is not able to be activated by them or induce downstream IgSF CAM-dependent signalling, and inhibits signalling that normally occurs through activation of the cytosolic tail of IgSF CAM and IgSF CAM-dependent signalling resulting therefrom.

In one form of the invention, a modulator of the invention is an analogue, fragment or derivative of IgSF CAM that is an activator, an inhibitor, an allosteric modulator, or a non-functional mimic of the cytosolic tail of IgSF CAM. In a preferred form of the invention, a non-functional substitute is a modulator that mimics the cytosolic tail of IgSF CAM in the presence of certain co-located GPCRs, is not able to be activated by them or induce downstream IgSF CAM-dependent signalling, and inhibits signalling that normally occurs through activation of the cytosolic tail of RAGE and RAGE-dependent signalling resulting therefrom.

In one form of the invention, a modulator of the invention is an analogue, fragment or derivative of IgSF CAM that is an activator, an inhibitor, an allosteric modulator, or a non-functional mimic of the transmembrane domain of IgSF CAM or part thereof.

In one form of the invention, a non-functional substitute is a modulator that mimics the transmembrane domain of IgSF CAM in the presence of certain co-located GPCRs, is not able to be activated by them or induce downstream IgSF CAM-dependent signalling, and inhibits signalling that normally occurs through activation of the cytosolic tail of IgSF CAM and IgSF CAM-dependent signalling resulting therefrom.

In one form of the invention, a non-functional substitute is a modulator that mimics the transmembrane domain of IgSF CAM in the presence of certain co-located GPCRs, is not able to be activated by them or induce downstream IgSF CAM-dependent signalling, and inhibits signalling that normally occurs through activation of the cytosolic tail of RAGE and RAGE-dependent signalling resulting therefrom.

In one form of the invention, the modulator comprises a transmembrane domain of IgSF CAM or a part thereof and a fragment of the IgSF CAM ectodomain.

In one form of the invention, the modulator comprises a transmembrane domain of IgSF CAM or a part thereof and a fragment of the cytosolic tail of IgSF CAM.

In one form of the invention, the modulator comprises a transmembrane domain of IgSF CAM or part thereof and a fragment of the IgSF CAM ectodomain and a fragment of the cytosolic tail of IgSF CAM.

In one form of the invention, modulators of the invention contain a fragment of the ectodomain of IgSF CAM, which is not greater than 40, not greater than 20, not greater than 10 or not greater than 5 amino acids in length.

In one form, the present invention comprises modulators of RAGE ligand-independent activation of RAGE by certain activated co-located GPCRs, where these modulators are analogues, fragments or derivatives of IgSF CAM and that modulate transactivation of the cytosolic tail of RAGE triggered by activation of such certain activated co-located GPCRs, such as an angiotensin receptor.

In one form, the present invention comprises modulators of RAGE ligand-dependent activation of RAGE by its cognate ligand, where these modulators are analogues, fragments or derivatives of IgSF CAM.

In one form, the present invention comprises modulators of RAGE ligand-independent activation of RAGE by certain activated co-located GPCRs and RAGE ligand-dependent activation of RAGE by its cognate ligand, where these modulators are analogues, fragments or derivatives of IgSF CAM.

In one form, the present invention comprises modulators of IgSF CAM ligand-independent activation of the cytosolic tail of IgSF CAM by certain activated co-located GPCRs that bind to Ras GTPase-activating-like protein (IQGAP1) or other IgSF CAM-associated proteins, including protein kinase C zeta (PKCζ), Dock7, MyD88, TIRAP, IRAK4, ERK1/2, olfactory receptor 2T2, ADP/ATP translocase 2, Protein phosphatase 1G, Intercellular adhesion molecule 1, Protein DJ-1 (PARK7), Calponin-3, Drebrin, Filamin B, Ras-related protein Rab-13, Radixin/Ezrin/Moesin, Proteolipid protein 2, Coronin, S100 A11, Succinyl-CoA ligase [GDP-forming] subunit alpha, Hsc70-interacting protein, Apoptosis Inhibitor 5, neuropilin, cleavage stimulation factor, growth factor receptor-bound protein 2, sec61 beta subunit, or Nck1, or disrupt the binding of these elements to IgSF CAM, in order to modulate IgSF CAM transactivation by certain activated co-located GPCRs, such as an angiotensin receptor, such as AT₁R. In a preferred form of the invention, the modulators are analogues, fragments or derivatives of IgSF CAM. In a preferred form of the invention, the modulators are analogues, fragments or derivatives of the cytosolic tail of IgSF CAM. In a particularly preferred form of the invention, the modulators are analogues, fragments or derivatives of ALCAM₅₅₉₋₅₈₀. In another particularly preferred form of the invention, the modulators are analogues, fragments or derivatives of ALCAM₅₅₉₋₅₈₀ that differ by one, two, three, four, five, six, seven, eight, nine or ten amino acids. In another particularly preferred form of the invention, the modulator is ALCAM₅₅₉₋₅₈₀.

In one form, the present invention comprises modulators of RAGE ligand-independent activation of the cytosolic tail of RAGE by certain activated co-located GPCRs that bind to Ras GTPase-activating-like protein (IQGAP1) or other RAGE-associated proteins, including protein kinase C zeta (PKCζ), Dock7, MyD88, TIRAP, IRAK4, ERK1/2, olfactory receptor 2T2, ADP/ATP translocase 2, Protein phosphatase 1G, Intercellular adhesion molecule 1, Protein DJ-1 (PARK7), Calponin-3, Drebrin, Filamin B, Ras-related protein Rab-13, Radixin/Ezrin/Moesin, Proteolipid protein 2, Coronin, S100 A11, Succinyl-CoA ligase [GDP-forming] subunit alpha, Hsc70-interacting protein, Apoptosis Inhibitor 5, neuropilin, cleavage stimulation factor, growth factor receptor-bound protein 2, sec61 beta subunit, or Nck1, or disrupt the binding of these elements to RAGE, in order to modulate RAGE transactivation by certain activated co-located GPCRs, such as an angiotensin receptor, such as AT₁R, and where these modulators are analogues, fragments or derivatives of IgSF CAM. In a preferred form of the invention, the modulators are analogues, fragments or derivatives of the cytosolic tail of IgSF CAM. In a particularly preferred form of the invention, the modulators are analogues, fragments or derivatives of ALCAM₅₅₉₋₅₈₀. In another particularly preferred form of the invention, the modulators are analogues, fragments or derivatives of ALCAM₅₅₉₋₅₈₀ that differ by one, two, three, four, five, six, seven, eight, nine or ten amino acids. In another particularly preferred form of the invention, the modulator is ALCAM₅₅₉₋₅₈₀.

In one form of the invention, the modulators of the invention bind to the cytosolic elements of the certain activated co-located GPCR, IgSF CAM and/or elements complexed with either, including IQGAP-1, PKCζ, Dock7, MyD88, TIRAP, IRAK4, ERK1/2, olfactory receptor 2T2, ADP/ATP translocase 2, Protein phosphatase 1G, Intercellular adhesion molecule 1, Protein DJ-1 (PARK7), Calponin-3, Drebrin, Filamin B, Ras-related protein Rab-13, Radixin/Ezrin/Moesin, Proteolipid protein 2, Coronin, S100 A11, Succinyl-CoA ligase [GDP-forming] subunit alpha, Hsc70-interacting protein, Apoptosis Inhibitor 5, neuropilin, cleavage stimulation factor, growth factor receptor-bound protein 2, sec61 beta subunit, or Nck1 to modulate IgSF CAM ligand-independent signalling through the cytosolic tail of IgSF CAM, by modulating these signalling elements required for IgSF CAM transactivation by certain activated co-located GPCRs, such as an angiotensin receptor, such as AT₁R, and where these modulators are analogues, fragments or derivatives of IgSF CAM. In a preferred form of the invention, the modulators are analogues, fragments or derivatives of the cytosolic tail of IgSF CAM. In a particularly preferred form of the invention, the modulators are analogues, fragments or derivatives of ALCAM₅₅₉₋₅₈₀. In another particularly preferred form of the invention, the modulators are analogues, fragments or derivatives of ALCAM₅₅₉₋₅₈₀ that differ by one, two, three, four, five, six, seven, eight, nine or ten amino acids. In another particularly preferred form of the invention, the modulator is ALCAM₅₅₉₋₅₈₀.

In one form of the invention, the modulators of the invention bind to the cytosolic elements of the certain activated co-located GPCR, RAGE and/or elements complexed with either, including IQGAP-1, PKCζ, Dock7, MyD88, TIRAP, IRAK4, ERK1/2, olfactory receptor 2T2, ADP/ATP translocase 2, Protein phosphatase 1G, Intercellular adhesion molecule 1, Protein DJ-1 (PARK7), Calponin-3, Drebrin, Filamin B, Ras-related protein Rab-13, Radixin/Ezrin/Moesin, Proteolipid protein 2, Coronin, S100 A11, Succinyl-CoA ligase [GDP-forming] subunit alpha, Hsc70-interacting protein, Apoptosis Inhibitor 5, neuropilin, cleavage stimulation factor, growth factor receptor-bound protein 2, sec61 beta subunit, or Nck1 to modulate RAGE ligand-independent signalling through the cytosolic tail of RAGE, by modulating these signalling elements required for RAGE transactivation by certain activated co-located GPCRs, such as an angiotensin receptor, such as AT₁R, and where these modulators are analogues, fragments or derivatives of IgSF CAM. In a preferred form of the invention, the modulators are analogues, fragments or derivatives of the cytosolic tail of IgSF CAM. In a particularly preferred form of the invention, the modulators are analogues, fragments or derivatives of ALCAM₅₅₉₋₅₈₀. In another particularly preferred form of the invention, the modulators are analogues, fragments or derivatives of ALCAM₅₅₉₋₅₈₀ that differ by one, two, three, four, five, six, seven, eight, nine or ten amino acids. In another particularly preferred form of the invention, the modulator is ALCAM₅₅₉₋₅₈₀.

In one form of the invention, modulators of IgSF CAM ligand-independent activation of IgSF CAM by certain activated co-located GPCRs also modulate IgSF CAM ligand-dependent activation of the cytosolic tail of IgSF CAM, by binding to cytosolic elements of IgSF CAM and/or elements that complex with IgSF CAM in the cytosol (such as IQGAP-1, PKCζ, Dock7, MyD88, IRAK4, TIRAP, ERK1/2, olfactory receptor 2T2, ADP/ATP translocase 2, Protein phosphatase 1G, Intercellular adhesion molecule 1, Protein DJ-1 (PARK7), Calponin-3, Drebrin, Filamin B, Ras-related protein Rab-13, Radixin/Ezrin/Moesin, Proteolipid protein 2, Coronin, S100 A11, Succinyl-CoA ligase [GDP-forming] subunit alpha, Hsc70-interacting protein, Apoptosis Inhibitor 5, neuropilin, cleavage stimulation factor, growth factor receptor-bound protein 2, sec61 beta subunit, or Nck1) to inhibit IgSF CAM ligand-mediated signalling through these elements. In a preferred form of the invention, the modulators are analogues, fragments or derivatives of IgSF CAM. In a preferred form of the invention, the modulators are analogues, fragments or derivatives of the cytosolic tail of IgSF CAM. In a particularly preferred form of the invention, the modulators are analogues, fragments or derivatives of ALCAM₅₅₉₋₅₈₀. In another particularly preferred form of the invention, the modulators are analogues, fragments or derivatives of ALCAM₅₅₉₋₅₈₀ that differ by one, two, three, four, five, six, seven, eight, nine or ten amino acids. In another particularly preferred form of the invention, the modulator is ALCAM₅₅₉₋₅₈₀.

In one form of the invention, modulators of RAGE ligand-independent activation of RAGE by certain activated co-located GPCRs also modulate RAGE ligand-dependent activation of the cytosolic tail of RAGE, by binding to cytosolic elements of RAGE and/or elements that complex with RAGE in the cytosol (such as IQGAP-1, PKCζ, Dock7, MyD88, IRAK4, TIRAP, ERK1/2, olfactory receptor 2T2, ADP/ATP translocase 2, Protein phosphatase 1G, Intercellular adhesion molecule 1, Protein DJ-1 (PARK7), Calponin-3, Drebrin, Filamin B, Ras-related protein Rab-13, Radixin/Ezrin/Moesin, Proteolipid protein 2, Coronin, S100 A11, Succinyl-CoA ligase [GDP-forming] subunit alpha, Hsc70-interacting protein, Apoptosis Inhibitor 5, neuropilin, cleavage stimulation factor, growth factor receptor-bound protein 2, sec61 beta subunit, or Nck1) to inhibit RAGE ligand-mediated signalling through these elements, and where the modulators are analogues, fragments or derivatives of IgSF CAM. In a preferred form of the invention, the modulators are analogues, fragments or derivatives of the cytosolic tail of IgSF CAM. In a particularly preferred form of the invention, the modulators are analogues, fragments or derivatives of ALCAM₅₅₉₋₅₈₀. In another particularly preferred form of the invention, the modulators are analogues, fragments or derivatives of ALCAM₅₅₉₋₅₈₀ that differ by one, two, three, four, five, six, seven, eight, nine or ten amino acids. In another particularly preferred form of the invention, the modulator is ALCAM₅₅₉₋₅₈₀.

In some embodiments, the modulator is introduced by gene delivery (such as by using a virus or artificial non-viral gene delivery such as electroporation, microinjection, gene gun, impalefection, hydrostatic pressure, continuous infusion, sonication, lipofection, liposomes, nanobubbles and polymeric gene carriers) and the peptide fragment, biologically-active analogue or derivative being generated by the cell as a consequence of transcriptional and translational processes.

In some embodiments, the modulator has a modified capacity to form a complex with certain co-located GPCRs, such as AT₁R, or elements that complex with them. For example, the IgSF CAM analogue, fragment or derivative may be distinguished from a wild-type IgSF CAM polypeptide or fragment sequence by the substitution, addition, or deletion of at least one amino acid residue or addition or substitution of unusual or non-conventional amino-acids or non-amino acid residues.

In one aspect of the invention, the modulator of the present invention includes isolated or purified peptides which comprise, consist, or consists essentially of an amino acid sequence represented by Formula I:

Z1MZ2 (I)

wherein:

Z1 is absent or is selected from at least one of a proteinaceous moiety comprising from about 1 to about 50 amino acid residues; and

M is the amino acid sequence as set forth in SEQ ID NO: 6, or an analogue, fragment or derivative thereof; and

Z2 is absent or is a proteinaceous moiety comprising from about 1 to about 50 amino acid residues.

In some embodiments of the invention described above, the modulator (such as a fragment of the IgSF CAM cytosolic tail, an analogue or derivative thereof as broadly described above and elsewhere herein) is able to penetrate a cell membrane. In non-limiting examples of this type, the modulator is conjugated, fused or otherwise linked to a cell membrane penetration molecule (e.g., the HIV TAT motif, as set forth in SEQ ID NO: 20 below).

SEQ ID NO: 20:
[YGRKKRRQRRR].

In some forms of the invention, the modulator is a non-peptide molecule that shares with the peptide modulator described above the capacity to bind to and/or interfere with elements associated with IgSF CAM ligand-independent activation of IgSF CAM by certain activated co-located GPCRs. These non-peptide modulators may or may not contain structural similarities to functionally important domains contained in peptide modulators.

In some forms of the invention, the modulator is a non-peptide molecule that shares with the peptide modulator described above the capacity to bind to and/or interfere with elements associated with RAGE ligand-independent activation of RAGE by certain activated co-located GPCRs. These non-peptide modulators may or may not contain structural similarities to functionally important domains contained in peptide modulators.

In preferred forms of the invention, the modulator is an inhibitor.

In certain forms of the invention, in addition to being an inhibitor of IgSF CAM ligand-independent activation of IgSF CAM by a certain activated co-located GPCR, the modulator is an inhibitor of the certain co-located GPCR and/or an inhibitor of the certain co-located GPCR signalling pathway.

In certain forms of the invention, in addition to being an inhibitor of RAGE ligand-independent activation of RAGE by a certain activated co-located GPCR, the modulator is an inhibitor of the certain co-located GPCR and/or an inhibitor of the certain co-located GPCR signalling pathway.

In certain forms of the invention, in addition to being an inhibitor of IgSF CAM ligand-independent activation of IgSF CAM by a certain activated co-located GPCR, the modulator is an inhibitor of IgSF CAM ligand-dependent activation of IgSF CAM and/or an inhibitor of constitutively-active IgSF CAM and/or an inhibitor of an IgSF CAM signalling pathway.

In certain forms of the invention, in addition to being an inhibitor of RAGE ligand-independent activation of RAGE by a certain activated co-located GPCR, the modulator is an inhibitor of RAGE ligand-dependent activation of RAGE and/or an inhibitor of constitutively-active RAGE and/or an inhibitor of a RAGE signalling pathway.

In certain forms of the invention, where the certain co-located GPCR is AT₁R, in addition to being an inhibitor of IgSF CAM ligand-independent activation of IgSF CAM, the modulator is an AT₁R inhibitor and/or an inhibitor of an AT₁R signalling pathway.

In certain forms of the invention, where the certain co-located GPCR is AT₁R, in addition to being an inhibitor of RAGE ligand-independent activation of RAGE, the modulator is an AT₁R inhibitor and/or an inhibitor of an AT₁R signalling pathway.

In certain forms of the invention, in addition to being an inhibitor of IgSF CAM ligand-independent activation of IgSF CAM by activated angiotensin receptor, preferably activated AT₁R, the modulator is an inhibitor of IgSF CAM ligand-dependent activation of IgSF CAM and/or an inhibitor of constitutively-active IgSF CAM and/or an inhibitor of an IgSF CAM signalling pathway.

In certain forms of the invention, in addition to being an inhibitor of RAGE ligand-independent activation of RAGE by activated angiotensin receptor, preferably activated AT₁R, the modulator is an inhibitor of RAGE ligand-dependent activation of RAGE and/or an inhibitor of constitutively-active RAGE and/or an inhibitor of a RAGE signalling pathway.

In certain forms of the invention, in addition to being an inhibitor of IgSF CAM ligand-independent activation of IgSF CAM by a certain activated co-located GPCR, the modulator is an inhibitor of the certain co-located GPCR and/or an inhibitor of the certain co-located GPCR signalling pathway and an inhibitor of IgSF CAM ligand-dependent activation of IgSF CAM and/or an inhibitor of constitutively-active IgSF CAM and/or an inhibitor of an IgSF CAM signalling pathway.

In certain forms of the invention, in addition to being an inhibitor of RAGE ligand-independent activation of RAGE by a certain activated co-located GPCR, the modulator is an inhibitor of the certain co-located GPCR and/or an inhibitor of the certain co-located GPCR signalling pathway and an inhibitor of RAGE ligand-dependent activation of RAGE and/or an inhibitor of constitutively-active RAGE and/or an inhibitor of a RAGE signalling pathway.

In certain forms of the invention, in addition to being an inhibitor of IgSF CAM ligand-independent activation of IgSF CAM by activated angiotensin receptor, preferably activated AT₁R, the modulator is an AT₁R inhibitor and/or an inhibitor of an AT₁R signalling pathway and an inhibitor of IgSF CAM ligand-dependent activation of IgSF CAM and/or an inhibitor of constitutively-active IgSF CAM and/or an inhibitor of an IgSF CAM signalling pathway.

In certain forms of the invention, in addition to being an inhibitor of RAGE ligand-independent activation of RAGE by activated angiotensin receptor, preferably activated AT₁R, the modulator is an AT₁R inhibitor and/or an inhibitor of an AT₁R signalling pathway and an inhibitor of RAGE ligand-dependent activation of RAGE and/or an inhibitor of constitutively-active RAGE and/or an inhibitor of a RAGE signalling pathway.

In certain forms of the invention, the modulator is a non-functional substitute for the cytosolic tail of IgSF CAM or a part thereof, which is not able to be activated by a co-located GPCR or facilitate downstream IgSF CAM-dependent signalling and inhibits signalling that occurs through the cytosolic tail of IgSF CAM and IgSF CAM-dependent signalling.

In certain forms of the invention, the modulator is a non-functional substitute for the cytosolic tail of IgSF CAM or a part thereof, which is not able to be activated by a co-located GPCR or facilitate downstream IgSF CAM-dependent signalling and inhibits signalling that occurs through the cytosolic tail of RAGE and RAGE-dependent signalling.

In certain forms of the invention, the modulator is a non-functional substitute for the transmembrane domain of IgSF CAM or a part thereof, which is not able to be activated by a co-located GPCR or facilitate downstream IgSF CAM-dependent signalling and inhibits signalling that occurs through the cytosolic tail of IgSF CAM and IgSF CAM-dependent signalling.

In certain forms of the invention, the modulator is a non-functional substitute for the transmembrane domain of IgSF CAM or a part thereof, which is not able to be activated by a co-located GPCR or facilitate downstream IgSF CAM-dependent signalling and inhibits signalling that occurs through the cytosolic tail of RAGE and RAGE-dependent signalling.

In certain forms of the invention, the modulator comprises a transmembrane domain of IgSF CAM or a part thereof and a fragment of the IgSF CAM ectodomain. In certain forms of the invention, the modulator comprises a transmembrane domain of IgSF CAM or a part thereof and a fragment of the cytosolic tail of IgSF CAM.

In certain forms of the invention, the modulator comprises a transmembrane domain of IgSF CAM or part thereof and a fragment of the IgSF CAM ectodomain and a fragment of the cytosolic tail of IgSF CAM.

In certain forms of the invention, the modulators of IgSF CAM ligand-independent activation of IgSF CAM by certain activated co-located GPCRs contain a fragment of the ligand-binding ectodomain of IgSF CAM, which is not greater than 40, not greater than 20, not greater than 10 or not greater than 5 amino acids in length.

In certain forms of the invention, the modulators of RAGE ligand-independent activation of RAGE by certain activated co-located GPCRs contain a fragment of the ligand-binding ectodomain of IgSF CAM, which is not greater than 40, not greater than 20, not greater than 10 or not greater than 5 amino acids in length.

5. Methods for Modulating Ligand-Independent Activation of an IgSF CAM or RAGE

In a related aspect, the present invention provides methods for modulating ligand-independent activation of an IgSF CAM by an activated certain co-located GPCR, such as angiotensin receptor, such as AT₁R, in a cell or tissue of an animal or of animal origin (which may or may not be of a human or of human origin) using a modulator as described herein.

In a related aspect, the present invention provides methods for modulating ligand-independent activation of RAGE by an activated certain co-located GPCR, such as angiotensin receptor, such as AT₁R, in a cell or tissue of an animal or of animal origin (which may or may not be of a human or of human origin) using a modulator as described herein that is an analogue, fragment or derivative of IgSF CAM.

In some aspects these methods include truncating or mutating an IgSF CAM such that it is unable to bind IgSF CAM ligands to its ectodomain, or that binding IgSF CAM ligands to its ectodomain is impaired by exposing the cell to a modulator that modulates the binding of IgSF CAM ligands to IgSF CAM.

In some forms of the invention, the modulation of the IgSF CAM ligand-independent signalling pathway, is distinct from and/or significantly more than the modulation of the IgSF CAM ligand-dependent signalling pathway.

In some forms of the invention, the inhibition of the IgSF CAM ligand-independent signalling pathway, is distinct from and/or significantly more than the inhibition of the IgSF CAM ligand-dependent signalling pathway.

The method may comprise administering a modulator to a patient.

6. Methods for Modulating Ligand-Dependent Activation of an IgSF CAM

In a related aspect, the present invention provides methods for modulating IgSF CAM ligand-dependent activation of an IgSF CAM by a cognate ligand in a cell or tissue of an animal or of animal origin (which may or may not be of a human or of human origin) using a modulator as described herein.

In a related aspect, the present invention provides methods for modulating RAGE ligand-dependent activation of RAGE by a cognate ligand in a cell or tissue of an animal or of animal origin (which may or may not be of a human or of human origin) using a modulator as described herein that is an analogue, fragment or derivative of IgSF CAM.

The method may comprise administering a modulator to a patient.

7. Methods for Modulating Both IgSF CAM Ligand-Dependent and IgSF CAM Ligand-Independent Activation of an IgSF CAM

In another related aspect, the present invention provides methods for inhibiting an IgSF CAM ligand-dependent activation of an IgSF CAM by IgSF CAM ligands (including AGE-modified proteins, lipids or DNA, members of the S100 calgranulin family of proteins, HMGB1, amyloid and Mac-1) and subsequent downstream signalling pathways in a cell, tissue or animal in addition to modulating an IgSF CAM ligand-independent activation of an IgSF CAM by certain activated co-located GPCRs.

In one aspect of the invention, these methods comprise using a modulator as described herein, including fragments, analogues or derivatives of the cytosolic tail of an IgSF CAM, to prevent or inhibit activation of both an IgSF CAM-ligand dependent activation of an IgSF CAM and an IgSF CAM ligand-independent activation of an IgSF CAM by certain activated co-located GPCRs. In one aspect of the invention, IgSF CAM-dependent signalling is impaired by exposing the cell to an inhibitor that inhibits the binding of signalling elements to the cytosolic tail of an IgSF CAM resulting in inhibition of both an IgSF CAM ligand-mediated activation of an IgSF CAM and an IgSF CAM ligand-independent activation of an IgSF CAM by certain activated co-located GPCRs.

In one aspect of the invention, these methods comprise using a modulator as described herein, including fragments, analogues or derivatives of the cytosolic tail of RAGE, to prevent activation of both an IgSF CAM-ligand dependent activation of an IgSF CAM and an IgSF CAM ligand-independent activation of an IgSF CAM by certain activated co-located GPCRs.

In one aspect of the invention, IgSF CAM-dependent signalling is impaired by exposing the cell to an inhibitor that inhibits the binding of signalling elements to the cytosolic tail of an IgSF CAM resulting in inhibition of both an IgSF CAM ligand-mediated activation of an IgSF CAM and an IgSF CAM ligand-independent activation of an IgSF CAM by certain activated co-located GPCRs. In one aspect of the invention, these methods comprise using a modulator as described herein, including fragments, analogues or derivatives of a IgSF CAM, to take the place of the transmembrane domain of an IgSF CAM and therein prevent activation of both an IgSF CAM-ligand dependent activation of an IgSF CAM and an IgSF CAM ligand-independent activation of an IgSF CAM by certain activated co-located GPCRs.

8. Methods for Modulating Both RAGE Ligand-Dependent and RAGE Ligand-Independent Activation of RAGE Using Modulators that are Analogues, Fragments or Derivatives of IgSF CAM

In another related aspect, the present invention provides methods for inhibiting RAGE ligand-dependent activation of RAGE by RAGE ligands (including AGE-modified proteins, lipids or DNA, members of the S100 calgranulin family of proteins, HMGB1, amyloid and Mac-1) and subsequent downstream signalling pathways in a cell, tissue or animal in addition to modulating RAGE ligand-independent activation of RAGE by certain activated co-located GPCRs where the modulator is an analogue, fragment or derivative of IgSF CAM.

In one aspect of the invention, these methods comprise using a modulator as described herein where the modulator is an analogue, fragment or derivative of IgSF CAM, to prevent or inhibit activation of both RAGE-ligand dependent activation of RAGE and RAGE ligand-independent activation of RAGE by certain activated co-located GPCRs. In one aspect of the invention, RAGE-dependent signalling is impaired by exposing the cell to an inhibitor that inhibits the binding of signalling elements to the cytosolic tail of RAGE resulting in inhibition of both RAGE ligand-mediated activation of RAGE and RAGE ligand-independent activation of RAGE by certain activated co-located GPCRs.

In one aspect of the invention, these methods comprise using a modulator as described herein where the modulator is an analogue, fragment or derivative of IgSF CAM, to prevent activation of both RAGE-ligand dependent activation of RAGE and RAGE ligand-independent activation of RAGE by certain activated co-located GPCRs.

In one aspect of the invention, RAGE-dependent signalling is impaired by exposing the cell to an inhibitor that inhibits the binding of signalling elements to the cytosolic tail of RAGE resulting in inhibition of both RAGE ligand-mediated activation of RAGE and RAGE ligand-independent activation of RAGE by certain activated co-located GPCRs. In one aspect of the invention, these methods comprise using a modulator as described herein where the modulator is an analogue, fragment or derivative of IgSF CAM, to take the place of the transmembrane domain of RAGE and therein prevent activation of both RAGE-ligand dependent activation of RAGE and RAGE ligand-independent activation of RAGE by certain activated co-located GPCRs.

In specific embodiments, the modulator comprises, consists, or consists essentially of an amino acid sequence as set forth in SEQ ID NO: 1, or an analogue, fragment or derivative thereof.

In specific embodiments, the modulator comprises, consists, or consists essentially of an amino acid sequence as set forth in SEQ ID NO: 1, or an analogue, fragment or derivative thereof that differs by one, two, three, four, five, six, seven, eight, nine, ten, eleven, twelve, thirteen, fourteen, fifteen, sixteen, seventeen, eighteen, nineteen or twenty amino acids.

In specific embodiments, the modulator comprises, consists, or consists essentially of an amino acid sequence as set forth in SEQ ID NO: 2, or an analogue, fragment or derivative thereof.

In specific embodiments, the modulator comprises, consists, or consists essentially of an amino acid sequence as set forth in SEQ ID NO: 2, or an analogue, fragment or derivative thereof that differs by one, two, three, four, five, six, seven, eight, nine, ten, eleven, twelve, thirteen, fourteen, fifteen, sixteen, seventeen, eighteen, nineteen or twenty amino acids.

In specific embodiments, the modulator comprises, consists, or consists essentially of an amino acid sequence as set forth in SEQ ID NO: 3, or an analogue, fragment or derivative thereof.

In specific embodiments, the modulator comprises, consists, or consists essentially of an amino acid sequence as set forth in SEQ ID NO: 3, or an analogue, fragment or derivative thereof that differs by one, two, three, four, five, six, seven, eight, nine, ten, eleven, twelve, thirteen, fourteen, fifteen, sixteen, seventeen, eighteen, nineteen or twenty amino acids.

In specific embodiments, the modulator comprises, consists, or consists essentially of an amino acid sequence as set forth in SEQ ID NO: 4, or an analogue, fragment or derivative thereof.

In specific embodiments, the modulator comprises, consists, or consists essentially of an amino acid sequence as set forth in SEQ ID NO: 4, or an analogue, fragment or derivative thereof that differs by one, two, three, four, five, six, seven, eight, nine, or ten amino acids.

In specific embodiments, the modulator comprises, consists, or consists essentially of an amino acid sequence as set forth in SEQ ID NO: 5, or an analogue, fragment or derivative thereof.

In specific embodiments, the modulator comprises, consists, or consists essentially of an amino acid sequence as set forth in SEQ ID NO: 5, or an analogue, fragment or derivative thereof that differs by one, two, three, four, five, six, seven, eight, nine, or ten amino acids.

In specific embodiments, the modulator comprises, consists, or consists essentially of an amino acid sequence as set forth in SEQ ID NO: 6, or an analogue, fragment or derivative thereof.

In specific embodiments, the modulator comprises, consists, or consists essentially of an amino acid sequence as set forth in SEQ ID NO: 6, or an analogue, fragment or derivative thereof that differs by one, two, three, four, five, six, seven, eight, nine, ten, eleven, twelve, thirteen, fourteen, fifteen, sixteen, seventeen, eighteen, nineteen or twenty amino acids.

In one aspect of the invention, the modulator comprises the cytosolic domain of a IgSF CAM or a part thereof, which is not greater than 40, not greater than 20, not greater than 10 or not greater than 5 amino acids in length.

In one aspect of the invention, these methods comprise using a modulator as described herein that is a fragment, analogue or derivative of RAGE, to take the place of the transmembrane domain of an IgSF CAM and therein prevent activation of both an IgSF CAM-ligand dependent activation of an IgSF CAM and an IgSF CAM ligand-independent activation of an IgSF CAM by certain activated co-located GPCRs. In one aspect of the invention, the modulator comprises the cytosolic domain of RAGE or a part thereof, which is not greater than 40, not greater than 20, not greater than 10 or not greater than 5 amino acids in length.

In one aspect of the invention, these methods comprise using a modulator as described herein that is a fragment, analogue or derivative of IgSF CAM, to take the place of the transmembrane domain of an IgSF CAM and therein prevent activation of both an IgSF CAM-ligand dependent activation of an IgSF CAM and an IgSF CAM ligand-independent activation of an IgSF CAM by certain activated co-located GPCRs. In one aspect of the invention, the modulator comprises the cytosolic domain of IgSF CAM or a part thereof, which is not greater than 40, not greater than 20, not greater than 10 or not greater than 5 amino acids in length.

In one aspect, inhibition of the IgSF CAM ligand-dependent activation of an IgSF CAM occurs at the same time as inhibition of the IgSF CAM ligand-independent activation of an IgSF CAM by certain activated co-located GPCR.

In one aspect, inhibition of the RAGE ligand-dependent activation of RAGE occurs at the same time as inhibition of the RAGE ligand-independent activation of RAGE by certain activated co-located GPCR where the modulator is an analogue, fragment or derivative of IgSF CAM.

In one aspect, these methods comprise silencing, truncating, modifying or mutating an IgSF CAM such that an IgSF CAM, or analogues, fragments or derivatives thereof, are a non-functional substitute for the cytosolic tail of wild type IgSF CAM or a part thereof, which are unable to be activated by either ligand-dependent or ligand-independent pathways or facilitate downstream signalling and so inhibit signalling that occurs through the cytosolic tail of an IgSF CAM and IgSF CAM-dependent signalling.

In one aspect, these methods comprise silencing, truncating, modifying or mutating an IgSF CAM such that an IgSF CAM, or analogues, fragments or derivatives thereof, are a non-functional substitute for the cytosolic tail of wild type IgSF CAM or a part thereof, which are unable to be activated by either ligand-dependent or ligand-independent pathways or facilitate downstream signalling and so inhibit signalling that occurs through the cytosolic tail of RAGE and RAGE-dependent signalling.

In one aspect, these methods comprise silencing, truncating, modifying or mutating RAGE such that RAGE, or analogues, fragments or derivatives thereof, are a non-functional substitute for the cytosolic tail of wild type IgSF CAM or a part thereof, which is unable to be activated by either ligand-dependent or ligand-independent pathways or facilitate downstream signalling and so inhibit signalling that occurs through the cytosolic tail of an IgSF CAM and IgSF CAM-dependent signalling.

In one aspect, the modulators of an IgSF CAM ligand-independent activation of an IgSF CAM by certain activated co-located GPCRs contain a fragment of the ligand-binding ectodomain of human wild-type IgSF CAM, which is not greater than 40, not greater than 20, not greater than 10 or not greater than 5 amino acids in length.

In one aspect, the modulators of an IgSF CAM ligand-independent activation of an IgSF CAM by certain activated co-located GPCRs contain a fragment of the ligand-binding ectodomain of human wild-type RAGE, which is not greater than 40, not greater than 20, not greater than 10 or not greater than 5 amino acids in length.

In one aspect, these methods comprise silencing, truncating, modifying or mutating IgSF CAM such that an IgSF CAM, or analogues, fragments or derivatives thereof, modulate common elements involved in signalling mediated by the cytosolic tail of an IgSF CAM (such as PKCζ, Diaph1, MyD88, TIRAP, NFκB). Association with activation of an IgSF CAM by either IgSF CAM ligand-dependent or IgSF CAM ligand-independent activation pathways.

In one aspect, these methods comprise silencing, truncating, modifying or mutating RAGE such that RAGE, or analogues, fragments or derivatives thereof, modulates common elements involved in signalling mediated by the cytosolic tail of an IgSF CAM (such as PKCζ, Diaph1, MyD88, TIRAP, NFκB). Association with activation of an IgSF CAM by either IgSF CAM ligand-dependent or IgSF CAM ligand-independent activation pathways.

In one aspect, these methods comprise the use of a modulator that modulates an IgSF CAM ligand-independent activation of an IgSF CAM by activated certain co-located GPCR, such as angiotensin receptor, such as AT₁R, in addition to a modulator that modulates an IgSF CAM ligand-dependent activation of an IgSF CAM (such as by a modulator that modulates the binding of an IgSF CAM ligands to the IgSF CAM ectodomain).

The method may comprise administering a modulator to a patient.

9. Methods for Modulating IgSF CAM Ligand-Independent Activation of an IgSF CAM by Certain Activated Co-Located GPCRs while Also Modulating IgSF CAM-Independent Signalling Via Certain Co-Located GPCRs.

In one aspect, the invention provides a method for modulating an IgSF CAM-independent, certain co-located GPCR signalling pathway induced following activation by a cognate ligand as well as modulating an IgSF CAM ligand-independent activation of an IgSF CAM by a certain activated co-located GPCR.

In one form, the invention provides a method for modulating an IgSF CAM-independent, certain co-located GPCR signalling pathway induced following activation by a cognate ligand at the same time as modulating an IgSF CAM ligand-independent activation of an IgSF CAM by a certain activated co-located GPCR.

The method may comprise administering a modulator to a patient.

10. Methods for Modulating Ligand-Dependent Activation of an IgSF CAM

In a related aspect, the present invention provides methods for modulating ligand-dependent activation of an IgSF CAM by a cognate ligand in a cell or tissue of an animal or of animal origin (which may or may not be of a human or of human origin).

The method may comprise administering a modulator to a patient.

11. Methods for Modulating Both Ligand-Dependent and Ligand-Independent Activation of an IgSF CAM

In another related aspect, the present invention provides methods for inhibiting ligand-dependent activation of an IgSF CAM by cognate ligand and subsequent downstream signalling pathways in a cell, tissue or animal in addition to modulating ligand-independent activation of an IgSF CAM by certain activated co-located GPCRs.

In one aspect of the invention, these methods comprise using a modulator as described herein to take the place of the cytosolic tail of an IgSF CAM in binding interactions and therein prevent activation of both ligand-dependent activation of an IgSF CAM and ligand-independent activation of an IgSF CAM by certain activated co-located GPCRs. In a preferred form of the invention, the modulator is a fragment, analogue or derivative of the cytosolic tail of an IgSF CAM. In one aspect of the invention, signalling is impaired by exposing the cell to an inhibitor that inhibits the binding of signalling elements to the cytosolic tail of an IgSF CAM resulting in inhibition of both ligand-mediated activation of an IgSF CAM and ligand-independent activation of an IgSF CAM by certain activated co-located GPCRs.

In one aspect of the invention, these methods comprise using a modulator as described herein to take the place of the cytosolic tail of an IgSF CAM in binding interactions and therein prevent activation of both ligand-dependent activation of an IgSF CAM and ligand-independent activation of an IgSF CAM by certain activated co-located GPCRs. In a preferred form of the invention, the modulator is a fragment, analogue or derivative of the cytosolic tail of RAGE. In one aspect of the invention, signalling is impaired by exposing the cell to an inhibitor that inhibits the binding of signalling elements to the cytosolic tail of an IgSF CAM resulting in inhibition of both ligand-mediated activation of an IgSF CAM and ligand-independent activation of an IgSF CAM by certain activated co-located GPCRs.

In one aspect of the invention, these methods comprise using a modulator as described herein to take the place of the transmembrane domain of an IgSF CAM and therein prevent activation of both ligand dependent activation of an IgSF CAM and ligand-independent activation of an IgSF CAM by certain activated co-located GPCRs. In a preferred form of the invention, the modulator is a fragment, analogue or derivative of the cytosolic tail of an IgSF CAM. In one aspect of the invention, the modulator comprises the cytosolic domain of an IgSF CAM or a part thereof, which is not greater than 40, not greater than 20, not greater than 10 or not greater than 5 amino acids in length.

In one aspect of the invention, these methods comprise using a modulator as described herein to take the place of the transmembrane domain of an IgSF CAM and therein prevent activation of both ligand dependent activation of an IgSF CAM and ligand-independent activation of an IgSF CAM by certain activated co-located GPCRs. In a preferred form of the invention, the modulator is a fragment, analogue or derivative of the cytosolic tail of RAGE. In one aspect of the invention, the modulator comprises the cytosolic domain of RAGE or a part thereof, which is not greater than 40, not greater than 20, not greater than 10 or not greater than 5 amino acids in length.

In one aspect, inhibition of the ligand-dependent activation of an IgSF CAM occurs at the same time as inhibition of the ligand-independent activation of an IgSF CAM by certain activated co-located GPCR.

In one aspect, these methods comprise silencing, truncating, modifying or mutating an IgSF CAM such that an IgSF CAM, or analogues, fragments or derivatives thereof, are a non-functional substitute for the cytosolic tail of wild type an IgSF CAM or a part thereof, which are unable to be activated by either ligand-dependent or ligand-independent pathways or facilitate downstream signalling and so inhibit signalling that occurs through the cytosolic tail of an IgSF CAM dependent signalling.

In one aspect, these methods comprise silencing, truncating, modifying or mutating RAGE such that RAGE, or analogues, fragments or derivatives thereof, are a non-functional substitute for the cytosolic tail of wild type IgSF CAM or a part thereof, which is unable to be activated by either ligand-dependent or ligand-independent pathways or facilitate downstream signalling and so inhibit signalling that occurs through the cytosolic tail of an IgSF CAM dependent signalling.

In one aspect, the modulators of ligand-independent activation of an IgSF CAM by certain activated co-located GPCRs contain a fragment of the ligand-binding ectodomain of human wild-type IgSF CAM which is not greater than 40, not greater than 20, not greater than 10 or not greater than 5 amino acids in length.

In one aspect, the modulators of ligand-independent activation of an IgSF CAM by certain activated co-located GPCRs contain a fragment of the ligand-binding ectodomain of human wild-type RAGE which is not greater than 40, not greater than 20, not greater than 10 or not greater than 5 amino acids in length.

In one aspect, these methods comprise silencing, truncating, modifying or mutating an IgSF CAM such that an IgSF CAM, or analogues, fragments or derivatives thereof, modulates common elements involved in signalling mediated by the cytosolic tail of an IgSF CAM. Association with activation of IgSF CAM by either ligand-dependent or ligand-independent activation pathways.

In one aspect, these methods comprise silencing, truncating, modifying or mutating RAGE such that RAGE, or analogues, fragments or derivatives thereof, modulate common elements involved in signalling mediated by the cytosolic tail of an IgSF CAM. Association with activation of IgSF CAM by either ligand-dependent or ligand-independent activation pathways.

In one aspect, these methods comprise the use of a modulator that modulates ligand-independent activation of an IgSF CAM by activated certain co-located GPCR, such as angiotensin receptor, such as AT₁R, in addition to a modulator that modulates ligand-dependent activation of an IgSF CAM (such as by a modulator that modulates the binding of ligands to the IgSF CAM ectodomain).

The method may comprise administering a modulator to a patient.

12. Methods of Screening Candidate Agents

In one form, the present invention comprises methods of screening candidate agents, such as a fragment or derivative of RAGE, such as RAGE₃₇₀₋₃₉₀ or such as the mCherry-TAT-S391A-RAGE₃₆₂₋₄₀₄ peptide (A Peptide), or such as a fragment or derivative of IgSF CAM such as ALCAM₅₅₉₋₅₈₀, for their ability to modulate (i.e. activate, inhibit or allosterically modulate), IgSF CAM ligand-independent activation of IgSF CAM by activated certain co-located GPCR, such as angiotensin receptor, such as AT₁R (also known as IgSF CAM ligand-independent transactivation of IgSF CAM). These methods generally comprise, consist or consist essentially of:
a. contacting an IgSF CAM polypeptide with a GPCR polypeptide in the presence of a candidate agent, such as RAGE₃₇₀₋₃₉₀ or such as the mCherry-TAT-S391A-RAGE₃₆₂₋₄₀₄ peptide (A Peptide) or such as ALCAM₅₅₉₋₅₈₀, where the GPCR polypeptide is constitutively active and/or is activated by addition of an agonist, partial agonist or allosteric modulator of that GPCR; and
b. detecting whether the candidate agent, such as RAGE₃₇₀₋₃₉₀ or such as the mCherry-TAT-S391A-RAGE₃₆₂₋₄₀₄ peptide (A Peptide) or such as ALCAM₅₅₉₋₅₈₀, is a modulator of IgSF CAM ligand-independent activation of IgSF CAM by activated co-located GPCR by detecting an effect indicative of modulation of IgSF CAM activation by the presence of the candidate agent, such as RAGE₃₇₀₋₃₉₀ or such as the mCherry-TAT-S391A-RAGE₃₆₂₋₄₀₄ peptide (A Peptide) or such as ALCAM₅₅₉₋₅₈₀, and/or by detecting IgSF CAM-dependent signalling that is modulated by the presence of the candidate agent, such as RAGE₃₇₀₋₃₉₀ or such as the mCherry-TAT-S391A-RAGE₃₆₂₋₄₀₄ peptide (A Peptide) or such as ALCAM₅₅₉₋₅₈₀.

In one form, the present invention comprises methods of screening candidate agents for their ability to modulate IgSF CAM ligand-independent activation of IgSF CAM by activated certain co-located GPCR, comprising the steps:
a. contacting an IgSF CAM polypeptide with a GPCR polypeptide in the presence of a candidate agent, where the GPCR polypeptide is constitutively active and/or is activated by addition of an agonist, partial agonist or allosteric modulator of that GPCR; and
b. detecting whether the candidate agent is a modulator of IgSF CAM ligand-independent activation of IgSF CAM by activated co-located GPCR by detecting an effect indicative of modulation of IgSF CAM activation by the presence of the candidate agent, and/or by detecting IgSF CAM-dependent signalling that is modulated by the presence of the candidate agent.

In one form of the invention, the candidate agent is an analogue, fragment or derivative of RAGE.

In a preferred form of the invention, the candidate agent is a fragment or derivative of RAGE.

In one form of the invention, the candidate agent is an analogue, fragment or derivative of a member of the IgSF CAM superfamily (an IgSF CAM).

In a preferred form of the invention, the candidate agent is a fragment or derivative of a member of the IgSF CAM superfamily (an IgSF CAM).

In a particularly preferred form of the invention, the candidate agent is RAGE₃₇₀₋₃₉₀.

In another particularly preferred form of the invention, the candidate agent is the mCherry-TAT-S391A-RAGE₃₆₂₋₄₀₄ peptide (A Peptide).

In another particularly preferred form of the invention, the candidate agent is ALCAM₅₅₉₋₅₈₀.

In a preferred form of the invention, the activated certain co-located GPCR is an angiotensin receptor.

In a particularly preferred form of the invention, the activated certain co-located GPCR is AMR.

In some embodiments, the screening methods further comprise detecting whether the candidate agent, such as a fragment or derivative of RAGE, such as RAGE₃₇₀₋₃₉₀ or such as the mCherry-TAT-S391A-RAGE₃₆₂₋₄₀₄ peptide (A Peptide), or such as a fragment or derivative of IgSF CAM such as ALCAM₅₅₉₋₅₈₀, is a modulator (such as activator, inhibitor or allosteric modulator) of the certain co-located GPCR, such as angiotensin receptor, such as an AT₁R, or a signalling pathway of the certain co-located GPCR, such as an angiotensin receptor signalling pathway, such as an AT₁R signalling pathway, in the presence or absence of IgSF CAM. In some embodiments, the candidate agent, such as a fragment or derivative of RAGE, such as RAGE₃₇₀₋₃₉₀ or such as the mCherry-TAT-S391A-RAGE₃₆₂₋₄₀₄ peptide (A Peptide), or such as a fragment or derivative of IgSF CAM such as ALCAM₅₅₉₋₅₈₀, that results in greater modulation of the signal when the IgSF CAM polypeptide is present compared to when it is absent is selective for modulating IgSF CAM-ligand independent activation of IgSF CAM by activated co-located GPCR over IgSF CAM-independent signalling resulting from activation of the co-located GPCR.

In one form, the invention comprises peptides identified as modulators by said methods.

In one form, the invention comprises compounds identified as modulators by said methods.

In some embodiments, the screening methods further comprise detecting whether the candidate agent, such as a fragment or derivative of RAGE, such as RAGE₃₇₀₋₃₉₀ or such as the mCherry-TAT-S391A-RAGE₃₆₂₋₄₀₄ peptide (A Peptide), or such as a fragment or derivative of IgSF CAM such as ALCAM₅₅₉₋₅₈₀ is a modulator (such as activator, inhibitor, allosteric modulator or functional substitute) of IgSF CAM or an IgSF CAM signalling pathway in the presence or absence of the certain co-located GPCR, such as an angiotensin receptor, such as AT₁R. In some embodiments, the candidate agent, such as a fragment or derivative of RAGE, such as RAGE₃₇₀₋₃₉₀ or such as the mCherry-TAT-S391A-RAGE₃₆₂₋₄₀₄ peptide (A Peptide), or such as a fragment or derivative of IgSF CAM such as ALCAM₅₅₉₋₅₈₀, that results in greater modulation of the IgSF CAM-dependent signal when the GPCR polypeptide is present compared to when it is absent is selective for modulating IgSF CAM-ligand independent activation of IgSF CAM by activated co-located GPCR.

In some embodiments, the screening methods further comprise detecting whether the candidate agent, such as a fragment or derivative of RAGE, such as RAGE₃₇₀₋₃₉₀ or such as the mCherry-TAT-S391A-RAGE₃₆₂₋₄₀₄ peptide (A Peptide), or such as a fragment or derivative of IgSF CAM such as ALCAM₅₅₉₋₅₈₀, is a modulator (such as activator, inhibitor, allosteric modulator or functional substitute) of an IgSF CAM polypeptide or an IgSF CAM signalling pathway as well as the certain co-located GPCR, such as angiotensin receptor, such as an AT₁R, or a signalling pathway of the certain co-located GPCR, such as an angiotensin receptor signalling pathway, such as an AT₁R signalling pathway.

In some embodiments, the screening method further comprises the step of using an inhibitor of IgSF CAM ligand binding to the IgSF CAM ectodomain that as such inhibits activation of IgSF CAM in an IgSF CAM ligand-dependent manner.

In some embodiments, the screening method further comprises use of an IgSF CAM polypeptide that is mutated and/or truncated such that it is not able to bind IgSF CAM ligands to its ectodomain and as such is not able to be activated in an IgSF CAM ligand-dependent manner.

In some embodiments, binding of IgSF CAM ligands to the ectodomain of IgSF CAM is impaired by exposing the cell to a modulator that modulates the binding of IgSF CAM ligands to IgSF CAM.

In some embodiments the use of an IgSF CAM polypeptide that is mutated and/or truncated such that it is not able to bind IgSF CAM ligands and as such is not able to be activated in an IgSF CAM ligand-dependent manner occurs before, after or in parallel with a screen involving an IgSF CAM polypeptide that is able to bind IgSF CAM ligands.

Suitably, a candidate agent or a derivative of a candidate agent, such as a fragment or derivative of RAGE, such as RAGE₃₇₀₋₃₉₀ or such as the mCherry-TAT-S391A-RAGE₃₆₂₋₄₀₄ peptide (A Peptide), or such as a fragment or derivative of IgSF CAM such as ALCAM₅₅₉₋₅₈₀, which modulates IgSF CAM ligand-independent activation of IgSF CAM by activated certain co-located GPCR, such as angiotensin receptor, such as AT₁R, and that suitably modulates a certain co-located GPCR, such as angiotensin receptor, such as AT₁R, and/or a signalling pathway of the certain co-located GPCR, such as an angiotensin receptor signalling pathway, such as an AT₁R signalling pathway and/or that inhibits IgSF CAM ligand-dependent activation of IgSF CAM and/or inhibits constitutively-active IgSF CAM and/or an IgSF CAM signalling pathway, is particularly useful for treating, preventing or managing an IgSF CAM-related disorder.

In certain embodiments, the screening method assesses proximity of the IgSF CAM polypeptide to the certain co-located GPCR, such as angiotensin receptor, such as AT₁R, using a proximity screening assay. In illustrative examples of this type, the IgSF CAM polypeptide is coupled (e.g., conjugated or otherwise linked) to a first reporter component and the certain co-located GPCR, such as angiotensin receptor, such as AT₁R, is coupled (e.g., conjugated or otherwise linked) to a second reporter component. Proximity of the first and second reporter components generates a signal capable of detection by the detector. The first and second reporter components constitute a complementary pair, in the sense that the first reporter component may be interchanged with the second reporter component without appreciably affecting the functioning of the invention. The first and second reporter components can be the same or different.

In one embodiment, the proximity screening assay is that described in patent WO2008055313 (Dimerix Bioscience Pty Ltd; also U.S. Pat. Nos. 8,283,127, 8,568,997, EP2080012, CA2669088, CN101657715), also known as Receptor Heteromer Investigation Technology or Receptor-HIT (Jaeger et al., 2014). With this method, IgSF CAM is coupled to a first reporter component, the certain co-located GPCR, such as angiotensin receptor, such as AMR, is unlabeled with respect to the proximity screening assay, and a GPCR-interacting group is linked to the complementary second reporter component, whose interaction with the complex is modulated upon binding a ligand selective for the unlabeled GPCR or the heteromer complex specifically. Preferred examples of GPCR-interacting groups are arrestins, G proteins and ligands. Alternatively, the certain co-located GPCR, such as angiotensin receptor, such as AT₁R, is coupled to a first reporter component, IgSF CAM is unlabeled with respect to the proximity screening assay, and an IgSF CAM-interacting group is linked to the complementary second reporter component, whose interaction with the complex is modulated upon binding a ligand selective for the unlabeled IgSF CAM or the heteromer complex specifically. Preferred examples of IgSF CAM-interacting groups are proteins interacting with the cytosolic tail of IgSF CAM, such as IQGAP-1, Diaphanous 1, Dock7, MyD88, TIRAP, IRAK4, ERK1/2, and PKCζ (Jules et al., 2013; Ramasamy et al., 2016).

Reporter components can include enzymes, luminescent or bioluminescent molecules, fluorescent molecules, and transcription factors or other molecules coupled to IgSF CAM, the certain co-located GPCR or the interacting group by linkers incorporating enzyme cleavage sites. In short any known molecule, organic or inorganic, proteinaceous or non-proteinaceous or complexes thereof, capable of emitting a detectable signal as a result of their spatial proximity.

Preferably, signal generated by the proximity of the first and second reporter components in the presence of the reporter component initiator is selected from the group consisting of: luminescence, fluorescence and colorimetric change.

In some embodiments, the luminescence is produced by a bioluminescent protein selected from the group consisting of luciferase, galactosidase, lactamase, peroxidase, or any protein capable of luminescence in the presence of a suitable substrate.

Preferable combinations of first and second reporter components include a luminescent reporter component with a fluorescent reporter component, a luminescent reporter component with a non-fluorescent quencher, a fluorescent reporter component with a non-fluorescent quencher, first and second fluorescent reporter components capable of resonance energy transfer. However, useful combinations of first and second reporter components are by no means limited to such.

In some embodiments, the screening methods further comprise detecting proximity of the first and second reporter components to one another to thereby determine whether the candidate agent, such as a fragment or derivative of RAGE, such as RAGE₃₇₀₋₃₉₀ or such as the mCherry-TAT-S391A-RAGE₃₆₂₋₄₀₄ peptide (A Peptide), or such as a fragment or derivative of IgSF CAM such as ALCAM₅₅₉₋₅₈₀, modulates the interaction between the IgSF CAM polypeptide and the certain co-located GPCR, such as angiotensin receptor, such as AMR. Generally, this is achieved when proximity of the first and second reporter components generates a proximity signal that is altered by the modulation by the candidate agent, such as a fragment or derivative of RAGE, such as RAGE₃₇₀₋₃₉₀ or such as the S391A-RAGE Peptide, or such as a fragment or derivative of IgSF CAM such as ALCAM₅₅₉₋₅₈₀, of the proximity between the IgSF CAM polypeptide and the certain co-located GPCR, such as angiotensin receptor, such as AMR.

One or both of the IgSF CAM and certain co-located GPCR, such as angiotensin receptor, such as AT₁R, may be in soluble form or expressed on the cell surface.

In some embodiments, the IgSF CAM and certain co-located GPCR, such as angiotensin receptor, such as AT₁R, are located in, partially in, or on a single membrane; for example, both are expressed at the surface of a host cell.

In another embodiment of the invention, the certain co-located GPCR, such as an angiotensin receptor, such as AT₁R, is pre-assembled with IgSF CAM in a pre-formed complex at the cell membrane.

In another embodiment of the invention, following activation of the certain co-located GPCR, such as angiotensin receptor, such as AT₁R, by engagement of cognate ligand, such as Ang II for AT₁R, signalling is triggered that involves the cytosolic tail of IgSF CAM.

In one embodiment of the invention, activation of the cytosolic tail of IgSF CAM is associated with changes in its structural conformation and/or affinity for binding partners.

In one embodiment of the invention, monitoring of the structural conformation of IgSF CAM and/or affinity for binding partners occurs when the cytosolic tail of IgSF CAM has been mutated and/or truncated such that it can no longer be activated by IgSF CAM ligands or by IgSF CAM ligand-independent activation of IgSF CAM by certain activated co-located GPCRs.

In one embodiment of the invention, monitoring structural conformation and/or affinity for binding partners occurs in the presence of agents that inhibit binding and/or activation of IgSF CAM by IgSF CAM ligands or IgSF CAM ligand-independent activation of IgSF CAM by certain activated co-located GPCRs.

In one embodiment of the invention, monitoring recruitment of binding partners occurs prior to activation of IgSF CAM by IgSF CAM ligands or IgSF CAM ligand-independent activation of IgSF CAM by certain activated co-located GPCRs.

In one embodiment of the invention, monitoring recruitment and activation of signalling mediators and/or binding partners to the IgSF CAM cytosolic tail occurs subsequent to activation of IgSF CAM by IgSF CAM ligands or IgSF CAM ligand-independent activation of IgSF CAM by certain activated co-located GPCRs.

In one embodiment of the invention, monitoring recruitment of binding partners following activation of IgSF CAM by IgSF CAM ligands or IgSF CAM ligand-independent activation of IgSF CAM by certain activated co-located GPCRs occurs in the presence of agents that inhibit binding and/or activation of IgSF CAM by IgSF CAM ligands.

Further embodiments of the invention comprise methods of screening candidate agents, such as a fragment or derivative of RAGE, such as RAGE₃₇₀₋₃₉₀ or such as the mCherry-TAT-S391A-RAGE₃₆₂₋₄₀₄ peptide (A Peptide), or such as a fragment or derivative of IgSF CAM such as ALCAM₅₅₉₋₅₈₀, for their ability to modulate (such as activate, inhibit or otherwise modulate) IgSF CAM ligand-independent activation of IgSF CAM by a certain co-located GPCR, such as angiotensin receptor, such as AT₁R, by detecting modulation of the IgSF CAM-mediated signalling. Such methods may include the step of measuring canonical activation of NFκB, by measuring one or more of the following:
Activity of IkB kinase (IKK) by monitoring in vitro phosphorylation of a substrate, such as GST-IκBa;
Detection of IkB Degradation Dynamics including phosphorylation/ubiquitination and/or degradation of IκB and/or IκB-α;
Detection of p65(Rel-A) phosphorylation/ubiquitination, such as by using antibodies, gel-shift, EMSA, or mass spectroscopy;
Detection of cytoplasmatic to nuclear shuttling/translocation of NFκB components/subunits, such as p65/phospho-p65;
Detection of NFκB subunit dimerization/complexation;
Detection of active NFκB components/subunits by binding to immobilized DNA sequence/oligonucleotide containing the NFκB response element/consensus NFκB binding, such as by using Electrophoretic mobility shift assay or gel shift assay, SELEX, protein-binding microarray, or sequencing-based approaches;
Chromatin-immunoprecipitation (ChIP) assays to detect NFκB in situ binding to DNA to the promoters and enhancers of specific genes;
In vitro kinase assay for NFκB kinase activity;
Measurement of NFκB transcriptional activity using NFκB reporter assays via transgene expression of reporter constructs, such as LacZ Fluc, eGFP SEAP, NF-gluc, using approaches such as plasmid transfection, reporter cell lines, mini-circles, retrovirus, or lentivirus;
Measuring changes in expression of downstream targets of NFκB (such as cytokines, growth factors, adhesion molecules and mitochondrial anti-apoptotic genes by real-time PCR, protein, or functional assays) (Note the pleiotropic nature of NFκB is reflected in its transcriptional targets that presently number over 500 (see http://www.bu.edu/nf-kb/dene-resources/tardet-genes/accessed 7 Dec. 2018) and;
Measuring changes in function or structure induced by NFκB-dependent signalling, such as POLKADOTS in T-cells, adhesion in endothelial cells, activation in leucocytes, or oncogenicity.

Additionally or alternately, such methods may include measuring signals arising from the non-canonical actions of NF-κB, by measuring one or more of the following:
Detection of NIK (NFκB-Inducing Kinase);
Detecting IKκα Activation/phosphorylation;
Detection of NIK kinase activity by ability to autophosphorylate or to phosphorylate a substrate by performing a kinase assay;
Generation of p52-containing NFκB dimers, such as p52/RelB;
Detection of Phospho-NFκB2 p100(Ser866/870);
Detection of partial degradation (called processing) of the precursor p100 into p52;
Detecting p52/RelB translocation into the nucleus;
Detecting p52/RelB binding to κB sites;
Measurement of NFκB transcriptional activity using NFκB reporter assays via transgene expression of reporter constructs, such as LacZ Fluc, eGFP SEAP, NF-gluc, using approaches such as plasmid transfection, reporter cell lines, mini-circles, retrovirus, or lentivirus;
Measuring changes in expression of downstream targets of non-canonical signalling of NFκB (such as CXCL12) by real-time PCR, protein expression or by functional assays.

In one embodiment, an effect on the IgSF CAM indicative of modulation of IgSF CAM activation is a change in intracellular trafficking such as that detected by a change in proximity of luciferase-conjugated IgSF CAM (such as IgSF CAM/Rluc8) to intracellular compartment markers such as fluorophore-labelled Rabs, such as Rab1, Rab4, Rab5, Rab6, Rab7, Rab8, Rab9 and/or Rab11 (such as Venus-Rab1, Venus-Rab4, Venus-Rab5, Venus-Rab6, Venus-Rab7, Venus-Rab8, Venus-Rab9 and/or Venus-Rab11), and/or a plasma membrane marker, such as a fluorophore-conjugated fragment of K-ras (such as Venus-K-ras) using bioluminescence resonance energy transfer (BRET) upon addition of a cognate ligand for the co-located GPCR (Tiulpakov et al., 2016).

In another embodiment, an effect on the IgSF CAM is a change in IgSF CAM-dependent signalling, such as detected by a change in proximity of luciferase-conjugated IgSF CAM (such as IgSF CAM-Rluc8) to an IgSF CAM-interacting group, such as fluorophore-labelled proteins interacting with the cytosolic tail of the IgSF CAM, such as IQGAP-1, protein kinase C zeta (PKCζ), Dock7, MyD88, TIRAP, ERK1/2, (Jules et al., 2013; Ramasamy et al., 2016), olfactory receptor 2T2, ADP/ATP translocase 2, Protein phosphatase 1G, Intercellular adhesion molecule 1, Protein DJ-1 (PARK7), Calponin-3, Drebrin, Filamin B, Ras-related protein Rab-13, Radixin/Ezrin/Moesin, Proteolipid protein 2, Coronin, S100 A11, Succinyl-CoA ligase [GDP-forming] subunit alpha, Hsc70-interacting protein, Apoptosis Inhibitor 5, neuropilin, cleavage stimulation factor, growth factor receptor-bound protein 2, sec61 beta subunit, or Nck1.

In another aspect, the present invention provides methods of identifying a candidate agent that is a modulator (such as activator, inhibitor, allosteric modulator or functional substitute), such as a fragment or derivative of RAGE, such as RAGE₃₇₀₋₃₉₀ or such as the mCherry-TAT-S391A-RAGE₃₆₂₋₄₀₄ peptide (A Peptide), or such as a fragment or derivative of IgSF CAM such as ALCAM₅₅₉₋₅₈₀, that modulates (i.e., activates, inhibits or otherwise modulates) IgSF CAM ligand-independent activation of IgSF CAM following activation of a certain co-located GPCR by a cognate ligand, such as AT₁R by AngII, or if the certain co-located GPCR is constitutively active, and that suitably modulates a certain co-located GPCR, such as an angiotensin receptor, such as AT₁R, and/or that modulates an IgSF CAM polypeptide or an IgSF CAM signalling pathway. In a preferred form of the invention, such a modulator is an inhibitor of one or both of the IgSF CAM or certain co-located GPCR, such as an angiotensin receptor, such as AT₁R, or of the IgSF CAM signalling pathway. In a particularly preferred form of the invention, the modulation of the IgSF CAM signalling pathway is distinct from and/or occurs to a significantly different extent to the modulation of classical certain co-located GPCR signalling pathways, such as AT₁R signalling pathways, such as the Gq signalling pathway. In a particularly preferred form of the invention, the inhibition of the IgSF CAM signalling pathway is distinct from and/or greater than the inhibition of classical certain co-located GPCR signalling pathways, such as AT₁R signalling pathways, such as the Gq signalling pathway.

In one form, the present invention comprises methods of screening candidate agents, where such candidate agents are fragments or derivatives of RAGE, such as RAGE₃₇₀₋₃₉₀ or such as the mCherry-TAT-S391A-RAGE₃₆₂₋₄₀₄ peptide (A Peptide), for their ability to modulate (i.e. inhibit or allosterically modulate), IgSF CAM ligand-dependent activation of IgSF CAM. These methods generally comprise, consist or consist essentially of:
a. contacting an IgSF CAM polypeptide, with or without the presence of a GPCR polypeptide, with a candidate agent, where such a candidate agent is a fragment or derivative of RAGE, such as RAGE₃₇₀₋₃₉₀ or such as the mCherry-TAT-S391A-RAGE₃₆₂₋₄₀₄ peptide (A Peptide); and
b. detecting whether the candidate agent, where such a candidate agent is a fragment or derivative of RAGE, such as RAGE₃₇₀₋₃₉₀ or such as the mCherry-TAT-S391A-RAGE₃₆₂₋₄₀₄ peptide (A Peptide), is a modulator of IgSF CAM ligand-dependent activation of IgSF CAM by detecting an effect indicative of modulation of IgSF CAM activation by the presence of the candidate agent, where such a candidate agent is a fragment or derivative of RAGE, such as RAGE₃₇₀₋₃₉₀ or such as the mCherry-TAT-S391A-RAGE₃₆₂₋₄₀₄ peptide (A Peptide), and/or by detecting IgSF CAM-independent signalling that is modulated by the presence of the candidate agent, where such a candidate agent is a fragment or derivative of RAGE, such as RAGE₃₇₀₋₃₉₀ or such as the mCherry-TAT-S391A-RAGE₃₆₂₋₄₀₄ peptide (A Peptide).

In one form, the present invention comprises methods of screening candidate agents, where such candidate agents are fragments or derivatives of RAGE, for their ability to modulate IgSF CAM ligand-dependent activation of IgSF CAM comprising the steps of:
a. contacting an IgSF CAM polypeptide, with or without the presence of a GPCR polypeptide, with a candidate agent, where such a candidate agent is a fragment or derivative of RAGE; and
b. detecting whether the candidate agent is a modulator of IgSF CAM ligand-dependent activation of IgSF CAM by detecting an effect indicative of modulation of IgSF CAM activation by the presence of the candidate agent, and/or by detecting IgSF CAM-independent signalling that is modulated by the presence of the candidate agent.

In some embodiments, the screening methods further comprise detecting whether the candidate agent, where such a candidate agent is a fragment or derivative of RAGE, such as RAGE₃₇₀₋₃₉₀ or such as the mCherry-TAT-S391A-RAGE₃₆₂₋₄₀₄ peptide (A Peptide), is a modulator (such as activator, inhibitor or allosteric modulator) of a certain co-located GPCR, such as angiotensin receptor, such as an AT₁R, or a signalling pathway of the certain co-located GPCR, such as an angiotensin receptor signalling pathway, such as an AT₁R signalling pathway, in the presence or absence of IgSF CAM.

In one form, the invention comprises peptides identified as modulators by said methods.

In one form, the invention comprises compounds identified as modulators by said methods.

In some embodiments, the screening methods further comprise detecting whether the candidate agent, where such a candidate agent is a fragment or derivative of RAGE, such as RAGE₃₇₀₋₃₉₀ or such as the mCherry-TAT-S391A-RAGE₃₆₂₋₄₀₄ peptide (A Peptide), is a modulator (such as activator, inhibitor, allosteric modulator or functional substitute) of IgSF CAM or an IgSF CAM signalling pathway in the presence or absence of a certain co-located GPCR, such as an angiotensin receptor, such as AT₁R. In some embodiments, the candidate agent, where such a candidate agent is a fragment or derivative of RAGE, such as RAGE₃₇₀₋₃₉₀ or such as the mCherry-TAT-S391A-RAGE₃₆₂₋₄₀₄ peptide (A Peptide), that results in greater modulation of the IgSF CAM-dependent signal when the GPCR polypeptide is absent compared to when it is present is selective for modulating IgSF CAM-ligand dependent activation of IgSF CAM.

In some embodiments, the screening methods further comprise detecting whether the candidate agent, where such a candidate agent is a fragment or derivative of RAGE, such as RAGE₃₇₀₋₃₉₀ or such as the mCherry-TAT-S391A-RAGE₃₆₂₋₄₀₄ peptide (A Peptide), is a modulator (such as activator, inhibitor, allosteric modulator or functional substitute) of an IgSF CAM polypeptide or an IgSF CAM signalling pathway as well as a certain co-located GPCR, such as angiotensin receptor, such as an AT₁R, or a signalling pathway of a certain co-located GPCR, such as an angiotensin receptor signalling pathway, such as an AT₁R signalling pathway.

In some embodiments, the screening method further comprises the step of using an inhibitor of IgSF CAM ligand binding to the IgSF CAM ectodomain that as such inhibits activation of IgSF CAM in an IgSF CAM ligand-dependent manner.

In some embodiments, the screening method further comprises use of an IgSF CAM polypeptide that is mutated and/or truncated such that it is not able to bind IgSF CAM ligands to its ectodomain and as such is not able to be activated in an IgSF CAM ligand-dependent manner.

In some embodiments, binding of IgSF CAM ligands to the ectodomain of IgSF CAM is impaired by exposing the cell to a modulator that modulates the binding of IgSF CAM ligands to IgSF CAM.

In some embodiments the use of an IgSF CAM polypeptide that is mutated and/or truncated such that it is not able to bind IgSF CAM ligands and as such is not able to be activated in an IgSF CAM ligand-dependent manner occurs before, after or in parallel with a screen involving an IgSF CAM polypeptide that is able to bind IgSF CAM ligands.

Suitably, a candidate agent or a derivative of a candidate agent, where such a candidate agent is a fragment or derivative of RAGE, such as RAGE₃₇₀₋₃₉₀ or such as the mCherry-TAT-S391A-RAGE₃₆₂₋₄₀₄ peptide (A Peptide), which modulates IgSF CAM ligand-dependent activation of IgSF CAM is particularly useful for treating, preventing or managing an IgSF CAM-related disorder.

In certain embodiments, the screening method assesses proximity of the IgSF CAM polypeptide to a certain co-located GPCR, such as angiotensin receptor, such as AT₁R, using a proximity screening assay. In illustrative examples of this type, the IgSF CAM polypeptide is coupled (e.g., conjugated or otherwise linked) to a first reporter component and a certain co-located GPCR, such as angiotensin receptor, such as AT₁R, is coupled (e.g., conjugated or otherwise linked) to a second reporter component. Proximity of the first and second reporter components generates a signal capable of detection by the detector. The first and second reporter components constitute a complementary pair, in the sense that the first reporter component may be interchanged with the second reporter component without appreciably affecting the functioning of the invention. The first and second reporter components can be the same or different.

In one embodiment, the proximity screening assay is that described in patent WO2008055313 (Dimerix Bioscience Pty Ltd; also U.S. Pat. Nos. 8,283,127, 8,568,997, EP2080012, CA2669088, CN101657715), also known as Receptor Heteromer Investigation Technology or Receptor-HIT (Jaeger et al., 2014). With this method, IgSF CAM is coupled to a first reporter component, a certain co-located GPCR, such as angiotensin receptor, such as AT₁R, is unlabeled with respect to the proximity screening assay, and a GPCR-interacting group is linked to the complementary second reporter component, whose interaction with the complex is modulated upon binding a ligand selective for an unlabeled GPCR or the heteromer complex specifically. Preferred examples of GPCR-interacting groups are arrestins, G proteins and ligands. Alternatively, a certain co-located GPCR, such as angiotensin receptor, such as AT₁R, is coupled to a first reporter component, IgSF CAM is unlabeled with respect to the proximity screening assay, and an IgSF CAM-interacting group is linked to the complementary second reporter component, whose interaction with the complex is modulated upon binding a ligand selective for the unlabeled IgSF CAM or the heteromer complex specifically. Preferred examples of IgSF CAM-interacting groups are proteins interacting with the cytosolic tail of IgSF CAM, such as IQGAP-1, Diaphanous 1, Dock7, MyD88, TIRAP, IRAK4, ERK1/2, and PKCζ (Jules et al., 2013; Ramasamy et al., 2016).

Reporter components can include enzymes, luminescent or bioluminescent molecules, fluorescent molecules, and transcription factors or other molecules coupled to IgSF CAM, a certain co-located GPCR or the interacting group by linkers incorporating enzyme cleavage sites. In short any known molecule, organic or inorganic, proteinaceous or non-proteinaceous or complexes thereof, capable of emitting a detectable signal as a result of their spatial proximity.

Preferably, signal generated by the proximity of the first and second reporter components in the presence of the reporter component initiator is selected from the group consisting of: luminescence, fluorescence and colorimetric change.

In some embodiments, the luminescence is produced by a bioluminescent protein selected from the group consisting of luciferase, galactosidase, lactamase, peroxidase, or any protein capable of luminescence in the presence of a suitable substrate.

Preferable combinations of first and second reporter components include a luminescent reporter component with a fluorescent reporter component, a luminescent reporter component with a non-fluorescent quencher, a fluorescent reporter component with a non-fluorescent quencher, first and second fluorescent reporter components capable of resonance energy transfer. However, useful combinations of first and second reporter components are by no means limited to such.

In some embodiments, the screening methods further comprise detecting proximity of the first and second reporter components to one another to thereby determine whether the candidate agent, where such a candidate agent is a fragment or derivative of RAGE, such as RAGE₃₇₀₋₃₉₀ or such as the mCherry-TAT-S391A-RAGE₃₆₂₋₄₀₄ peptide (A Peptide), modulates the interaction between the IgSF CAM polypeptide and a certain co-located GPCR, such as angiotensin receptor, such as AT₁R. Generally, this is achieved when proximity of the first and second reporter components generates a proximity signal that is altered by the modulation by the candidate agent, where such a candidate agent is a fragment or derivative of RAGE, such as RAGE₃₇₀₋₃₉₀ or such as the mCherry-TAT-S391A-RAGE₃₆₂₋₄₀₄ peptide (A Peptide), of the proximity between the IgSF CAM polypeptide and a certain co-located GPCR, such as angiotensin receptor, such as AT₁R.

One or both of the IgSF CAM and certain co-located GPCR, such as angiotensin receptor, such as AT₁R, may be in soluble form or expressed on the cell surface.

In some embodiments, the IgSF CAM and certain co-located GPCR, such as angiotensin receptor, such as AT₁R, are located in, partially in, or on a single membrane; for example, both are expressed at the surface of a host cell.

In another embodiment of the invention, a certain co-located GPCR, such as an angiotensin receptor, such as AT₁R, is pre-assembled with IgSF CAM in a pre-formed complex at the cell membrane.

In another embodiment of the invention, following activation of a certain co-located GPCR, such as angiotensin receptor, such as AT₁R, by engagement of cognate ligand, such as Ang II for AT₁R, signalling is triggered that involves the cytosolic tail of IgSF CAM.

In one embodiment of the invention, activation of the cytosolic tail of IgSF CAM is associated with changes in its structural conformation and/or affinity for binding partners.

In one embodiment of the invention, monitoring of the structural conformation of IgSF CAM and/or affinity for binding partners occurs when the cytosolic tail of IgSF CAM has been mutated and/or truncated such that it can no longer be activated by IgSF CAM ligands or by IgSF CAM ligand-independent activation of IgSF CAM by certain activated co-located GPCRs.

In one embodiment of the invention, monitoring structural conformation and/or affinity for binding partners occurs in the presence of agents that inhibit binding and/or activation of IgSF CAM by IgSF CAM ligands or IgSF CAM ligand-independent activation of IgSF CAM by certain activated co-located GPCRs.

In one embodiment of the invention, monitoring recruitment of binding partners occurs prior to activation of IgSF CAM by IgSF CAM ligands or IgSF CAM ligand-independent activation of IgSF CAM by certain activated co-located GPCRs.

In one embodiment of the invention, monitoring recruitment and activation of signalling mediators and/or binding partners to the IgSF CAM cytosolic tail occurs subsequent to activation of IgSF CAM by IgSF CAM ligands or IgSF CAM ligand-independent activation of IgSF CAM by certain activated co-located GPCRs.

In one embodiment of the invention, monitoring recruitment of binding partners following activation of IgSF CAM by IgSF CAM ligands or IgSF CAM ligand-independent activation of IgSF CAM by certain activated co-located GPCRs occurs in the presence of agents that inhibit binding and/or activation of IgSF CAM by IgSF CAM ligands.

Further embodiments of the invention comprise methods of screening candidate agents, where such a candidate agent is a fragment or derivative of RAGE, such as RAGE₃₇₀₋₃₉₀ or such as the mCherry-TAT-S391A-RAGE₃₆₂₋₄₀₄ peptide (A Peptide), for their ability to modulate (such as inhibit or otherwise modulate) IgSF CAM ligand-dependent activation of IgSF CAM by detecting modulation of the IgSF CAM-mediated signalling. Such methods may include the step of measuring canonical activation of NFκB, by measuring one or more of the following:
Activity of IkB kinase (IKK) by monitoring in vitro phosphorylation of a substrate, such as GST-IκBa;
Detection of IkB Degradation Dynamics including phosphorylation/ubiquitination and/or degradation of IκB and/or IκB-α;
Detection of p65(Rel-A) phosphorylation/ubiquitination, such as by using antibodies, gel-shift, EMSA, or mass spectroscopy;
Detection of cytoplasmatic to nuclear shuttling/translocation of NFκB components/subunits, such as p65/phospho-p65;
Detection of NFκB subunit dimerization/complexation;
Detection of active NFκB components/subunits by binding to immobilized DNA sequence/oligonucleotide containing the NFκB response element/consensus NFκB binding, such as by using Electrophoretic mobility shift assay or gel shift assay, SELEX, protein-binding microarray, or sequencing-based approaches;
Chromatin-immunoprecipitation (ChIP) assays to detect NFκB in situ binding to DNA to the promoters and enhancers of specific genes;
In vitro kinase assay for NFκB kinase activity;
Measurement of NFκB transcriptional activity using NFκB reporter assays via transgene expression of reporter constructs, such as LacZ Fluc, eGFP SEAP, NF-gluc, using approaches such as plasmid transfection, reporter cell lines, mini-circles, retrovirus, or lentivirus;
Measuring changes in expression of downstream targets of NFκB (such as cytokines, growth factors, adhesion molecules and mitochondrial anti-apoptotic genes by real-time PCR, protein, or functional assays) (Note the pleiotropic nature of NFκB is reflected in its transcriptional targets that presently number over 500 (see http://www.bu.edu/nf-kb/dene-resources/tardet-denes/accessed 7 Dec. 2018) and;
Measuring changes in function or structure induced by NFκB-dependent signalling, such as POLKADOTS in T-cells, adhesion in endothelial cells, activation in leucocytes, or oncogenicity.

Additionally or alternately, such methods may include measuring signals arising from the non-canonical actions of NF-κB, by measuring one or more of the following:
Detection of NIK (NFκB-Inducing Kinase);
Detecting IKκα Activation/phosphorylation;
Detection of NIK kinase activity by ability to autophosphorylate or to phosphorylate a substrate by performing a kinase assay;
Generation of p52-containing NFκB dimers, such as p52/RelB;
Detection of Phospho-NFκB2 p100(Ser866/870);
Detection of partial degradation (called processing) of the precursor p100 into p52;
Detecting p52/RelB translocation into the nucleus;
Detecting p52/RelB binding to κB sites;
Measurement of NFκB transcriptional activity using NFκB reporter assays via transgene expression of reporter constructs, such as LacZ Fluc, eGFP SEAP, NF-gluc, using approaches such as plasmid transfection, reporter cell lines, mini-circles, retrovirus, or lentivirus;
Measuring changes in expression of downstream targets of non-canonical signalling of NFκB (such as CXCL12) by real-time PCR, protein expression or by functional assays.

In one embodiment, an effect on the IgSF CAM indicative of modulation of IgSF CAM activation is a change in intracellular trafficking such as that detected by a change in proximity of luciferase-conjugated IgSF CAM (such as IgSF CAM/Rluc8) to intracellular compartment markers such as fluorophore-labelled Rabs, such as Rab1 Rab4, Rab5, Rab6, Rab7, Rab8, Rab9 and/or Rab11 (such as Venus-Rab1 Venus-Rab4, Venus-Rab5, Venus-Rab6, Venus-Rab7, Venus-Rab8, Venus-Rab9 and/or Venus-Rab11), and/or a plasma membrane marker, such as a fluorophore-conjugated fragment of K-ras (such as Venus-K-ras) using bioluminescence resonance energy transfer (BRET) upon addition of a cognate ligand for the co-located GPCR (Tiulpakov et al., 2016).

In another embodiment, an effect on the IgSF CAM is a change in IgSF CAM-dependent signalling, such as detected by a change in proximity of luciferase-conjugated IgSF CAM (such as IgSF CAM-Rluc8) to an IgSF CAM-interacting group, such as fluorophore-labelled proteins interacting with the cytosolic tail of the IgSF CAM, such as IQGAP-1, protein kinase C zeta (PKCζ), Dock7, MyD88, TIRAP, ERK1/2, (Jules et al., 2013; Ramasamy et al., 2016), olfactory receptor 2T2, ADP/ATP translocase 2, Protein phosphatase 1G, Intercellular adhesion molecule 1, Protein DJ-1 (PARK7), Calponin-3, Drebrin, Filamin B, Ras-related protein Rab-13, Radixin/Ezrin/Moesin, Proteolipid protein 2, Coronin, S100 A1l, Succinyl-CoA ligase [GDP-forming] subunit alpha, Hsc70-interacting protein, Apoptosis Inhibitor 5, neuropilin, cleavage stimulation factor, growth factor receptor-bound protein 2, sec61 beta subunit, or Nck1.

In another aspect, the present invention provides methods of identifying a candidate agent that is a modulator (such as activator, inhibitor, allosteric modulator or functional substitute), where such a modulator is a fragment or derivative of RAGE, such as RAGE₃₇₀₋₃₉₀ or such as the mCherry-TAT-S391A-RAGE₃₆₂₋₄₀₄ peptide (A Peptide), that modulates (i.e., activates, inhibits or otherwise modulates) IgSF CAM ligand-independent activation of IgSF CAM following activation of a certain co-located GPCR by a cognate ligand, such as AT₁R by AngII, or if the certain co-located GPCR is constitutively active, and that suitably modulates a certain co-located GPCR, such as an angiotensin receptor, such as AT₁R, and/or that modulates an IgSF CAM polypeptide or an IgSF CAM signalling pathway. In one form of the invention, such a modulator is an inhibitor of the IgSF CAM or of the IgSF CAM signalling pathway. In a particularly preferred form of the invention, the modulation of the IgSF CAM signalling pathway is distinct from and/or occurs to a significantly different extent to the modulation of classical certain co-located GPCR signalling pathways, such as AT₁R signalling pathways, such as the Gq signalling pathway. In a particularly preferred form of the invention, the inhibition of the IgSF CAM signalling pathway is distinct from and/or greater than the inhibition of classical certain co-located GPCR signalling pathways, such as AT₁R signalling pathways, such as the Gq signalling pathway.

In one form, the present invention comprises methods of screening candidate agents, where such candidate agents are fragments or derivatives of members of the IgSF CAM superfamily, such as ALCAM₅₅₉₋₅₈₀, for their ability to modulate (i.e. inhibit or allosterically modulate), IgSF CAM ligand-dependent activation of IgSF CAM. These methods generally comprise, consist or consist essentially of:
a. contacting an IgSF CAM polypeptide, with or without the presence of a GPCR polypeptide, with a candidate agent, where such a candidate agent is a fragment or derivative of a member of the IgSF CAM superfamily, such as ALCAM₅₅₉₋₅₈₀; and
b. detecting whether the candidate agent, where such a candidate agent is a fragment or derivative of a member of the IgSF CAM superfamily, such as ALCAM₅₅₉₋₅₈₀, is a modulator of IgSF CAM ligand-dependent activation of IgSF CAM by detecting an effect indicative of modulation of IgSF CAM activation by the presence of the candidate agent, where such a candidate agent is a fragment or derivative of a member of the IgSF CAM superfamily, such as ALCAM₅₅₉₋₅₈₀, and/or by detecting IgSF CAM-independent signalling that is modulated by the presence of the candidate agent, where such a candidate agent is a fragment or derivative of a member of the IgSF CAM superfamily, such as ALCAM₅₅₉₋₅₈₀.

In one form, the present invention comprises methods of screening candidate agents, where such candidate agents are fragments or derivatives of members of the IgSF CAM superfamily, for their ability to modulate IgSF CAM ligand-dependent activation of IgSF CAM comprising the steps of:
a. contacting an IgSF CAM polypeptide, with or without the presence of a GPCR polypeptide, with a candidate agent, where such a candidate agent is a fragment or derivative of a member of the IgSF CAM superfamily, such as ALCAM₅₅₉₋₅₈₀; and
b. detecting whether the candidate agent is a modulator of IgSF CAM ligand-dependent activation of IgSF CAM by detecting an effect indicative of modulation of IgSF CAM activation by the presence of the candidate agent, and/or by detecting IgSF CAM-independent signalling that is modulated by the presence of the candidate agent.

In some embodiments, the screening methods further comprise detecting whether the candidate agent, where such a candidate agent is a fragment or derivative of a member of the IgSF CAM superfamily, such as ALCAM₅₅₉₋₅₈₀, is a modulator (such as activator, inhibitor or allosteric modulator) of a certain co-located GPCR, such as angiotensin receptor, such as an AT₁R, or a signalling pathway of the certain co-located GPCR, such as an angiotensin receptor signalling pathway, such as an AT₁R signalling pathway, in the presence or absence of IgSF CAM.

In one form, the invention comprises peptides identified as modulators by said methods.

In one form, the invention comprises compounds identified as modulators by said methods.

In some embodiments, the screening methods further comprise detecting whether the candidate agent, where such a candidate agent is a fragment or derivative of a member of the IgSF CAM superfamily, such as ALCAM₅₅₉₋₅₈₀, is a modulator (such as activator, inhibitor, allosteric modulator or functional substitute) of IgSF CAM or an IgSF CAM signalling pathway in the presence or absence of a certain co-located GPCR, such as an angiotensin receptor, such as AMR. In some embodiments, the candidate agent, where such a candidate agent is a fragment or derivative of a member of the IgSF CAM superfamily, such as ALCAM₅₅₉₋₅₈₀, that results in greater modulation of the IgSF CAM-dependent signal when the GPCR polypeptide is absent compared to when it is present is selective for modulating IgSF CAM-ligand dependent activation of IgSF CAM.

In some embodiments, the screening methods further comprise detecting whether the candidate agent, where such a candidate agent is a fragment or derivative of a member of the IgSF CAM superfamily, such as ALCAM₅₅₉₋₅₈₀, is a modulator (such as activator, inhibitor, allosteric modulator or functional substitute) of an IgSF CAM polypeptide or an IgSF CAM signalling pathway as well as a certain co-located GPCR, such as angiotensin receptor, such as an AT₁R, or a signalling pathway of a certain co-located GPCR, such as an angiotensin receptor signalling pathway, such as an AT₁R signalling pathway.

In some embodiments, the screening method further comprises the step of using an inhibitor of IgSF CAM ligand binding to the IgSF CAM ectodomain that as such inhibits activation of IgSF CAM in an IgSF CAM ligand-dependent manner.

In some embodiments, the screening method further comprises use of an IgSF CAM polypeptide that is mutated and/or truncated such that it is not able to bind IgSF CAM ligands to its ectodomain and as such is not able to be activated in an IgSF CAM ligand-dependent manner.

In some embodiments, binding of IgSF CAM ligands to the ectodomain of IgSF CAM is impaired by exposing the cell to a modulator that modulates the binding of IgSF CAM ligands to IgSF CAM.

In some embodiments the use of an IgSF CAM polypeptide that is mutated and/or truncated such that it is not able to bind IgSF CAM ligands and as such is not able to be activated in an IgSF CAM ligand-dependent manner occurs before, after or in parallel with a screen involving an IgSF CAM polypeptide that is able to bind IgSF CAM ligands.

Suitably, a candidate agent or a derivative of a candidate agent, where such a candidate agent is a fragment or derivative of a member of the IgSF CAM superfamily, such as ALCAM₅₅₉₋₅₈₀, which modulates IgSF CAM ligand-dependent activation of IgSF CAM is particularly useful for treating, preventing or managing an IgSF CAM-related disorder.

In certain embodiments, the screening method assesses proximity of the IgSF CAM polypeptide to a certain co-located GPCR, such as angiotensin receptor, such as AT₁R, using a proximity screening assay. In illustrative examples of this type, the IgSF CAM polypeptide is coupled (e.g., conjugated or otherwise linked) to a first reporter component and a certain co-located GPCR, such as angiotensin receptor, such as AT₁R, is coupled (e.g., conjugated or otherwise linked) to a second reporter component. Proximity of the first and second reporter components generates a signal capable of detection by the detector. The first and second reporter components constitute a complementary pair, in the sense that the first reporter component may be interchanged with the second reporter component without appreciably affecting the functioning of the invention. The first and second reporter components can be the same or different.

In one embodiment, the proximity screening assay is that described in patent WO2008055313 (Dimerix Bioscience Pty Ltd; also U.S. Pat. Nos. 8,283,127, 8,568,997, EP2080012, CA2669088, CN101657715), also known as Receptor Heteromer Investigation Technology or Receptor-HIT (Jaeger et al., 2014). With this method, IgSF CAM is coupled to a first reporter component, a certain co-located GPCR, such as angiotensin receptor, such as AT₁R, is unlabeled with respect to the proximity screening assay, and a GPCR-interacting group is linked to the complementary second reporter component, whose interaction with the complex is modulated upon binding a ligand selective for an unlabeled GPCR or the heteromer complex specifically. Preferred examples of GPCR-interacting groups are arrestins, G proteins and ligands. Alternatively, a certain co-located GPCR, such as angiotensin receptor, such as AT₁R, is coupled to a first reporter component, IgSF CAM is unlabeled with respect to the proximity screening assay, and an IgSF CAM-interacting group is linked to the complementary second reporter component, whose interaction with the complex is modulated upon binding a ligand selective for the unlabeled IgSF CAM or the heteromer complex specifically. Preferred examples of IgSF CAM-interacting groups are proteins interacting with the cytosolic tail of IgSF CAM, such as IQGAP-1, Diaphanous 1, Dock7, MyD88, TIRAP, IRAK4, ERK1/2, and PKCζ (Jules et al., 2013; Ramasamy et al., 2016).

Reporter components can include enzymes, luminescent or bioluminescent molecules, fluorescent molecules, and transcription factors or other molecules coupled to IgSF CAM, a certain co-located GPCR or the interacting group by linkers incorporating enzyme cleavage sites. In short any known molecule, organic or inorganic, proteinaceous or non-proteinaceous or complexes thereof, capable of emitting a detectable signal as a result of their spatial proximity.

Preferably, signal generated by the proximity of the first and second reporter components in the presence of the reporter component initiator is selected from the group consisting of: luminescence, fluorescence and colorimetric change.

In some embodiments, the luminescence is produced by a bioluminescent protein selected from the group consisting of luciferase, galactosidase, lactamase, peroxidase, or any protein capable of luminescence in the presence of a suitable substrate.

Preferable combinations of first and second reporter components include a luminescent reporter component with a fluorescent reporter component, a luminescent reporter component with a non-fluorescent quencher, a fluorescent reporter component with a non-fluorescent quencher, first and second fluorescent reporter components capable of resonance energy transfer. However, useful combinations of first and second reporter components are by no means limited to such.

In some embodiments, the screening methods further comprise detecting proximity of the first and second reporter components to one another to thereby determine whether the candidate agent, where such a candidate agent is a fragment or derivative of a member of the IgSF CAM superfamily, such as ALCAM₅₅₉₋₅₈₀, modulates the interaction between the IgSF CAM polypeptide and a certain co-located GPCR, such as angiotensin receptor, such as AT₁R. Generally, this is achieved when proximity of the first and second reporter components generates a proximity signal that is altered by the modulation by the candidate agent, where such a candidate agent is a fragment or derivative of a member of the IgSF CAM superfamily, such as ALCAM₅₅₉₋₅₈₀, of the proximity between the IgSF CAM polypeptide and a certain co-located GPCR, such as angiotensin receptor, such as AT₁R.

One or both of the IgSF CAM and certain co-located GPCR, such as angiotensin receptor, such as AT₁R, may be in soluble form or expressed on the cell surface.

In some embodiments, the IgSF CAM and certain co-located GPCR, such as angiotensin receptor, such as AT₁R, are located in, partially in, or on a single membrane; for example, both are expressed at the surface of a host cell.

In another embodiment of the invention, a certain co-located GPCR, such as an angiotensin receptor, such as AT₁R, is pre-assembled with IgSF CAM in a pre-formed complex at the cell membrane.

In another embodiment of the invention, following activation of a certain co-located GPCR, such as angiotensin receptor, such as AT₁R, by engagement of cognate ligand, such as Ang II for AT₁R, signalling is triggered that involves the cytosolic tail of IgSF CAM.

In one embodiment of the invention, activation of the cytosolic tail of IgSF CAM is associated with changes in its structural conformation and/or affinity for binding partners.

In one embodiment of the invention, monitoring of the structural conformation of IgSF CAM and/or affinity for binding partners occurs when the cytosolic tail of IgSF CAM has been mutated and/or truncated such that it can no longer be activated by IgSF CAM ligands or by IgSF CAM ligand-independent activation of IgSF CAM by certain activated co-located GPCRs.

In one embodiment of the invention, monitoring structural conformation and/or affinity for binding partners occurs in the presence of agents that inhibit binding and/or activation of IgSF CAM by IgSF CAM ligands or IgSF CAM ligand-independent activation of IgSF CAM by certain activated co-located GPCRs.

In one embodiment of the invention, monitoring recruitment of binding partners occurs prior to activation of IgSF CAM by IgSF CAM ligands or IgSF CAM ligand-independent activation of IgSF CAM by certain activated co-located GPCRs.

In one embodiment of the invention, monitoring recruitment and activation of signalling mediators and/or binding partners to the IgSF CAM cytosolic tail occurs subsequent to activation of IgSF CAM by IgSF CAM ligands or IgSF CAM ligand-independent activation of IgSF CAM by certain activated co-located GPCRs.

In one embodiment of the invention, monitoring recruitment of binding partners following activation of IgSF CAM by IgSF CAM ligands or IgSF CAM ligand-independent activation of IgSF CAM by certain activated co-located GPCRs occurs in the presence of agents that inhibit binding and/or activation of IgSF CAM by IgSF CAM ligands.

Further embodiments of the invention comprise methods of screening candidate agents, where such a candidate agent is a fragment or derivative of a member of the IgSF CAM superfamily, such as ALCAM₅₅₉₋₅₈₀, for their ability to modulate (such as inhibit or otherwise modulate) IgSF CAM ligand-dependent activation of IgSF CAM by detecting modulation of the IgSF CAM-mediated signalling. Such methods may include the step of measuring canonical activation of NFκB, by measuring one or more of the following:
Activity of IkB kinase (IKK) by monitoring in vitro phosphorylation of a substrate, such as GST-IκBα;
Detection of IkB Degradation Dynamics including phosphorylation/ubiquitination and/or degradation of IκB and/or IκB-α;
Detection of p65(Rel-A) phosphorylation/ubiquitination, such as by using antibodies, gel-shift, EMSA, or mass spectroscopy;
Detection of cytoplasmatic to nuclear shuttling/translocation of NFκB components/subunits, such as p65/phospho-p65;
Detection of NFκB subunit dimerization/complexation;
Detection of active NFκB components/subunits by binding to immobilized DNA sequence/oligonucleotide containing the NFκB response element/consensus NFκB binding, such as by using Electrophoretic mobility shift assay or gel shift assay, SELEX, protein-binding microarray, or sequencing-based approaches;
Chromatin-immunoprecipitation (ChIP) assays to detect NFκB in situ binding to DNA to the promoters and enhancers of specific genes;
In vitro kinase assay for NFκB kinase activity;
Measurement of NFκB transcriptional activity using NFκB reporter assays via transgene expression of reporter constructs, such as LacZ Fluc, eGFP SEAP, NF-gluc, using approaches such as plasmid transfection, reporter cell lines, mini-circles, retrovirus, or lentivirus;
Measuring changes in expression of downstream targets of NFκB (such as cytokines, growth factors, adhesion molecules and mitochondrial anti-apoptotic genes by real-time PCR, protein, or functional assays) (Note the pleiotropic nature of NFκB is reflected in its transcriptional targets that presently number over 500 (see http://www.bu.edu/nf-kb/dene-resources/tardet-genes/accessed 2 Dec. 2018) and;
Measuring changes in function or structure induced by NFκB-dependent signalling, such as POLKADOTS in T-cells, adhesion in endothelial cells, activation in leucocytes, or oncogenicity.

Additionally or alternately, such methods may include measuring signals arising from the non-canonical actions of NF-κB, by measuring one or more of the following:
Detection of NIK (NFκB-Inducing Kinase);
Detecting IKκα Activation/phosphorylation;
Detection of NIK kinase activity by ability to autophosphorylate or to phosphorylate a substrate by performing a kinase assay;
Generation of p52-containing NFκB dimers, such as p52/RelB;
Detection of Phospho-NFκB2 p100(Ser866/870);
Detection of partial degradation (called processing) of the precursor p100 into p52;
Detecting p52/RelB translocation into the nucleus;
Detecting p52/RelB binding to KB sites;
Measurement of NFκB transcriptional activity using NFκB reporter assays via transgene expression of reporter constructs, such as LacZ Fluc, eGFP SEAP, NF-gluc, using approaches such as plasmid transfection, reporter cell lines, mini-circles, retrovirus, or lentivirus;
Measuring changes in expression of downstream targets of non-canonical signalling of NFκB (such as CXCL12) by real-time PCR, protein expression or by functional assays.

In one embodiment, an effect on the IgSF CAM indicative of modulation of IgSF CAM activation is a change in intracellular trafficking such as that detected by a change in proximity of luciferase-conjugated IgSF CAM (such as IgSF CAM/Rluc8) to intracellular compartment markers such as fluorophore-labelled Rabs, such as Rab1, Rab4, Rab5, Rab6, Rab7, Rab8, Rab9 and/or Rab11 (such as Venus-Rab1, Venus-Rab4, Venus-Rab5, Venus-Rab6, Venus-Rab7, Venus-Rab8, Venus-Rab9 and/or Venus-Rab11), and/or a plasma membrane marker, such as a fluorophore-conjugated fragment of K-ras (such as Venus-K-ras) using bioluminescence resonance energy transfer (BRET) upon addition of a cognate ligand for the co-located GPCR (Tiulpakov et al., 2016).

In another embodiment, an effect on the IgSF CAM is a change in IgSF CAM-dependent signalling, such as detected by a change in proximity of luciferase-conjugated IgSF CAM (such as IgSF CAM-Rluc8) to an IgSF CAM-interacting group, such as fluorophore-labelled proteins interacting with the cytosolic tail of the IgSF CAM, such as IQGAP-1, protein kinase C zeta (PKCζ), Dock7, MyD88, TIRAP, ERK1/2, (Jules et al., 2013; Ramasamy et al., 2016), olfactory receptor 2T2, ADP/ATP translocase 2, Protein phosphatase 1G, Intercellular adhesion molecule 1, Protein DJ-1 (PARK7), Calponin-3, Drebrin, Filamin B, Ras-related protein Rab-13, Radixin/Ezrin/Moesin, Proteolipid protein 2, Coronin, S100 A11, Succinyl-CoA ligase [GDP-forming] subunit alpha, Hsc70-interacting protein, Apoptosis Inhibitor 5, neuropilin, cleavage stimulation factor, growth factor receptor-bound protein 2, sec61 beta subunit, or Nck1.

In another aspect, the present invention provides methods of identifying a candidate agent that is a modulator (such as activator, inhibitor, allosteric modulator or functional substitute), where such a candidate agent is a fragment or derivative of a member of the IgSF CAM superfamily, such as ALCAM₅₅₉₋₅₈₀, that modulates (i.e., activates, inhibits or otherwise modulates) IgSF CAM ligand-independent activation of IgSF CAM following activation of a certain co-located GPCR by a cognate ligand, such as AT₁R by AngII, or if the certain co-located GPCR is constitutively active, and that suitably modulates a certain co-located GPCR, such as an angiotensin receptor, such as AT₁R, and/or that modulates an IgSF CAM polypeptide or an IgSF CAM signalling pathway. In one form of the invention, such a modulator is an inhibitor of the IgSF CAM or of the IgSF CAM signalling pathway. In a particularly preferred form of the invention, the modulation of the IgSF CAM signalling pathway is distinct from and/or occurs to a significantly different extent to the modulation of classical certain co-located GPCR signalling pathways, such as AT₁R signalling pathways, such as the Gq signalling pathway. In a particularly preferred form of the invention, the inhibition of the IgSF CAM signalling pathway is distinct from and/or greater than the inhibition of classical certain co-located GPCR signalling pathways, such as AT₁R signalling pathways, such as the Gq signalling pathway.

In one form, the present invention comprises methods of screening candidate agents, where candidate agents are fragments or derivatives of members of the IgSF CAM superfamily, such as ALCAM₅₅₉₋₅₈₀, for their ability to modulate (i.e. activate, inhibit or allosterically modulate) RAGE ligand-independent activation of RAGE by activated certain co-located GPCR, such as angiotensin receptor, such as AT₁R, or such as a certain complement receptor, such as C5a receptor 1 (also known as RAGE ligand-independent transactivation of RAGE). These methods generally comprise, consist or consist essentially of:
a. Contacting a RAGE polypeptide with a GPCR polypeptide in the presence of a candidate agent, where the candidate agent is a fragment or derivative of a member of the IgSF CAM superfamily, such as ALCAM₅₅₉₋₅₈₀, where the GPCR polypeptide is constitutively active and/or is activated by addition of an agonist, partial agonist or allosteric modulator of that GPCR; and
b. detecting whether the candidate agent, where the candidate agent is a fragment or derivative of a member of the IgSF CAM superfamily, such as ALCAM₅₅₉₋₅₈₀, is a modulator of RAGE ligand-independent activation of RAGE by activated co-located GPCR by detecting an effect indicative of modulation of RAGE activation by the presence of the candidate agent, where the candidate agent is a fragment or derivative of a member of the IgSF CAM superfamily, such as ALCAM₅₅₉₋₅₈₀, and/or by detecting RAGE-dependent signalling that is modulated by the presence of the candidate agent, where the candidate agent is a fragment or derivative of a member of the IgSF CAM superfamily, such as ALCAM₅₅₉₋₅₈₀.

In one form, the present invention comprises methods of screening candidate agents, where candidate agents are fragments or derivatives of members of the IgSF CAM superfamily, for their ability to modulate RAGE ligand-independent activation of RAGE by activated certain co-located GPCR, comprising the steps of:
a. Contacting a RAGE polypeptide with a GPCR polypeptide in the presence of a candidate agent, where the GPCR polypeptide is constitutively active and/or is activated by addition of an agonist, partial agonist or allosteric modulator of that GPCR; and
b. detecting whether the candidate agent is a modulator of RAGE ligand-independent activation of RAGE by activated co-located GPCR by detecting an effect indicative of modulation of RAGE activation by the presence of the candidate agent, and/or by detecting RAGE-dependent signalling that is modulated by the presence of the candidate agent.

In some embodiments, the screening methods further comprise detecting whether the candidate agent, where the candidate agent is a fragment or derivative of a member of the IgSF CAM superfamily, such as ALCAM₅₅₉₋₅₈₀, is a modulator (such as activator, inhibitor or allosteric modulator) of the certain co-located GPCR, such as angiotensin receptor, such as an AT₁R or such as a certain complement receptor, such as C5a receptor 1 or a signalling pathway of the certain co-located GPCR, such as an angiotensin receptor signalling pathway, such as an AT₁R signalling pathway or such as a certain C5a receptor 1 signalling pathway, such as a C5a receptor 1 signalling pathway, in the presence or absence of RAGE. In some embodiments, the candidate agent, where the candidate agent is a fragment or derivative of a member of the IgSF CAM superfamily, such as ALCAM₅₅₉₋₅₈₀, that results in greater modulation of the signal when the RAGE polypeptide is present compared to when it is absent is selective for modulating RAGE-ligand independent activation of RAGE by activated co-located GPCR over RAGE-independent signalling resulting from activation of the co-located GPCR.

In one form, the invention comprises peptides identified as modulators by said methods.

In one form, the invention comprises compounds identified as modulators by said methods.

In some embodiments, the screening methods further comprise detecting whether the candidate agent, where the candidate agent is a fragment or derivative of a member of the IgSF CAM superfamily, such as ALCAM₅₅₉₋₅₈₀, is a modulator (such as activator, inhibitor, allosteric modulator or functional substitute) of RAGE or a RAGE signalling pathway in the presence or absence of the certain co-located GPCR, such as an angiotensin receptor, such as AT₁R, or such as a certain complement receptor, such as C5a receptor 1. In some embodiments, the candidate agent, where the candidate agent is a fragment or derivative of a member of the IgSF CAM superfamily, such as ALCAM₅₅₉₋₅₈₀, that results in greater modulation of the RAGE-dependent signal when the GPCR polypeptide is present compared to when it is absent is selective for modulating RAGE-ligand independent activation of RAGE by activated co-located GPCR.

In some embodiments, the screening methods further comprise detecting whether the candidate agent, where the candidate agent is a fragment or derivative of a member of the IgSF CAM superfamily, such as ALCAM₅₅₉₋₅₈₀, is a modulator (such as activator, inhibitor, allosteric modulator or functional substitute) of a RAGE polypeptide or a RAGE signalling pathway as well as the certain co-located GPCR, such as angiotensin receptor, such as an AT₁R, or such as a certain complement receptor, such as C5a receptor 1, or a signalling pathway of the certain co-located GPCR, such as an angiotensin receptor signalling pathway, such as an AT₁R signalling pathway or such as a certain complement receptor signalling pathway, such as a C5a receptor 1 signalling pathway.

In some embodiments, the screening method further comprises the step of using an inhibitor of RAGE ligand binding to the RAGE ectodomain that as such inhibits activation of RAGE in a RAGE ligand-dependent manner.

In some embodiments, the screening method further comprises use of a RAGE polypeptide that is mutated and/or truncated such that it is not able to bind RAGE ligands to its ectodomain and as such is not able to be activated in a RAGE ligand-dependent manner.

In some embodiments, binding of RAGE ligands to the ectodomain of RAGE is impaired by exposing the cell to a modulator that modulates the binding of RAGE ligands to RAGE.

In some embodiments the use of a RAGE polypeptide that is mutated and/or truncated such that it is not able to bind RAGE ligands and as such is not able to be activated in a RAGE ligand-dependent manner occurs before, after or in parallel with a screen involving a RAGE polypeptide that is able to bind RAGE ligands.

Suitably, a candidate agent or a derivative of a candidate agent, where the candidate agent or derivative of the candidate agent is a fragment or derivative of a member of the IgSF CAM superfamily, such as ALCAM₅₅₉₋₅₈₀, which modulates RAGE ligand-independent activation of RAGE by activated certain co-located GPCR, such as angiotensin receptor, such as an AT₁R or such as a certain complement receptor, such as C5a receptor 1, and that suitably modulates a certain co-located GPCR, such as angiotensin receptor, such as an AT₁R or such as a certain complement receptor, such as C5a receptor 1 and/or a signalling pathway of the certain co-located GPCR, such as an angiotensin receptor signalling pathway, such as an AT₁R signalling pathway or such as a certain complement receptor signalling pathway, such as a C5a signalling pathway and/or that inhibits RAGE ligand-dependent activation of RAGE and/or inhibits constitutively-active RAGE and/or a RAGE signalling pathway, is particularly useful for treating, preventing or managing a RAGE-related disorder.

In certain embodiments, the screening method assesses proximity of the RAGE polypeptide to the certain co-located GPCR, such as angiotensin receptor, such as AT₁R, or such as a certain complement receptor, such as C5a receptor 1, using a proximity screening assay. In illustrative examples of this type, the RAGE polypeptide is coupled (e.g., conjugated or otherwise linked) to a first reporter component and the certain co-located GPCR, such as angiotensin receptor, such as an AT₁R or such as a certain complement receptor, such as C5a receptor 1, is coupled (e.g., conjugated or otherwise linked) to a second reporter component. Proximity of the first and second reporter components generates a signal capable of detection by the detector. The first and second reporter components constitute a complementary pair, in the sense that the first reporter component may be interchanged with the second reporter component without appreciably affecting the functioning of the invention. The first and second reporter components can be the same or different.

In one embodiment, the proximity screening assay is that described in patent WO2008055313 (Dimerix Bioscience Pty Ltd; also U.S. Pat. Nos. 8,283,127, 8,568,997, EP2080012, CA2669088, CN101657715), also known as Receptor Heteromer Investigation Technology or Receptor-HIT (Jaeger et al., 2014). With this method, RAGE is coupled to a first reporter component, the certain co-located GPCR, such as angiotensin receptor, such as AT₁R or such as a certain complement receptor, such as C5a receptor 1 is unlabeled with respect to the proximity screening assay, and a GPCR-interacting group is linked to the complementary second reporter component, whose interaction with the complex is modulated upon binding a ligand selective for the unlabeled GPCR or the heteromer complex specifically. Preferred examples of GPCR-interacting groups are arrestins, G proteins and ligands. Alternatively, the certain co-located GPCR, such as angiotensin receptor, such as AT₁R or such as a certain complement receptor, such as C5a receptor 1 is coupled to a first reporter component, RAGE is unlabeled with respect to the proximity screening assay, and a RAGE-interacting group is linked to the complementary second reporter component, whose interaction with the complex is modulated upon binding a ligand selective for the unlabeled RAGE or the heteromer complex specifically. Preferred examples of RAGE-interacting groups are proteins interacting with the cytosolic tail of RAGE, such as IQGAP-1, Diaphanous 1, Dock7, MyD88, TIRAP, IRAK4, ERK1/2, and PKCζ (Jules et al., 2013; Ramasamy et al., 2016).

Reporter components can include enzymes, luminescent or bioluminescent molecules, fluorescent molecules, and transcription factors or other molecules coupled to RAGE, the certain co-located GPCR or the interacting group by linkers incorporating enzyme cleavage sites. In short any known molecule, organic or inorganic, proteinaceous or non-proteinaceous or complexes thereof, capable of emitting a detectable signal as a result of their spatial proximity.

Preferably, signal generated by the proximity of the first and second reporter components in the presence of the reporter component initiator is selected from the group consisting of: luminescence, fluorescence and colorimetric change.

In some embodiments, the luminescence is produced by a bioluminescent protein selected from the group consisting of luciferase, galactosidase, lactamase, peroxidase, or any protein capable of luminescence in the presence of a suitable substrate.

Preferable combinations of first and second reporter components include a luminescent reporter component with a fluorescent reporter component, a luminescent reporter component with a non-fluorescent quencher, a fluorescent reporter component with a non-fluorescent quencher, first and second fluorescent reporter components capable of resonance energy transfer. However, useful combinations of first and second reporter components are by no means limited to such.

In some embodiments, the screening methods further comprise detecting proximity of the first and second reporter components to one another to thereby determine whether the candidate agent, where the candidate agent is a fragment or derivative of a member of the IgSF CAM superfamily, such as ALCAM₅₅₉₋₅₈₀, modulates the interaction between the RAGE polypeptide and the certain co-located GPCR, such as angiotensin receptor, such as AT₁R or such as a certain complement receptor, such as C5a receptor 1. Generally, this is achieved when proximity of the first and second reporter components generates a proximity signal that is altered by the modulation by the candidate agent, where the candidate agent is a fragment or derivative of a member of the IgSF CAM superfamily, such as ALCAM₅₅₉₋₅₈₀, of the proximity between the RAGE polypeptide and the certain co-located GPCR, such as angiotensin receptor, such as AT₁R or such as a certain complement receptor, such as C5a receptor 1.

One or both of the RAGE and certain co-located GPCR, such as angiotensin receptor, such as AT₁R or such as a certain complement receptor, such as C5a receptor 1, may be in soluble form or expressed on the cell surface.

In some embodiments, the RAGE and certain co-located GPCR, such as angiotensin receptor, such as AT₁R or such as a certain complement receptor, such as C5a receptor 1, are located in, partially in, or on a single membrane; for example, both are expressed at the surface of a host cell.

In another embodiment of the invention, the certain co-located GPCR, such as an angiotensin receptor, such as AT₁R or such as a certain complement receptor, such as C5a receptor 1, is pre-assembled with RAGE in a pre-formed complex at the cell membrane.

In another embodiment of the invention, following activation of the certain co-located GPCR, such as angiotensin receptor, such as AT₁R or such as a certain complement receptor, such as C5a receptor 1, by engagement of cognate ligand, such as Ang II for AT₁R or C5a for C5a receptor 1, signalling is triggered that involves the cytosolic tail of RAGE.

In one embodiment of the invention, activation of the cytosolic tail of RAGE is associated with changes in its structural conformation and/or affinity for binding partners.

In one embodiment of the invention, monitoring of the structural conformation of RAGE and/or affinity for binding partners occurs when the cytosolic tail of RAGE has been mutated and/or truncated such that it can no longer be activated by RAGE ligands or by RAGE ligand-independent activation of RAGE by certain activated co-located GPCRs.

In one embodiment of the invention, monitoring structural conformation and/or affinity for binding partners occurs in the presence of agents that inhibit binding and/or activation of RAGE by RAGE ligands or RAGE ligand-independent activation of RAGE by certain activated co-located GPCRs.

In one embodiment of the invention, monitoring recruitment of binding partners occurs prior to activation of RAGE by RAGE ligands or RAGE ligand-independent activation of RAGE by certain activated co-located GPCRs.

In one embodiment of the invention, monitoring recruitment and activation of signalling mediators and/or binding partners to the RAGE cytosolic tail occurs subsequent to activation of RAGE by RAGE ligands or RAGE ligand-independent activation of RAGE by certain activated co-located GPCRs.

In one embodiment of the invention, monitoring recruitment of binding partners following activation of RAGE by RAGE ligands or RAGE ligand-independent activation of RAGE by certain activated co-located GPCRs occurs in the presence of agents that inhibit binding and/or activation of RAGE by RAGE ligands.

Further embodiments of the invention comprise methods of screening candidate agents, where candidate agents are fragments or derivatives of members of the IgSF CAM superfamily, such as ALCAM₅₅₉₋₅₈₀, for their ability to modulate (such as activate, inhibit or otherwise modulate) RAGE ligand-independent activation of RAGE by a certain co-located GPCR, such as angiotensin receptor, such as AT₁R or such as a certain complement receptor, such as C5a receptor 1, by detecting modulation of the RAGE-mediated signalling. Such methods may include the step of measuring canonical activation of NFκB, by measuring one or more of the following:
Activity of IkB kinase (IKK) by monitoring in vitro phosphorylation of a substrate, such as GST-IκBα;
Detection of IkB Degradation Dynamics including phosphorylation/ubiquitination and/or degradation of IκB and/or IκB-α;
Detection of p65(Rel-A) phosphorylation/ubiquitination, such as by using antibodies, gel-shift, EMSA, or mass spectroscopy;
Detection of cytoplasmatic to nuclear shuttling/translocation of NFκB components/subunits, such as p65/phospho-p65;
Detection of NFκB subunit dimerization/complexation;
Detection of active NFκB components/subunits by binding to immobilized DNA sequence/oligonucleotide containing the NFκB response element/consensus NFκB binding, such as by using Electrophoretic mobility shift assay or gel shift assay, SELEX, protein-binding microarray, or sequencing-based approaches;
Chromatin-immunoprecipitation (ChIP) assays to detect NFκB in situ binding to DNA to the promoters and enhancers of specific genes;
In vitro kinase assay for NFκB kinase activity;
Measurement of NFκB transcriptional activity using NFκB reporter assays via transgene expression of reporter constructs, such as LacZ Fluc, eGFP SEAP, NF-gluc, using approaches such as plasmid transfection, reporter cell lines, mini-circles, retrovirus, or lentivirus;
Measuring changes in expression of downstream targets of NFκB (such as cytokines, growth factors, adhesion molecules and mitochondrial anti-apoptotic genes by real-time PCR, protein, or functional assays) (Note the pleiotropic nature of NFκB is reflected in its transcriptional targets that presently number over 500 (see http://www.bu.edu/nf-kb/dene-resources/tardet-denes/accessed 7 Dec. 2018) and;
Measuring changes in function or structure induced by NFκB-dependent signalling, such as POLKADOTS in T-cells, adhesion in endothelial cells, activation in leucocytes, or oncogenicity.

Additionally or alternately, such methods may include measuring signals arising from the non-canonical actions of NF-κB, by measuring one or more of the following:
Detection of NIK (NFκB-Inducing Kinase);
Detecting IKκα Activation/phosphorylation;
Detection of NIK kinase activity by ability to autophosphorylate or to phosphorylate a substrate by performing a kinase assay;
Generation of p52-containing NFκB dimers, such as p52/RelB;
Detection of Phospho-NFκB2 p100(Ser866/870);
Detection of partial degradation (called processing) of the precursor p100 into p52;
Detecting p52/RelB translocation into the nucleus;
Detecting p52/RelB binding to KB sites;
Measurement of NFκB transcriptional activity using NFκB reporter assays via transgene expression of reporter constructs, such as LacZ Fluc, eGFP SEAP, NF-gluc, using approaches such as plasmid transfection, reporter cell lines, mini-circles, retrovirus, or lentivirus;
Measuring changes in expression of downstream targets of non-canonical signalling of NFκB (such as CXCL12) by real-time PCR, protein expression or by functional assays.

In one embodiment, an effect on the RAGE indicative of modulation of RAGE activation is a change in intracellular trafficking such as that detected by a change in proximity of luciferase-conjugated RAGE (such as RAGE/Rluc8) to intracellular compartment markers such as fluorophore-labelled Rabs, such as Rab1, Rab4, Rab5, Rab6, Rab7, Rab8, Rab9 and/or Rab11 (such as Venus-Rab1, Venus-Rab4, Venus-Rab5, Venus-Rabb, Venus-Rab7, Venus-Rabb, Venus-Rab9 and/or Venus-Rab11), and/or a plasma membrane marker, such as a fluorophore-conjugated fragment of K-ras (such as Venus-K-ras) using bioluminescence resonance energy transfer (BRET) upon addition of a cognate ligand for the co-located GPCR (Tiulpakov et al., 2016).

In another embodiment, an effect on the RAGE is a change in RAGE-dependent signalling, such as detected by a change in proximity of luciferase-conjugated RAGE (such as RAGE-Rluc8) to a RAGE-interacting group, such as fluorophore-labelled proteins interacting with the cytosolic tail of the RAGE, such as IQGAP-1, protein kinase C zeta (PKCζ), Dock7, MyD88, TIRAP, ERK1/2, (Jules et al., 2013; Ramasamy et al., 2016), olfactory receptor 2T2, ADP/ATP translocase 2, Protein phosphatase 1G, Intercellular adhesion molecule 1, Protein DJ-1 (PARK7), Calponin-3, Drebrin, Filamin B, Ras-related protein Rab-13, Radixin/Ezrin/Moesin, Proteolipid protein 2, Coronin, S100 A11, Succinyl-CoA ligase [GDP-forming] subunit alpha, Hsc70-interacting protein, Apoptosis Inhibitor 5, neuropilin, cleavage stimulation factor, growth factor receptor-bound protein 2, sec61 beta subunit, or Nck1.

In another aspect, the present invention provides methods of identifying a candidate agent that is a modulator (such as activator, inhibitor, allosteric modulator or functional substitute), where the modulator is a fragment or derivative of a member of the IgSF CAM superfamily, such as ALCAM₅₅₉₋₅₈₀, that modulates (i.e., activates, inhibits or otherwise modulates) RAGE ligand-independent activation of RAGE following activation of a certain co-located GPCR by a cognate ligand, such as AT₁R or such as a certain complement receptor, such as C5a receptor 1 or if the certain co-located GPCR is constitutively active, and that suitably modulates a certain co-located GPCR, such as an angiotensin receptor, such as AMR or such as a certain complement receptor, such as C5a receptor 1, and/or that modulates a RAGE polypeptide or a RAGE signalling pathway. In a preferred form of the invention, such a modulator is an inhibitor of one or both of the RAGE or certain co-located GPCR, such as an angiotensin receptor, such as AT₁R or such as a certain complement receptor, such as C5a receptor 1, or of the RAGE signalling pathway. In a particularly preferred form of the invention, the modulation of the RAGE signalling pathway is distinct from and/or occurs to a significantly different extent to the modulation of classical certain co-located GPCR signalling pathways, such as AT₁R signalling pathways, such as the Gq signalling pathway, or C5a receptor 1 signalling pathways, such as the Gi signalling pathway. In a particularly preferred form of the invention, the inhibition of the RAGE signalling pathway is distinct from and/or greater than the inhibition of classical certain co-located GPCR signalling pathways, such as AT₁R signalling pathways, such as the Gq signalling pathway, or such as C5a receptor 1 signalling pathways, such as the Gi signalling pathway.

The present invention includes modulators identified by any of the aforementioned methods and the use of such modulators to modulate activity as described herein.

The present invention also includes pharmaceutical compositions containing said modulators, and the use of said pharmaceutical compositions for the treatment or prevention of an ailment in a patient in need of such treatment.

The present invention includes the use of a modulator of the present invention in the manufacture of a medicament to treat an ailment.

Throughout this specification, unless the context requires otherwise, an activated GPCR means a GPCR that is in an active state that may result from the binding of an agonist, partial agonist and/or allosteric modulator, and/or as a consequence of constitutive activity that does not necessitate ligand binding.

Throughout this specification, unless the context requires otherwise, the certain activated co-located GPCRs of the invention are GPCRs that are expressed in the same cell as an IgSF CAM and for which an effect on an IgSF CAM, indicative of modulation of an IgSF CAM activation and/or modulation of induction of IgSF CAM-dependent signalling, is detected upon activation by cognate ligands of the certain co-located GPCRs or when the GPCRs are constitutively active.

### BRIEF DESCRIPTION OF THE DRAWINGS

FIG. 1.

FIG. 1A. The induction of p65 expression in CHO cells (which lack AT1R) exposed to Ang II (1 μM) for 2 hours in the presence or absence of transfection with pCI neo (empty vector) or an IgSF CAM, specifically murine ALCAM, or the cytosolic tail of human ALCAM₅₅₁₋₅₈₃, compared to their respective untreated control shown as fold change. Grey columns are untreated, white columns are Ang II-treated. Individual replicates are shown.

FIG. 1B. The induction of p65 expression in AT1R-CHO cells (which express human AT1R) exposed to Ang II (1 μM) for 2 hours in the presence or absence of transfection with pCI neo (empty vector) or an IgSF CAM, specifically full length human ALCAM₁₋₅₈₃, compared to their respective untreated control shown as fold change. Grey columns are untreated, white columns are Ang II-treated. Individual replicates are shown.

FIG. 1C. The induction of p65 expression in AT1R-CHO cells exposed to Ang II (1 μM) for 2 hours in the presence or absence of transfection with pCI neo (empty vector) or an IgSF CAM, specifically full length murine ALCAM₁₋₅₈₃, compared to their respective untreated control shown as fold change. Grey columns are untreated, white columns are Ang II-treated. Individual replicates are shown.

FIG. 1D. The induction of p65 expression in AT1R-CHO cells exposed to Ang II (1 μM) for 2 hours in the presence or absence of transfection with pCI neo (empty vector) or an IgSF CAM, specifically full-length chicken EpCAM, compared to their respective untreated control shown as fold change. Grey columns are untreated, white columns are Ang II-treated. Individual replicates are shown.

FIG. 1E. The induction of p65 expression in AT1R-CHO cells exposed to Ang II (1 μM) for 2 hours in the presence or absence of transfection with pCI neo (empty vector) or the cytosolic tail of an IgSF CAM, specifically the cytosolic tail of ALCAM₅₅₁₋₅₈₃, compared to their respective untreated control shown as fold change and its modulation by cotransfection with RAGE₃₇₀₋₃₉₀. Grey columns are untreated, white columns are Ang II-treated. Individual replicates are shown.

FIG. 1F. The induction of p65 expression in AT1R-CHO cells exposed to Ang II (1 μM) for 2 hours in the presence or absence of transfection with pCI neo (empty vector) or the cytosolic tail of an IgSF CAM, specifically the cytosolic tails of ALCAM, BCAM, MCAM, EpCAM and CADM4 or pCIneo (empty vector) compared to their respective untreated control shown as fold change. Grey columns are untreated, white columns are Ang II-treated. Individual replicates are shown.

FIG. 1G. The induction of p65 expression in AT1R-CHO cells exposed to Ang II (1 μM) for 2 hours in the presence or absence of transfection with pCI neo (empty vector) or the cytosolic tail of an IgSF CAM, specifically the cytosolic tails of EpCAM and CADM4, or the cytosolic tail of RAGE (RAGE₃₇₀₋₄₀₄), compared to their respective untreated control shown as fold change. Grey columns are untreated, white columns are Ang II-treated. Individual replicates are shown.

FIG. 1H. The induction of PCNA expression in AT1R-CHO cells exposed to Ang II (1 μM) for 2 hours in the presence or absence of transfection with the pCI neo (empty vector) or the cytosolic tail of an IgSF CAM, specifically the cytosolic tails of EpCAM and CADM4 compared to their respective untreated control shown as fold change. Grey columns are untreated, white columns are Ang II-treated. Individual replicates are shown.

FIG. 2.

FIG. 2A. The induction of ICAM-1 expression in adult retinal pigment epithelial (ARPE) cells exposed to Ang II (1 μM) in the presence or absence of transfection of pCI neo (empty vector) or a fragment of an IgSF CAM, more specifically the cytosolic tail of an IgSF CAM, even more specifically the cytosolic tail of ALCAM where the cytosolic tail of ALCAM is residues 551-583, and its modulation by co-transfection with a fragment of RAGE, specifically RAGE₃₇₀₋₃₉₀, compared to their respective untreated control shown as fold change. Grey columns are untreated, white columns are AngII-treated and striped bars are treated with RAGE₃₇₀₋₃₉₀+AngII. Individual replicates are shown.

FIG. 2B. The induction of ICAM-1 expression in adult retinal pigment epithelial (ARPE) cells exposed to Ang II (1 μM) in the presence or absence of transfection of pCI neo (empty vector) or a fragment of an IgSF CAM, more specifically the cytosolic tail of an IgSF CAM, even more specifically the cytosolic tail of MCAM where the cytosolic tail of MCAM is residues 584-637, and its modulation by co-transfection with a fragment of RAGE, specifically RAGE₃₇₀₋₃₉₀, compared to their respective untreated control shown as fold change. Grey columns are untreated, white columns are AngII-treated and striped bars are treated with RAGE₃₇₀₋₃₉₀+AngII. Individual replicates are shown.

FIG. 2C. The induction of ICAM-1 expression in adult retinal pigment epithelial (ARPE) cells exposed to Ang II (1 μM) in the presence or absence of transfection of pCI neo (empty vector) or a fragment of an IgSF CAM, more specifically an IgSF CAM cytosolic tail, even more specifically the cytosolic tail of ALCAM where the cytosolic tail of ALCAM is residues 551-583 or the cytosolic tail of BCAM where the cytosolic tail of BCAM is residues 569-628, and its modulation by treatment with a fragment of RAGE, specifically the mCherry-TAT-S391A-RAGE₃₆₂₋₄₀₄ oligopeptide (A Peptide), compared to their respective untreated control shown as fold change. Grey columns are untreated, white columns are AngII-treated and striped bars are treated with mCherry-TAT-S391A-RAGE₃₆₂₋₄₀₄+AngII. Individual replicates are shown.

FIG. 3

FIG. 3A. The induction of p65 expression in CHO cells (which lack AT1R) exposed to Ang II (1 μM) in the presence or absence of transfection with pCI neo (empty vector) or a fragment of an IgSF CAM, specifically the cytosolic tail of human ALCAM (hALCAM₅₅₁₋₅₈₃ or hALCAM₅₅₉₋₅₈₀), compared to their respective untreated control shown as fold change. Grey columns are untreated and white columns are AngII-treated. Individual replicates are shown.

FIG. 3B. The induction of p65 expression in AT1-CHO cells exposed to Ang II (1 μM) in the presence of transfection with: pCI neo (empty vector) or an IgSF CAM, specifically full length mouse ALCAM (murine ALCAM₁₋₅₈₃); or a derivative of an IgSF CAM, specifically the cytosolic tail of human ALCAM omitting all serine and threonine residues (hALCAM₅₅₉₋₅₈₀); or murine ALCAM₁₋₅₈₃ together with ALCAM₅₅₉₋₅₈₀. Data are compared to their respective untreated control shown as fold change. Grey columns are untreated and white columns are AngII-treated. Individual replicates are shown.

FIG. 3C. The induction of PCNA expression in AT1-CHO cells exposed to Ang II (1 μM) in the presence of transfection with: pCI neo (empty vector) or an IgSF CAM, specifically full length mouse ALCAM (murine ALCAM₁₋₅₈₃); or a derivative of an IgSF CAM, specifically the cytosolic tail of human ALCAM omitting all serine and threonine residues (hALCAM₅₅₉₋₅₈₀); or murine ALCAM₁₋₅₈₃ together with ALCAM₅₅₉₋₅₈₀. Data are compared to their respective untreated control shown as fold change. Grey columns are untreated and white columns are AngII-treated. Individual replicates are shown.

FIG. 3D. The induction of p65 expression in AT1-CHO cells exposed to Ang II (1 μM) in the presence of transfection with: pCI neo (empty vector) or full length chicken EpCAM; or a derivative of an IgSF CAM, specifically the cytosolic tail of human ALCAM omitting all serine and threonine residues (hALCAM₅₅₉₋₅₈₀); or chicken EpCAM together with ALCAM₅₅₉₋₅₈₀. Data are compared to their respective untreated control shown as fold change. Grey columns are untreated and white columns are AngII-treated. Individual replicates are shown.

FIG. 3E. The induction of p65 expression in AT1-CHO cells exposed to Ang II (1 μM) in the presence of transfection with: pCI neo (empty vector) or the full length human ALCAM (specifically ALCAM₁₋₅₈₃ (SEQUENCE ID NO #9) or a derivative of an IgSF CAM, specifically the cytosolic tail of human ALCAM omitting all serine and threonine residues (hALCAM₅₅₉₋₅₈₀); or human ALCAM₁₋₅₈₃ together with human ALCAM₅₅₉₋₅₈₀. Data are compared to their respective untreated control shown as fold change. Grey columns are untreated and white columns are AngII-treated. Individual replicates are shown.

FIG. 3F. The induction of PCNA expression in AT1-CHO cells exposed to Ang II (1 μM) in the presence of transfection with: pCI neo (empty vector) or the full length human ALCAM (specifically ALCAM₁₋₅₈₃ (SEQUENCE ID NO #9) or a derivative of an IgSF CAM, specifically the cytosolic tail of human ALCAM omitting all serine and threonine residues (hALCAM₅₅₉₋₅₈₀); or human ALCAM₁₋₅₈₃ together with human ALCAM₅₅₉₋₅₈₀. Grey columns are untreated and white columns are AngII-treated. Individual replicates are shown. Data are compared to their respective untreated control shown as fold change.

FIG. 3G. The induction of p65 expression in AT1-CHO cells exposed to Ang II (1 μM) in the presence of transfection with the cytosolic tail of human ALCAM (specifically ALCAM₅₅₁₋₅₈₃ (SEQUENCE ID NO #1) in addition to transfection with a derivative of an IgSF CAM, specifically the cytosolic tail of human ALCAM omitting all serine and threonine residues (hALCAM₅₅₉₋₅₈₀), or a derivative of the RAGE cytosolic tail, specifically RAGE₃₇₉₋₃₉₀ (SEQ ID NO: 21). Grey columns are untreated and white columns are Ang II-treated. Individual replicates are shown. Data are compared to their respective untreated control shown as fold change.

FIG. 3H. The induction of p65 expression in AT1-CHO cells exposed to Ang II (1 μM) in the presence of transfection with the cytosolic tail of human BCAM (specifically BCAM₅₆₉₋₆₂₈ (SEQUENCE ID NO #2) in addition to transfection with a derivative of an IgSF CAM, specifically the cytosolic tail of human ALCAM omitting all serine and threonine residues (hALCAM₅₅₉₋₅₈₀), or a derivative of the RAGE cytosolic tail, specifically RAGE₃₇₉₋₃₉₀ (SEQ ID NO: 21). Grey columns are untreated and white columns are AngII-treated. Individual replicates are shown. Data are compared to their respective untreated control shown as fold change.

FIG. 3I. The induction of p65 expression in AT1-CHO cells exposed to Ang II (1 μM) in the presence of transfection with the cytosolic tail of human MCAM (specifically MCAM₅₈₄₋₆₃₇ (SEQUENCE ID NO #3)) in addition to transfection with a derivative of an IgSF CAM, specifically the cytosolic tail of human ALCAM omitting all serine and threonine residues (hALCAM₅₅₉₋₅₈₀), or a derivative of the RAGE cytosolic tail, specifically RAGE₃₇₉₋₃₉₀ (SEQ ID NO: 21). Grey columns are untreated and white columns are AngII-treated. Individual replicates are shown. Data are compared to their respective untreated control shown as fold change.

FIG. 3J. The induction of p65 expression in AT1-CHO cells exposed to Ang II (1 μM) in the presence of transfection with: pCI neo (empty vector); the cytosolic tail of human EpCAM (specifically EpCAM₂₈₉₋₃₁₄ (SEQUENCE ID NO #4)) in addition to transfection with a derivative of an IgSF CAM, specifically the cytosolic tail of human ALCAM omitting all serine and threonine residues (hALCAM₅₅₉₋₅₈₀), or a derivative of the RAGE cytosolic tail, specifically RAGE₃₇₉₋₃₉₀ (SEQ ID NO: 21). Grey columns are untreated and white columns are AngII-treated. Individual replicates are shown. Data are compared to their respective untreated control shown as fold change.

FIG. 3K. The induction of p65 expression in AT1-CHO cells exposed to Ang II (1 μM) in the presence of transfection with the cytosolic tail of human CADM4 (specifically CADM4₃₄₆₋₃₈₈ (SEQUENCE ID NO #5)) with or without additional transfection with a derivative of the RAGE cytosolic tail, specifically RAGE₃₇₉₋₃₉₀ (SEQ ID NO: 21). Grey columns are untreated and white columns are AngII-treated. Individual replicates are shown. Data are compared to their respective untreated control shown as fold change.

FIG. 4

FIG. 4A. The induction of PCNA expression in CHO cells exposed to the IgSF ligand, S100A8/A9 (1 μM) for 2 hours in the presence or absence of transfection of or pCIneo (empty vector) or an IgSF CAM, specifically full length murine ALCAM₁₋₅₈₃, and its modulation by co-transfection with a fragment of cytosolic tail of ALCAM, specifically ALCAM₅₅₉₋₅₈₀ or a fragment of the cytosolic tail of RAGE, specifically RAGE₃₇₀₋₃₉₀, compared to their respective untreated control shown as fold change. Grey columns are untreated, white columns are S100A8/A9-treated. Individual replicates are shown.

FIG. 4B. The induction of PCNA expression in CHO cells exposed to the IgSF ligand S100A8/A9 (1 μM) for 2 hours in the presence or absence of transfection of pCIneo (empty vector) or full length RAGE, and its modulation by co-transfection with a fragment of cytosolic tail of ALCAM, specifically ALCAM₅₅₉₋₅₈₀ compared their respective untreated control shown as fold change. Grey columns are untreated, white columns are S100A8/A9-treated. Individual replicates are shown.

FIG. 5

FIG. 5A. The induction of p65 expression in AT1R-CHO cells exposed to Ang II (1 μM) for two hours in the presence of transfection of a derivative of an IgSF CAM, specifically the cytosolic tail of human ALCAM omitting all serine and threonine residues (hALCAM₅₅₉₋₅₈₀ (SEQ ID NO: 6), S391A-RAGE₃₆₂₋₄₀₄ or a derivative of the RAGE cytosolic tail, specifically RAGE₃₇₉₋₃₉₀ (SEQ ID NO: 21) compared to their respective untreated control shown as fold change. Grey columns are untreated, white columns are AngII-treated. Individual replicates are shown.

FIG. 5B. The induction of p65 expression in AT1R-CHO cells exposed to Ang II (1 μM) for two hours in the presence or absence of transfection of pCIneo (empty vector) or full length murine ALCAM, and its modulation by co-transfection with a fragment of RAGE, specifically RAGE₃₇₀₋₃₉₀ compared to their respective untreated control shown as fold change. Grey columns are untreated, white columns are AngII-treated. Individual replicates are shown.

FIG. 6

FIG. 6A. The induction of p65 expression in AT1R-CHO cells exposed to Ang II (1 μM) for two hours in the presence or absence of transfection of pCI neo (empty vector) or full length human RAGE, and its modulation by co-transfection with a derivative of an IgSF CAM, specifically the cytosolic tail of human ALCAM omitting all serine and threonine residues (hALCAM₅₅₉₋₅₈₀). Grey columns are untreated, white columns are AngII-treated. Individual replicates are shown. Data are compared to their respective untreated control shown as fold change.

FIG. 6B. The induction of ICAM-1 expression in adult retinal pigment epithelial (ARPE) cells exposed to C5a (1 μM) in the presence or absence of transfection with: pCIneo (empty vector) or a derivative of an IgSF CAM, specifically the cytosolic tail of ALCAM omitting all serine and threonine residues (ALCAM₅₅₉₋₅₈₀); both the C5a receptor 1 (C5aR1) and full length RAGE (RAGE,-404); or C5aR1, full length RAGE (RAGE₁₋₄₀₄) and ALCAM₅₅₉₋₅₈₀. Grey columns are untreated and white columns are C5a-treated. Individual replicates are shown. Data are compared to their respective untreated control shown as fold change.

FIG. 7

FIG. 7A. The induction of p65 expression in AT1R-CHO cells exposed to Ang II (1 μM) for two hours in the presence or absence of transfection of full length human mutant S391A-RAGE, and its modulation by co-transfection with pCI neo (empty vector) or a fragment of RAGE, specifically RAGE₃₇₀₋₄₀₄, or a fragment of an IgSF CAM, specifically the cytosolic tail of human ALCAM or CADM4 compared to their respective untreated control shown as fold change. Grey columns are untreated, white columns are AngII-treated. Individual replicates are shown.

FIG. 7B. The induction of PCNA expression in CHO cells exposed to Ang II (1 μM) for two hours in the presence or absence of transfection of pCI neo (empty vector) or full length human mutant S391A-RAGE, and its modulation by co-transfection with a fragment of RAGE, specifically RAGE₃₇₀₋₄₀₄, or a fragment of an IgSF CAM, specifically the cytosolic tail of human ALCAM, compared to their respective untreated control shown as fold change. Grey columns are untreated, white columns are AngII-treated. Individual replicates are shown.

FIG. 8

FIG. 8A. Arginine vasopressin (AVP)-induced recruitment of β-arrestin2/Venus proximal to ALCAM/Rluc8 in the presence of vasopressin receptor 2 (V2R). AVP-induced recruitment of βarrestin2/Venus to V2R/Rluc8 included as a control.

FIG. 8B. Sphingosine-1-phosphate (S1P)-induced recruitment of β-arrestin2/Venus proximal to ALCAM/Rluc8 in the presence of S1P receptor 1 (S1PR1). S1P-induced recruitment of βarrestin2/Venus to S1PR1/Rluc8 included as a control.

FIG. 8C. Isoproterenol (Isop)-induced recruitment of β-arrestin2/Venus proximal to ALCAM/Rluc8 in the presence of β2 Adrenergic receptor (β2AR). Isop-induced recruitment of βarrestin2/Venus to β2AR/Rluc8 included as a control.

FIG. 8D. Orexin A (OxA)-induced recruitment of β-arrestin2/Venus proximal to ALCAM/Rluc8 in the presence of Orexin receptor 2 (OxR2). OxA-induced recruitment of βarrestin2/Venus to OxR2/Rluc8 included as a control.

FIG. 8E. Thyrotrophin-releasing hormone (TRH)-induced recruitment of β-arrestin2/Venus proximal to ALCAM/Rluc8 in the presence of Thyrotrophin-releasing hormone receptor 1 (TRHR1). TRH-induced recruitment of βarrestin2/Venus to TRHR1/Rluc8 included as a control.

FIG. 8E. Thyrotrophin-releasing hormone (TRH)-induced recruitment of β-arrestin2/Venus proximal to ALCAM/Rluc8 in the presence of Thyrotrophin-releasing hormone receptor 1 (TRHR1). TRH-induced recruitment of βarrestin2/Venus to TRHR1/Rluc8 included as a control.

FIG. 8F. CC chemokine ligand 3 (CCL3)-induced recruitment of β-arrestin2/Venus proximal to ALCAM/Rluc8 in the presence of CC chemokine receptor 1 (CCR1). CCL3-induced recruitment of βarrestin2/Venus to CCR1/Rluc8 included as a control.

FIG. 8G. CC chemokine ligand 2 (CCL2)-induced recruitment of β-arrestin2/Venus proximal to ALCAM/Rluc8 in the presence of CC chemokine receptor 2 (CCR2). CCL2-induced recruitment of βarrestin2/Venus to CCR2/Rluc8 included as a control.

FIG. 8H. CC chemokine ligand 20 (CCL20)-induced recruitment of β-arrestin2/Venus proximal to ALCAM/Rluc8 in the presence of CC chemokine receptor 6 (CCR6). CCL20-induced recruitment of βarrestin2/Venus to CCR6/Rluc8 included as a control.

FIG. 8I. CC chemokine ligand 19 (CCL19)-induced recruitment of β-arrestin2/Venus proximal to ALCAM/Rluc8 in the presence of CC chemokine receptor 7 (CCR7). CCL19-induced recruitment of βarrestin2/Venus to CCR7/Rluc8 included as a control.

FIG. 8J. CXC chemokine ligand 8 (CXCL8)-induced recruitment of β-arrestin2/Venus proximal to ALCAM/Rluc8 in the presence of CXC chemokine receptor 2 (CXCR2). CXCL8-induced recruitment of βarrestin2/Venus to CXCR2/Rluc8 included as a control.

FIG. 8K. CXC chemokine ligand 16 (CXCL16)-induced recruitment of β-arrestin2/Venus proximal to ALCAM/Rluc8 in the presence of CXC chemokine receptor 6 (CXCR6). CXCL16-induced recruitment of βarrestin2/Venus to CXCR6/Rluc8 included as a control.

FIG. 8L. Somatostatin (SST)-induced recruitment of β-arrestin2/Venus proximal to ALCAM/Rluc8 in the presence of somatostatin receptor 3 (SSTR3). SST-induced recruitment of βarrestin2/Venus to SSTR3/Rluc8 included as a control.

FIG. 9

FIG. 9A. Thyrotrophin-releasing hormone (TRH)-induced change in BRET ratio observed between ALCAM/Rluc8 and Venus-tagged Thyrotrophin-releasing hormone receptor 1 (TRHR1/Venus; 100 ng cDNA transfected/well of a 6-well plate). Lack of TRH-induced change in BRET ratio when ALCAM/Rluc8 expressed in absence of TRHR1/Venus as a control.

FIG. 9B. BRET saturation curve with ALCAM/Rluc8 and TRHR1/Venus.

FIG. 9C. Angiotensin II (AngII)-induced change in BRET ratio observed between ALCAM/Rluc8 and Venus-tagged Angiotensin II receptor 1 (AT₁/Venus; 100 ng cDNA transfected/well of a 6-well plate). Lack of AngII-induced change in BRET ratio when ALCAM/Rluc8 expressed in absence of AT₁/Venus as a control.

FIG. 9D. BRET saturation curves with ALCAM/Rluc8 and AT₁/Venus in cells co-transfected with pcDNA3 or ALCAM cDNA.

FIG. 9E. BRET saturation curves with ALCAM/Rluc8 and AT₁/Venus in cells co-transfected with pcDNA3 or RAGE cDNA.

FIG. 9F. CXC chemokine ligand 12 (CXCL12) treatment reduces proximity of β-arrestin2/Venus to ALCAM/Rluc8 in the presence of CXC chemokine receptor 4 (CXCR4). CXCL12-induced recruitment of βarrestin2/Venus to CXCR4/Rluc8 included as a control.

FIG. 9G. CCL2-induced change in BRET ratio observed between ALCAM/Rluc8 and CCR2/Venus (100 ng cDNA transfected/well of a 6-well plate). Lack of CCL2-induced change in BRET ratio when ALCAM/Rluc8 expressed in absence of CCR2/Venus as a control.

FIG. 9H. BRET saturation curves with ALCAM/Rluc8 and CCR2/Venus in cells co-transfected with pcDNA3 or RAGE cDNA or ALCAM cDNA.

FIG. 9I. BRET saturation curves with ALCAM/Rluc8 and CXCR6/Venus in cells co-transfected with pcDNA3 or RAGE cDNA or ALCAM cDNA.

FIG. 9J. BRET saturation curves with ALCAM/Rluc8 and β2AR/Venus in cells co-transfected with pcDNA3 or RAGE cDNA or ALCAM cDNA.

FIG. 9K. BRET saturation curves with ALCAM/Rluc8 and AT₁/Venus in cells co-transfected with pcDNA3 or EPCAM cDNA.

FIG. 9L. BRET saturation curves with ALCAM/Rluc8 and CCR2/Venus in cells co-transfected with pcDNA3 or EPCAM cDNA.

FIG. 10

FIG. 10A. AngII-induced recruitment of β-arrestin2/Venus proximal to human ALCAM/Rluc8 in the presence, but not in the absence, of AT₁.

FIG. 10B. AngII-induced recruitment of β-arrestin2/Venus proximal to mouse ALCAM/Rluc8 in the presence, but not in the absence, of AT₁.

FIG. 10C. AngII-induced recruitment of β-arrestin2/Rluc8 proximal to mouse ALCAM/Venus in the presence, but not in the absence, of AT₁.

FIG. 11

FIG. 11A. AngII-induced recruitment of β-arrestin2/Venus proximal to EPCAM/Rluc8 in the presence, but not in the absence, of AT₁.

FIG. 11B. AngII-induced recruitment of β-arrestin2/Rluc8 proximal to EPCAM/Venus in the presence, but not in the absence, of AT₁.

FIG. 11C. AngII-induced change in BRET ratio observed between EPCAM/Rluc8 and AT₁/Venus (100 ng cDNA transfected/well of 6-well plate). Lack of AngII-induced change in BRET ratio when EPCAM/Rluc8 expressed in absence of AT₁/Venus as a control.

FIG. 11D. BRET saturation curves with EPCAM/Rluc8 and AT₁/Venus in cells co-transfected with pcDNA3 or RAGE cDNA or ALCAM cDNA.

FIG. 11E. BRET saturation curves with EPCAM/Rluc8 and CCR2/Venus in cells co-transfected with pcDNA3 or RAGE cDNA or ALCAM cDNA.

FIG. 11F. BRET saturation curves with EPCAM/Rluc8 and AT₁/Venus in cells co-transfected with pcDNA3 or EPCAM cDNA.

FIG. 11G. BRET saturation curves with EPCAM/Rluc8 and CCR2/Venus in cells co-transfected with pcDNA3 or EPCAM cDNA.

FIG. 12

FIG. 12A. AngII-induced recruitment of β-arrestin2/Venus proximal to CADM4/Rluc8 in the presence, but not in the absence, of AT₁.

FIG. 12B. AngII-induced recruitment of β-arrestin2/Rluc8 proximal to CADM4/Venus in the presence, but not in the absence, of AT₁.

FIG. 12C. AngII-induced change in BRET ratio observed between CADM4/Rluc8 and AT₁/Venus (100 ng cDNA transfected/well of 6-well plate). Lack of AngII-induced change in BRET ratio when CADM4/Rluc8 expressed in absence of AT₁/Venus as a control.

FIG. 12D. BRET saturation curves with CADM4/Rluc8 and AT₁/Venus in cells co-transfected with pcDNA3 or RAGE cDNA or ALCAM cDNA.

FIG. 12E. BRET saturation curves with CADM4/Rluc8 and CCR2/Venus in cells co-transfected with pcDNA3 or RAGE cDNA or ALCAM cDNA.

FIG. 13

FIG. 13A. BRET saturation curves with RAGE/Rluc8 and AT₁/Venus in cells co-transfected with pcDNA3 or ALCAM cDNA.

FIG. 13B. BRET saturation curves with RAGE/Rluc8 and CCR2/Venus in cells co-transfected with pcDNA3 or ALCAM cDNA.

FIG. 13C. BRET saturation curves with RAGE/Rluc8 and CXCR6/Venus in cells co-transfected with pcDNA3 or ALCAM cDNA.

FIG. 13D. BRET saturation curves with RAGE/Rluc8 and AT₁/Venus in cells co-transfected with pcDNA3 or EPCAM cDNA.

FIG. 13E. BRET saturation curves with RAGE/Rluc8 and CCR2/Venus in cells co-transfected with pcDNA3 or EPCAM cDNA.

### EXAMPLES

In each of the following examples independently, the following general materials and methods apply, unless the context requires otherwise.

### Cell Culture

Adult retinal pigment epithelial (ARPE) cells were cultured in Dulbecco's modified Eagle's medium (DMEM)/F12 endothelial cell growth supplement (ECGS) supplemented media. Chinese Hamster Ovary (CHO) cells were cultured using F12 media (10% FCS with 2 mM glutamine). Human microvascular endothelial cells (HMEC) were cultured in MCDB 131 medium (10% FCS with 10 mM glutamine, EGF and hydrocortisone).

### Generation of Transgenic Chinese Hamster Ovary Cells

100 ng of AT₁R-Rluc8 construct was transfected into CHO cells using Lipofectamine 2000 (Thermo). Stable transfectants were selected using G418. AT₁R-CHO were then transiently transfected with the IgSF CAM and/or RAGE constructs using Lipofectamine 2000 (Invitrogen) and incubated for 16h.

### Generation of Oligonucleotides

Oligonucleotides were designed and ordered to generate the ALCAM, BCAM and MCAM intracellular (cytosolic) domains. These included a 5′ NheI site, Kozak sequence and initiating Methionine and then DNA sequences corresponding to ALCAM residues 552-583, BCAM residues 569-628 and MCAM residues 584-637 respectively (Note that as residue 551 of ALCAM is Methionine, the cytosolic tail of ALCAM effectively corresponded to residues 551-583). The pCIneo parental vector was digested with NheI and NotI restriction enzymes, and the DNA of the fragments of the ALCAM, BCAM and MCAM tails/cytosolic domains were ligated into the digested plasmid. After transformation and recovery, colonies were screened and individual clones sequenced. The sequence of the insert was confirmed by DNA sequencing (Micromon, Monash University). A full-length clone of Mouse ALCAM (BC027280) was purchased from Origene. The untagged clone was supplied in the vector pCMV6. Overlapping DNA sequences were ordered to generate the ALCAM₅₅₉₋₅₈₀ fragment oligonucleotide. These included a 5′ NheI site, Kozak sequence and initiating Methionine and then DNA sequences corresponding to ALCAM residues 559-580. The pCIneo parental vector was digested with NheI and NotI restriction enzymes, and the ALCAM₅₅₉₋₅₈₀ construct DNA was ligated into the digested plasmid. After transformation and recovery, colonies were screened and individual clones sequenced.

### Cellular Expression of Pro-Inflammatory Markers and Mediators by Quantitative Real-Time PCR

After 2 hours of exposure to Ang II (1 μM) cells were placed in Trizol, mRNA extracted and cDNA synthesized. Changes in the gene expression of the NFκB subunit, p65 (RelA) or NFκB-activated target genes (e.g ICAM-1) were estimated by quantitative real-time RT-PCR, performed using the TaqMan system based on real-time detection of accumulated fluorescence (ABI Prism 7700, Perkin-Elmer Inc, PE Biosystems, Foster City, Calif., USA). Gene expression was normalized to 18S mRNA and reported as fold change compared to the level of expression in untreated control mice/cells, which were given an arbitrary value of 1.

### Bioluminescence Resonance Energy Transfer (BRET)

BRET is an established technology for studying protein-protein proximity in live cells, particularly involving GPCRs (Pfleger and Eidne, 2006). One protein of interest was linked to a bioluminescent donor enzyme, Rluc8, a variant of Renilla luciferase, and a second linked to an acceptor fluorophore, Venus, a variant of green fluorescent protein. If in close proximity (<10 nm), energy resulting from the rapid oxidation of a cell-permeable coelenterazine substrate by the donor can transfer to the acceptor, which in turn fluoresces at a longer characteristic wavelength.

Plasmids were transiently co-expressed in human embryonic kidney (HEK) 293FT cells and BRET measurements taken at 37° C. using a CLARIOstar plate reader (BMG Labtech, Mornington, Victoria, Australia) with 420-480 nm (‘donor emission’) and 520-620 nm (‘acceptor emission’) filters.

The BRET ratio was calculated by subtracting the ratio of ‘acceptor emission’ over ‘donor emission’ for a cell sample expressing Rluc8-tagged protein alone from the same ratio for a cell sample expressing both Rluc8 and Venus-tagged proteins. Alternatively, the ligand-induced BRET signal was calculated by subtracting the ratio of ‘acceptor emission’ over ‘donor emission’ for a vehicle-treated cell sample from the same ratio for a second aliquot of the same cells treated with agonist.

For the BRET kinetic assays, the final pre-treatment reading is presented at the zero time point (time of ligand/vehicle addition). For the BRET saturation assays, fluorescence after light excitation was measured on an EnVision 2102 multi-label plate reader (PerkinElmer, Glen Waverley, Victoria, Australia) using a 485/14 excitation filter, 535/25 emission filter and D505 mirror. The fluorescence/luminescence ratio was generated by dividing the fluorescence values in arbitrary units (obtained with the EnVision) by the luminescence values also in arbitrary units (obtained as part of the BRET assay).

For Receptor-HIT assays, cells were transfected with a Rluc8-tagged CAM and β-arrestin2/Venus, or a Venus-tagged CAM and β-arrestin2/Rluc8. GPCRs untagged with respect to the BRET system were then co-expressed in the HEK293FT cells, or the cells were transfected with pcDNA3 as a control. These cells were then treated with an appropriate cognate agonist selective for the co-expressed GPCR, in order to promote recruitment of the BRET-tagged β-arrestin2 to that GPCR. A ligand-induced BRET signal was indicative of recruitment of the BRET-tagged β-arrestin2 proximal to the BRET-tagged CAM, thereby indicating close proximity between the CAM and the activated GPCR.

### Statistics

Continuous data are expressed as mean±SEM. Differences in the mean among groups were compared using 2-way ANOVA. Pair-wise multiple comparisons were made with Student-Newman-Keuls post-hoc analysis to detect significant differences between groups. P<0.05 was considered statistically significant.

### Example 1. Transactivation of the Cytosolic Tail of ALCAM by a Co-Located GPCR

This example shows that expression of the cytosolic tail of an IgSF CAM, specifically ALCAM₅₅₁₋₅₈₃ (SEQ ID NO: 1), BCAM₅₆₉₋₆₂₈ (SEQ ID NO: 2), MCAM₅₈₄₋₆₃₇ (SEQ ID NO: 3), EpCAM₂₈₉₋₃₁₄ (SEQ ID NO: 4) or CADM4₃₄₆₋₃₈₈ (SEQ ID NO: 5) enables Ang II to induce expression of the key pro-inflammatory transcription factor, p65-NFκB, in Chinese Hamster Ovary (CHO) cells expressing AT₁R, providing evidence for IgSF CAM ligand-independent transactivation of the cytosolic tail of an IgSF CAM, following activation of a GPCR by its cognate ligand, specifically AT1R by Ang II.

CHO cells express few cell surface receptors, and specifically do not express endogenous AT₁R or IgSF CAMs on their surface, making them an ideal system to explore the role of the AT₁R-IgSF CAM interaction. In addition, CHO cells do not express toll-like receptors (TLRs) that potentially have the capacity to bind ligands that also activate IgSF CAMs, and be activated by them (e.g. S100 proteins), resulting in activation of NFκB.

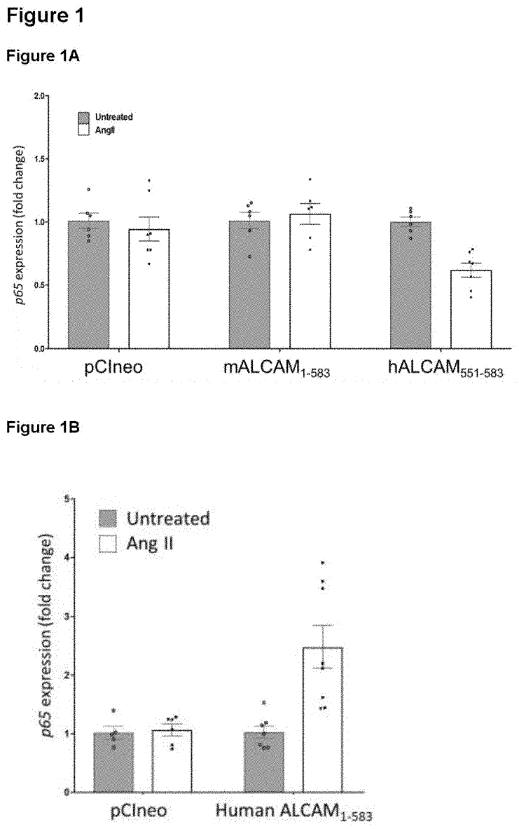

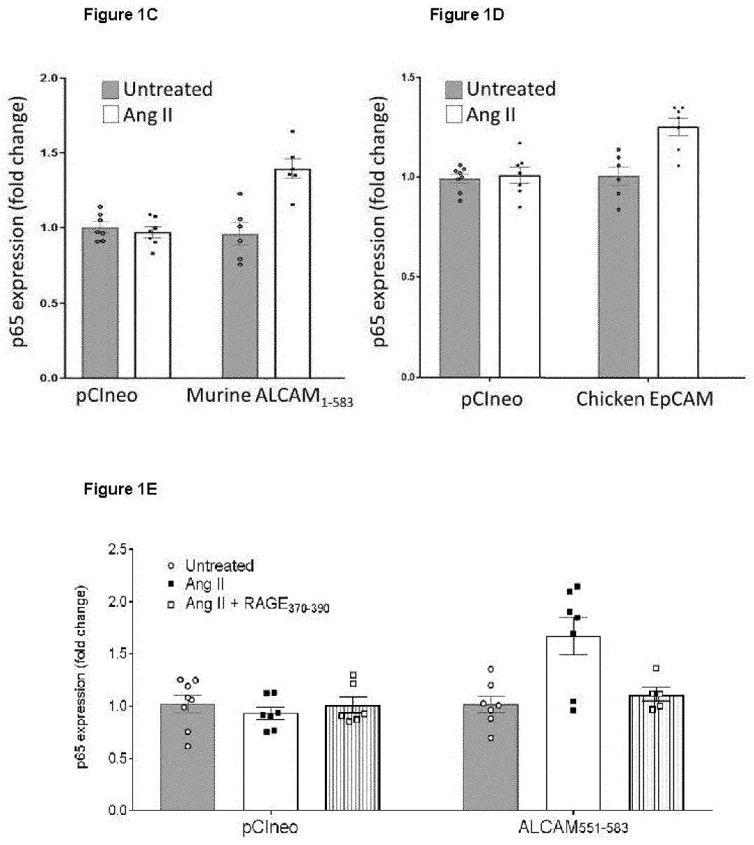

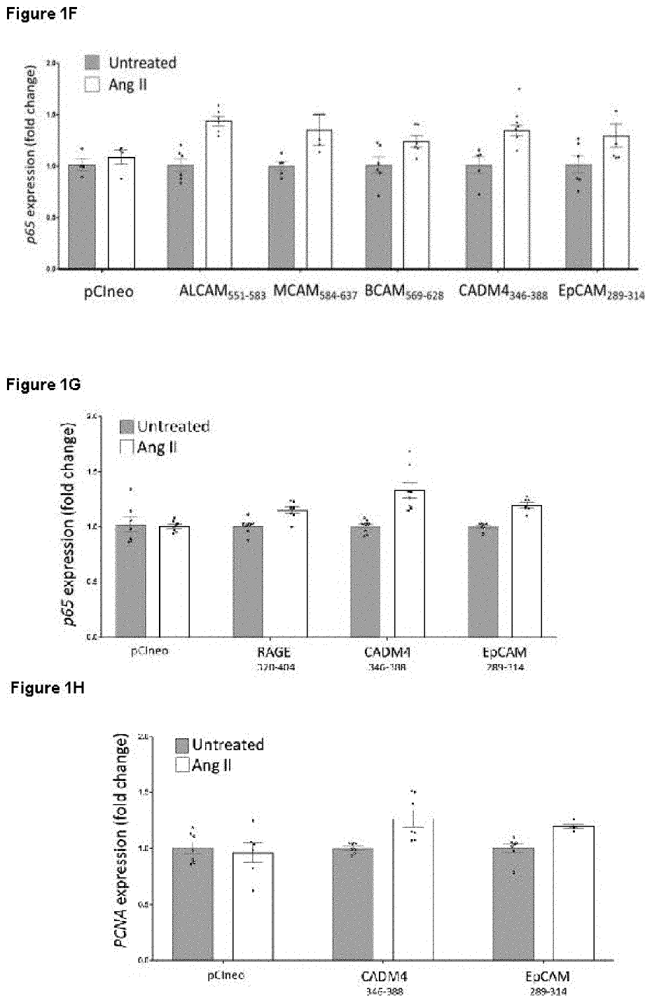

In the absence of expression of the AT1 receptor, Ang II (1 μM) is unable to induce proinflammatory signaling, specifically the induction of p65 gene expression, in CHO cells (FIG. 1A), even in the presence of expression of IgSF CAMs or their cytosolic tails.

Stable transfection of CHO cells with the human AT₁R gene (SEQ ID NO: 15) alone, generates AT₁R-CHO cells, and confers classical responsiveness to exogenous Ang II (1 μM), but not the ability for Ang II to induce expression of the pro-inflammatory transcription factor, p65-NFκB in AT₁R-CHO cells.

Transfection of AT₁R-CHO cells with an IgSF CAM, specifically full length human ALCAM₁₋₅₈₃ (SEQ ID NO: 9), confers the ability of Ang II to induce expression of the pro-inflammatory transcription factor, p65-NFκB when compared to empty plasmid alone (pCIneo; FIG. 1B).

Transfection of AT₁R-CHO cells with an IgSF CAM, specifically full length murine ALCAM₁₋₅₈₃ (SEQ ID NO: 16), confers the ability of Ang II to induce expression of the pro-inflammatory transcription factor, p65-NFκB when compared to empty plasmid alone (pCIneo; FIG. 1C). Transfection of AT₁R-CHO cells with an IgSF CAM, specifically full length chicken EpCAM (SEQ ID NO: 17), also confers the ability of Ang II to induce expression of the pro-inflammatory transcription factor, p65-NFκB when compared to empty plasmid alone (pCIneo; FIG. 1D). Together these data exemplify that the transactivation mechanism described in this invention is not specific to the human species.

Transfection of AT₁R-CHO cells with the cytosolic tail of a IgSF CAM, specifically human ALCAM₅₅₁₋₅₈₃ (SEQ ID NO: 1), also confers the ability of Ang II to induce expression of the pro-inflammatory transcription factor, p65-NFκB when compared to empty plasmid alone (pCIneo vector; FIG. 1E).

Transfection of AT1R-CHO cells with the cytosolic tail of another IgSF CAM, specifically ALCAM₅₅₁₋₅₈₃ (SEQ ID NO: 1), BCAM₅₆₉₋₆₂₈ (SEQ ID NO: 2), MCAM₅₈₄₋₆₃₇ (SEQ ID NO: 3), EpCAM₂₈₉₋₃₁₄ (SEQ ID NO: 4) or CADM4₃₄₆₋₃₈₈ (SEQ ID NO: 5) also confers the ability of Ang II to induce expression of the key pro-inflammatory transcription factor, p65-NFκB (FIG. 1F), when compared to empty plasmid alone (pCIneo vector).

Transfection of AT1R-CHO cells with another IgSF CAM cytosolic tail, specifically EpCAM₂₈₉₋₃₁₄ (SEQ ID NO: 4) or CADM4₃₄₆₋₃₈₈ (SEQ ID NO: 5) also confers the ability of Ang II to induce expression of the key pro-inflammatory transcription factor, p65-NFκB (FIG. 1G), and the p65-NFκB dependent induction of expression of proliferating cell nuclear antigen (PCNA) when compared to empty plasmid alone (pCIneo vector; Figure IH).

Serving as a positive control, transactivation of the cytosolic tail of RAGE₃₇₀₋₄₀₄ following activation of the AT1R by Ang II in AT1R-CHO cells, also induces the expression of the key pro-inflammatory transcription factor, p65-NFκB (Figure IG).

As the IgSF CAM ligand-binding ectodomain is absent, this example demonstrates that the transactivation of any of the family of IgSF CAMs by activated co-located GPCR, specifically AT1R, is therefore IgSF CAM-ligand independent.

Although members of the same IgSF CAM family, the cytosolic tails of these proteins share limited sequence homology between each other. They also share limited sequence homology with the cytosolic tail of RAGE, with the one exception of CADM4 (sequence=QEGEAREAFLNGS) and RAGE₃₇₉₋₃₉₂ (sequence=QEEEEERAELNQS).

### Example 2. Modulation of IGSF CAM Ligand-Independent Transactivation of an IGSF CAM by an Activated Co-Located GPCR in Human ARPE Cells

This example describes using specific components of an IgSF CAM cytosolic tail, specifically ALCAM₅₅₁₋₅₈₃ (SEQ ID NO: 1), BCAM₅₆₉₋₆₂₈ (SEQ ID NO: 2), or MCAM₅₈₄₋₆₃₇ (SEQ ID NO: 3) to modulate IgSF CAM ligand-independent signalling induced in human ARPE cells following activation of a GPCR by its cognate ligand, specifically AT1 receptor by Ang II in human ARPE cells.

Unlike CHO cells, ARPE cells have a replete renin angiotensin aldosterone system including endogenous expression of the AT1 receptor. By contrast, endogenous expression of RAGE and IgSF CAMs is low or absent.

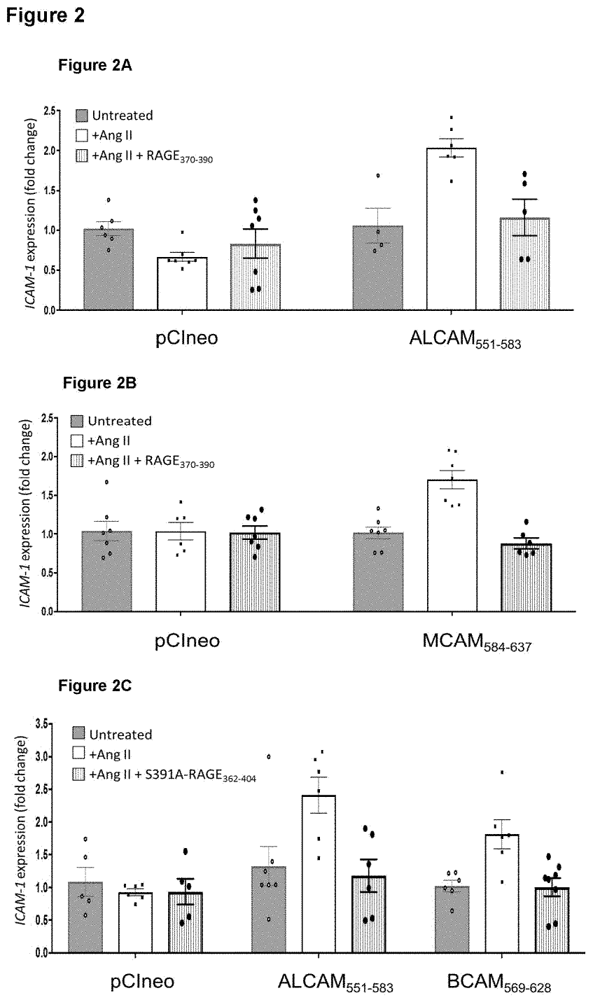

Transfection of ARPE cells with only the cytosolic tail of an IgSF CAM, specifically ALCAM₅₅₁₋₅₈₃ (SEQ ID NO: 1), confers the ability of Ang II to induce pro-inflammatory signalling, exemplified by the NFKB-dependent induction in ICAM-1 gene expression, when compared to empty plasmid alone (pCIneo vector, FIG. 2A).

Transfection of ARPE cells with only the cytosolic tail an IgSF CAM, specifically MCAM₅₈₄₋₆₃₇ (SEQ ID NO: 3) also confers the ability of Ang II to induce pro-inflammatory signalling, exemplified by the NFKB-dependent induction in ICAM-1 gene expression, when compared to empty plasmid alone (pCIneo vector, FIG. 2B).

Transfection of ARPE cells with only the cytosolic tail an IgSF CAM, specifically BCAM₅₆₉₋₆₂₈ (SEQ ID NO: 2) or ALCAM₅₅₁₋₅₈₃ (SEQ ID NO: 1), confers the ability of Ang II to induce pro-inflammatory signalling, exemplified by the NFKB-dependent induction in ICAM-1 gene expression, when compared to empty plasmid alone (pCIneo vector; FIG. 2C).

### Example 3. Inhibition of Activation of IGSF CAMs with a Selectively Truncated Form of the Cytosolic Tail of an IGSF CAM

This example shows that a selectively-truncated construct of the cytosolic tail of an IgSF CAM, specifically ALCAM₅₅₉₋₅₈₀, is able to inhibit IgSF CAM ligand-independent transactivation of the cytosolic tail of an IgSF CAM in CHO cells.

The cytoplasmic domain of human ALCAM contains two serines and two threonines. These are known to be dispensable for ALCAM-mediated adhesion (Zimmerman, Nelissen et al. 2004) and are not considered to be targets for PKC-mediated phosphorylation. However, without wishing to be bound by theory, the inventors believe these residues play a structural role in facilitating signalling mediated by the cytoplasmic tail leading to the induction of NFKB. Therefore an ALCAM construct was generated in which these serines and threonines were specifically omitted as a consequence of selective truncation of the cytosolic tail, generating ALCAM₅₅₉₋₅₈₀ (SEQ ID NO: 6).

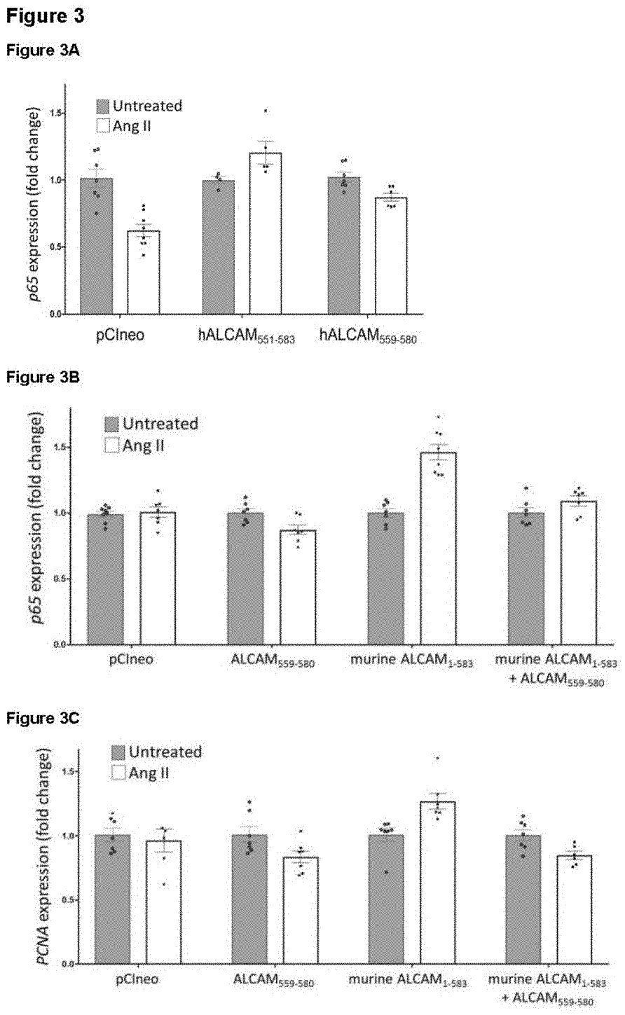

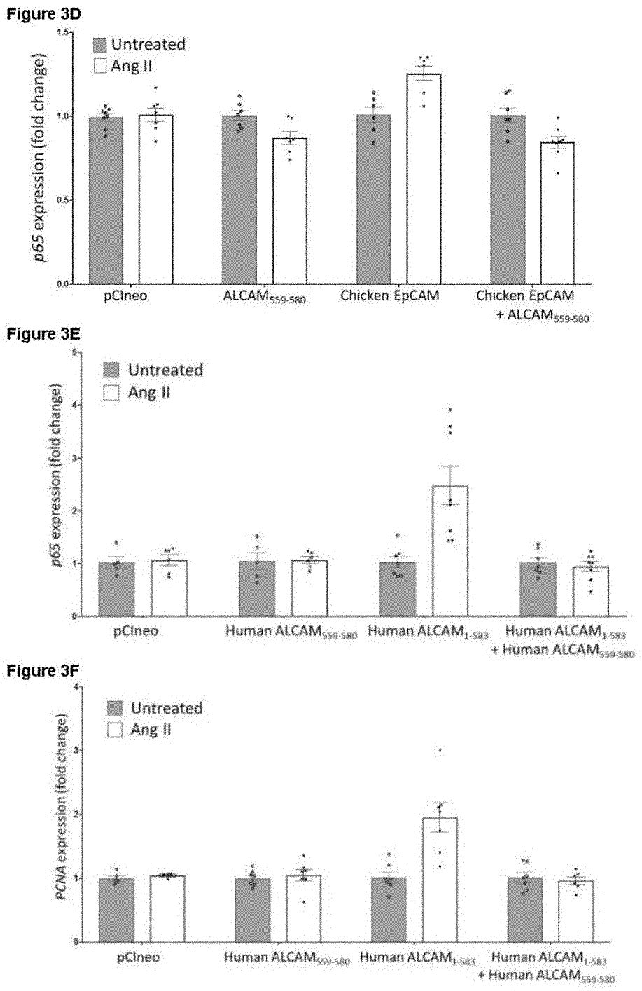

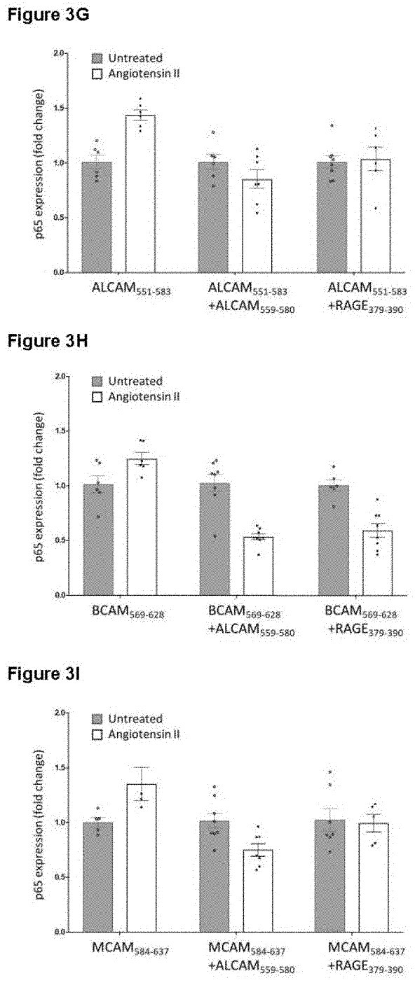

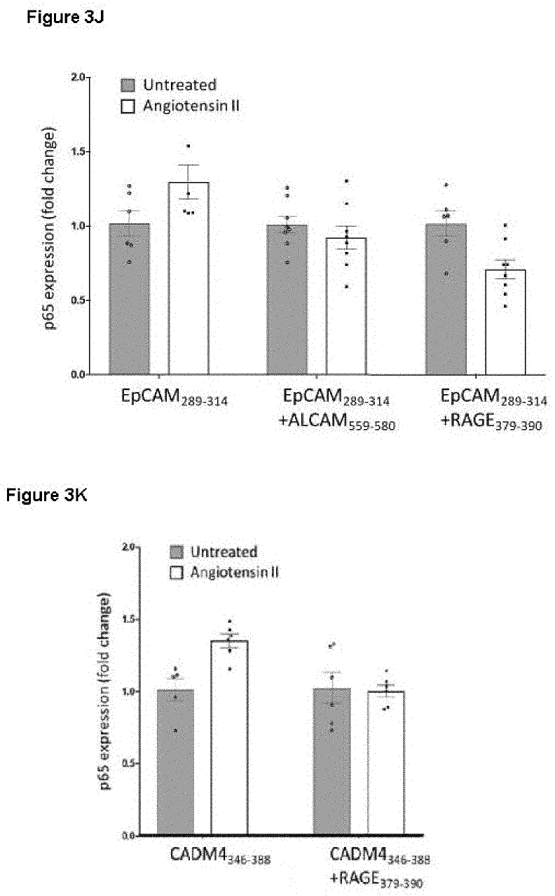

In AT1R-CHO cells expressing human AT1 receptor, cotransfected with ALCAM₅₅₉₋₅₈₀ (SEQ ID NO: 6), activation of the AT1 receptor by its cognate ligand, Ang II, failed to increase the expression of p65, confirming that this construct did not contain transactivatable targets, unlike the full cytoplasmic domain of ALCAM, specifically ALCAM₅₅₁₋₅₈₃ (SEQ ID NO:1; FIG. 3A).

Co-transfection with AT₁R-CHO cells with a selectively-truncated construct of the cytosolic tail of an IgSF CAM, specifically ALCAM₅₅₉₋₅₈₀ (SEQ ID NO: 6) prevents induction in the expression of the key pro-inflammatory transcription factor, p65-NFκB (FIG. 3B) and the p65-NFκB dependent induction of expression of proliferating cell nuclear antigen (PCNA; FIG. 3C) induced by Ang II via AT1R-dependent transactivation of full length ALCAM₁₋₅₈₃ when compared to empty plasmid alone (pCIneo vector).

Co-transfection with AT₁R-CHO cells with a selectively-truncated construct of the cytosolic tail of an IgSF CAM, specifically ALCAM₅₅₉₋₅₈₀ (SEQ ID NO: 6) prevents induction in the expression of the key pro-inflammatory transcription factor, p65-NFκB (FIG. 3D) induced by Ang II via AT1R-dependent transactivation of full length chicken EpCAM (SEQ ID NO: 17) when compared to empty plasmid alone (pCIneo vector).

Co-transfection with AT₁R-CHO cells with a selectively-truncated construct of the cytosolic tail of an IgSF CAM, specifically ALCAM₅₅₉₋₅₈₀ (SEQ ID NO: 6) prevents induction in the expression of the key pro-inflammatory transcription factor, p65-NFκB (FIG. 3E) and the p65-NFκB dependent induction of expression of proliferating cell nuclear antigen (FIG. 3F) induced by Ang II via AT1R-dependent transactivation of cytosolic tail of human ALCAM₅₅₁₋₅₈₃ (SEQ ID NO: 1) when compared to empty plasmid alone (pCIneo vector).

Co-transfection with AT₁R-CHO cells with a selectively-truncated construct of the cytosolic tail of an IgSF CAM, specifically ALCAM₅₅₉₋₅₈₀ (SEQ ID NO: 6) prevents induction in the expression of the key pro-inflammatory transcription factor, p65-NFκB (FIG. 3G) induced by Ang II via AT1R-dependent transactivation of the cytosolic tail of human ALCAM, specifically ALCAM₅₅₁₋₅₈₃ (SEQ ID NO: 1) when compared to empty plasmid alone (pCIneo vector).

Co-transfection with AT₁R-CHO cells with a selectively-truncated construct of the cytosolic tail of an IgSF CAM, specifically ALCAM₅₅₉₋₅₈₀ (SEQ ID NO: 6) prevents induction in the expression of the key pro-inflammatory transcription factor, p65-NFκB (FIG. 3H) induced by Ang II via AT1R-dependent transactivation of the cytosolic tail of human BCAM, specifically BCAM₅₆₉₋₆₂₈ (SEQ ID NO: 2) when compared to empty plasmid alone (pCIneo vector).

Co-transfection with AT₁R-CHO cells with a selectively-truncated construct of the cytosolic tail of an IgSF CAM, specifically ALCAM₅₅₉₋₅₈₀ (SEQ ID NO: 6) prevents induction in the expression of the key pro-inflammatory transcription factor, p65-NFκB (FIG. 3I) induced by Ang II via AT1R-dependent transactivation of the cytosolic tail of human MCAM, specifically MCAM₅₈₄₋₆₃₇ (SEQ ID NO: 3) when compared to empty plasmid alone (pCIneo vector).

Co-transfection with AT₁R-CHO cells with a selectively-truncated construct of the cytosolic tail of an IgSF CAM, specifically ALCAM₅₅₉₋₅₈₀ (SEQ ID NO: 6) prevents induction in the expression of the key pro-inflammatory transcription factor, p65-NFκB (FIG. 3J) induced by Ang II via AT1R-dependent transactivation of the cytosolic tail of human EpCAM, specifically EpCAM₂₈₉₋₃₁₄ (SEQ ID NO: 4) when compared to empty plasmid alone (pCIneo vector).

These findings demonstrate the ability of peptides derived from the cytosolic tail of an IgSF CAM, specifically ALCAM₅₅₉₋₅₈₀ (SEQ ID NO: 6), to modulate pro-inflammatory signalling mediated by an IgSF CAM, specifically ALCAM, BCAM, MCAM, and EpCAM. Furthermore, this example demonstrates that IgSF CAM-ligand independent activation of an IgSF CAM, specifically ALCAM, BCAM, MCAM, and EpCAM, by activated co-located GPCR is inhibited by a fragment of the cytosolic tail of ALCAM, specifically ALCAM₅₅₉₋₅₈₀ (SEQ ID NO: 6).

### Example 4. Modulation of Ligand-Mediated Activation of IGSF CAMs with a Selectively Truncated Form of the Cytosolic Tail of ALCAM or RAGE₃₇₀₋₃₉₀, as Well as Modulation of Ligand-Mediated Activation of RAGE with a Selectively Truncated Form of the Cytosolic Tail of ALCAM

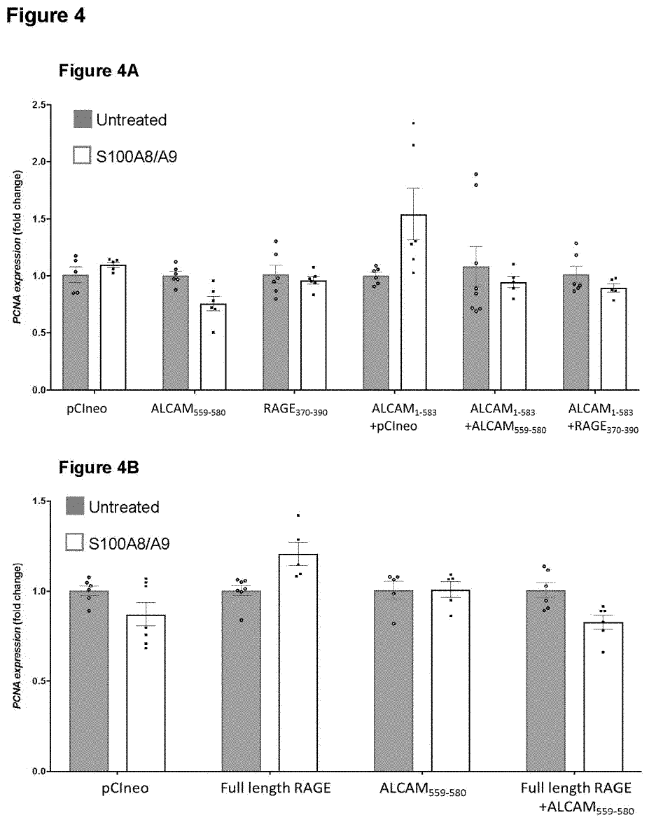

The ectodomain of IgSF CAMs may also be activated by extracellular ligands, triggering intracellular signalling mediated by their cytosolic tail. For example, the ectodomain of full length ALCAM may be activated by S100A8/A9 leading to NFKB-dependent induction of expression of proliferating cell nuclear antigen (PCNA; FIG. 4A). This ligand-dependent signalling is not observed following transfection with inhibitory ALCAM or RAGE constructs in which the ectodomain has been deleted.

Ligand-dependent signalling via an activated IgSF CAM, specifically murine ALCAM, is inhibited by ALCAM₅₅₉₋₅₈₀ (SEQ ID NO: 6).

Ligand-dependent signalling via an activated IgSF CAM, specifically ALCAM, is also inhibited by truncated peptides derived from the RAGE cytosolic tail, specifically RAGE₃₇₀₋₃₉₀ (SEQ ID NO: 7).

The ectodomain of RAGE may also be activated by extracellular ligands, triggering intracellular signalling mediated by its cytosolic tail. For example, RAGE may be activated by S100A8/A9 leading to NFKB-dependent induction of expression of proliferating cell nuclear antigen (PCNA; FIG. 4B). This ligand-dependent signalling via full length RAGE is also inhibited by ALCAM₅₅₉₋₅₈₀ (SEQ ID NO: 6).

These data demonstrate that ligand-dependent signalling mediated by full length IgSF CAMs, specifically activation of ALCAM₁₋₅₈₃ by S100A8/A9, can be modulated by peptides derived from the cytosolic tail of IgSF CAMs, specifically ALCAM₅₅₉₋₅₈₀ (SEQ ID NO: 6), or peptides derived from the cytosolic tail of RAGE, specifically RAGE₃₇₀₋₃₉₀.

Furthermore, this example demonstrates that ligand-dependent activation of full length RAGE, specifically RAGE₁₋₄₀₄ by S100A8/A9, can also be modulated by a selectively-truncated construct of the cytosolic tail of an IgSF CAM, specifically ALCAM₅₅₉₋₅₈₀ (SEQ ID NO: 6).

### Example 5. Modulation of Activation of IGSF CAMs with a Selectively Truncated Form of the Cytosolic Tail of RAGE

This example describes using specific components of the RAGE cytosolic tail, specifically RAGE₃₇₀₋₃₉₀ (SEQ ID NO: 7) to modulate ligand-dependent activation of an IgSF CAM, specifically by S100AA8/A9, as well as ligand-independent transactivation of an IgSF CAM induced following activation of a GPCR by its cognate ligand, specifically AT1 receptor by Ang II.

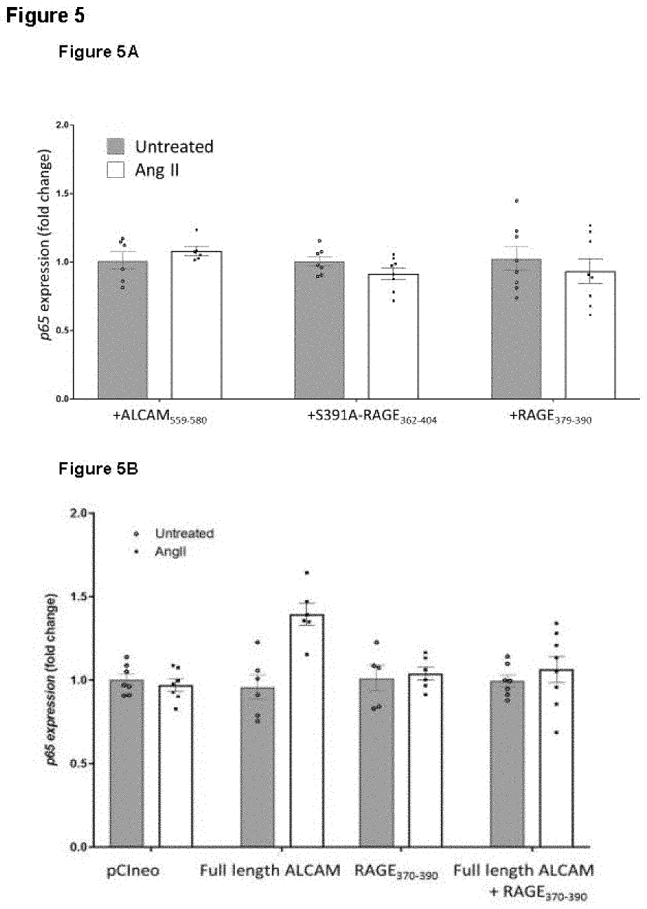

The RAGE cytosolic tail is not able to mediate proinflammatory signalling when residue Serine391 has been mutated or deleted. Consequently, when RAGE₃₇₀₋₃₉₀ (SEQ ID NO: 7) or RAGE₃₇₉₋₃₉₀ (SEQ ID NO: 21) is expressed in AT1R-CHO cells, no induction of p65 expression is observed following exposure to S100A8/9 (FIG. 4A) or Ang II (FIGS. 5A and B).

Co-transfection of AT₁R-CHO cells with a selectively-truncated construct of the cytosolic tail of RAGE, specifically RAGE₃₇₉₋₃₉₀ (SEQ ID NO: 21) prevents induction of the expression of the key pro-inflammatory transcription factor, p65-NFκB induced by Ang II via AT1R-dependent transactivation of the cytosolic tail of an IgSF CAM, specifically ALCAM₅₅₁₋₅₈₃ (SEQ ID NO: 1), BCAM₅₆₉₋₆₂₈ (SEQ ID NO: 2), MCAM₅₈₄₋₆₃₇ (SEQ ID NO: 3), EpCAM₂₈₉₋₃₁₄ (SEQ ID NO: 4) or CADM4₃₄₆₋₃₈₈ (SEQ ID NO: 5), when compared to empty plasmid alone (pCIneo vector; FIGS. 3G-K).

Transfection of ARPE cells with only the cytosolic tail of an IgSF CAM, specifically ALCAM₅₅₁₋₅₈₃ (SEQ ID NO: 1), confers the ability of Ang II to induce pro-inflammatory signalling, exemplified by the NFKB-dependent induction in ICAM-1 gene expression, when compared to empty plasmid alone (pCIneo vector). This signalling is inhibited by RAGE₃₇₀₋₃₉₀ (SEQ ID NO: 7; FIG. 2A).

Transfection of ARPE cells with only the cytosolic tail an IgSF CAM, specifically MCAM₅₈₄₋₆₃₇ (SEQ ID NO: 3) also confers the ability of Ang II to induce pro-inflammatory signalling, exemplified by the NFKB-dependent induction in ICAM-1 gene expression, when compared to empty plasmid alone (pCIneo vector). This signalling is also inhibited by RAGE₃₇₀₋₃₉₀ (SEQ ID NO: 7; FIG. 2B).

Transfection of ARPE cells with only the cytosolic tail an IgSF CAM, specifically BCAM₅₆₉₋₆₂₈ (SEQ ID NO: 2) also confers the ability of Ang II to induce pro-inflammatory signalling, exemplified by the NFKB-dependent induction in ICAM-1 gene expression, when compared to empty plasmid alone (pCIneo vector). This signalling is also inhibited by a S391A-RAGE₃₆₂₋₄₀₄ oligopeptide encompassing the entire cytosolic tail of RAGE in which the serine391 residue required for transactivation has been mutated to alanine (S391A-RAGE₃₆₂₋₄₀₄ SEQ ID NO: 8 FIG. 2C). The S391A-RAGE₃₆₂₋₄₀₄ oligopeptide also inhibited proinflammatory signalling induced by Ang II in ARPE cells mediated by the cytosolic tail of ALCAM (SEQ ID NO: 1; FIG. 2C).

Taken together, these examples demonstrate that the IgSF CAM-ligand independent activation of IgSF CAM by activated co-located GPCR is inhibited by a derivative of RAGE.

Transfection of AT₁R-CHO cells with an IgSF CAM, specifically full length murine ALCAM₁₋₆₈₃ (SEQ ID NO: 16), confers the ability of Ang II to induce expression of the pro-inflammatory transcription factor, p65-NFκB when compared to empty plasmid alone (pCIneo). This ligand-independent transactivation is also inhibited by RAGE₃₇₀₋₃₆₀ (FIG. 5B).

Transfection of AT₁R-CHO cells with the cytosolic tail of an IgSF CAM, specifically human ALCAM₆₆₁₋₆₈₃ (SEQ ID NO: 1), confers the ability of Ang II to induce expression of the pro-inflammatory transcription factor, p65-NFκB when compared to empty plasmid alone (pCIneo). This ligand-independent transactivation is also inhibited by RAGE₃₇₀₋₃₆₀ (FIG. 1E).

These findings demonstrate the ability of peptides derived from the RAGE cytosolic tail to modulate pro-inflammatory signalling mediated by the cytosolic tail of an IgSF CAM, and specifically the cytosolic tail of ALCAM. Furthermore, this example demonstrates that IgSF CAM-ligand independent transactivation of IgSF CAM by activated co-located GPCR is inhibited by a fragment of RAGE.

### Example 6. Modulation of RAGE Ligand-Independent Activation of RAGE by an Activated Co-Located GPCR in Human ARPE Cells with a Selectively Truncated Form of the Cytosolic Tail of ALCAM

This example describes using a derivative of an IgSF CAM cytosolic tail, specifically ALCAM₆₆₆₋₆₈₀ (SEQ ID NO: 6) to modulate RAGE ligand-independent signalling induced via transactivation of full length RAGE₁₋₄₀₄ in human ARPE cells following activation of a GPCR by its cognate ligand.

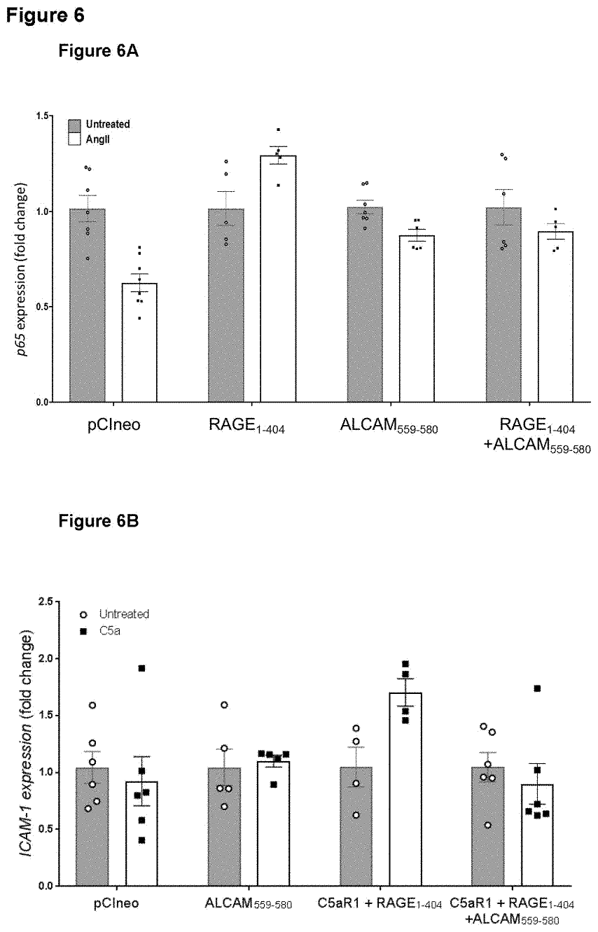

Expression of ALCAM₅₅₉₋₅₈₀ (SEQ ID NO: 6) inhibits the induction of p65 expression mediated by full length human RAGE₁₋₄₀₄ following its transactivation by an activated co-located GPCR, specifically AT1 receptor activated by Ang II in AT1R-CHO cells (FIG. 6A).

C5aR1 (SEQ ID NO: 18) is the receptor for complement 5a. Activation of the C5aR1 by its cognate ligand, C5a, increases the expression of ICAM-1 in ARPE, and this expression is increased in the presence of full length RAGE₁₋₄₀₄ (FIG. 6B).

Co-expression of a selectively truncated form of the cytosolic tail of ALCAM, specifically ALCAM₅₅₉₋₅₈₀ (SEQ ID NO: 6) inhibits RAGE-dependent induction of the expression of ICAM-1 following the transactivation of RAGE by the C5a receptor 1.

These data demonstrate that constructs derived from the cytosolic tail of IgSF CAMs, specifically ALCAM₅₅₉₋₅₈₀ (SEQ ID NO: 6), can modulate RAGE-dependent signalling initiated following activation of a co-located GPCR, specifically AT1R and C5aR1.

### Example 7. Functional Competition Between Full Length RAGE and the Cytosolic Tails of IGSF CAMs

This example describes competition between the cytosolic tail of IgSF CAMs and the cytosolic tail of full length RAGE, with respect to transactivation by a co-located GPCR and the induction of downstream pro-inflammatory signalling.

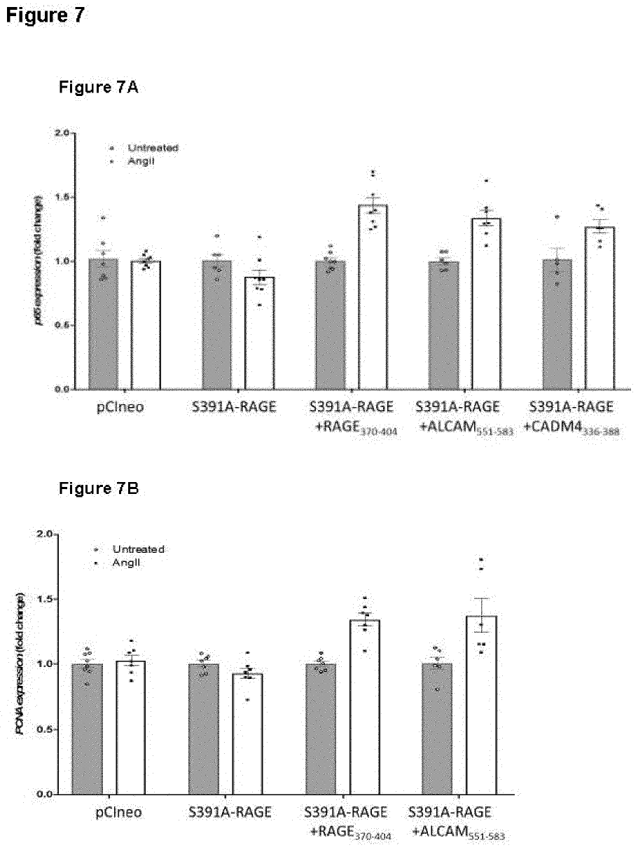

Transfection of AT₁R-CHO cells with S391A-RAGE₁₋₄₀₄ fails to confer the ability of Ang II to induce expression of the pro-inflammatory transcription factor, p65-NFκB when compared to empty plasmid alone (pCIneo) as S391A-RAGE is unable to be transactivated by a co-located GPCR, specifically the AT1R by Ang II, as indicated by the expression of the key pro-inflammatory transcription factor, p65-NFκB (FIG. 7A), and the p65-NFκB dependent induction of expression of proliferating cell nuclear antigen (PCNA) when compared to empty plasmid alone (pCIneo vector; FIG. 7B).

In the presence of S391A-RAGE₁₋₄₀₄, over-expression of the cytosolic tail of an IgSF CAM, specifically ALCAM₅₅₁₋₅₈₃ (SEQ ID NO: 1) or CADM4₃₃₆₋₃₈₈ (SEQ ID NO: 5) is able to overcome inhibition of transactivation by full length mutant S391A-RAGE and be transactivated themselves (FIGS. 7A and 7B). In particular, ALCAM₅₅₁₋₅₈₃ (SEQ ID NO: 1) was transactivated by a co-located GPCR, specifically the AT1R by Ang II, as indicated by the expression of the key pro-inflammatory transcription factor, p65-NFκB (FIG. 7A), and the p65-NFκB dependent induction of expression of proliferating cell nuclear antigen when compared to empty plasmid alone (pCIneo vector; FIG. 7B).

By contrast, transactivation of the cytosolic tail of an IgSF CAM, specifically ALCAM₅₅₁₋₅₈₃ (SEQ ID NO: 1) or BCAM₅₆₉₋₆₂₈ (SEQ ID NO: 2), in ARPE cells, is inhibited by the S391A-RAGE₃₆₂₋₄₀₄ oligopeptide (SEQ ID NO: 8; FIG. 2C).

This example demonstrates that RAGE and IgSF CAMs share common intracellular signalling pathways mediated by their respective cytosolic tails.

### Example 8. BRET Indicates Close Proximity of IGSF CAM to Certain Activated GPCRS when Co-Expressed in Live Cells

In this example, we demonstrate close proximity between ALCAM and certain activated co-located GPCRs.

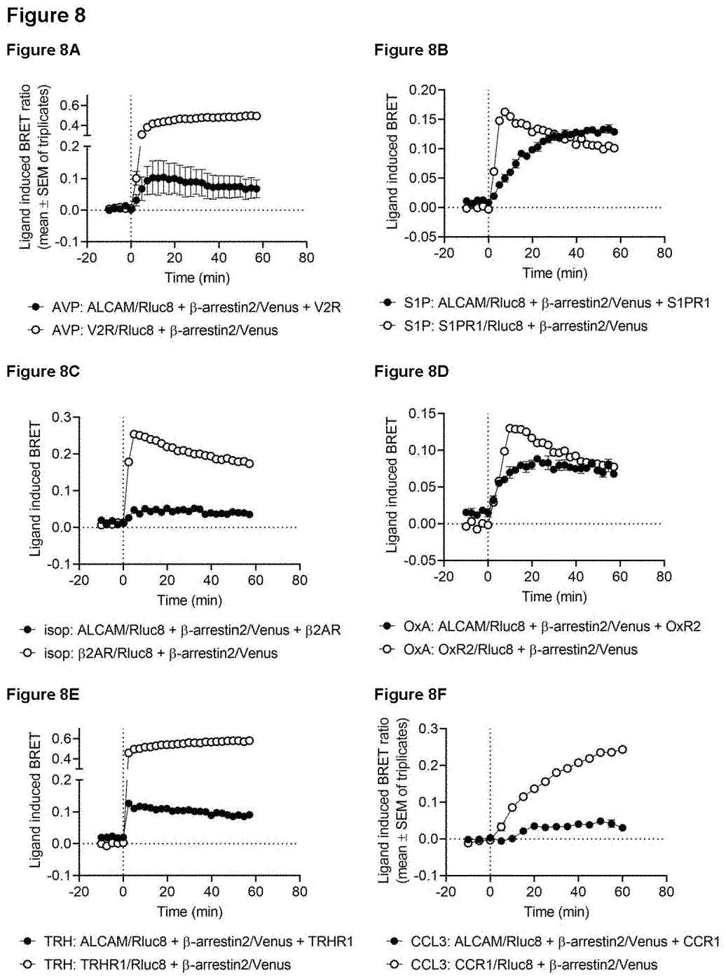

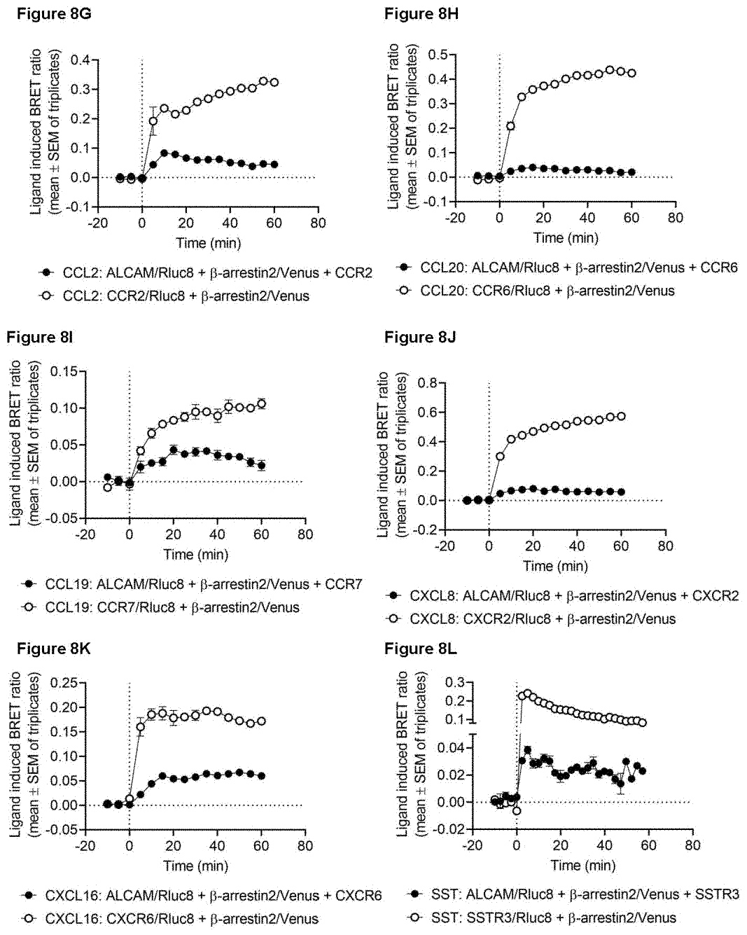

Treatment of cells co-expressing Rluc8-labelled GPCR and β-arrestin2/Venus with an appropriate cognate agonist for that GPCR resulted in the induction of a robust ligand-induced BRET signal consistent with recruitment of β-arrestin2 to the activated Rluc8-labelled GPCR. This was observed for V2R with AVP (FIG. 8A), 51PR1 with S1P (FIG. 8B), β2AR with isoproterenol (FIG. 8C), OxR2 with OxA (FIG. 8D), TRHR1 with TRH (FIG. 8E), CCR1 with CCL3 (FIG. 8F), CCR2 with CCL2 (FIG. 8G), CCR6 with CCL20 (FIG. 8H), CCR7 with CCL19 (FIG. 8I), CXCR2 with CXCL8 (FIG. 8J), CXCR6 with CXCL16 (FIG. 8K) and SSTR3 with SST (FIG. 8L).

Receptor-HIT: Treatment of cells co-expressing Rluc8-labelled ALCAM (ALCAM/Rluc8) and β-arrestin2/Venus in the presence of V2R (FIG. 8A), S1PR1 (FIG. 8B), β2AR (FIG. 8C), OxR2 (FIG. 8D), TRHR1 (FIG. 8E), CCR1 (FIG. 8F), CCR2 (FIG. 8G), CCR6 (FIG. 8H), CCR7 (FIG. 8I), CXCR2 (FIG. 8J), CXCR6 (FIG. 8K) and SSTR3 (FIG. 8L) with the appropriate cognate agonist for that GPCR resulted in the induction of a clear ligand-induced BRET signal consistent with recruitment of β-arrestin2 to the GPCR. This in turn is indicative of ALCAM proximity to the activated GPCR.

### Example 9. BRET Indicates Close Proximity of IGSF CAM to Certain Activated GPCRS when Co-Expressed in Live Cells and this Proximity is Modulated by GPCR Ligand, as Well as Co-Expression of Untagged IGSF CAM or RAGE

In this example, we demonstrate close proximity between ALCAM and certain activated co-located GPCRs. Furthermore, we demonstrate that this proximity can be modulated by treatment with the cognate ligand for the GPCR. It can also be modulated with co-expression of untagged IgSF CAM, specifically ALCAM, or RAGE.

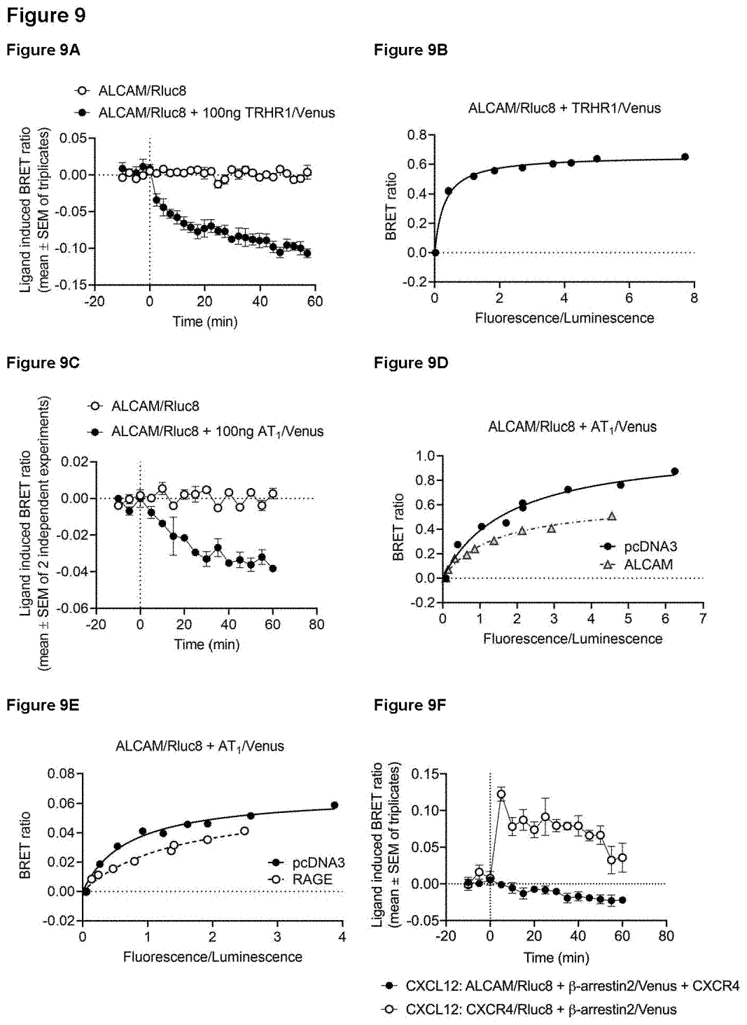

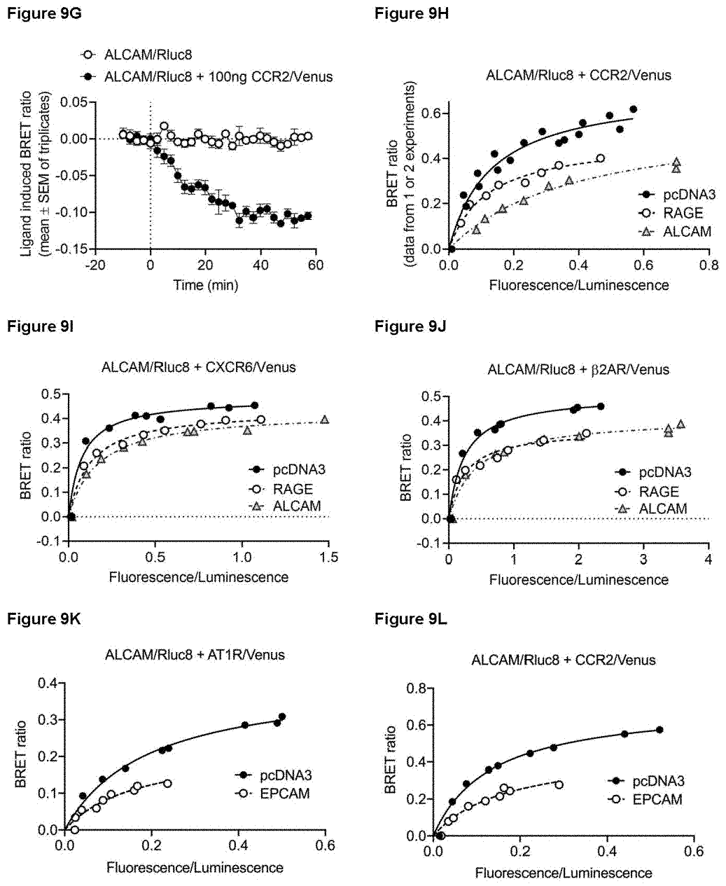

Treatment of cells expressing ALCAM/Rluc8 and TRHR1/Venus with TRH reduced the BRET signal between them (FIG. 9A), consistent with a decrease in proximity or relative orientation of the Rluc8 and Venus. Furthermore, co-expression of ALCAM/Rluc8 and TRHR1/Venus resulted in a saturation curve indicative of close proximity (FIG. 9B).

Treatment of cells expressing ALCAM/Rluc8 and AT₁/Venus with AngII reduced the BRET signal between them (FIG. 9C), consistent with a decrease in proximity or relative orientation of the Rluc8 and Venus. Furthermore, co-expression of ALCAM/Rluc8 and AT₁/Venus resulted in a saturation curve indicative of close proximity (FIGS. 9D and 9E). This curve was flattened by co-expression with untagged ALCAM (FIG. 9D) or RAGE (FIG. 9E), consistent with ALCAM or RAGE competing with ALCAM/Rluc8 for interaction with AT₁/Venus.

Treatment of cells co-expressing Rluc8-labelled ALCAM (ALCAM/Rluc8) and β-arrestin2/Venus in the presence of CXCR4 (FIG. 9F) with CXCL12 resulted in the induction of a clear ligand-induced decrease in BRET signal consistent with a reduction in proximity between β-arrestin2 and ALCAM, or a conformational change resulting in less resonance energy transfer between Rluc8 and Venus. This in turn is indicative of a change in proximity of ALCAM to the activated GPCR to which the β-arrestin2/Venus is recruited.

Treatment of cells expressing ALCAM/Rluc8 and CCR2/Venus with CCL2 reduced the BRET signal between them (FIG. 9G), consistent with a decrease in proximity or relative orientation of the Rluc8 and Venus. Furthermore, co-expression of ALCAM/Rluc8 and CCR2/Venus (FIG. 9H), CXCR6/Venus (FIG. 9I) or β2AR/Venus (FIG. 9J) resulted in saturation curves indicative of close proximity that were flattened by co-expression with untagged ALCAM or RAGE, consistent with ALCAM or RAGE competing with ALCAM/Rluc8 for interaction with the Venus-tagged GPCR.

Co-expression of ALCAM/Rluc8 and AT₁/Venus (FIG. 9K) or CCR2/Venus (FIG. 9L) resulted in saturation curves indicative of close proximity that were flattened by co-expression with untagged EpCAM, consistent with EpCAM competing with ALCAM/Rluc8 for interaction with the Venus-tagged GPCR.

This example demonstrated that there is specific proximity between IgSF CAM, specifically ALCAM, and certain GPCRs.

### Example 10. Bret Indicates Close Proximity of IGSF CAM, from Different Species, to a GPCR, and that it is Observed Using Different BRET Orientations

In this example, we demonstrate that proximity to AT₁ is observed with both human and mouse ALCAM, as well as EpCAM, and with two configurations of BRET donor and acceptor.

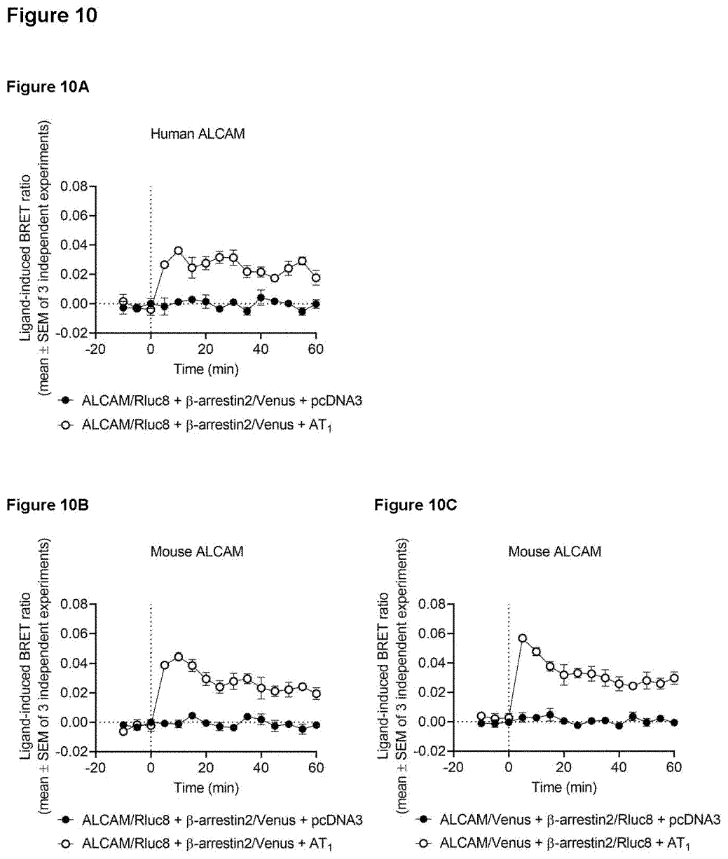

Receptor-HIT: Treatment of cells co-expressing Rluc8-labelled ALCAM (ALCAM/Rluc8) and β-arrestin2/Venus resulted in an AngII-induced BRET signal in the presence, but not in the absence, of AT₁ with both human ALCAM (FIG. 10A) and mouse ALCAM (FIG. 10B). Treatment of cells co-expressing Venus-labelled mouse ALCAM (ALCAM/Venus) and β-arrestin2/Rluc8 also resulted in an AngII-induced BRET signal in the presence, but not in the absence, of AT₁ (FIG. 10C).

This example demonstrates that both human and mouse ALCAM exhibit proximity to AT₁, indicating that it is observed with different species. This example also demonstrates that both BRET donor/acceptor orientations can detect proximity between IgSF CAM, specifically ALCAM, and GPCR, specifically AT₁.

### Example 11. Bret Indicates Close Proximity of EPCAM to a GPCR and that it is Observed Using Different BRET Orientations

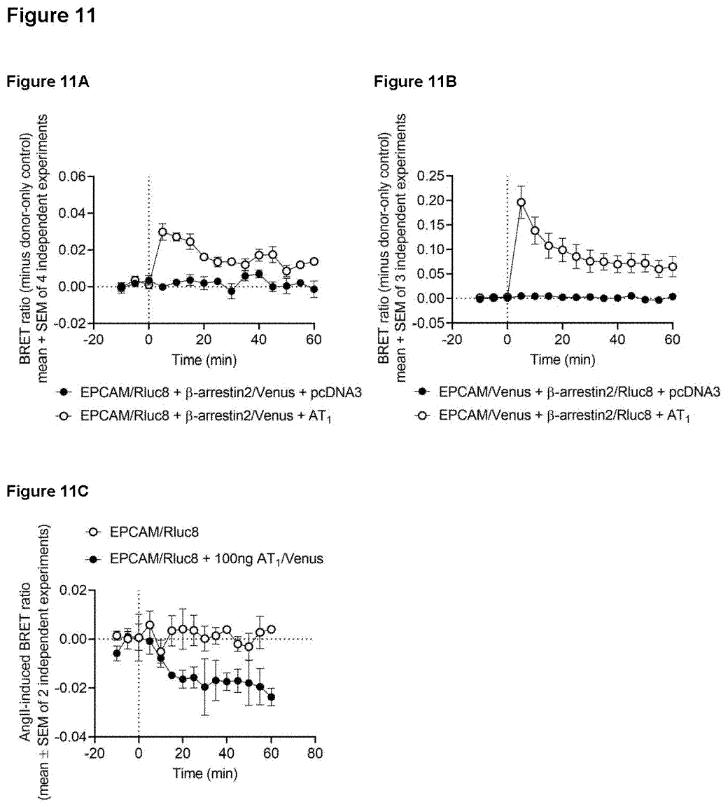

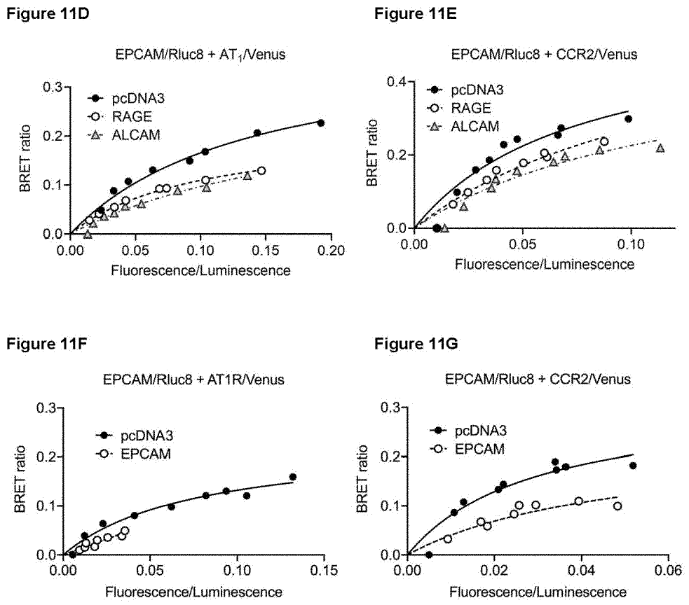

Receptor-HIT: Treatment of cells co-expressing Rluc8-labelled EpCAM (EpCAM/Rluc8) and β-arrestin2/Venus resulted in an AngII-induced BRET signal in the presence, but not in the absence, of AT₁ (FIG. 11A). Treatment of cells co-expressing Venus-labelled EpCAM (EpCAM/Venus) and β-arrestin2/Rluc8 also resulted in an AngII-induced BRET signal in the presence, but not in the absence, of AT₁ (FIG. 11B).

Treatment of cells expressing EpCAM/Rluc8 and AT₁/Venus with AngII reduced the BRET signal between them (FIG. 11C), consistent with a decrease in proximity or relative orientation of the Rluc8 and Venus. Co-expression of EpCAM/Rluc8 and AT₁/Venus (FIGS. 11D and 11F) or CCR2/Venus (FIGS. 11E and 11G) resulted in saturation curves indicative of close proximity. These curves were flattened by co-expression with untagged ALCAM or RAGE (FIGS. 11D and 11E) or EpCAM (FIGS. 11F and 11G), consistent with ALCAM, RAGE or EpCAM competing with EpCAM/Rluc8 for interaction with AT₁/Venus.

This example demonstrates that EpCAM also exhibits specific proximity to certain GPCRs, and this is observed with both orientations of BRET donor and acceptor.

### Example 12. Bret Indicates Close Proximity of CADM4 to a GPCR and that it is Observed Using Different BRET Orientations

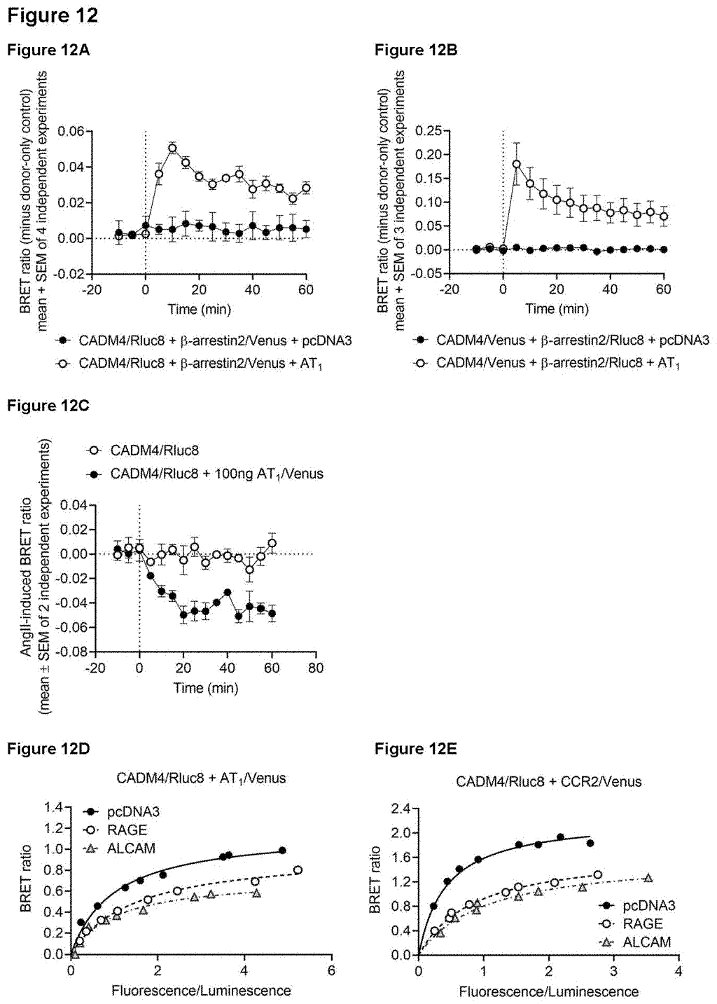

Receptor-HIT: Treatment of cells co-expressing Rluc8-labelled CADM4 (CADM4/Rluc8) and β-arrestin2/Venus resulted in an AngII-induced BRET signal in the presence, but not in the absence, of AT₁ (FIG. 12A). Treatment of cells co-expressing Venus-labelled CADM4 (CADM4/Venus) and β-arrestin2/Rluc8 also resulted in an AngII-induced BRET signal in the presence, but not in the absence, of AT₁ (FIG. 12B).

Treatment of cells expressing CADM4/Rluc8 and AT₁/Venus with AngII reduced the BRET signal between them (FIG. 12C), consistent with a decrease in proximity or relative orientation of the Rluc8 and Venus. Co-expression of CADM4/Rluc8 and AT₁/Venus (FIG. 12D) or CCR2/Venus (FIG. 12E) resulted in saturation curves indicative of close proximity. These curves were flattened by co-expression with untagged ALCAM or RAGE, consistent with ALCAM or RAGE competing with CADM4/Rluc8 for interaction with AT₁/Venus.

This example demonstrates that CADM4 also exhibits specific proximity to certain GPCRs, and this is observed with both orientations of BRET donor and acceptor.

### Example 13. Bret Indicates Close Proximity of RAGE to a GPCR that is Reduced by Co-Expression of IGSF CAM

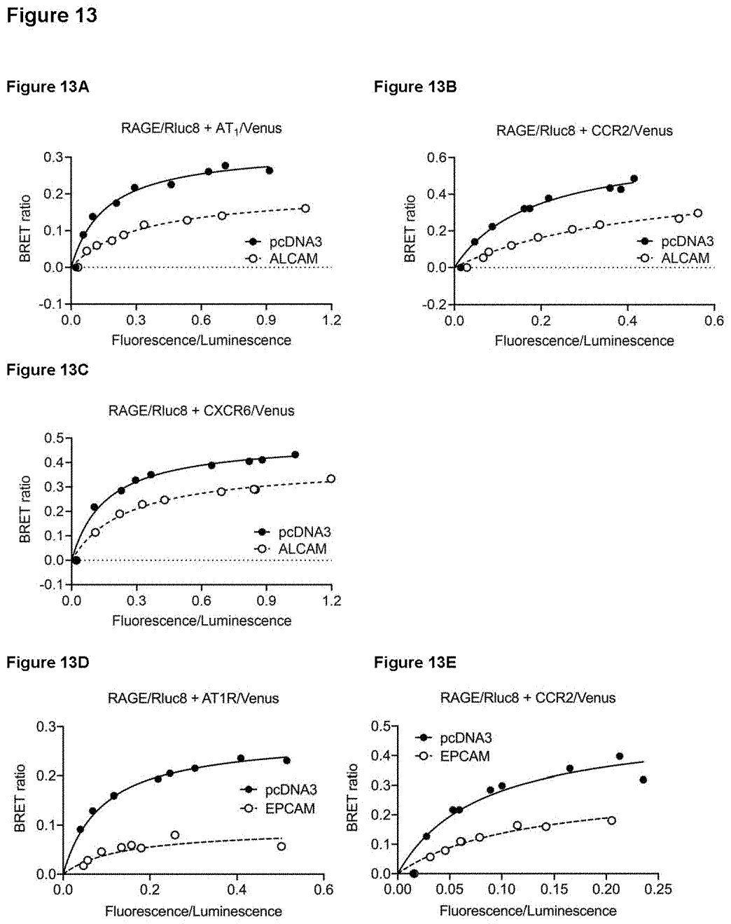

Co-expression of RAGE/Rluc8 and AT₁/Venus (FIGS. 13A and 13D) or CCR2/Venus (FIGS. 13B and 13E) or CXCR6/Venus (FIG. 13C) resulted in saturation curves indicative of close proximity. These curves were flattened by co-expression with untagged ALCAM (FIGS. 13A, 13B and 13C) or untagged EpCAM (FIGS. 13D and 13E), consistent with ALCAM or EpCAM competing with RAGE/Rluc8 for interaction with AT₁/Venus.

This example demonstrates that RAGE also exhibits specific proximity to certain GPCRs and this is specifically reduced by IgSF CAMs.

BRIEF DESCRIPTION OF THE SEQUENCES

| SEQUENCE ID | |
|---|---|
| NUMBER | SEQUENCE |
| SEQ ID NO: 1 | Polypeptide sequence of cytosolic tail of ALCAM |
| SEQ ID NO: 2 | Polypeptide sequence of cytosolic tail of BCAM |
| SEQ ID NO: 3 | Polypeptide sequence of cytosolic tail of MCAM |
| SEQ ID NO: 4 | Polypeptide sequence of cytosolic tail of EpCAM |
| SEQ ID NO: 5 | Polypeptide sequence of cytosolic tail of CADM4 |
| SEQ ID NO: 6 | Polypeptide sequence of ALCAM₅₅₉₋₅₈₀ |
| SEQ ID NO: 7 | Polypeptide sequence of RAGE₃₇₀₋₃₉₀ |
| SEQ ID NO: 8 | Polypeptide sequence of 5391A-RAGE₃₆₂₋₄₀₄ |
| SEQ ID NO: 9 | Full length polypeptide sequence of human ALCAM |
| SEQ ID NO: 10 | Full length polypeptide sequence of human BCAM |
| SEQ ID NO: 11 | Full length polypeptide sequence of human MCAM |
| SEQ ID NO: 12 | Full length polypeptide sequence of human EpCAM |
| SEQ ID NO: 13 | Full length polypeptide sequence of human CADM4 |
| SEQ ID NO: 14 | Full length polypeptide sequence of human RAGE |
| SEQ ID NO: 15 | Full length polypeptide sequence of human AT1R |
| SEQ ID NO: 16 | Full length polypeptide sequence of mouse ALCAM |
| SEQ ID NO: 17 | Full length polypeptide sequence of chicken EpCAM |
| SEQ ID NO: 18 | Full length polypeptide sequence of human C5aR1 |
| SEQ ID NO: 19 | Polypeptide sequence of RAGE₃₃₈₋₃₆₁ |
| SEQ ID NO: 20 | HIV TAT motif |
| SEQ ID NO: 21 | Polypeptide sequence of RAGE₃₇₉₋₃₉₀ |
| SEQ ID NO: 22 | Polypeptide sequence of RAGE₃₇₉₋₃₉₀ with initiating |
| | methionine. |
| SEQ ID NO: 23 | Polypeptide sequence of S391A-E392X-RAG_{E362-39}1 |
| SEQ ID NO: 24 | Polypeptide sequence of 5391X-RAGE₃₆₂₋₃₉₀ |
| SEQ ID NO: 25 | Polypeptide sequence of RAGE derivative |
| | Q379EEEEERAELN_{R390} |
| SEQ ID NO: 26 | Polypeptide sequence of RAGE derivative |
| | Q379EEEEERAELNK₃₉₀ |
| SEQ ID NO: 27 | Polypeptide sequence of RAGE derivative |
| | K379EEEEERAELNQ₃₉₀ |
| SEQ ID NO: 28 | Polypeptide sequence of RAGE derivative |
| | K379EEEEERAELNK₃₉₀ |
| SEQ ID NO: 29 | Polypeptide sequence of RAGE derivative |
| | K379EEEEERAELNR₃₉₀ |
| SEQ ID NO: 30 | Polypeptide sequence of RAGE₃₄₃₋₃₆₁ |
| | LALGILGGLGTAALLIGVI |
| SEQ ID NO: 31 | Polypeptide sequence of cytosolic tail of RAGE₃₆₂₋₄₀₄ |

SEQ ID NO: 1—Peptide sequence of cytosolic tail of ALCAM (corresponds to residues 551-583 of ALCAM, with initial Methionine already present):

MKKSKTASKHVNKDLGNMEENKKLEENNHKTEA

SEQ ID NO: 2—BCAM cytosolic tail sequence (corresponds to residues 569-628 of BCAM plus an initiating Methionine):

MYCVRRKGGPCCRQRREKGAPPPGEPGLSHSGSEQPEQTGLLMGGASGGA
RGGSGGFGDEC

SEQ ID NO: 3—MCAM cytosolic tail sequence (corresponds to residues 584-637 of MCAM plus an initiating Methionine):

MKKGKLPCRRSGKQEITLPPSRKSELVVEVKSDKLPEEMGLLQGSSGDKR
APGDQ

SEQ ID NO: 4—EpCAM cytosolic tail sequence (corresponds to residues 289-314 of EpCAM plus an initiating Methionine):

MSRKKRMAKYEKAEIKEMGEMHRELNA

SEQ ID NO: 5—CADM4 cytosolic tail sequence (corresponds to residues 346-388 of EpCAM plus an initiating Methionine):

MSVRQKGSYLTHEASGLDEQGEAREAFLNGSDGHKRKEEFFI

SEQ ID NO: 6—Peptide sequence of ALCAM₅₅₉₋₅₈₀ (corresponds to residues 559-580 of ALCAM plus an initiating Methionine): MKHVNKDLGNMEENKKLEENNHK

SEQ ID NO: 7—Peptide sequence of RAGE₃₇₀₋₃₉₀ (corresponds to residues 370-390 of RAGE plus an initiating Methionine): MGEERKAPENQEEEEERAELNQ

SEQ ID NO: 8—Peptide sequence of S391A-RAGE₃₆₂₋₄₀₄ (corresponds to residues 362-404 of RAGE with mutation of Serine 391 to Alanine plus an initiating Methionine):

MLWQRRQRRGEERKAPENQEEEEERAELNQAEEPEAGESSTGGP

SEQ ID NO: 9-Full length Human ALCAM (583 amino acids). GenBank:
AAB59499.1:
MESKGASSCRLLFCLLISATVFRPGLGWYTVNSAYGDTIIIPCRLDVPQNLMFGKWKYEKPD
GSPVFIAFRSSTKKSVQYDDVPEYKDRLNLSENYTLSISNARISDEKRFVCMLVTEDNVFEA
PTIVKVFKQPSKPEIVSKALFLETEQLKKLGDCISEDSYPDGNITWYRNGKVLHPLEGAVVIIF
KKEMDPVTQLYTMTSTLEYKTTKADIQMPFTCSVTYYGPSGQKTIHSEQAVFDIYYPTEQVT
IQVLPPKNAIKEGDNITLKCLGNGNPPPEEFLFYLPGQPEGIRSSNTYTLMDVRRNATGDYK
CSLIDKKSMIASTAITVHYLDLSLNPSGEVTRQIGDALPVSCTISASRNATVVWMKDNIRLRS
SPSFSSLHYQDAGNYVCETALQEVEGLKKRESLTLIVEGKPQIKMTKKTDPSGLSKTIICHVE
GFPKPAIMAMTGSGSVINQTEESPYINGRYYSKIISPEENVTLTCTAENQLERTVNSLNVSAI
SIPEHDEADEISDENREKVNDQAKLIVGIVVGLLLAALVAGVVYWLYMKKSKTASKHVNKDL
GNMEENKKLEENNHKTEA
SEQ ID NO: 10: Full length Human BCAM (628 amino acids). NP_005572.2.:
MEPPDAPAQARGAPRLLLLAVLLAAHPDAQAEVRLSVPPLVEVM RGKSVILDCTPTGTHDH
YMLEWFLTDRSGARPRLASAEMQGSELQVTMHDTRGRSPPYQLDSQGRLVLAEAQVGDE
RDYVCVVRAGAAGTAEATARLNVFAKPEATEVSPNKGTLSVMEDSAQEIATCNSRNGNPA
PKITWYRNGQRLEVPVEMNPEGYMTSRTVREASGLLSLTSTLYLRLRKDDRDASFHCAAH
YSLPEGRHGRLDSPTFHLTLHYPTEHVQFWVGSPSTPAGWVREGDTVQLLCRGDGSPSP
EYTLFRLQDEQEEVLNVNLEGNLTLEGVTRGQSGTYGCRVEDYDAADDVQLSKTLELRVA
YLDPLELSEGKVLSLPLNSSAVVNCSVHGLPTPALRWTKDSTPLGDGPMLSLSSITFDSNGT
YVCEASLPTVPVLSRTQNFTLLVQGSPELKTAEIEPKADGSWREGDEVTLICSARGHPDPKL
SWSQLGGSPAEPIPGRQGWVSSSLTLKVTSALSRDGISCEASNPHGNKRHVFHFGTVSPQ
TSQAGVAVMAVAVSVGLLLLVVAVF7YCVRRKGGPCCRQRREKGAPPPGEPGLSHSGSE
QPEQTGLLMGGASGGARGGSGGFGDEC
SEQ ID NO: 11: Full length Human MCAM (646 amino acids). NP_006491.2.:
MGLPRLVCAFLLAACCCCPRVAGVPGEAEQPAPELVEVEVGSTALLKCGLSQSQGNLSHV
DWFSVHKEKRTLIFRVRQGQGQSEPGEYEQRLSLQDRGATLALTQVTPQDERIFLCQGKR
PRSQEYRIQLRVYKAPEEPNIQVNPLGIPVNSKEPEEVATCVGRNGYPIPQVIWYKNGRPLK
EEKNRVHIQSSQTVESSGLYTLQSILKAQLVKEDKDAQFYCELNYRLPSGNHMKESREVTV
PVFYPTEKVWLEVEPVGMLKEGDRVEIRCLADGNPPPHFSISKQNPSTREAEEETTNDNGV
LVLEPARKEHSGRYECQGLDLDTMISLLSEPQELLVNYVSDVRVSPAAPERQEGSSLTLTC
EAESSQDLEFQWLREETGQVLERGPVLQLHDLKREAGGGYRCVASVPSIPGLNRTQLVNV
AIFGPPWMAFKERKVWVKENMVLNLSCEASGHPRPTISWNVNGTASEQDQDPQRVLSTLN
VLVTPELLETGVECTASNDLGKNTSILFLELVNLTTLTPDSNTTTGLSTSTASPHTRANSTST
ERKLPEPESRGVVIVAVIVCILVLAVLGAVLYFLYKKGKLPCRRSGKQEITLPPSRKSELVVEV
KSDKLPEEMGLLQGSSGDKRAPGDQGEKYIDLRH
SEQ ID NO: 12: Full length Human EpCAM (314 amino acids).
MAPPQVLAFGLLLAAATATFAAAQEECVCENYKLAVNCFVNNNRQCQCTSVGAQNTVICSK
LAAKCLVMKAEMNGSKLGRRAKPEGALQNNDGLYDPDCDESGLFKAKQCNGTSMCWCV
NTAGVRRTDKDTEITCSERVRTYWIIIELKHKAREKPYDSKSLRTALQKEITTRYQLDPKFITSI
LYENNVITIDLVQNSSQKTQNDVDIADVAYYFEKDVKGESLFHSKKMDLTVNGEQLDLDPG
QTLIYYVDEKAPEFSMQGLKAGVIAVIVVVVIAVVAGIWLVISRKKRMAKYEKAEIKEMGEM
HRELNA
SEQ ID NO: 13: Full length Human CADM4 (388 amino acids).
MGRARRFQWPLLLLWAAAAGPGAGQEVQTENVTVAEGGVAEITCRLHQYDGSIVVIQNPA
RQTLFFNGTRALKDERFQLEEFSPRRVRIRLSDARLEDEGGYFCQLYTEDTHHQIATLTVLV
APENPVVEVREQAVEGGEVELSCLVPRSRPAATLRWYRDRKELKGVSSSQENGKVWSVA
STVRFRVDRKDDGGIIICEAQNQALPSGHSKQTQYVLDVQYSPTARIHASQAVVREGDTLVL
TCAVTGNPRPNQIRWNRGNESLPERAEAVGETLTLPGLVSADNGTYTCEASNKHGHARAL
YVLVVYDPGAVVEAQTSVPYAIVGG1LALLVFLIICVLVGMVWCSVRQKGSYLTHEASGLDE
QGEAREAFLNGSDGHKRKEEFFI
SEQ ID NO: 14-Full length polypeptide sequence of RAGE
(404 amino acids), UniProtKB Accession No. Q15109:
MAAGTAVGAWVLVLSLWGAVVGAQNITARIGEPLVLKCKGAPKKPPQRLEWKLNTGRTEA
WKVLSPQGGGPWDSVARVLPNGSLFLPAVGIQDEGIFRCQAMNRNGKETKSNYRVRVYQI
PGKPEIVDSASELTAGVPNKVGTCVSEGSYPAGTLSWHLDGKPLVPNEKGVSVKEQTRRH
PETGLFTLQSELMVTPARGGDPRPTFSCSFSPGLPRHRALRTAPIQPRVWEPVPLEEVQLV
VEPEGGAVAPGGTVTLTCEVPAQPSPQIHWMKDGVPLPLPPSPVLILPEIGPQDQGTYSCV
ATHSSHGPQESRAVSISIIEPGEEGPTAGSVGGSGLGTLALALGILGGLGTAALLIGVILWQR
RQRRGEERKAPENQEEEEERAELNQSEEPEAGESSTGGP
SEQ ID NO: 15-Full length polypeptide sequence of human AT1R,
UniProtKB Accession No. P30556:
MILNSSTEDGIKRIQDDCPKAGRHNYIFVMIPTLYSIIFVVGIFGNSLVVIVIYFYMKLKTVASVF
LLNLALADLCFLLTLPLWAVYTAMEYRWPFGNYLCKIASASVSFNLYASVFLLTCLSIDRYLAI
VHPMKSRLRRTMLVAKVTCIIIWLLAGLASLPAI1HRNVFFIENTNITVCAFHYESQNSTLPIGL
GLTKNILGFLFPFLIILTSYTLIWKALKKAYEIQKNKPRNDDIFKIIMAIVLFFFFSWIPHQIFTFLD
VLIQLGIIRDCRIADIVDTAMPITICIAYFNNCLNPLFYGFLGKKFKRYFLQLLKYIPPKAKSHSN
LSTKMSTLSYRPSDNVSSSTKKPAPCFEVE
SEQ ID NO: 16-Full length polypeptide sequence of mouse (murine)
ALCAM, UniProtKB Accession No. Q61490:
MASKVSPSCRLVFCLLISAAVLRPGLGWYTVNSAYGDTIVMPCRLDVPQNLMFGKWKYEK
PDGSPVFIAFRSSTKKSVQYDDVPEYKDRLSLSENYTLSIANAKISDEKRFVCMLVTEDNVF
EAPTLVKVFKQPSKPEIVNKAPFLETDQLKKLGDCISRDSYPDGNITWYRNGKVLQPVEGEV
AILFKKEIDPGTQLYTVTSSLEYKTTRSDIQMPFTCSVTYYGPSGQKTIYSEQEIFDIYYPTEQ
VTIQVLPPKNAIKEGDNITLQCLGNGNPPPEEFMFYLPGQPEGIRSSNTYTLTDVRRNATGD
YKCSLIDKRNMAASTTITVHYLDLSLNPSGEVTKQIGDTLPVSCTISASRNATVVWMKDNIRL
RSSPSFSSLHYQDAGNYVCETALQEVEGLKKRESLTLIVEGKPQIKMTKKTDPSGLSKTIICH
VEGFPKPAIHWTITGSGSVINQTEESPYINGRYYSKIIISPEENVTLTCTAENQLERTVNSLNV
SAISIPEHDEADDISDENREKVNDQAKLIVGIVVGLLLAALVAGVVYWLYMKKSKTASKHVNK
DLGNMEENKKLEENNHKTEA
SEQ ID NO: 17-Full length polypeptide sequence of chicken EpCAM,
UniProtKB Accession No. A0A1D5PWY3 with two polymorphisms
(E94G and T158I):
MELLRGAALLLLLCAAACAQDSCTCTKNKRVTNCKLIDNVCHCNSIGSSVSVNCEILTSKCLL
MKAEMANTKSGRREKPKDALQDTDGLYDPECGNNGLFKAKQCNGTTCWCVNTAGVRRT
DKHDTDLKCNQLVRTTWIIIEMRHAERKTPLNAESLIRYLKDTITSRYMLDGRYISGVVYENP
TITIDLKQNSSDKTPGDVDITDVAYYFEKDVKDDSIFLNNKLNMNIDNEELKFDNMMVYYVDE
VPPEFSMKSLTAGVIAVIVIVVLAIVAGIIGLVLSRRRKGKYVKAEMKEMNEMHRGLNA
SEQ ID NO: 18-Full length polypeptide sequence of human C5aR1,
UniProtKB Acession No. P21730:
MNSFNYTTPDYGHYDDKDTLDLNTPVDKTSNTLRVPDILALVIFAVVFLVGVLGNALVVVVVT
AFEAKRTINAIWFLNLAVADFLSCLALPILFTSIVQHHHWPFGGAACSILPSLILLNMYASILLL
ATISADRFLLVFKPIWCQNFRGAGLAWIACAVAWGLALLLTIPSFLYRVVREEYFPPKVLCGV
DYSHDKRRERAVAIVRLVLGFLWPLLTLTICYTFILLRTWSRRATRSTKTLKVVVAVVASFFIF
WLPYQVTGIMMSFLEPSSPTFLLLNKLDSLCVSFAYINCCINPIIYVVAGQGFQGRLRKSLPS
LLRNVLTEESVVRESKSFTRSTVDTMAQKTQAV
SEQ ID NO: 31-Peptide sequence of RAGE₃₆₂₋₄₀₄ (correspomds to residues
362-404 of RAGE): LWQRRQRRGEERKAPENQEEEEERAELNQSEEPEAGESSTGGP

### CONCLUSIONS

Activation of certain co-located GPCRs by their cognate ligands, such as activation of AT1R by Ang II, triggers inflammation through pathways distinct from classical canonical signalling via GPCRs that induce, for example, calcium influx, inositol phosphate synthesis and activation of PKA. Here, the inventors show that ligand-independent activation of the cytosolic tail of IgSF CAM, specifically ALCAM, BCAM and MCAM can trigger activation of NFκB and NFκB-dependent signalling following activation of certain co-located GPCRs by their cognate ligands.

Even though the ectodomain has historically been considered to be essential for functions of IgSF CAMs and their superfamily members, without wishing to be bound by theory, the inventors believe the ligand-independent activation of the cytosolic tail of IgSF CAM superfamily members by certain activated co-located GPCRs is an important mechanism inducing downstream effector activation and signalling.

The inventors show that in CHO cells and ARPE cells proinflammatory signalling mediated by the cytosolic tail of IgSF CAM superfamily members can be selectively inhibited by non-signalling peptides derived from the cytosolic tail of RAGE, specifically RAGE₃₇₀₋₃₉₀ and S391A-RAGE₃₆₂₋₄₀₄. These peptides are able to inhibit proinflammatory signalling following the activation of the AT1 receptor by Ang II that is mediated by the cytosolic tail of IgSF CAMs, specifically ALCAM, BCAM and MCAM.

Furthermore, the inventors demonstrate that non-signaling peptides derived from the cytosolic tail of IgSF CAMs, specifically ALCAM, have the capacity to modulate signalling mediated by full length RAGE.

The disclosure of every patent, patent application, and publication cited herein is hereby incorporated herein by reference in its entirety.

The citation of any reference herein should not be construed as an admission that such reference is available as “Prior Art” to the instant application.

Throughout this specification, unless the context requires otherwise, the word “comprise” or variations such as “comprises” or “comprising”, will be understood to imply the inclusion of a stated integer or group of integers but not the exclusion of any other integer or group of integers.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

As used herein, “isolated” when describing a peptide modulator of the invention means a peptide described herein that is not in a natural state (e.g. it is disassociated from a larger protein molecule or cellular debris in which it naturally occurs or is normally associated with), or is a non-naturally occurring fragment of a naturally occurring protein (e.g. the peptide comprises less than 25%, preferably less than 10% and most preferably less than 5% of the naturally occurring protein). Isolated also may mean that the amino acid sequence of the peptide does not occur in nature, for example, because the sequence is modified from a naturally occurring sequence (e.g. by alteration of certain amino acids, including basic (i.e. cationic) amino acids such as arginine, tryptophan, or lysine), or because the sequence does not contain flanking amino acids which are present in nature. The term “isolated” may mean that the peptide or amino acid sequence is a man-made sequence or polypeptide and may be non-naturally occurring.

Likewise, “isolated” as used in connection with nucleic acids which encode peptides embraces all of the foregoing, e.g. the isolated nucleic acids are disassociated from adjacent nucleotides with which they are associated in nature, and can be produced recombinantly, synthetically, by purification from biological extracts, and the like. Isolated nucleic acids can contain a portion that encodes one of the foregoing peptides and another portion that codes for another peptide or protein. The isolated nucleic acids also can be labeled. The nucleic acids include codons that are preferred for animal, bacterial, plant, or fungal usage. In certain embodiments, the isolated nucleic acid is a vector, such as an expression vector, which includes a nucleic acid that encodes one of the foregoing isolated peptides. A general method for the construction of any desired DNA sequence is provided, e.g., in Brown J. et al. (1979), Methods in Enzymology, 68:109; Sambrook J, Maniatis T (1989), supra.

The term “amino acid” or “residue” as used herein includes any one of the twenty naturally-occurring amino acids, the D-form of any one of the naturally-occurring amino acids, non-naturally occurring amino acids, and derivatives, analogues and mimetics thereof. Any amino acid, including naturally occurring amino acids, may be purchased commercially or synthesized by methods known in the art. Examples of non-naturally-occurring amino acids include norleucine (“Nle”), norvaline (“Nva”), β-Alanine, L- or D-naphthalanine, ornithine (“Orn”), homoarginine (homoArg) and others well known in the peptide art, including those described in M. Bodanzsky, “Principles of Peptide Synthesis,” 1st and 2nd revised ed., Springer-Verlag, New York, N.Y., 1984 and 1993, and Stewart and Young, “Solid Phase Peptide Synthesis,” 2nd ed., Pierce Chemical Co., Rockford, Ill., 1984, both of which are incorporated herein by reference.

Common amino acids may be referred to by their full name, standard single-letter notation (IUPAC), or standard three-letter notation for example: A, Ala, alanine; C, Cys, cysteine; D, Asp, aspartic acid (aspartate); E, Glu, glutamic acid (glutamate); F, Phe, phenylalanine; G, Gly, glycine; H, His, histidine; I, Ile isoleucine; K, Lys, lysine; L, Leu, leucine; M, Met, methionine; N, Asn, asparagine; P, Pro, proline; Q, Gln, glutamine; R, Arg, arginine; S, Ser, serine; T, Thr, threonine; V, Val, valine; W, Trp, tryptophan; X, Hyp, hydroxyproline; Y, Tyr, tyrosine. Any and all of the amino acids in the compositions herein can be naturally occurring, synthetic, and derivatives or mimetics thereof.

Non-peptide analogues of peptides, e.g., those that provide a stabilized structure or lessened biodegradation, are also contemplated. Peptide mimetic analogues can be prepared based on a selected peptide by replacement of one or more residues by non-peptide moieties. Preferably, the non-peptide moieties permit the peptide to retain its natural conformation, or stabilize a preferred, e.g., bioactive, conformation. One example of methods for preparation of non-peptide mimetic analogues from peptides is described in Nachman et al., Regul. Pept. 57:359-370 (1995). The term “peptide” as used herein embraces all of the foregoing.

As mentioned above, the peptide of the present invention may be composed either of naturally occurring amino acids, i.e. L-amino acids, or of D-amino acids, i.e. of an amino acid sequence comprising D-amino acids in retro-inverso order as compared to the native sequence. The term “retro-inverso” refers to an isomer of a linear peptide in which the direction of the sequence is reversed and the chirality of each amino acid residue is inverted. Thus, any sequence herein, being present in L-form is also inherently disclosed herein as a D-enantiomeric (retro-inverso) peptide sequence. D-enantiomeric (retro-inverso) peptide sequences according to the invention can be constructed, e.g. by synthesizing a reverse of the amino acid sequence for the corresponding native L-amino acid sequence. In D-retro-inverso enantiomeric peptides, e.g. a component of the isolated peptide, the positions of carbonyl and amino groups in each single amide bond are exchanged, while the position of the side-chain groups at each alpha carbon is preserved.

Preparation of a component of the isolated peptide modulators of embodiments of the invention as defined above having D-enantiomeric amino acids can be achieved by chemically synthesizing a reverse amino acid sequence of the corresponding naturally occurring L-form amino acid sequence or by any other suitable method known to a skilled person. Alternatively, the D-retro-inverso-enantiomeric form of a peptide or a component thereof may be prepared using chemical synthesis as disclosed above utilizing an L-form of an peptide or a component thereof as a matrix for chemical synthesis of the D-retro-inverso-enantiomeric form.

Various changes may be made including the addition of various side groups that do not affect the manner in which a peptide modulator of embodiments of the invention functions, or which favourably affect the manner in which a peptide modulator of embodiments of the invention functions. Such changes may involve adding or subtracting charge groups, substituting amino acids, adding lipophilic moieties that do not affect binding but that affect the overall charge characteristics of the peptide modulator of embodiments of the invention facilitating delivery across the blood-brain barrier, etc. For each such change, no more than routine experimentation is required to test whether the molecule functions according to the invention. One simply makes the desired change or selects the desired peptide and applies it in a fashion as described in detail in the examples.

In one form of the invention, the term “sequence identity” as defined herein means that the sequences are compared as follows. To determine the percent identity of two amino acid sequences, the sequences can be aligned for optimal comparison purposes (e.g., gaps can be introduced in the sequence of a first amino acid sequence). The amino acids at corresponding amino acid positions can then be compared. When a position in the first sequence is occupied by the same amino acid as the corresponding position in the second sequence, then the molecules are identical at that position. The percent identity between the two sequences is a function of the number of identical positions shared by the sequences. For example, where a particular peptide is said to have a specific percent identity to a reference polypeptide of a defined length, the percent identity is relative to the reference peptide. Thus, a peptide that is 50% identical to a reference polypeptide that is 100 amino acids long can be a 50 amino acid polypeptide that is completely identical to a 50 amino acid long portion of the reference polypeptide. It might also be a 100 amino acid long polypeptide, which is 50% identical to the reference polypeptide over its entire length. Such a determination of percent identity of two sequences can be accomplished using a mathematical algorithm.

A preferred, non-limiting example of a mathematical algorithm utilized for the comparison of two sequences is the algorithm of Karlin et al. (1993), PNAS USA, 90:5873-5877. Such an algorithm is incorporated into the NBLAST program, which can be used to identify sequences having the desired identity to the amino acid sequence of the invention. To obtain gapped alignments for comparison purposes, Gapped BLAST can be utilized as described in Altschul et al., (1997), Nucleic Acids Res, 25:3389-3402. When utilizing BLAST and Gapped BLAST programs, the default parameters of the respective programs (e.g., NBLAST) can be used. The sequences further may be aligned using Version 9 of the Genetic Computing Group's GAP (global alignment program), using the default (BLOSUM62) matrix (values −4 to +11) with a gap open penalty of −12 (for the first null of a gap) and a gap extension penalty of −4 (per each additional consecutive null in the gap). After alignment, percentage identity is calculated by expressing the number of matches as a percentage of the number of amino acids in the claimed sequence. The described methods of determination of the percent identity of two amino acid sequences can be applied correspondingly to nucleic acid sequences.

In one embodiment a peptide modulator of embodiments of the invention may be linked directly or via a linker. A “linker” in the present context is usually a peptide, oligopeptide or polypeptide and may be used to link multiples of the peptides to one another. The peptides of the invention selected to be linked to one another can be identical sequences, or are selected from any of the peptides of the invention. A linker can have a length of 1-10 amino acids, more preferably a length of 1 to 5 amino acids and most preferably a length of 1 to 3 amino acids. In certain embodiments, the linker is not required to have any secondary structure forming properties, i.e. does not require a α-helix or β-sheet structure forming tendency, e.g. if the linker is composed of at least 35% of glycine residues. As mentioned hereinbefore, a linker can be a cleavable peptide such as an MMP peptide which can be cleaved intracellularly by normal cellular processes, effectively raising the intracellular dose of the previously linked peptides, while keeping the extracellular dose low enough to not be considered toxic. The use of a(n) intracellularly/endogenously cleavable peptide, oligopeptide, or polypeptide sequence as a linker permits the peptides to separate from one another after delivery into the target cell. Cleavable oligo- or polypeptide sequences in this context also include protease cleavable oligo- or polypeptide sequences, wherein the protease cleavage site is typically selected dependent on the protease endogenously expressed by the treated cell. The linker as defined above, if present as an oligo- or polypeptide sequence, can be composed either of D-amino acids or of naturally occurring amino acids, i.e. L-amino acids. As an alternative to the above, coupling or fusion of the peptides can be accomplished via a coupling or conjugating agent, e.g. a cross-linking reagent.

There are several intermolecular cross-linking reagents which can be utilized, see for example, Means and Feeney, Chemical Modification of Proteins, Holden-Day, 1974, pp. 39-43. Among these reagents are, for example, N-succinimidyl 3-(2-pyridyldithio)propionate (SPDP) or N,N′-(1,3-phenylene)bismaleimide; N,N′-ethylene-bis-(iodoacetamide) or other such reagent having 6 to 11 carbon methylene bridges; and 1,5-difluoro-2,4-dinitrobenzene. Other cross-linking reagents useful for this purpose include: p,p′-difluoro-m,m′-dinitrodiphenylsulfone; dimethyl adipimidate; phenol-1,4-disulfonylchloride; hexamethylenediisocyanate or diisothiocyanate, or azophenyl-p-diisocyanate; glutaraldehyde and disdiazobenzidine. Cross-linking reagents may be homobifunctional, i.e., having two functional groups that undergo the same reaction. A preferred homobifunctional cross-linking reagent is bismaleimidohexane (BMH). BMH contains two maleimide functional groups, which react specifically with sulfhydryl-containing compounds under mild conditions (pH 6.5-7.7). The two maleimide groups are connected by a hydrocarbon chain. Therefore, BMH is useful for irreversible cross-linking of proteins (or polypeptides) that contain cysteine residues. Cross-linking reagents may also be heterobifunctional. Heterobifunctional cross-linking reagents have two different functional groups, for example an amine-reactive group and a thiol-reactive group, that will cross-link two proteins having free amines and thiols, respectively. Examples of heterobifunctional cross-linking reagents are succinimidyl 4-(N-maleimidomethyl)cyclohexane-1-carboxylate (SMCC), m-maleimidobenzoyl-N-hydroxysuccinimide ester (MBS), and succinimide 4-(p-maleimidophenyl)butyrate (SMPB), an extended chain analogue of MBS. The succinimidyl group of these cross-linking reagents with a primary amine, and the thiol-reactive maleimide forms a covalent bond with the thiol of a cysteine residue. Because cross-linking reagents often have low solubility in water, a hydrophilic moiety, such as a sulfonate group, may be added to the cross-linking reagent to improve its water solubility. Sulfo-MBS and sulfo-SMCC are examples of cross-linking reagents modified for water solubility. Many cross-linking reagents yield a conjugate that is essentially non-cleavable under cellular conditions. Therefore, some cross-linking reagents contain a covalent bond, such as a disulfide, that is cleavable under cellular conditions. For example, Traut's reagent, dithiobis (succinimidylpropionate) (DSP), and N-succinimidyl 3-(2-pyridyldithio)propionate (SPDP) are well-known cleavable cross-linkers. The use of a cleavable cross-linking reagent permits the peptides to be separated after delivery into the target cell, if desired, provided the cell is capable of cleaving a particular sequence of the crosslinker reagent. For this purpose, direct disulfide linkage may also be useful. Chemical cross-linking may also include the use of spacer arms. Spacer arms provide intramolecular flexibility or adjust intramolecular distances between conjugated moieties and thereby may help preserve biological activity. A spacer arm may be in the form of a protein (or polypeptide) moiety that includes spacer amino acids, e.g. proline. Alternatively, a spacer arm may be part of the cross-linking reagent, such as in “long-chain SPDP” (Pierce Chem. Co., Rockford, Ill., cat. No. 21651H). Numerous cross-linking reagents, including the ones discussed above, are commercially available. Detailed instructions for their use are readily available from the commercial suppliers. A general reference on protein cross-linking and conjugate preparation is: Wong, Chemistry of Protein Conjugation and Cross-Linking, CRC Press (1991).

In one embodiment, peptide modulators may also contain a “derivative”, “variant”, or “functional fragment”, i.e. a sequence of a peptide that is derived from the naturally occurring (L-amino-acid) sequence of a peptide of the invention as defined above by way of substitution(s) of one or more amino acids at one or more sites of the amino acid sequence, by way of deletion(s) of one or more amino acids at any site of the naturally occurring sequence, and/or by way of insertion(s) of one or more amino acids at one or more sites of the naturally occurring peptide sequence. “Derivatives” shall retain their biological activity if used as peptides of the invention. Derivatives in the context of the present invention may also occur in the form of their L- or D-amino-acid sequences as defined above, or both.

If substitution(s) of amino acid(s) are carried out for the preparation of a derivative of the peptides of the invention, conservative (amino acid) substitutions are preferred. Conservative (amino acid) substitutions typically include substitutions within the following groups: glycine and alanine; valine, isoleucine and leucine; aspartic acid (aspartate) and glutamic acid (glutamate); asparagine and glutamine; serine and threonine; lysine and arginine; and phenylalanine and tyrosine. Thus, preferred conservative substitution groups are aspartate-glutamate; asparagine-glutamine; valine-leucine-isoleucine; alanine-valine; and phenylalanine-tyrosine. By such mutations e.g. stability and/or effectiveness of a peptide may be enhanced. If mutations are introduced into the peptide, the peptide remains (functionally) homologous, e.g. in sequence, in function, and in antigenic character or other function. Such mutated components of the peptide can possess altered properties that may be advantageous over the non-altered sequences of the peptides of the invention for certain applications (e.g. increased pH optimum, increased temperature stability etc.).

In one embodiment, a derivative of the peptide of the invention is defined as having substantial identity with the non-modified sequences of the peptide of the invention. Particularly preferred are amino acid sequences which have at least 30% sequence identity, preferably at least 50% sequence identity, even preferably at least 60% sequence identity, even preferably at least 75% sequence identity, even more preferably at least 80%, yet more preferably 90% sequence identity and most preferably at least 95% or even 99% sequence identity to the naturally occurring analogue. Appropriate methods for synthesis or isolation of a functional derivative of the peptides of the invention as well as for determination of percent identity of two amino acid sequences are described above. Additionally, methods for production of derivatives of the peptides as disclosed above are well known and can be carried out following standard methods which are well known by a person skilled in the art (see e.g., Sambrook J, Maniatis T (1989)).

As a further embodiment, the invention provides pharmaceutical compositions or medicaments comprising the modulators as defined herein. In certain embodiments, such pharmaceutical compositions or medicaments comprise the modulators as well as an optional linker, as defined herein.

Additionally, such a pharmaceutical composition or medicament can comprise a pharmaceutically acceptable carrier, adjuvant, or vehicle. A “pharmaceutically acceptable carrier, adjuvant, or vehicle” according to the invention refers to a non-toxic carrier, adjuvant or vehicle that does not destroy the pharmacological activity or physiological targeting of the modulator with which it is formulated. Pharmaceutically acceptable carriers, adjuvants or vehicles that can be used in the pharmaceutical compositions of this invention include, but are not limited to those that can be applied cranially or intracranially, or that can cross the blood-brain barrier (BBB). Notwithstanding this, the pharmaceutical compositions of the invention can include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of the present invention may be administered orally, parenterally, by inhalation spray, topically, rectally, nasally, buccally, vaginally, cerebrally, or via an implanted reservoir.

The term parenteral as used herein includes subcutaneous, intravenous, intramuscular, intra-articular, intra-synovial, intrasternal, intrathecal, intrahepatic, intralesional and intracranial injection or infusion techniques. The pharmaceutical compositions are administered orally, intraperitoneally or intravenously. Sterile injectable forms of the pharmaceutical compositions of this invention may be aqueous or oleaginous suspension. These suspensions can be formulated according to techniques known in the art using suitable dispersing or wetting agents and suspending agents. The sterile injectable preparation can also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

As such, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutically acceptable compositions herein may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavouring or colouring agents may also be added.

Alternatively, the pharmaceutical composition as defined herein may be administered in the form of suppository for rectal administration. Such a suppository can be prepared by mixing the agent with a suitable non-irritating excipient that is solid at room temperature but liquid at rectal temperature and, therefore, will melt in the rectum to release the drug. Such materials include cocoa butter, beeswax and polyethylene glycols.

The pharmaceutical composition as defined herein may also be administered topically, especially when the target of treatment includes areas or organs readily accessible by topical application, including diseases of the brain, other intra-cranial tissues, the eye, or the skin. Suitable formulations are readily prepared for each of these areas or organs.

For topical applications, the pharmaceutical composition as defined herein may be formulated in a suitable ointment containing modulators as identified herein, suspended or dissolved in one or more carriers. Carriers for topical administration of the peptide include, but are not limited to, mineral oil, liquid petrolatum, white petrolatum, propylene glycol, polyoxyethylene, polyoxypropylene compound, emulsifying wax and water. Alternatively, the pharmaceutical composition as defined herein can be formulated in a suitable lotion or cream containing the peptide suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

The pharmaceutical composition as defined herein may also be administered by nasal aerosol or inhalation. Such a composition may be prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. The pharmaceutically acceptable composition or medicament herein is formulated for oral or parenteral administration, e.g. by injection.

For treatment purposes, a non-toxic, effective amount of the modulator may be used for preparation of a pharmaceutical composition as defined above. Therefore, an amount of the modulator may be combined with the carrier material(s) to produce a composition as defined above.

The pharmaceutical composition is typically prepared in a single (or multiple) dosage form, which will vary depending upon the host treated and the particular mode of administration. Usually, the pharmaceutical composition is formulated so that a dosage range per dose of 0.0001 to 100 mg/kg body weight/day of the peptide can be administered to a patient receiving the pharmaceutical composition. Preferred dosage ranges per dose vary from 0.01 mg/kg body weight/day to 50 mg/kg body weight/day, even further preferred dosage ranges per dose range from 0.1 mg/kg body weight/day to 10 mg/kg body weight/day.

However, dosage ranges and treatment regimens as mentioned above may be adapted suitably for any particular patient dependent upon a variety of factors, including the activity of the specific modulator employed, the age, body weight, general health, sex, diet, time of administration, rate of excretion, drug combination, the judgment of the treating physician and the severity of the particular disease being treated. In this context, administration may be carried with in an initial dosage range, which may be varied over the time of treatment, e.g. by increasing or decreasing the initial dosage range within the range as set forth above. Alternatively, administration may be carried out in a continuous manner by administering a specific dosage range, thereby maintaining the initial dosage range over the entire time of treatment. Both administration forms may furthermore be combined, e.g. if the dosage range is to be adapted (increased or decreased) between various sessions of the treatment but kept constant within the single session so that dosage ranges of the various sessions differ from each other.

When used therapeutically, the modulators of the invention are administered in therapeutically effective amounts. In general, a therapeutically effective amount means an amount necessary to delay the onset of, inhibit the progression of, or halt altogether the particular condition being treated. Generally, a therapeutically effective amount will vary with the subject's age and condition, as well as the nature and extent of the disease in the subject, all of which can be determined by one of ordinary skill in the art. The dosage may be adjusted by the individual physician, particularly in the event of any complications being experienced.

In one aspect, the invention provides for the use of the IgSF CAM modulators described herein for the manufacture of a medicament for treating, preventing or managing an IgSF CAM-related disorder in a patient in need of such treatment.

In one aspect, the invention provides a method for treating, preventing or managing an IgSF CAM-related disorder in a patient in need of such treatment, the method comprising administration of an effective amount of a modulator of an IgSF CAM.

In one aspect, the invention provides a method for treating, preventing or managing an IgSF CAM-related disorder in a patient in need of such treatment, characterised in that the IgSF CAM-related disorder is selected from the group: cardiovascular disorders; digestive disorders; cancers; neurological disorders; respiratory disorders; connective tissue disorders; kidney disorders; genital disorders; skin disorders; eye disorders; and endocrine disorders.

In one aspect, the invention provides a method for treating, preventing or managing an IgSF CAM-related disorder in a patient in need of such treatment, characterised in that the disorder is a cardiovascular disorder selected from the group: atherosclerosis, ischaemic heart disease, myocarditis, endocarditis, cardiomyopathy, acute rheumatic fever, chronic rheumatic heart disease, cerebrovascular disease/stroke, heart failure, vascular calcification, peripheral vascular disease, and lymphangitis.

In one aspect, the invention provides a method for treating, preventing or managing an IgSF CAM-related disorder in a patient in need of such treatment, characterised in that the disorder is a digestive system disorder selected from the group: periodontitis, oesophagitis, gastritis, gastro-duodenal ulceration, Crohn's disease, ulcerative colitis, ischaemic colitis, enteritis and enterocolitis, peritonitis, alcoholic liver disease, hepatitis, toxic liver disease, biliary cirrhosis, hepatic fibrosis/cirrhosis, non-alcoholic fatty liver disease/non-alcoholic steatohepatitis (NAFLD/NASH), liver trauma and recovery from liver injury, trauma or surgery.

In one aspect, the invention provides a method for treating, preventing or managing an IgSF CAM-related disorder in a patient in need of such treatment, characterised in that the disorder is a cancer selected from the group: malignant neoplasms of lip, oral cavity and pharynx, malignant neoplasms of digestive organs, malignant neoplasms of respiratory and intrathoracic organs, malignant neoplasms of bone and articular cartilage, melanoma and other malignant neoplasms of skin, malignant neoplasms of mesothelial and soft tissue, malignant neoplasm of breast, malignant neoplasms of female genital organs, malignant neoplasms of male genital organs, malignant neoplasms of urinary tract, malignant neoplasms of eye, brain and other parts of central nervous system, malignant neoplasms of thyroid and other endocrine glands, malignant neoplasms of lymphoid, haematopoietic and related tissue, malignant neoplasms of ill-defined, secondary and/or unspecified sites.

In one aspect, the invention provides a method for treating, preventing or managing an IgSF CAM-related disorder in a patient in need of such treatment, characterised in that the disorder is a neurological disorder and is selected from the group: inflammatory diseases of the central nervous system, systemic atrophies primarily affecting the central nervous system, extrapyramidal and movement disorders, Parkinson's disease, demyelinating diseases of the central nervous system, Alzheimer's disease, circumscribed brain atrophy, Lewy body disease, epilepsy, migraine, neuropathic pain, diabetic neuropathy, polyneuropathies, glioma development and progression, spinal cord trauma, and ischaemic brain injury/stroke, brain trauma and recovery from brain injury, trauma or surgery.

In one aspect, the invention provides a method for treating, preventing or managing an IgSF CAM-related disorder in a patient in need of such treatment, characterised in that the disorder is a mental disorder and is selected from the group: dementia, Alzheimer's disease, vascular dementia, addiction, schizophrenia, major affective disorder, depression, mania, bipolar disorder, and anxiety disorder.

In one aspect, the invention provides a method for treating, preventing or managing an IgSF CAM-related disorder in a patient in need of such treatment, characterised in that the disorder is a respiratory (pulmonary) disorder and is selected from the group: Acute upper respiratory infections, rhinitis, nasopharyngitis, sinusitis, laryngitis, influenza and pneumonia, acute bronchitis, acute bronchiolitis, asthma, chronic obstructive pulmonary disease (COPD), bronchiectasis, emphysema, chronic lung diseases due to external agents, Acute Respiratory Distress Syndrome (ARDS), pulmonary eosinophilia, and pleuritic, lung trauma and recovery from lung injury, trauma or surgery.

In one aspect, the invention provides a method for treating, preventing or managing an IgSF CAM-related disorder in a patient in need of such treatment, characterised in that the disorder is a connective tissue disorder and is selected from the group: osteoarthritis, infectious arthritis, rheumatoid arthritis, psoriatic and enteropathic arthropathies, juvenile arthritis, gout and other crystal arthropathies, diabetic arthropathy, polyarteritis nodosa, Churg-Strauss, mucocutaneous lymph node syndrome [Kawasaki], hypersensitivity angiitis, Goodpasture syndrome, thrombotic microangiopathy, Wegener granulomatosis, Aortic arch syndrome [Takayasu], giant cell arteritis, polymyalgia rheumatica, microscopic polyangiitis, hypocomplementaemic vasculitis, systemic lupus erythematosus, dermatopolymyositis, polymyositis, systemic sclerosis, CR(E)ST syndrome, Sicca syndrome [Sjögren], mixed connective tissue disease, Behcet disease, traumatic muscle damage, sprain, strain, and fracture.

In one aspect, the invention provides a method for treating, preventing or managing an IgSF CAM-related disorder in a patient in need of such treatment, characterised in that the disorder is a kidney disorder and is selected from the group: glomerulonephritis, nephritis, diabetic kidney disease, interstitial nephritis, obstructive and reflux nephropathy, acute renal failure, and chronic kidney disease.

In one aspect, the invention provides a method for treating, preventing or managing an IgSF CAM-related disorder in a patient in need of such treatment, characterised in that the disorder is a genital disorder and is selected from the group: prostatitis, prostatic hypertrophy, prostatic dysplasia, salpingitis, oophoritis, pelvic inflammatory disease (PID), polycystic ovarian syndrome, cervicitis, cervical dysplasia, vaginitis, vulvitis.

In one aspect, the invention provides a method for treating, preventing or managing an IgSF CAM-related disorder in a patient in need of such treatment, characterised in that the disorder is a skin disorder selected from the group: dermatitis, eczema, pemphigus/pemphygoid, psoriasis, Pityriasis rosea, lichen planus, urticarial, erythrema multiforme, erythema nordosum, sunburn, keratosis, photoageing skin ulceration, superficial skin injury, and open wound.

In one aspect, the invention provides a method for treating, preventing or managing an IgSF CAM-related disorder in a patient in need of such treatment, characterised in that the disorder is an eye disorder selected from the group: keratitis, conjunctivitis, retinitis, glaucoma, scleritis, episcleritis, chorioretinal inflammation, diabetic retinopathy, macular oedema, retinopathy of prematurity, and optic neuritis, eye trauma and recovery from eye injury, trauma or surgery.

In one aspect, the invention provides a method for treating, preventing or managing an IgSF CAM-related disorder in a patient in need of such treatment, characterised in that the disorder is an endocrine disorder selected from the group: diabetes mellitus, insulin resistance, impaired glucose tolerance and thyroiditis.

In one aspect, the invention provides a method for treating, preventing or managing an IgSF CAM-related disorder the method comprising administration of an effective amount of a combination of a modulator of an IgSF CAM with a modulator of the co-located GPCR and/or a modulator of the co-located GPCR signalling pathway, preferably wherein the modulator of the co-located GPCR and/or the modulator of the co-located GPCR signalling pathway is administered at a lower dose than normally administered for the treatment of a disorder related to the co-located GPCR, or wherein the modulator of the co-located GPCR and/or the modulator of the co-located GPCR signalling pathway is administered at a lower dose than normally administered for the treatment of a disorder related to IgSF CAM.

As mentioned above, one aspect of the invention relates to nucleic acid sequences and their derivatives which code for an isolated peptide modulator or variant thereof and other nucleic acid sequences which hybridize to a nucleic acid molecule consisting of the above described nucleotide sequences, under stringent conditions. The term “stringent conditions” as used herein refers to parameters with which the art is familiar. Nucleic acid hybridization parameters may be found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. More specifically, stringent conditions, as used herein, refers to hybridization at 65° C. in hybridization buffer (3.5×SSC, 0.02% Ficoll, 0.02% Polyvinyl pyrrolidone, 0.02% Bovine Serum Albumin, 25 mMNaH2PO4 (pH7), 0.5% SDS, 2 mM EDTA). SSC is 0.15 M Sodium Chloride/0.15 M Sodium Citrate, pH 7; SDS is Sodium Dodecyl Sulphate; and EDTA is Ethylene diaminetetraacetic acid. After hybridization, the membrane upon which the DNA is transferred is washed at 2×SSC at room temperature and then at 0.1×SSC/0.1×SDS at 65° C.

The present invention furthermore provides kits comprising the abovementioned pharmaceutical composition (in one or more containers) in at least one of the above formulations and an instruction manual or information brochure regarding instructions and/or information with respect to application of the pharmaceutical composition.

### Type-1 Angiotensin II Receptor (AT₁R) Polypeptides

The G protein-dependent signalling by AT₁R is vital for normal cardiovascular homeostasis, yet detrimental in chronic dysfunction, which associates with cell death and tissue fibrosis, and leads to cardiac hypertrophy and heart failure (Ma et al., 2010).

Despite its high medical relevance and decades of research, the structure of AT₁R and the binding mode of well established AT₁R blockers (ARBs) were only recently elucidated (Zhang et al., 2015). The structure indicated that the extracellular part of AT₁R consists of the N-terminal segment ECL1 (Glu91-Phe96 of the human AT₁R) linking helices II and III, ECL2 (His166 to Ile191 of the human AT₁R) linking helices IV and V, and ECL3 (IIe270 to Cys274 of the human AT₁R) linking helices VI to VII. Two disulphide bonds help to shape the extracellular side of AT₁R with Cys18-Cys 274 connecting the N terminus and ECL3, and Cys101-Cys180 connecting helix III and ECL2 (similar to the chemokine receptor CXCR4, which shares around 36% sequence identity with AT₁R).

In specific embodiments of the present invention, the AT₁R polypeptide comprises a AT₁R protein sequence or shares at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity or similarity with an AT₁R protein sequence.

In some embodiments, the AT₁R protein sequence corresponds to a mammalian AT1R protein sequence. Suitable AT₁R sequences may suitably be from mammal selected from the group comprising human (UniProtKB Accession No. P30556), sheep (UniProtKB Accession No. 077590), cow (UniProtKB Accession No. P25104), rabbit (UniProtKB Accession No. P34976), guinea pig (UniProtKB Accession No. Q9WV26), pig (UniProtKB Accession No. P30555), chimpanzee (UniProtKB Accession No. Q9GLN9), gerbil (UniProtKB Accession No. 035210, rat (UniProtKB Accession No. P29089), mouse (UniProtKB Accession No. P29754), cat (UniProtKB Accession No. M3VVA2), Tasmanian devil (UniProtKB Accession No. G3WOM6), horse (UniProtKB Accession No. F7D1N0), and panda (UniProtKB Accession No. D2HWD9).

In some preferred embodiments, the AT₁R protein sequence corresponds to a human AT₁R protein sequence. In some embodiments, the AT₁R polypeptide comprises a human full-length wild-type AT₁R protein sequence (UniProtKB Accession No. P30556), as set forth below, or a functional fragment of the wild-type AT₁R protein sequence.

[SEQ ID NO: 15]
MILNSSTEDGIKRIQDDCPKAGRHNYIFVMIPTLYSIIFWGIFGNSLVVI
VIYFYMKLKTVASVFLLNLALADLCFLLTLPLWAVYTAMEYRWPFGNYLC
KIASASVSFNLYASVFLLTCLSIDRYLAIVHPMKSRLRRTMLVAKVTCII
IWLLAGLASLPAIIHRNVFFIENTNITVCAFHYESQNSTLPIGLGLTKNI
LGFLFPFLIILTSYTLIWKALKKAYEIQKNKPRNDDIFKIIMAIVLFFFF
SWIPHQIFTFLDVLIQLGIIRDCRIADIVDTAMPITICIAYFNNCLNPLF
YGFLGKKFKRYFLQLLKYIPPKAKSHSNLSTKMSTLSYRPSDNVSSSTKK
PAPCFEVE.

In one form of the invention, the AT₁R polypeptide comprises a truncated form of a mammalian wild-type AT₁R protein sequence. For example, the AT₁R polypeptide sequence may comprise the human wild-type AT₁R protein sequence with a C-terminal truncation (e.g., amino acid residues 320-359 may be truncated). Alternatively or in addition, the AT₁R polypeptide sequence may comprise the wild-type AT₁R protein sequence with a N-terminal truncation. Alternatively or in addition to a C-terminal or N-terminal truncation, a truncation may be performed to remove an internal section of the wild-type AT₁R protein sequence (e.g., amino acid residues 7-16 may be truncated). By way of a non-limiting illustrative example, a AT₁R polypeptide suitable for using with the present invention comprised amino acid residues 2-6 and 17-319 of the human wild-type AT₁R protein sequence as set forth in SEQ ID NO: 15.

### Constructs and Nucleotide Sequences Encoding AT₁R Polypeptides

The present invention also encompasses isolated polynucleotide sequences and constructs encoding AT₁R polypeptides as broadly described above and elsewhere herein. Also contemplated are host cells comprising those polynucleotide sequences or constructs.

In some embodiments, the polynucleotide sequences comprise a sequence that corresponds to a human AT₁R nucleotide (i.e., corresponding to the AGTR1 gene) sequence as set forth for example in GenBank Accession Nos. KR711424.1, KR711423.1, KR711422.1, KR711421.1, KJ896399.1, KJ896398.1, NM_032049.3, NM_031850.3, NM_004835.4, NM_000685.4, NM_009585.3, DQ895601.2, BC068494.1, BCO22447.1, DQ892388.2, and AK291541.1. In representative examples of this type, the polynucleotide comprises an AT₁R nucleotide sequence that shares at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity with any one of these sequences.

In some embodiments, an AT₁R polynucleotide coding sequence comprises a nucleotide sequence that encodes a polypeptide having at least 50%, 55%, 60%, 65%, 70%, 75%, 80%, 85%, 90%, 92%, 95%, 99% or 100% sequence identity to a wild type mammalian AT₁R polynucleotide, or a fragment thereof. In some embodiments, the AT₁R polynucleotide comprises a nucleotide sequence that hybridises to an open reading frame for a wild type mammalian AT₁R protein, or a fragment thereof under low, medium or high stringency conditions.

Those skilled in the field of the invention will appreciate that the invention described herein is susceptible to variations and modifications other than those specifically described. It is to be understood that the invention includes all such functional variations and modifications. The invention also includes all of the steps, features, compositions and compounds referred to or indicated in this specification, individually or collectively, and any and all combinations or any two or more of said steps or features. The present invention is not to be limited in scope by the specific embodiments described herein, which are intended for the purpose of exemplification only. Functionally-equivalent products, compositions and methods are clearly within the scope of the invention, as described herein. Furthermore, the present invention is performed without undue experimentation using, unless otherwise indicated, conventional techniques of molecular biology, microbiology, neurobiology, virology, recombinant DNA technology, peptide synthesis in solution, solid phase peptide synthesis, and immunology, or techniques cited herein.

### REFERENCES

Ahmad, A., Bhattacharya, S., Sridhar, A., Iqbal, A. M. & Mariani, T. J. (2016) Recurrent copy number variants associated with bronchopulmonary dysplasia. Pediatric research, 79, 940-945.
Aho, V., Ollila, H. M., Rantanen, V., Kronholm, E., Surakka, I., van Leeuwen, W. M. A., Lehto, M., Matikainen, S., Ripatti, S., Harma, M., Sallinen, M., Salomaa, V., Jauhiainen, M., Alenius, H., Paunio, T. & Porkka-Heiskanen, T. (2013) Partial Sleep Restriction Activates Immune Response-Related Gene Expression Pathways: Experimental and Epidemiological Studies in Humans. PLoS ONE, 8, e77184
Alexander, S., Mathie, A. & Peter, J. (2011) Guide to Receptors and Channels (GRAC), 5th edition. Br. J. Pharmacol., 164, S1-S324.
Allende, M. L., Bektas, M., Lee, B. G., Bonifacino, E., Kang, J., Tuymetova, G., Chen, W., Saba, J. D. & Proia, R. L. (2011) Sphingosine-1-phosphate lyase deficiency produces a pro-inflammatory response while impairing neutrophil trafficking. Journal of Biological Chemistry, 286, 7348-7358.
Antoniak, S., Owens, A. P., Baunacke, M., Williams, J. C., Lee, R. D., Weithauser, A., Sheridan, P. A., Malz, R., Luyendyk, J. P. & Esserman, D. A. (2013) PAR-1 contributes to the innate immune response during viral infection. The Journal of clinical investigation, 123, 1310-1322.
Arita, M., Ohira, T., Sun, Y.-P., Elangovan, S., Chiang, N. & Serhan, C. N. (2007) Resolvin E1 selectively interacts with leukotriene B4 receptor BLT1 and ChemR23 to regulate inflammation. The Journal of Immunology, 178, 3912-3917.
Arnold Egloff S A, Du L, Loomans H A, Starchenko A, Su P F, Ketova T, Knoll P B, Wang J, Haddad A Q, Fadare O, et al. Shed urinary ALCAM is an independent prognostic biomarker of three-year overall survival after cystectomy in patients with bladder cancer. Oncotarget. 2017; 8(1):722-41.
Arnold Egloff, S. A., L. Du, H. A. Loomans, A. Starchenko, P. F. Su, T. Ketova, P. B. Knoll, J. Wang, A. Q. Haddad, O. Fadare, J. M. Cates, Y. Lotan, Y. Shyr, P. E. Clark and A. Zijlstra (2017). “Shed urinary ALCAM is an independent prognostic biomarker of three-year overall survival after cystectomy in patients with bladder cancer.” Oncotarget 8(1): 722-741.
Atukeren P, Turk O, Yanar K, Kemerdere R, Sayyahmelli S, Eren B, and Tanriverdi T. Evaluation of ALCAM, PECAM-1 and selectin levels in intracranial meningiomas. Clinical neurology and neurosurgery. 2017; 160(21-6.
Atukeren, P., O. Turk, K. Yanar, R. Kemerdere, S. Sayyahmelli, B. Eren and T. Tanriverdi (2017). “Evaluation of ALCAM, PECAM-1 and selectin levels in intracranial meningiomas.” Clin Neurol Neurosurg 160: 21-26.
Awojoodu, A. O., Ogle, M. E., Sefcik, L. S., Bowers, D. T., Martin, K., Brayman, K. L., Lynch, K. R., Peirce-Cottler, S. M. & Botchwey, E. (2013) Sphingosine 1-phosphate receptor 3 regulates recruitment of anti-inflammatory monocytes to microvessels during implant arteriogenesis. Proceedings of the National Academy of Sciences, 110, 13785-13790.
Ayer, L. M., Wilson, S. M., Traves, S. L., Proud, D. & Giembycz, M. A. (2008) 4,5-Dihydro-1H-imidazol-2-yl)-[4-(4-isopropoxy-benzyl)-phenyl]-amine (RO1138452) is a selective, pseudo-irreversible orthosteric antagonist at the prostacyclin (IP)-receptor expressed by human airway epithelial cells: IP-receptor-mediated inhibition of CXCL9 and CXCL10 release. Journal of Pharmacology and Experimental Therapeutics, 324, 815-826.
Babusyte, A., Kotthoff, M., Fiedler, J. & Krautwurst, D. (2013) Biogenic amines activate blood leukocytes via trace amine-associated receptors TAAR1 and TAAR2. Journal of leukocyte biology, 93, 387-394.
Bader, M., Alenina, N., Andrade-Navarro, M. A. & Santos, R. A. (2014) MAS and its related G protein-coupled receptors, Mrgprs. Pharmacol. Rev., 66, 1080-1105.
Ballatore C., Huryn D. M. and Smith A. B. Carboxylic Acid (Bio)Isosteres in Drug Design. ChemMedChem, 2013, 8: 385-395
Baroni, A., Perfetto, B., Canozo, N., Braca, A., Farina, E., Melito, A., De Maria, S. & Carteni, M. (2008) Bombesin: A possible role in wound repair. Peptides, 29, 1157-1166.
Bartolini, A., S. Cardaci, S. Lamba, D. Oddo, C. Marchio, P. Cassoni, C. A. Amoreo, G. Corti, A. Testori, F. Bussolino, R. Pasqualini, W. Arap, D. Cora, F. Di Nicolantonio and S. Marchio (2016). “BCAM and LAMAS Mediate the Recognition between Tumor Cells and the Endothelium in the Metastatic Spreading of KRAS-Mutant Colorectal Cancer.” Clin Cancer Res 22(19): 4923-4933.
Bathgate, R., Halls, M., Van Der Westhuizen, E., Callander, G., Kocan, M. & Summers, R. (2013) Relaxin family peptides and their receptors. Physiological reviews, 93, 405-480.
Benigni A, Coma D, Zoja C, et al. (2009) Disruption of the angiotensin II type 1 receptor promotes longevity in mice. J Clin Invest, 119: 524-530
Benya, R. V., Matkowskyj, K. A., Danilkovich, A. & Hecht, G. (1998) Galanin Causes CI—Secretion in the Human Colon: Potential Significance of Inflammation-Associated NF-κB Activation on Galanin-1 Receptor Expression and Function. Annals of the New York Academy of Sciences, 863, 64-77.
Bossard, C., Souaze, F., Jerry, A., Bezieau, S., Mosnier, J.-F., Forgez, P. & Laboisse, C. L. (2007) Over-expression of neurotensin high-affinity receptor 1 (NTS1) in relation with its ligand neurotensin (NT) and nuclear ß-catenin in inflammatory bowel disease-related oncogenesis. Peptides, 28, 2030-2035.
Boyd, J. H., Holmes, C. L., Wang, Y., Roberts, H. & Walley, K. R. (2008) Vasopressin decreases sepsis-induced pulmonary inflammation through the V2R. Resuscitation, 79, 325-331.
Brezillon, S., Lannoy, V., Franssen, J.-D., Le Poul, E., Dupriez, V., Lucchetti, J., Detheux, M. & Parmentier, M. (2003) Identification of natural ligands for the orphan G protein-coupled receptors GPR7 and GPR8. Journal of Biological Chemistry, 278, 776-783.
Briscoe, C. P., Tadayyon, M., Andrews, J. L., Benson, W. G., Chambers, J. K., Eilert, M. M., Ellis, C., Elshourbagy, N. A., Goetz, A. S. & Minnick, D. T. (2003) The orphan G protein-coupled receptor GPR40 is activated by medium and long chain fatty acids. Journal of Biological chemistry, 278, 11303-11311.
Brown, A. J., Goldsworthy, S. M., Barnes, A. A., Eilert, M. M., Tcheang, L., Daniels, D., Muir, A. I., Wigglesworth, M. J., Kinghorn, I. & Fraser, N.J. (2003) The Orphan G protein-coupled receptors GPR41 and GPR43 are activated by propionate and other short chain carboxylic acids. Journal of Biological Chemistry, 278, 11312-11319.
Bucher, M., Hobbhahn, J., Taeger, K. & Kurtz, A. (2002) Cytokine-mediated downregulation of vasopressin V1A receptors during acute endotoxemia in rats. American Journal of Physiology-Regulatory, Integrative and Comparative Physiology, 282, R979-R984.
Calonge, M., de Salamanca, A. E., Siemasko, K. F., Diebold, Y., Gao, J., Juarez-Campo, M. & Stern, M. E. (2005) Variation in the Expression of Inflammatory Markers and Neuroreceptors in Human Conjunctival Epithelial Cells. The Ocular Surface, 3, S-145-S-148.
Candido, R., et al. (2002) Prevention of accelerated atherosclerosis by angiotensin-converting enzyme inhibition in diabetic apolipoprotein E-deficient mice. Circulation, 106: 246-253.
Candido, R., et al. (2004) Irbesartan but not amlodipine suppresses diabetes-associated atherosclerosis. Circulation, 109: 1536-1542
Cantagrel, V., Lossi, A., Boulanger, S., Depetris, D., Mattei, M., Gecz, J., Schwartz, C., Van Maldergem, L. & Villard, L. (2004) Disruption of a new X linked gene highly expressed in brain in a family with two mentally retarded males. Journal of medical genetics, 41, 736-742.
Cantarella, G., Scollo, M., Lempereur, L., Saccani-Jotti, G., Basile, F. & Bernardini, R. (2011) Endocannabinoids inhibit release of nerve growth factor by inflammation-activated mast cells. Biochemical pharmacology, 82, 380-388.
Capra, V., Ravasi, S., Accomazzo, M. R., Citro, S., Grimoldi, M., Abbracchio, M. P. & Rovati, G. E. (2005) CysLT1 receptor is a target for extracellular nucleotide-induced heterologous desensitization: a possible feedback mechanism in inflammation. Journal of Cell Science, 118, 5625-5636.
Caronti, B., Calderaro, C., Passarelli, F., Palladini, G. & Pontieri, F. E. (1998) Dopamine receptor mRNAs in the rat lymphocytes. Life sciences, 62, 1919-1925.
Carrillo-Vico, A., GARCIA, S., Calvo, J. R. & Guerrero, J. M. (2003) Melatonin counteracts the inhibitory effect of PGE2 on IL-2 production in human lymphocytes via its mt1 membrane receptor. The FASEB Journal, 17, 755-757.
Caruso, C., Durand, D., Schioth, H. B., Rey, R., Seilicovich, A. & Lasaga, M. (2007) Activation of melanocortin 4 receptors reduces the inflammatory response and prevents apoptosis induced by lipopolysaccharide and interferon-gamma in astrocytes. Endocrinology, 148, 4918-4926.
Chen, H. F., Jeung, E. B., Stephenson, M. & Leung, P. C. (1999) Human peripheral blood mononuclear cells express gonadotropin-releasing hormone (GnRH), GnRH receptor, and interleukin-2 receptor gamma-chain messenger ribonucleic acids that are regulated by GnRH in vitro. The Journal of clinical endocrinology and metabolism, 84, 743-750.
Chen, T.-Y., Hwang, T.-L., Lin, C.-Y., Lin, T.-N., Lai, H.-Y., Tsai, W.-P. & Lin, H.-H. (2011) EMR2 receptor ligation modulates cytokine secretion profiles and cell survival of lipopolysaccharide-treated neutrophils. Chang Gung Med J, 34, 468-477.
Chen, Y., Corriden, R., Inoue, Y., Yip, L., Hashiguchi, N., Zinkernagel, A., Nizet, V., Insel, P. A. & Junger, W. G. (2006) ATP release guides neutrophil chemotaxis via P2Y2 and A3 receptors. Science, 314, 1792-1795.
Chhajlani, V. (1996) Distribution of cDNA for melanocortin receptor subtypes in human tissues. Biochemistry and molecular biology international, 38, 73-80.
Colomb, F., W. Wang, D. Simpson, M. Zafar, R. Beynon, J. M. Rhodes and L. G. Yu (2017). “Galectin-3 interacts with the cell-surface glycoprotein CD146 (MCAM, MUC18) and induces secretion of metastasis-promoting cytokines from vascular endothelial cells.” J Biol Chem 292(20): 8381-8389.
Consortium, I.G.o.A.S. (2013) Identification of multiple risk variants for ankylosing spondylitis through high-density genotyping of immune-related loci. Nature genetics, 45, 730-738.
Cook, I. H., Evans, J., Maldonado-Perez, D., Critchley, H. O., Sales, K. J. &Jabbour, H. N. (2010) Prokineticin (PROK1) modulates interleukin (IL)-11 expression via prokineticin receptor 1 (PROKR1) and the calcineurin/NFAT signalling pathway. Molecular human reproduction, 16, 158-169.
Cuddihy, R. M., Dutton, C. M. & Bahn, R. S. (1995) A polymorphism in the extracellular domain of the thyrotropin receptor is highly associated with autoimmune thyroid disease in females. Thyroid, 5, 89-95.
Czepielewski, R. S., Porto, B. N., Rizzo, L. B., Roesler, R., Abujamra, A. L., Pinto, L. G., Schwartsmann, G., de Queiroz Cunha, F. & Bonorino, C. (2012) Gastrin-releasing peptide receptor (GRPR) mediates chemotaxis in neutrophils. Proceedings of the National Academy of Sciences, 109, 547-552.
D'Amato, M., Bruce, S., Bresso, F., Zucchelli, M., Ezer, S., Pulkkinen, V., Lindgren, C., Astegiano, M., Rizzetto, M. & Gionchetti, P. (2007) Neuropeptide s receptor 1 gene polymorphism is associated with susceptibility to inflammatory bowel disease. Gastroenterology, 133, 808-817.
D'Andrea, G., D'Arrigo, A., Facchinetti, F., Del Giudice, E., Colavito, D., Bernardini, D. & Leon, A. (2012) Octopamine, unlike other trace amines, inhibits responses of astroglia-enriched cultures to lipopolysaccharide via a β-adrenoreceptor-mediated mechanism. Neuroscience letters, 517, 36-40.
da Silveira, K. D., Coelho, F. M., Vieira, A. T., Sachs, D., Barroso, L. C., Costa, V. V., Bretas, T. L. B., Bader, M., de Sousa, L. P. & da Silva, T. A. (2010) Anti-inflammatory effects of the activation of the angiotensin-(1-7) receptor, MAS, in experimental models of arthritis. The Journal of Immunology, 185, 5569-5576.
D'Andrea, G., Terrazzino, S., Fortin, D., Farruggio, A., Rinaldi, L. & Leon, A. (2003) HPLC electrochemical detection of trace amines in human plasma and platelets and expression of mRNA transcripts of trace amine receptors in circulating leukocytes. Neuroscience letters, 346, 89-92.
Davidson, C., Asaduzzaman, M., Arizmendi, N., Polley, D., Wu, Y., Gordon, J., Hollenberg, M., Cameron, L. & Vliagoftis, H. (2013) Proteinase-activated receptor-2 activation participates in allergic sensitization to house dust mite allergens in a murine model. Clinical & Experimental Allergy, 43, 1274-1285.
De Grandis, M., B. Cassinat, J. J. Kiladjian, C. Chomienne and W. El Nemer (2015). “Lu/BCAM-mediated cell adhesion as biological marker of JAK2V617F activity in erythrocytes of polycythemia vera patients.” Am J Hematol 90(7): E137-138.
Dixit, V. D., Schaffer, E. M., Pyle, R. S., Collins, G. D., Sakthivel, S. K., Palaniappan, R., Lillard, J. W. & Taub, D. D. (2004) Ghrelin inhibits leptin- and activation-induced proinflammatory cytokine expression by human monocytes and T cells. The Journal of clinical investigation, 114, 57-66.
Dorsch, M., Qiu, Y., Soler, D., Frank, N., Duong, T., Goodearl, A., O'Neil, S., Lora, J. & Fraser, C. C. (2005) PK1/EG-VEGF induces monocyte differentiation and activation. Journal of Leukocyte Biology, 78, 426-434.
Drazen, D. L. & Nelson, R. J. (2001) Melatonin receptor subtype MT2 (Mel 1b) and not mt1 (Mel 1a) is associated with melatonin-induced enhancement of cell-mediated and humoral immunity. Neuroendocrinology, 74, 178-184.
Ehrenfeld, P., Millan, C., Matus, C., Figueroa, J., Burgos, R., Nualart, F., Bhoola, K. & Figueroa, C. (2006) Activation of kinin B1 receptors induces chemotaxis of human neutrophils. Journal of leukocyte biology, 80, 117-124.
Ekholm, M., Kahan, T., Jorneskog, G., Broijersen, A. & Wallen, N. H. (2009) Angiotensin II infusion in man is proinflammatory but has no short-term effects on thrombin generation in vivo. Thromb Res, 124: 110-115.
El Nemer, W., P. Gane, Y. Colin, V. Bony, C. Rahuel, F. Galacteros, J. P. Cartron and C. Le Van Kim (1998). “The Lutheran blood group glycoproteins, the erythroid receptors for laminin, are adhesion molecules.” J Biol Chem 273(27): 16686-16693.
Elliott, S. E., Parchim, N. F., Kellems, R. E., Xia, Y., Soffici, A. R. & Daugherty, P. S. (2016) A pre-eclampsia-associated Epstein-Barr virus antibody cross-reacts with placental GPR50. Clinical Immunology, 168, 64-71.
Engel, K. M., Schröck, K., Teupser, D., Holdt, L. M., Tonjes, A., Kern, M., Dietrich, K., Kovacs, P., Krugel, U. & Scheidt, H. A. (2011) Reduced food intake and body weight in mice deficient for the G protein-coupled receptor GPR82. PLoS One, 6, e29400.
Farzan, M., Choe, H., Martin, K., Marcon, L., Hofmann, W., Karlsson, G., Sun, Y., Barrett, P., Marchand, N. & Sullivan, N. (1997) Two orphan seven-transmembrane segment receptors which are expressed in CD4-positive cells support simian immunodeficiency virus infection. The Journal of experimental medicine, 186, 405-411.
Ferreira, M., Barcelos, L. S., Campos, P. P., Vasconcelos, A. C., Teixeira, M. M. & Andrade, S. P. (2004) Sponge-induced angiogenesis and inflammation in PAF receptor-deficient mice (PAFR-KO). British journal of pharmacology, 141, 1185-1192.
Fleischmann, A., Läderach, U., Friess, H., Buechler, M. W. & Reubi, J. C. (2000) Bombesin receptors in distinct tissue compartments of human pancreatic diseases. Laboratory investigation, 80, 1807-1817.
Fornari, T. A., Donate, P. B., Macedo, C., Sakamoto-Hojo, E. T., Donadi, E. A. & Passos, G. A. (2011) Development of type 1 diabetes mellitus in nonobese diabetic mice follows changes in thymocyte and peripheral T lymphocyte transcriptional activity. Clinical and Developmental Immunology, 2011.
Foster, H. R., Fuerst, E., Branchett, W., Lee, T. H., Cousins, D. J. & Woszczek, G. (2016) Leukotriene E4 is a full functional agonist for human cysteinyl leukotriene type 1 receptor-dependent gene expression. Scientific reports, 6.
Frasch, S. C., Berry, K. Z., Fernandez-Boyanapalli, R., Jin, H.-S., Leslie, C., Henson, P. M., Murphy, R. C. & Bratton, D. L. (2008) NADPH oxidase-dependent generation of lysophosphatidylserine enhances clearance of activated and dying neutrophils via G2A. Journal of Biological Chemistry, 283, 33736-33749.
Freire-Garabal, M., Nunez, M., Balboa, J., López-Delgado, P., Gallego, R., García-Caballero, T., Fern á ndez-Roel, M., Brenlla, J. & Rey-Méndez, M. (2003) Serotonin upregulates the activity of phagocytosis through 5-HT1A receptors. British journal of pharmacology, 139, 457-463.
Fujita, T., Matsuoka, T., Honda, T., Kabashima, K., Hirata, T. & Narumiya, S. (2011) A GPR40 agonist GW9508 suppresses CCL5, CCL17, and CXCL10 induction in keratinocytes and attenuates cutaneous immune inflammation. Journal of Investigative Dermatology, 131, 1660-1667.
Fujita, T., Tozaki-Saitoh, H. & Inoue, K. (2009) P2Y1 receptor signalling enhances neuroprotection by astrocytes against oxidative stress via IL-6 release in hippocampal cultures. Glia, 57, 244-257.
Galiègue, S., Mary, S., Marchand, J., Dussossoy, D., Carrière, D., Carayon, P., Bouaboula, M., Shire, D., Le Fur, G. & Casellas, P. (1995) Expression of Central and Peripheral Cannabinoid Receptors in Human Immune Tissues and Leukocyte Subpopulations. European Journal of Biochemistry, 232, 54-61.
Gantz, I., Muraoka, A., Yang, Y.-K., Samuelson, L. C., Zimmerman, E. M., Cook, H. & Yamada, T. (1997) Cloning and chromosomal localization of a gene (GPR18) encoding a novel seven transmembrane receptor highly expressed in spleen and testis. Genomics, 42, 462-466.
Gao, Z.-G., Ding, Y. & Jacobson, K. A. (2010) P2Y 13 receptor is responsible for ADP-mediated degranulation in RBL-2H3 rat mast cells. Pharmacological research, 62, 500-505.
Gatto, D., Wood, K. & Brink, R. (2011) EB12 operates independently of but in cooperation with CXCR5 and CCR7 to direct B cell migration and organization in follicles and the germinal center. The Journal of Immunology, 187, 4621-4628.
Gaveriaux, C., Peluso, J., Simonin, F., Laforet, J. & Kieffer, B. (1995) Identification of kappa- and delta-opioid receptor transcripts in immune cells. FEBS Lett, 369, 272-276.
Gazel, A., Rosdy, M., Bertino, B., Tornier, C., Sahuc, F. & Blumenberg, M. (2006) A characteristic subset of psoriasis-associated genes is induced by oncostatin-M in reconstituted epidermis. Journal of investigative dermatology, 126, 2647-2657.
Gervais, F. G., Cruz, R. P., Chateauneuf, A., Gale, S., Sawyer, N., Nantel, F., Metters, K. M. & O'Neill, G. P. (2001) Selective modulation of chemokinesis, degranulation, and apoptosis in eosinophils through the PGD 2 receptors CRTH2 and DP. Journal of Allergy and Clinical Immunology, 108, 982-988.
Getting, S. J., Gibbs, L., Clark, A. J., Flower, R. J. & Perretti, M. (1999) POMC gene-derived peptides activate melanocortin type 3 receptor on murine macrophages, suppress cytokine release, and inhibit neutrophil migration in acute experimental inflammation. The Journal of Immunology, 162, 7446-7453.
Giannini, E., Lattanzi, R., Nicotra, A., Campese, A. F., Grazioli, P., Screpanti, I., Balboni, G., Salvadori, S., Sacerdote, P. & Negri, L. (2009) The chemokine Bv8/prokineticin 2 is up-regulated in inflammatory granulocytes and modulates inflammatory pain. Proceedings of the National Academy of Sciences, 106, 14646-14651.
Grafe, M., et al. (1997) Angiotensin II-induced leukocyte adhesion on human coronary endothelial cells is mediated by E-selectin. Circ Res, 81: 804-811.
Granados-Soto, V., Arguelles, C. F., Rocha-Gonzalez, H. I., Godinez-Chaparro, B., Flores-Murrieta, F. J. & Villalón, C. M. (2010) The role of peripheral 5-HT1A, 5-HT1B, 5-HT1D, 5-HT1E and 5-HT1F serotonergic receptors in the reduction of nociception in rats. Neuroscience, 165, 561-568.
Grässel, S., Opolka, A., Anders, S., Straub, R. H., Grifka, J., Luger, T. A. & Bohm, M. (2009) The melanocortin system in articular chondrocytes: Melanocortin receptors, pro-opiomelanocortin, precursor proteases, and a regulatory effect of α-melanocyte-stimulating hormone on proinflammatory cytokines and extracellular matrix components. Arthritis & Rheumatism, 60, 3017-3027.
Greene T. W. et al. Protective groups in organic synthesis, 1991, Wiley, New York.
Guerraty M A, Grant G R, Karanian J W, Chiesa O A, Pritchard W F, and Davies P F. Side-specific expression of activated leukocyte adhesion molecule (ALCAM; CD166) in pathosusceptible regions of swine aortic valve endothelium. The Journal of heart valve disease. 2011; 20(2):165-7.
Guerraty, M. A., G. R. Grant, J. W. Karanian, O. A. Chiesa, W. F. Pritchard and P. F. Davies (2011). “Side-specific expression of activated leukocyte adhesion molecule (ALCAM; CD166) in pathosusceptible regions of swine aortic valve endothelium.” J Heart Valve Dis 20(2): 165-167.
Guezguez B, et al. FEBS Lett. (2006) A dileucine motif targets MCAM-I cell adhesion molecule to the basolateral membrane in MDCK cells. FEBS Lett. 2006 Jun. 26; 580(15):3649-56.
Guezguez, B., Vigneron, P., Alais, S., Jaffredo, T., Gavard, J., Mège, R. M. and Dunon, D. (2006). “A dileucine motif targets MCAM-I cell adhesion molecule to the basolateral membrane in MDCK cells.” FEBS Lett 580(15): 3649-3656.
Hansen A G, Arnold S A, Jiang M, Palmer T D, Ketova T, Merkel A, Pickup M, Samaras S, Shyr Y, Moses H L, et al. ALCAM/CD166 is a TGF-beta-responsive marker and functional regulator of prostate cancer metastasis to bone. Cancer research. 2014; 74(5):1404-15.
Hansen, A. G., S. A. Arnold, M. Jiang, T. D. Palmer, T. Ketova, A. Merkel, M. Pickup, S. Samaras, Y. Shyr, H. L. Moses, S. W. Hayward, J. A. Sterling and A. Zijlstra (2014). “ALCAM/CD166 is a TGF-beta-responsive marker and functional regulator of prostate cancer metastasis to bone.” Cancer Res 74(5): 1404-1415.
Hansen, W., Westendorf, A., Toepfer, T., Mauel, S., Geffers, R., Gruber, A. & Buer, J. (2010) Inflammation in vivo is modulated by GPR83 isoform-4 but not GPR83 isoform-1 expression in regulatory T cells. Genes and immunity, 11, 357-361.
Hartmann, K., Henz, B. M., Krüger-Krasagakes, S., Köhl, J., Burger, R., Guhl, S., Haase, I., Lippert, U. & Zuberbier, T. (1997) C3a and C5a stimulate chemotaxis of human mast cells. Blood, 89, 2863-2870.
Hartmeyer, M., Scholzen, T., Becher, E., Bhardwaj, R., Schwarz, T. & Luger, T. (1997) Human dermal microvascular endothelial cells express the melanocortin receptor type 1 and produce increased levels of IL-8 upon stimulation with alpha-melanocyte-stimulating hormone. The Journal of Immunology, 159, 1930-1937.
Haworth, O., Cernadas, M. & Levy, B. D. (2011) NK cells are effectors for resolvin E1 in the timely resolution of allergic airway inflammation. The Journal of Immunology, 186, 6129-6135.
Hebron K E, Li E Y, Arnold Egloff S A, von Lersner A K, Taylor C, Houkes J, Flaherty D K, Eskaros A, Stricker T P, and Zijlstra A. Alternative splicing of ALCAM enables tunable regulation of cell-cell adhesion through differential proteolysis. Scientific reports. 2018; 8(1):3208.
Hebron, K. E., E. Y. Li, S. A. Arnold Egloff, A. K. von Lersner, C. Taylor, J. Houkes, D. K. Flaherty, A. Eskaros, T. P. Stricker and A. Zijlstra (2018). “Alternative splicing of ALCAM enables tunable regulation of cell-cell adhesion through differential proteolysis.” Sci Rep 8(1): 3208.
Hess B, Kutzner C, Van Der Spoel D, Lindahl E. GROMACS 4: Algorithms for highly efficient, load-balanced, and scalable molecular simulation. J Chem Theory Comput, 2008, 4: 435
Heublein, S., Lenhard, M., Vrekoussis, T., Schoepfer, J., Kuhn, C., Friese, K., Makrigiannakis, A., Mayr, D. & Jeschke, U. (2012) The G-protein-coupled estrogen receptor (GPER) is expressed in normal human ovaries and is upregulated in ovarian endometriosis and pelvic inflammatory disease involving the ovary. Reproductive Sciences, 19, 1197-1204.
Hill, J., Duckworth, M., Murdock, P., Rennie, G., Sabido-David, C., Ames, R. S., Szekeres, P., Wilson, S., Bergsma, D. J. & Gloger, I. S. (2001) Molecular cloning and functional characterization of MCH2, a novel human MCH receptor. Journal of Biological Chemistry, 276, 20125-20129.
Hong X, Michalski C W, Kong B, Zhang W, Raggi M C, Sauliunaite D, De Oliveira T, Friess H, and Kleeff J. ALCAM is associated with chemoresistance and tumor cell adhesion in pancreatic cancer. Journal of surgical oncology. 2010; 101(7):564-9.
Hong, K. W., Shin, M. S., Ahn, Y. B., Lee, H. J. & Kim, H. D. (2015) Genomewide association study on chronic periodontitis in Korean population: results from the Yangpyeong health cohort. Journal of clinical periodontology, 42, 703-710.
Hong, X., C. W. Michalski, B. Kong, W. Zhang, M. C. Raggi, D. Sauliunaite, T. De Oliveira, H. Friess and J. Kleeff (2010). “ALCAM is associated with chemoresistance and tumor cell adhesion in pancreatic cancer.” J Surg Oncol 101(7): 564-569.
Hogue, R., Farooq, A., Ghani, A., Gorelick, F. & Mahal, W. Z. (2014) Lactate reduces liver and pancreatic injury in Toll-like receptor- and inflammasome-mediated inflammation via GPR81-mediated suppression of innate immunity. Gastroenterology, 146, 1763-1774.
Horton, J., Yamamoto, S. & Bryant-Greenwood, G. (2012) Relaxin augments the inflammatory IL6 response in the choriodecidua. Placenta, 33, 399-407.
Huang, J., A. Filipe, C. Rahuel, P. Bonnin, L. Mesnard, C. Guerin, Y. Wang, C. Le Van Kim, Y. Colin and P. L. Tharaux (2014). “Lutheran/basal cell adhesion molecule accelerates progression of crescentic glomerulonephritis in mice.” Kidney Int 85(5): 1123-1136.
Ichimonji, I., Tomura, H., Mogi, C., Sato, K., Aoki, H., Hisada, T., Dobashi, K., Ishizuka, T., Mori, M. & Okajima, F. (2010) Extracellular acidification stimulates IL-6 production and Ca2+ mobilization through proton-sensing OGR1 receptors in human airway smooth muscle cells. American Journal of Physiology-Lung Cellular and Molecular Physiology, 299, L567-L577.
Ignatov, A., Robert, J., Gregory-Evans, C. & Schaller, H. (2006) RANTES stimulates Ca2+ mobilization and inositol trisphosphate (IP3) formation in cells transfected with G protein-coupled receptor 75. British journal of pharmacology, 149, 490-497.
Improta, G., Carpino, F., Petrozza, V., Guglietta, A., Tabacco, A. & Broccardo, M. (2003) Central effects of selective NK 1 and NK 3 tachykinin receptor agonists on two models of experimentally-induced colitis in rats. Peptides, 24, 903-911.
Inaguma S, Lasota J, Wang Z, Czapiewski P, Langfort R, Rys J, Szpor J, Waloszczyk P, Okon K, Biernat W, et al. Expression of ALCAM (CD166) and PD-L1 (CD274) independently predicts shorter survival in malignant pleural mesothelioma. Human pathology. 2018; 71(1-7.
Inaguma, S., J. Lasota, Z. Wang, P. Czapiewski, R. Langfort, J. Rys, J. Szpor, P. Waloszczyk, K. Okon, W. Biernat, H. Ikeda, D. S. Schrump, R. Hassan and M. Miettinen (2018). “Expression of ALCAM (CD166) and PD-L1 (CD274) independently predicts shorter survival in malignant pleural mesothelioma.” Hum Pathol 71: 1-7.
Inbe, H., Watanabe, S., Miyawaki, M., Tanabe, E. & Encinas, J. A. (2004) Identification and characterization of a cell-surface receptor, P2Y15, for AMP and adenosine. Journal of Biological Chemistry, 279, 19790-19799.
Irukayama-Tomobe, Y., Tanaka, H., Yokomizo, T., Hashidate-Yoshida, T., Yanagisawa, M. & Sakurai, T. (2009) Aromatic D-amino acids act as chemoattractant factors for human leukocytes through a G protein-coupled receptor, GPR109B. Proceedings of the National Academy of Sciences, 106, 3930-3934.
işeri, S. Ö., Şener, G., Sa\check{g}lam, B., Gedik, N., Ercan, F. & Ye\check{g}en, B.Ç. (2005) Oxytocin ameliorates oxidative colonic inflammation by a neutrophil-dependent mechanism. Peptides, 26, 483-491.
Ishihara, H., Connolly, A. J., Zeng, D., Kahn, M. L., Zheng, Y. W., Timmons, C., Tram, T. & Coughlin, S. R. (1997) Protease-activated receptor 3 is a second thrombin receptor in humans.
Iwasa, T., Matsuzaki, T., Tungalagsuvd, A., Munkhzaya, M., Kawami, T., Niki, H., Kato, T., Kuwahara, A., Uemura, H., Yasui, T. & Irahara, M. (2014) Hypothalamic Kiss1 and RFRP gene expressions are changed by a high dose of lipopolysaccharide in female rats. Hormones and Behavior, 66, 309-316.
Izeboud, C. A., Vermeulen, R. M., Zwart, A., Voss, H.-P., van Miert, A. S. J. P. A. M. & Witkamp, R. F. (2000) Stereoselectivity at the β2-adrenoceptor on macrophages is a major determinant of the anti-inflammatory effects of β2-agonists. Naunyn-Schmiedeberg's Archives of Pharmacology, 362, 184-189.
Jacoby, D. S., and Rader, D. J. (2003) Renin-angiotensin system and atherothrombotic disease: from genes to treatment. Arch Intern Med, 163: 1155-64
Jaeger, W. C., Armstrong, S. P., Hill, S. J. and Pfleger, K. D. G., Biophysical detection of diversity and bias in GPCR function. Front Endocrinol, 2014, 5: 26.
Jaffré, F., Bonnin, P., Callebert, J., Debbabi, H., Setola, V., Doly, S., Monassier, L., Mettauer, B., Blaxall, B. C. & Launay, J.-M. (2009) Serotonin and angiotensin receptors in cardiac fibroblasts coregulate adrenergic-dependent cardiac hypertrophy. Circulation research, 104, 113-123.
Jenne, C. N., Enders, A., Rivera, R., Watson, S. R., Bankovich, A. J., Pereira, J. P., Xu, Y., Roots, C. M., Beilke, J. N. & Banerjee, A. (2009) T-bet-dependent S1P5 expression in N K cells promotes egress from lymph nodes and bone marrow. The Journal of experimental medicine, 206, 2469-2481.
Jimenez-Andrade, J. M., Zhou, S., Du, J., Yamani, A., Grady, J. J., Castalieda-Hernandez, G. & Carlton, S. M. (2004) Pro-nociceptive role of peripheral galanin in inflammatory pain. Pain, 110, 10-21.
Johns, D. G., Ao, Z., Naselsky, D., Herold, C. L., Maniscalco, K., Sarov-Blat, L., Steplewski, K., Aiyar, N. & Douglas, S. A. (2004) Urotensin-II-mediated cardiomyocyte hypertrophy: effect of receptor antagonism and role of inflammatory mediators. Naunyn-Schmiedeberg's archives of pharmacology, 370, 238-250.
Johnson, J. P. (1999). “Cell adhesion molecules in the development and progression of malignant melanoma.” Cancer Metastasis Rev 18(3): 345-357.
Jossart, C., Mulumba, M., Granata, R., Gallo, D., Ghigo, E., Marleau, S., Servant, M. J. & Ong, H. (2013) Pyroglutamylated RF-amide peptide (QRFP) gene is regulated by metabolic endotoxemia. Molecular Endocrinology, 28, 65-79.
Jouve, N., R. Bachelier, N. Despoix, M. G. Blin, M. K. Matinzadeh, S. Poitevin, M. Aurrand-Lions, K. Fallague, N. Bardin, M. Blot-Chabaud, F. Vely, F. Dignat-George and A. S. Leroyer (2015). “CD146 mediates VEGF-induced melanoma cell extravasation through FAK activation.” Int J Cancer 137(1): 50-60.
Jules J, Maiguel D, Hudson B I, Alternative Splicing of the RAGE Cytoplasmic Domain Regulates Cell Signalling and Function. PLoS ONE, 2013, 8: e78267.
Jurisic, G., Sundberg, J., Bleich, A., Leiter, E., Broman, K., Buechler, G., Alley, L., Vestweber, D. & Detmar, M. (2010) Quantitative lymphatic vessel trait analysis suggests Vcam1 as candidate modifier gene of inflammatory bowel disease. Genes and immunity, 11, 219-231.
Kabashima, K., Saji, T., Murata, T., Nagamachi, M., Matsuoka, T., Segi, E., Tsuboi, K., Sugimoto, Y., Kobayashi, T. & Miyachi, Y. (2002) The prostaglandin receptor EP4 suppresses colitis, mucosal damage and CD4 cell activation in the gut. The Journal of clinical investigation, 109, 883-893.
Kanazawa, M., Watanabe, S., Tana, C., Komuro, H., Aoki, M. & Fukudo, S. (2011) Effect of 5-HT4 receptor agonist mosapride citrate on rectosigmoid sensorimotor function in patients with irritable bowel syndrome. Neurogastroenterology & Motility, 23, 754-e332.
Kawamata, Y., Fujii, R., Hosoya, M., Harada, M., Yoshida, H., Miwa, M., Fukusumi, S., Habata, Y., Itoh, T. & Shintani, Y. (2003) AG protein-coupled receptor responsive to bile acids. Journal of Biological Chemistry, 278, 9435-9440.
Kazemian, P., Kazemi-Bajestani, S. M., Alherbish, A., Steed, J. & Oudit, G. Y. (2012) The use of ω-3 poly-unsaturated fatty acids in heart failure: a preferential role in patients with diabetes. Cardiovascular drugs and therapy, 26, 311-320.
Keermann, M., Köks, S., Reimann, E., Prans, E., Abram, K. & Kingo, K. (2015) Transcriptional landscape of psoriasis identifies the involvement of IL36 and IL36RN. BMC genomics, 16, 1.
Kikkawa, Y., T. Ogawa, R. Sudo, Y. Yamada, F. Katagiri, K. Hozumi, M. Nomizu and J. H. Miner (2013). “The lutheran/basal cell adhesion molecule promotes tumor cell migration by modulating integrin-mediated cell attachment to laminin-511 protein.” J Biol Chem 288(43): 30990-31001.
Kim M N, Hong J Y, Shim D H, Sol I S, Kim Y S, Lee J H, Kim K W, Lee J M, and Sohn M H. Activated Leukocyte Cell Adhesion Molecule Stimulates the T-Cell Response in Allergic Asthma. American journal of respiratory and critical care medicine. 2018; 197(8):994-1008.
Kim Y S, Kim M N, Lee K E, Hong J Y, Oh M S, Kim S Y, Kim K W, and Sohn M H. Activated leucocyte cell adhesion molecule (ALCAM/CD166) regulates T cell responses in a murine model of food allergy. Clinical and experimental immunology. 2018; 192(2):151-64.
Kim, M. N., J. Y. Hong, D. H. Shim, I. S. Sol, Y. S. Kim, J. H. Lee, K. W. Kim, J. M. Lee and M. H. Sohn (2018). “Activated Leukocyte Cell Adhesion Molecule Stimulates the T-Cell Response in Allergic Asthma.” Am J Respir Crit Care Med 197(8): 994-1008.
Kim, S. V., Xiang, W. V., Kwak, C., Yang, Y., Lin, X. W., Ota, M., Sarpel, U., Rifkin, D. B., Xu, R. & Littman, D. R. (2013) GPR15-mediated homing controls immune homeostasis in the large intestine mucosa. Science, 340, 1456-1459
Kim, Y. S., M. N. Kim, K. E. Lee, J. Y. Hong, M. S. Oh, S. Y. Kim, K. W. Kim and M. H. Sohn (2018). “Activated leucocyte cell adhesion molecule (ALCAM/CD166) regulates T cell responses in a murine model of food allergy.” Clin Exp Immunol 192(2): 151-164.
Knowles, J. W., et al. (2000) Enhanced atherosclerosis and kidney dysfunction in eNOS(−/−) ApoE(−/−) mice are ameliorated by enalapril treatment. J Clin Invest, 105: 451-458
Kottyan, L. C., Collier, A. R., Cao, K. H., Niese, K. A., Hedgebeth, M., Radu, C. G., Witte, O. N., Hershey, G. K. K., Rothenberg, M. E. & Zimmermann, N. (2009) Eosinophil viability is increased by acidic pH in a cAMP- and GPR65-dependent manner. Blood, 114, 2774-2782.
Kozovska Z, Gabrisova V, and Kucerova L. Colon cancer: cancer stem cells markers, drug resistance and treatment. Biomedicine & pharmacotherapy=Biomedecine & pharmacotherapie. 2014; 68(8):911-6.
Kozovska, Z., V. Gabrisova and L. Kucerova (2014). “Colon cancer: cancer stem cells markers, drug resistance and treatment.” Biomed Pharmacother 68(8): 911-916.
Krishnamoorthy, S., Recchiuti, A., Chiang, N., Fredman, G. & Serhan, C. N. (2012) Resolvin D1 receptor stereoselectivity and regulation of inflammation and proresolving microRNAs. The American journal of pathology, 180, 2018-2027.
Krishnamoorthy, S., Recchiuti, A., Chiang, N., Yacoubian, S., Lee, C.-H., Yang, R., Petasis, N. A. & Serhan, C. N. (2010) Resolvin D1 binds human phagocytes with evidence for proresolving receptors. Proceedings of the National Academy of Sciences, 107, 1660-1665.
Kunikata, T., Yamane, H., Segi, E., Matsuoka, T., Sugimoto, Y., Tanaka, S., Tanaka, H., Nagai, H., Ichikawa, A. & Narumiya, S. (2005) Suppression of allergic inflammation by the prostaglandin E receptor subtype EP3. Nature immunology, 6, 524-531.
Kupp, L. I., Kosco, M. H., Schenkein, H. A. & Tew, J. G. (1991) Chemotaxis of germinal centers B cells in response to C5a. European journal of immunology, 21, 2697-2701.
Kwon, J. Y., Park, M. K., Seo, Y. R. & Song, J.-J. (2014) Genomic-based identification of novel potential biomarkers and molecular signalling networks in response to diesel exhaust particles in human middle ear epithelial cells. Molecular & Cellular Toxicology, 10, 95-105.
Lafrance, M., Roussy, G., Belleville, K., Maeno, H., Beaudet, N., Wada, K. & Sarret, P. (2010) Involvement of NTS2 receptors in stress-induced analgesia. Neuroscience, 166, 639-652.
Laird, J. M., Oliver, T., Lopez-Garcia, J. A., Maggi, C. A. & Cervero, F. (2001) Responses of rat spinal neurons to distension of inflamed colon: role of tachykinin NK2 receptors. Neuropharmacology, 40, 696-701.
Lamas, O., Martinez, J. A. & Marti, A. (2003) Effects of a β3-adrenergic agonist on the immune response in diet-induced (cafeteria) obese animals. Journal of Physiology and Biochemistry, 59, 183-191.
Lattin, J. E., Schroder, K., Su, A. I., Walker, J. R., Zhang, J., Wiltshire, T., Saijo, K., Glass, C. K., Hume, D. A. & Kellie, S. (2008) Expression analysis of G Protein-Coupled Receptors in mouse macrophages. Immunome research, 4, 1.
Laukova, M., Vargovic, P., Krizanova, O. & Kvetnansky, R. (2010) Repeated Stress Down-Regulates β2- and α2C-Adrenergic Receptors and Up-Regulates Gene Expression of IL-6 in the Rat Spleen. Cellular and Molecular Neurobiology, 30, 1077-1087.
Lazennec, G. & Richmond, A. (2010) Chemokines and chemokine receptors: new insights into cancer-related inflammation. Trends in molecular medicine, 16, 133-144.
Le Poul, E., Loison, C., Struyf, S., Springael, J.-Y., Lannoy, V., Decobecq, M.-E., Brezillon, S., Dupriez, V., Vassart, G. & Van Damme, J. (2003) Functional characterization of human receptors for short chain fatty acids and their role in polymorphonuclear cell activation. Journal of Biological Chemistry, 278, 25481-25489.
Le, Y., Gong, W., Li, B., Dunlop, N. M., Shen, W., Su, S. B., Richard, D. Y. & Wang, J. M. (1999) Utilization of two seven-transmembrane, G protein-coupled receptors, formyl peptide receptor-like 1 and formyl peptide receptor, by the synthetic hexapeptide WKYMVm for human phagocyte activation. The Journal of Immunology, 163, 6777-6784.
Lecuyer M A, Saint-Laurent O, Bourbonniere L, Larouche S, Larochelle C, Michel L, Charabati M, Abadier M, Zandee S, Haghayegh Jahromi N, et al. Dual role of ALCAM in neuroinflammation and blood-brain barrier homeostasis. Proceedings of the National Academy of Sciences of the United States of America. 2017; 114(4):E524-E33.
Lecuyer, M. A., O. Saint-Laurent, L. Bourbonniere, S. Larouche, C. Larochelle, L. Michel, M. Charabati, M. Abadier, S. Zandee, N. Haghayegh Jahromi, E. Cowing, C. Pittet, R. Lyck, B. Engelhardt and A. Prat (2017). “Dual role of ALCAM in neuroinflammation and blood-brain barrier homeostasis.” Proc Natl Acad Sci USA 114(4): E524-E533.
Lee, B.-C., Cheng, T., Adams, G. B., Attar, E. C., Miura, N., Lee, S. B., Saito, Y., Olszak, I., Dombkowski, D. & Olson, D. P. (2003) P2Y-like receptor, GPR105 (P2Y14), identifies and mediates chemotaxis of bone-marrowhematopoietic stem cells. Genes & development, 17, 1592-1604.
Lee, B.-Y., Cho, S., Shin, D. H. & Kim, H. (2011) Genome-wide association study of copy number variations associated with pulmonary function measures in Korea Associated Resource (KARE) cohorts. Genomics, 97, 101-105.
Lee, M. A., Bohm, M., Paul, M., and Ganten, D. (1993) Tissue renin-angiotensin systems. Their role in cardiovascular disease. Circulation, 87: IV7-13
Levite, M., Chowers, Y., Ganor, Y., Besser, M., Hershkovits, R. & Cahalon, L. (2001) Dopamine interacts directly with its D3 and D2 receptors on normal human T cells, and activates β1 integrin function. European journal of immunology, 31, 3504-3512.
Li C., Pazgier M., Li J., Li C., Liu M., Zou G., Li Z., Chen J., Tarasov S. G., Lu W. Y., Lu W. Limitations of peptide retro-inverso isomerization in molecular mimicry. J Biol Chem, 2010, 285: 19572-19581
Li X. C., and Zhuo J, L. (2008) Nuclear factor-kappaB as a hormonal intracellular signalling molecule: focus on angiotensin II-induced cardiovascular and renal injury. Current opinion in nephrology and hypertension. 17: 37-43
Li, X. & Tai, H. H. (2013) Activation of thromboxane A2 receptor (TP) increases the expression of monocyte chemoattractant protein-1 (MCP-1)/chemokine (C—C motif) ligand 2 (CCL2) and recruits macrophages to promote invasion of lung cancer cells. PLoS One, 8, e54073.
Liang, M., Niu, J., Zhang, L., Deng, H., Ma, J., Zhou, W., Duan, D., Zhou, Y., Xu, H. & Chen, L. (2016) Gene expression profiling reveals different molecular patterns in G-protein coupled receptor signalling pathways between early- and late-onset preeclampsia. Placenta, 40, 52-59.
Lin, C.-I., Chen, C.-N., Lin, P.-W., Chang, K.-J., Hsieh, F.-J. & Lee, H. (2007) Lysophosphatidic acid regulates inflammation-related genes in human endothelial cells through LPA 1 and LPA 3. Biochemical and biophysical research communications, 363, 1001-1008.
Lin, E.-J. D., Sainsbury, A., Lee, N. J., Boey, D., Couzens, M., Enriquez, R., Slack, K., Bland, R., During, M. J. & Herzog, H. (2006) Combined deletion of Y1, Y2, and Y4 receptors prevents hypothalamic neuropeptide Y overexpression-induced hyperinsulinemia despite persistence of hyperphagia and obesity. Endocrinology, 147, 5094-5101.
Ling, P., Ngo, K., Nguyen, S., Thurmond, R. L., Edwards, J. P., Karlsson, L. & Fung-Leung, W. P. (2004) Histamine H4 receptor mediates eosinophil chemotaxis with cell shape change and adhesion molecule upregulation. British journal of pharmacology, 142, 161-171.
Liu, C., Kuei, C., Sutton, S., Chen, J., Bonaventure, P., Wu, J., Nepomuceno, D., Kamme, F., Tran, D.-T. & Zhu, J. (2005) INSL5 is a high affinity specific agonist for GPCR142 (GPR100). Journal of Biological Chemistry, 280, 292-300.
Liu, S., Qian, Y., Li, L., Wei, G., Guan, Y., Pan, H., Guan, X., Zhang, L., Lu, X. & Zhao, Y. (2013) Lgr4 gene deficiency increases susceptibility and severity of dextran sodium sulfate-induced inflammatory bowel disease in mice. Journal of Biological Chemistry, 288, 8794-8803
Lu, M. C., Lai, N. S., Yu, H. C., Huang, H. B., Hsieh, S. C. & Yu, C. L. (2010) Anti-citrullinated protein antibodies bind surface-expressed citrullinated Grp78 on monocyte/macrophages and stimulate tumor necrosis factor α production. Arthritis & Rheumatism, 62, 1213-1223
Lundequist, A. & Boyce, J. A. (2011) LPA5 is abundantly expressed by human mast cells and important for lysophosphatidic acid induced MIP-1β release. PLoS One, 6, e18192.
Maekawa, A., Balestrieri, B., Austen, K. F. & Kanaoka, Y. (2009) GPR17 is a negative regulator of the cysteinyl leukotriene 1 receptor response to leukotriene D4. Proceedings of the National Academy of Sciences, 106, 11685-11690.
Malki, A., Fiedler, J., Fricke, K., Ballweg, I., Pfaffl, M. W. & Krautwurst, D. (2015)
Class I odorant receptors, TAS1R and TAS2R taste receptors, are markers for subpopulations of circulating leukocytes. Journal of leukocyte biology, 97, 533-545.
Mao, Y., Zhang, M., Tuma, R. F. & Kunapuli, S. P. (2010) Deficiency of PAR4 attenuates cerebral ischemia/reperfusion injury in mice. Journal of Cerebral Blood Flow & Metabolism, 30, 1044-1052
Marazziti, D., Ori, M., Nardini, M., Rossi, A., Nardi, I. & Cassano, G. B. (2001) mRNA expression of serotonin receptors of type 2C and 5A in human resting lymphocytes. Neuropsychobiology, 43, 123-126.
Martinez, F. O., Gordon, S., Locati, M. & Mantovani, A. (2006) Transcriptional profiling of the human monocyte-to-macrophage differentiation and polarization: new molecules and patterns of gene expression. The Journal of Immunology, 177, 7303-7311.
Marvar, P. J., et al. (2010) Central and peripheral mechanisms of T-lymphocyte activation and vascular inflammation produced by angiotensin II-induced hypertension. Circ Res, 107: 263-270
Mas, V., Maluf, D., Archer, K. J., Potter, A., Suh, J., Gehrau, R., Descalzi, V. & Villamil, F. (2011) Transcriptome at the time of hepatitis C virus recurrence may predict the severity of fibrosis progression after liver transplantation. Liver Transplantation, 17, 824-835
Maslowski, K. M., Vieira, A. T., Ng, A., Kranich, J., Sierro, F., Yu, D., Schilter, H. C., Rolph, M. S., Mackay, F. & Artis, D. (2009) Regulation of inflammatory responses by gut microbiota and chemoattractant receptor GPR43. Nature, 461, 1282-1286.
Matavelli, L. C., Huang, J. & Siragy, H. M. (2011) Angiotensin AT2 receptor stimulation inhibits early renal inflammation in renovascular hypertension. Hypertension, 57, 308-313.
Matloubian, M., Lo, C. G., Cinamon, G., Lesneski, M. J., Xu, Y., Brinkmann, V., Allende, M. L., Proia, R. L. & Cyster, J. G. (2004) Lymphocyte egress from thymus and peripheral lymphoid organs is dependent on S1P receptor 1. Nature, 427, 355-360.
Matsumoto, M., Saito, T., Takasaki, J., Kamohara, M., Sugimoto, T., Kobayashi, M., Tadokoro, M., Matsumoto, S.-i., Ohishi, T. & Furuichi, K. (2000) An evolutionarily conserved G-protein coupled receptor family, SREB, expressed in the central nervous system. Biochemical and biophysical research communications, 272, 576-582.
Matsumura, T., Oyama, M., Kozuka-Hata, H., Ishikawa, K., Inoue, T., Muta, T., Semba, K. & Inoue, J.-i. (2010) Identification of BCAP-L as a negative regulator of the TLR signalling-induced production of IL-6 and IL-10 in macrophages by tyrosine phosphoproteomics. Biochemical and Biophysical Research Communications, 400, 265-270.
Matteucci, C., Minutolo, A., Sinibaldi-Vallebona, P., Palamara, A. T., Rasi, G., Mastino, A. & Garaci, E. (2010) Transcription profile of human lymphocytes following in vitro treatment with thymosin alpha-1. Annals of the New York Academy of Sciences, 1194, 6-19.
McPherson, J. A., Barringhaus, K. G., Bishop, G. G., Sanders, J. M., Rieger, J. M., Hesselbacher, S. E., Gimple, L. W., Powers, E. R., Macdonald, T. & Sullivan, G. (2001) Adenosine A2A receptor stimulation reduces inflammation and neointimal growth in a murine carotid ligation model. Arteriosclerosis, Thrombosis, and Vascular Biology, 21, 791-796.
McQuiston, T., Luberto, C. & Del Poeta, M. (2011) Role of sphingosine-1-phosphate (SIP) and S1P receptor 2 in the phagocytosis of Cryptococcus neoformans by alveolar macrophages. Microbiology, 157, 1416-1427.
Mellado, M., Fernández-AgullÓ, T., Rodríguez-Frade, J. M., Garcia San Frutos, M., de la Peña, P., Martínez-A, C. & Montoya, E. (1999) Expression analysis of the thyrotropin-releasing hormone receptor (TRHR) in the immune system using agonist anti-TRHR monoclonal antibodies. FEBS Letters, 451, 308-314.
Mitić, K., Stanojević, S., Kuštrimovio, N., Vujić, V. & Dimitrijević, M. (2011) Neuropeptide Y modulates functions of inflammatory cells in the rat: Distinct role for Y1, Y2 and Y5 receptors. Peptides, 32, 1626-1633.
Mitsuhashi, M., Mitsuhashi, T. & Payan, D. (1989) Multiple signalling pathways of histamine H2 receptors. Identification of an H2 receptor-dependent Ca2+ mobilization pathway in human HL-60 promyelocytic leukemia cells. Journal of Biological Chemistry, 264, 18356-18362.
Moore, D. J., Chambers, J. K., Wahlin, J.-P., Tan, K. B., Moore, G. B., Jenkins, O., Emson, P. C. & Murdock, P. R. (2001) Expression pattern of human P2Y receptor subtypes: a quantitative reverse transcription-polymerase chain reaction study. Biochimica et Biophysica Acta (BBA)-Gene Structure and Expression, 1521, 107-119.
Moriyama, M., Sato, T., Inoue, H., Fukuyama, S., Teranishi, H., Kangawa, K., Kano, T., Yoshimura, A. & Kojima, M. (2005) The neuropeptide neuromedin U promotes inflammation by direct activation of mast cells. The Journal of experimental medicine, 202, 217-224.
Muir, A. I., Chamberlain, L., Elshourbagy, N. A., Michalovich, D., Moore, D. J., Calamari, A., Szekeres, P. G., Sarau, H. M., Chambers, J. K. & Murdock, P. (2001) AXOR12, a novel human G protein-coupled receptor, activated by the peptide KISS-1. Journal of Biological Chemistry, 276, 28969-28975.
Nagamachi, M., Sakata, D., Kabashima, K., Furuyashiki, T., Murata, T., Segi-Nishida, E., Soontrapa, K., Matsuoka, T., Miyachi, Y. & Narumiya, S. (2007) Facilitation of Th1-mediated immune response by prostaglandin E receptor EP1. The Journal of experimental medicine, 204, 2865-2874.
Németh, Z. H., Lutz, C. S., Csóka, B., Deitch, E. A., Leibovich, S. J., Gause, W. C., Tone, M., Pacher, P., Vizi, E. S. & Haskó, G. (2005) Adenosine augments IL-10 production by macrophages through an A2B receptor-mediated posttranscriptional mechanism. The Journal of Immunology, 175, 8260-8270.
Niedernberg, A., Tunaru, S., Blaukat, A., Ardati, A. & Kostenis, E. (2003) Sphingosine 1-phosphate and dioleoylphosphatidic acid are low affinity agonists for the orphan receptor GPR63. Cellular Signalling, 15, 435-446.
Nishio, R., Matsumori, A., Shioi, T., Wang, W., Yamada, T., Ono, K. & Sasayama, S. (1998) Denopamine, a β1-adrenergic agonist, prolongs survival in a murine model of congestive heart failure induced by viral myocarditis: suppression of tumor necrosis factor-α production in the heart. Journal of the American College of Cardiology, 32, 808-815.
Novitzky-Basso, I., F. Spring, D. Anstee, D. Tripathi and F. Chen (2018). “Erythrocytes from patients with myeloproliferative neoplasms and splanchnic venous thrombosis show greater expression of Lu/BCAM.” Int J Lab Hematol 40(4): 473-477.
Ohshima, S., Yamaguchi, N., Nishioka, K., Mima, T., Ishii, T., Umeshita-Sasai, M., Kobayashi, H., Shimizu, M., Katada, Y. & Wakitani, S. (2002) Enhanced local production of osteopontin in rheumatoid joints. The Journal of Rheumatology, 29, 2061-2067.
Okamoto, K., Imbe, H., Morikawa, Y., Itoh, M., Sekimoto, M., Nemoto, K. & Senba, E. (2002) 5-HT2A receptor subtype in the peripheral branch of sensory fibers is involved in the potentiation of inflammatory pain in rats. Pain, 99, 133-143.
Osborn, O., McNelis, J., Sanchez-Alavez, M., Talukdar, S., Lu, M., Li, P., Thiede, L., Morinaga, H., Kim, J. J. & Heinrichsdorff, J. (2012) G protein-coupled receptor 21 deletion improves insulin sensitivity in diet-induced obese mice. The Journal of clinical investigation, 122, 2444-2453.
Othman, M. A., Grygalewicz, B., Pienkowska-Grela, B., Rincic, M., Rittscher, K., Melo, J. B., Carreira, I. M., Meyer, B., Marzena, W. & Liehr, T. (2015) Novel Cryptic Rearrangements in Adult B-Cell Precursor Acute Lymphoblastic Leukemia Involving the MLL Gene. Journal of Histochemistry & Cytochemistry, 0022155415576201.
Parker, H., Habib, A., Rogers, G., Gribble, F. & Reimann, F. (2009) Nutrient-dependent secretion of glucose-dependent insulinotropic polypeptide from primary murine K cells. Diabetologia, 52, 289-298.
Pasternack, S. M., von Kugelgen, I., A1 Aboud, K., Lee, Y.-A., Ruschendorf, F., Voss, K., Hillmer, A. M., Molderings, G. J., Franz, T. & Ramirez, A. (2008) G protein-coupled receptor P2Y5 and its ligand LPA are involved in maintenance of human hair growth. Nature genetics, 40, 329-334.
Patel, D. D., Wee, S. F., Whichard, L. P., Bowen, M. A., Pesando, J. M., Aruffo, A. and Haynes, B. F. (1995). Identification and characterization of a 100-kD ligand for CD6 on human thymic epithelial cells. J. Exp. Med. 181, 1563-1568
Peluso, J., LaForge, K. S., Matthes, H. W., Kreek, M. J., Kieffer, B. L. & Gaveriaux-Ruff, C. (1998) Distribution of nociceptin/orphanin F Q receptor transcript in human central nervous system and immune cells. Journal of neuroimmunology, 81, 184-192.
Penna E, Orso F, Cimino D, Vercellino I, Grassi E, Quaglino E, Turco E, and Taverna D. miR-214 coordinates melanoma progression by upregulating ALCAM through TFAP2 and miR-148b downmodulation. Cancer research. 2013; 73(13):4098-111.
Penna, E., F. Orso, D. Cimino, I. Vercellino, E. Grassi, E. Quaglino, E. Turco and D. Taverna (2013). “miR-214 coordinates melanoma progression by upregulating ALCAM through TFAP2 and miR-148b downmodulation.” Cancer Res 73(13): 4098-4111.
Piao D, Jiang T, Liu G, Wang B, Xu J, and Zhu A. Clinical implications of activated leukocyte cell adhesion molecule expression in breast cancer. Molecular biology reports. 2012; 39(1):661-8.
Piao, D., T. Jiang, G. Liu, B. Wang, J. Xu and A. Zhu (2012). “Clinical implications of activated leukocyte cell adhesion molecule expression in breast cancer.” Mol Biol Rep 39(1): 661-668.
Pillai, S. G., Cousens, D. J., Barnes, A. A., Buckley, P. T., Chiano, M. N., Hosking, L. K., Cameron, L.-A., Fling, M. E., Foley, J. J. & Green, A. (2004) A coding polymorphism in the CYSLT2 receptor with reduced affinity to LTD4 is associated with asthma. Pharmacogenetics and Genomics, 14, 627-633.
Poloso, N. J., Urquhart, P., Nicolaou, A., Wang, J. & Woodward, D. F. (2013) PGE 2 differentially regulates monocyte-derived dendritic cell cytokine responses depending on receptor usage (EP 2/EP 4). Molecular immunology, 54, 284-295.
Powell, W. S. & Rokach, J. (2013) The eosinophil chemoattractant 5-oxo-ETE and the OXE receptor. Progress in lipid research, 52, 651-665.
Qu, L., Fan, N., Ma, C., Wang, T., Han, L., Fu, K., Wang, Y., Shimada, S. G., Dong, X. & LaMotte, R. H. (2014) Enhanced excitability of MRGPRA3- and MRGPRD-positive nociceptors in a model of inflammatory itch and pain. Brain, 137, 1039-1050.
Quigley, D. A., To, M. D., Perez-Losada, J., Pelorosso, F. G., Mao, J.-H., Nagase, H., Ginzinger, D. G. & Balmain, A. (2009) Genetic architecture of mouse skin inflammation and tumour susceptibility. Nature, 458, 505-508.
Rai V, Maldonado A Y, Burz D S, Reverdatto S, Schmidt A M and Shekhtman A; Signal Transduction in Receptor for Advanced Glycation End Products (RAGE), J Biol Chem, 2012, 287: 5133-44
Rajagopalan, S., Kurz, S., Munzel, T., Tarpey, M., Freeman, B. A., Griendling, K. K. and Harrison, D. G., (1996) Angiotensin II-mediated hypertension in the rat increases vascular superoxide production via membrane NADH/NADPH oxidase activation. Contribution to alterations of vasomotor tone, J Clin Invest., 97: 1916-23
Ramasamy R, Shekhtman A, Schmidt A M, The multiple faces of RAGE-opportunities for therapeutic intervention in aging and chronic disease. Expert Opin Ther Targets, 2016, 20: 431-446
Rao V et al. (2006) Role for Macrophage Metalloelastase in Glomerular Basement Membrane Damage Associated with Alport Syndrome, American Journal of Pathology, 169: 32-46.
Rauch S J, Rosenkranz A C, Bohm A, Meyer-Kirchrath J, Hohlfeld T, Schror K, and Rauch B H. Cholesterol induces apoptosis-associated loss of the activated leukocyte cell adhesion molecule (ALCAM) in human monocytes. Vascular pharmacology. 2011; 54(3-6):93-9.
Rauch, S. J., A. C. Rosenkranz, A. Bohm, J. Meyer-Kirchrath, T. Hohlfeld, K. Schror and B. H. Rauch (2011). “Cholesterol induces apoptosis-associated loss of the activated leukocyte cell adhesion molecule (ALCAM) in human monocytes.” Vascul Pharmacol 54(3-6): 93-99.
Rebeck, G. W., Maynard, K. I., Hyman, B. T. & Moskowitz, M. A. (1994) Selective 5-HT1D alpha serotonin receptor gene expression in trigeminal ganglia: implications for antimigraine drug development. Proceedings of the National Academy of Sciences, 91, 3666-3669.
Rees, S., den Daas, I., Foord, S., Goodson, S., Bull, D., Kilpatrick, G. & Lee, M. (1994) Cloning and characterisation of the human 5-HT5A serotonin receptor. FEBS letters, 355, 242-246.
Robinson, L. J., Tourkova, I., Wang, Y., Sharrow, A. C., Landau, M. S., Yaroslayskiy, B. B., Sun, L., Zaidi, M. & Blair, H. C. (2010) FSH-receptor isoforms and FSH-dependent gene transcription in human monocytes and osteoclasts. Biochemical and biophysical research communications, 394, 12-17.
Rompler, H., Schulz, A., Pitra, C., Coop, G., Przeworski, M., Paabo, S. & Schoneberg, T. (2005) The rise and fall of the chemoattractant receptor GPR33. Journal of Biological Chemistry.
Rossi, L., Lemoli, R. M. & Goodell, M. A. (2013) Gpr171, a putative P2Y-like receptor, negatively regulates myeloid differentiation in murine hematopoietic progenitors. Experimental hematology, 41, 102-112.
Roy A, Kucukural A, Zhang Y. I-TASSER: a unified platform for automated protein structure and function prediction. Nature Protocols, 2010, 5: 725-738
Rubic, T., Lametschwandtner, G., Jost, S., Hinteregger, S., Kund, J., Carballido-Perrig, N., Schwärzler, C., Junt, T., Voshol, H. & Meingassner, J. G. (2008) Triggering the succinate receptor GPR91 on dendritic cells enhances immunity. Nature immunology, 9, 1261-1269.
Ruma, I. M., E. W. Putranto, E. Kondo, H. Murata, M. Watanabe, P. Huang, R. Kinoshita, J. Futami, Y. Inoue, A. Yamauchi, I. W. Sumardika, C. Youyi, K. Yamamoto, Y. Nasu, M. Nishibori, T. Hibino and M. Sakaguchi (2016). “MCAM, as a novel receptor for S100A8/A9, mediates progression of malignant melanoma through prominent activation of NF-kappaB and ROS formation upon ligand binding.” Clin Exp Metastasis 33(6): 609-627.
Saban, R., Saban, M. R., Nguyen, N.-B., Lu, B., Gerard, C., Gerard, N. P. & Hammond, T. G. (2000) Neurokinin-1 (NK-1) receptor is required in antigen-induced cystitis. The American journal of pathology, 156, 775-780.
Sakamoto, Y., Inoue, H., Kawakami, S., Miyawaki, K., Miyamoto, T., Mizuta, K. & Itakura, M. (2006) Expression and distribution of Gpr119 in the pancreatic islets of mice and rats: predominant localization in pancreatic polypeptide-secreting PP-cells. Biochemical and biophysical research communications, 351, 474-480.
Sampaio, A. L., Rae, G. A. & Maria das Gracas, M. (2004) Effects of endothelin ETA receptor antagonism on granulocyte and lymphocyte accumulation in LPS-induced inflammation. Journal of leukocyte biology, 76, 210-216.
Sarkar, C., Das, S., Chakroborty, D., Chowdhury, U. R., Basu, B., Dasgupta, P. S. & Basu, S. (2006) Cutting edge: stimulation of dopamine D4 receptors induce T cell quiescence by up-regulating Krüppel-like factor-2 expression through Inhibition of ERK1/ERK2 phosphorylation. The Journal of Immunology, 177, 7525-7529.
Sasaki, Y., Hoshi, M., Akazawa, C., Nakamura, Y., Tsuzuki, H., Inoue, K. & Kohsaka, S. (2003) Selective expression of Gi/o-coupled ATP receptor P2Y12 in microglia in rat brain. Glia, 44, 242-250.
Sato, K. Z., Fujii, T., Watanabe, Y., Yamada, S., Ando, T., Kazuko, F. & Kawashima, K. (1999) Diversity of mRNA expression for muscarinic acetylcholine receptor subtypes and neuronal nicotinic acetylcholine receptor subunits in human mononuclear leukocytes and leukemic cell lines. Neuroscience letters, 266, 17-20.
Satoh, A., Shimosegawa, T., Satoh, K., Ito, H., Kohno, Y., Masamune, A., Fujita, M. & Toyota, T. (2000) Activation of adenosine A1-receptor pathway induces edema formation in the pancreas of rats. Gastroenterology, 119, 829-836.
Schaub, A., Futterer, A. & Pfeffer, K. (2001) PUMA-G, an IFN-gamma-inducible gene in macrophages is a novel member of the seven transmembrane spanning receptor superfamily. Eur J Immunol, 31, 3714-3725.
Schiffmann, E., Corcoran, B. A. & Wahl, S. M. (1975) N-formylmethionyl peptides as chemoattractants for leucocytes. Proceedings of the National Academy of Sciences, 72, 1059-1062.
Schmidhuber, S. M., Rauch, I., Kofler, B. & Brain, S. D. (2009) Evidence that the modulatory effect of galanin on inflammatory edema formation is mediated by the galanin receptor 3 in the murine microvasculature. Journal of molecular neuroscience: MN, 37, 177-181
Schmitz, F., Schrader, H., Otte, J.-M., Schmitz, H., Stüber, E., Herzig, K.-H. & Schmidt, W. E. (2001) Identification of CCK-B/gastrin receptor splice variants in human peripheral blood mononuclear cells. Regulatory peptides, 101, 25-33.
Schuelert, N. & McDougall, J. J. (2011) The abnormal cannabidiol analogue 0-1602 reduces nociception in a rat model of acute arthritis via the putative cannabinoid receptor GPR55. Neuroscience letters, 500, 72-76.
Shen, Z.-J., Hu, J., Esnault, S., Dozmorov, I. & Malter, J. S. (2015) RNA Seq profiling reveals a novel expression pattern of TGF-8 target genes in human blood eosinophils. Immunology letters, 167, 1-10.
Shen. J., Huang. Y. M., Wang. M., et al. (2016) Renin-angiotensin system blockade for the risk of cancer and death. J Renin Angiotensin Aldosterone Syst. 8, 17(3)
Shih, I. M. (1999). “The role of CD146 (Mel-CAM) in biology and pathology.” J Pathol 189(1): 4-11.
Smedbakken L, Jensen J K, Hallen J, Atar D, Januzzi J L, Halvorsen B, Aukrust P, and Ueland T. Activated leukocyte cell adhesion molecule and prognosis in acute ischemic stroke. Stroke. 2011; 42(9):2453-8.
Smedbakken, L., J. K. Jensen, J. Hallen, D. Atar, J. L. Januzzi, B. Halvorsen, P. Aukrust and T. Ueland (2011). “Activated leukocyte cell adhesion molecule and prognosis in acute ischemic stroke.” Stroke 42(9): 2453-2458.
Smith J R, Chipps T J, Ilias H, Pan Y, and Appukuttan B. Expression and regulation of activated leukocyte cell adhesion molecule in human retinal vascular endothelial cells. Experimental eye research. 2012; 104(89-93.
Smith, J. R., T. J. Chipps, H. Ilias, Y. Pan and B. Appukuttan (2012). “Expression and regulation of activated leukocyte cell adhesion molecule in human retinal vascular endothelial cells.” Exp Eye Res 104: 89-93.
Sohn, S.-H., Chung, H.-S., Ko, E., Jeong, H.-j., Kim, S.-H., Jeong, J.-H., Kim, Y., Shin, M., Hong, M. & Bae, H. (2009) The genome-wide expression profile of Nelumbinis semen on lipopolysaccharide-stimulated BV-2 microglial cells. Biological and Pharmaceutical Bulletin, 32, 1012-1020.
Solinski, H. J., Petermann, F., Rothe, K., Boekhoff, I., Gudermann, T. & Breit, A. (2013) Human Mas-Related G Protein-Coupled Receptors-X1 Induce Chemokine Receptor 2 Expression in Rat Dorsal Root Ganglia Neurons and Release of Chemokine Ligand 2 from the Human LAD-2 Mast Cell Line. PLoS ONE, 8, e58756.
Sonobe, Y., Nakane, H., Watanabe, T. & Nakano, K. (2004) Regulation of Con A-dependent cytokine production from CD4+ and CD8+T lymphocytes by autosecretion of histamine. Inflammation Research, 53, 87-92.
Sonoda, N., Katabuchi, H., Tashiro, H., Ohba, T., Nishimura, R., Minegishi, T. & Okamura, H. (2005) Expression of variant luteinizing hormone/chorionic gonadotropin receptors and degradation of chorionic gonadotropin in human chorionic villous macrophages. Placenta, 26, 298-307.
Soro-Paavonen, A., Watson, A M., Thomas, M. C., et al. (2008) Receptor for advanced glycation end products (RAGE) deficiency attenuates the development of atherosclerosis in diabetes, Diabetes, 57:2461-2469
Souza, D. G., Lomez, E. S. L., Pinho, V., Pesquero, J. B., Bader, M., Pesquero, J. L. & Teixeira, M. M. (2004) Role of Bradykinin B2 and B1 Receptors in the Local, Remote, and Systemic Inflammatory Responses That Follow Intestinal Ischemia and Reperfusion Injury. The Journal of Immunology, 172, 2542-2548.
Stefulj, J., Jernej, B., Cicin-Sain, L., Rinner, I. & Schauenstein, K. (2000) mRNA expression of serotonin receptors in cells of the immune tissues of the rat. Brain, behavior, and immunity, 14, 219-224.
Stockhammer, O. W., Rauwerda, H., Wittink, F. R., Breit, T. M., Meijer, A. H. & Spaink, H. P. (2010) Transcriptome analysis of Traf6 function in the innate immune response of zebrafish embryos. Molecular immunology, 48, 179-190.
Subramanian, H., Gupta, K., Guo, Q., Price, R. & Ali, H. (2011) Mas-related Gene X2 (MrgX2) Is a Novel G Protein-coupled Receptor for the Antimicrobial Peptide LL-37 in Human Mast Cells RESISTANCE TO RECEPTOR PHOSPHORYLATION, DESENSITIZATION, AND INTERNALIZATION. Journal of Biological Chemistry, 286, 44739-44749.
Sugimoto, T., Saito, M., Mochizuki, S., Watanabe, Y., Hashimoto, S. & Kawashima, H. (1994) Molecular cloning and functional expression of a cDNA encoding the human V1b vasopressin receptor. Journal of Biological Chemistry, 269, 27088-27092.
Sugo, T., Tachimoto, H., Chikatsu, T., Murakami, Y., Kikukawa, Y., Sato, S., Kikuchi, K., Nagi, T., Harada, M. & Ogi, K. (2006) Identification of a lysophosphatidylserine receptor on mast cells. Biochemical and biophysical research communications, 341, 1078-1087.
Sulaj A, Kopf S, Grone E, Grone H J, Hoffmann S, Schleicher E, Haring H U, Schwenger V, Herzig S, Fleming T, et al. ALCAM a novel biomarker in patients with type 2 diabetes mellitus complicated with diabetic nephropathy. Journal of diabetes and its complications. 2017; 31(6):1058-65.
Sulaj, A., S. Kopf, E. Grone, H. J. Grone, S. Hoffmann, E. Schleicher, H. U. Haring, V. Schwenger, S. Herzig, T. Fleming, P. P. Nawroth and R. von Bauer (2017). “ALCAM a novel biomarker in patients with type 2 diabetes mellitus complicated with diabetic nephropathy.” J Diabetes Complications 31(6): 1058-1065.
Sunuwar, L., Medini, M., Cohen, L., Sekler, I. & Hershfinkel, M. (2016) The zinc sensing receptor, ZnR/GPR39, triggers metabotropic calcium signalling in colonocytes and regulates occludin recovery in experimental colitis. Phil. Trans. R. Soc. B, 371, 20150420.
Suzuki, T., Won, K.-J., Horiguchi, K., Kinoshita, K., Hori, M., Torihashi, S., Momotani, E., Itoh, K., Hirayama, K. & Ward, S. M. (2004) Muscularis inflammation and the loss of interstitial cells of Cajal in the endothelin ETB receptor null rat. American Journal of Physiology-Gastrointestinal and Liver Physiology, 287, G638-G646.
Swan, C., Duroudier, N. P., Campbell, E., Zaitoun, A., Hastings, M., Dukes, G. E., Cox, J., Kelly, F. M., Wilde, J. & Lennon, M. G. (2013) Identifying and testing candidate genetic polymorphisms in the irritable bowel syndrome (IBS): association with TNFSF15 and TNFα. Gut, 62, 985-994.
Swaney, J., Chapman, C., Correa, L., Stebbins, K., Bundey, R., Prodanovich, P., Fagan, P., Baccei, C., Santini, A. & Hutchinson, J. (2010) A novel, orally active LPA1 receptor antagonist inhibits lung fibrosis in the mouse bleomycin model. British journal of pharmacology, 160, 1699-1713.
Swart, G. W. (2002). Activated leukocyte cell adhesion molecule (CD166/ALCAM):developmental and mechanistic aspects of cell clustering and cell migration. Eur. J. Cell Biol. 81, 313-321.
Takayama, K., Yuhki, K., Ono, K., Fujino, T., Hara, A., Yamada, T., Kuriyama, S., Karibe, H., Okada, Y., Takahata, O., Taniguchi, T., Iijima, T., Iwasaki, H., Narumiya, S. & Ushikubi, F. (2005) Thromboxane A2 and prostaglandin F2alpha mediate inflammatory tachycardia. Nat Med, 11, 562-566.
Takenouchi, R., Inoue, K., Kambe, Y. & Miyata, A. (2012) N-arachidonoyl glycine induces macrophage apoptosis via GPR18. Biochemical and biophysical research communications, 418, 366-371.
Taniyama, Y., Suzuki, T., Mikami, Y., Moriya, T., Satomi, S. & Sasano, H. (2005) Systemic distribution of somatostatin receptor subtypes in human: an immunohistochemical study. Endocrine journal, 52, 605-611.
Taquet, N., Philippe, C., Reimund, J.-M. & Muller, C. D. (2012) Inflammatory Bowel Disease G-Protein Coupled Receptors (GPCRs) Expression Profiling with Microfluidic Cards.
Taub, D. D., Eisenstein, T. K., Geller, E. B., Adler, M. W. & Rogers, T. J. (1991) Immunomodulatory activity of mu- and kappa-selective opioid agonists. Proceedings of the National Academy of Sciences, 88, 360-364.
Tayebati, S., Bronzetti, E., Morra Di Cella, S., Mulatero, P., Ricci, A., Rossodivita, I., Schena, M., Schiavone, D., Veglio, F. & Amenta, F. (2000) In situ hybridization and immunocytochemistry of alpha1-adrenoceptors in human peripheral blood lymphocytes. Journal of autonomic pharmacology, 20, 305-312.
Te Riet J, Helenius J, Strohmeyer N, Cambi A, Figdor C G, and Muller D J. Dynamic coupling of ALCAM to the actin cortex strengthens cell adhesion to CD6. Journal of cell science. 2014; 127(Pt 7):1595-606.
Te Riet, J., J. Helenius, N. Strohmeyer, A. Cambi, C. G. Figdor and D. J. Muller (2014). “Dynamic coupling of ALCAM to the actin cortex strengthens cell adhesion to CD6.” J Cell Sci 127(Pt 7): 1595-1606.
Ter Beek, W. P., Muller, E. S., Van Den Berg, M., Meijer, M. J., Biemond, I. & Lamers, C. B. (2008) Motilin receptor expression in smooth muscle, myenteric plexus, and mucosa of human inflamed and noninflamed intestine. Inflammatory bowel diseases, 14, 612-619.
Teuscher, C., Subramanian, M., Noubade, R., Gao, J. F., Offner, H., Zachary, J. F. & Blankenhorn, E. P. (2007) Central histamine H3 receptor signalling negatively regulates susceptibility to autoimmune inflammatory disease of the CNS. Proceedings of the National Academy of Sciences, 104, 10146-10151.
Thoene-Reineke, C., Rumschussel, K., Schmerbach, K. et al., Prevention and intervention studies with telmisartan, ramipril and their combination in different rat stroke models. PloS One, 2011, 6: e23646
Thomas, M. C., Pickering, R. J., Tsorotes, D., Koitka, A., Sheehy, K., Bernardi, S., Toffoli B., Nguyen-Huu, T. P., Head, G. A., Fu, Y., Chin-Dusting, J., Cooper, M. E., Tikellis C. (2010) Genetic Ace2 deficiency accentuates vascular inflammation and atherosclerosis in the ApoE knockout mouse. Circulation Research, 107: 888-97
Tichelaar, J. W., Wesselkamper, S. C., Chowdhury, S., Yin, H., Berclaz, P.-Y., Sartor, M. A., Leikauf, G. D. & Whitsett, J. A. (2007) Duration-dependent cytoprotective versus inflammatory effects of lung epithelial fibroblast growth factor-7 expression. Experimental lung research, 33, 385-417.
Tikellis, C, Wookey, P. J., Candido, R., Thomas, M. C. (2004) Improved islet morphology after blockade of the renin-angiotensin system in the ZDF rat, Diabetes, 53: 989-997
Tikellis, C., Pickering, R. J., Tsorotes, D., Huet, O., Chin-Dusting, J., Cooper, M. E., and Thomas, M. C. (2012) Activation of the Renin-Angiotensin system mediates the effects of dietary salt intake on atherogenesis in the apolipoprotein E knockout mouse, Hypertension, 60: 98-105.
Tiulpakov A, White C W, Abhayawardana R S, See H B, Chan A S, Seeber R M, Heng J I, Dedov I, Pavlos N J, Pfleger K D G, Mutations of vasopressin receptor 2 including novel L312S have differential effects on trafficking, Mol Endocrinol, 2016, 30: 889-904
Tobon-Velasco J C, Cuevas E, and Torres-Ramos M A. Receptor for AGEs (RAGE) as mediator of NF-kB pathway activation in neuroinflammation and oxidative stress. CNS & neurological disorders drug targets. 2014; 13(9):1615-26.
Tu, T., C. Zhang, H. Yan, Y. Luo, R. Kong, P. Wen, Z. Ye, J. Chen, J. Feng, F. Liu, J. Y. Wu and X. Yan (2015). “CD146 acts as a novel receptor for netrin-1 in promoting angiogenesis and vascular development.” Cell Res 25(3): 275-287.
Uhlen, M., FAGERberg, L., Hallström, B. M., Lindskog, C., Oksvold, P., Mardinoglu, A., Sivertsson, Å., Kampf, C., Sjöstedt, E. & Asplund, A. (2015) Tissue-based map of the human proteome. Science, 347, 1260419.
“van Kempen, L. C., Nelissen, J. M., Degen, W. G., Torensma, R., Weidle, U. H., van Kempen, L. C., Nelissen, J. M., Degen, W. G., Torensma, R., Weidle, U. H.,
Bloemers, H. P., Figdor, C. G. and Swart, G. W. (2001). Molecular basis for the homophilic activated leukocyte cell adhesion molecule (ALCAM)-ALCAM interaction. J. Biol. Chem. 276, 25783-25790.”
Vaughan, K. R., Stokes, L., Prince, L. R., Marriott, H. M., Meis, S., Kassack, M. U., Bingle, C. D., Sabroe, I., Surprenant, A. & Whyte, M. K. (2007) Inhibition of neutrophil apoptosis by ATP is mediated by the P2Y11 receptor. The Journal of Immunology, 179, 8544-8553.
Venkataraman, C. & Kuo, F. (2005) The G-protein coupled receptor, GPR84 regulates IL-4 production by T lymphocytes in response to CD3 crosslinking. Immunology letters, 101, 144-153.
von Bauer R, Oikonomou D, Sulaj A, Mohammed S, Hotz-Wagenblatt A, Grone H J, Arnold B, Falk C, Luethje D, Erhardt A, et al. CD166/ALCAM mediates proinflammatory effects of S100B in delayed type hypersensitivity. Journal of immunology. 2013; 191(1):369-77.
von Bauer, R., D. Oikonomou, A. Sulaj, S. Mohammed, A. Hotz-Wagenblatt, H. J. Grone, B. Arnold, C. Falk, D. Luethje, A. Erhardt, D. M. Stern, A. Bierhaus and P. P. Nawroth (2013). “CD166/ALCAM mediates proinflammatory effects of S100B in delayed type hypersensitivity.” J Immunol 191(1): 369-377.
Wade A, Thomas C, Kalmar B, Terenzio M, Garin J, Greensmith L, and Schiavo G. Activated leukocyte cell adhesion molecule modulates neurotrophin signaling. Journal of neurochemistry. 2012; 121(4):575-86.
Wade, A., C. Thomas, B. Kalmar, M. Terenzio, J. Garin, L. Greensmith and G. Schiavo (2012). “Activated leukocyte cell adhesion molecule modulates neurotrophin signaling.” J Neurochem 121(4): 575-586.
Wang J, Gu Z, Ni P, Qiao Y, Chen C, Liu X, Lin J, Chen N, and Fan Q. NF-kappaB P50/P65 hetero-dimer mediates differential regulation of CD166/ALCAM expression via interaction with micoRNA-9 after serum deprivation, providing evidence for a novel negative auto-regulatory loop. Nucleic acids research. 2011; 39(15):6440-55.
Wang Z, and Yan X. CD146, a multi-functional molecule beyond adhesion. Cancer letters. 2013; 330(2):150-62.
Wang, D. B., Dayton, R. D., Zweig, R. M. & Klein, R. L. (2010) Transcriptome analysis of a tau overexpression model in rats implicates an early pro-inflammatory response. Experimental neurology, 224, 197-206.
Wang, J., Simonavicius, N., Wu, X., Swaminath, G., Reagan, J., Tian, H. & Ling, L. (2006) Kynurenic acid as a ligand for orphan G protein-coupled receptor GPR35. Journal of Biological Chemistry, 281, 22021-22028.
Wang, J., Z. Gu, P. Ni, Y. Qiao, C. Chen, X. Liu, J. Lin, N. Chen and Q. Fan (2011). “NF-kappaB P50/P65 hetero-dimer mediates differential regulation of CD166/ALCAM expression via interaction with micoRNA-9 after serum deprivation, providing evidence for a novel negative auto-regulatory loop.” Nucleic Acids Res 39(15): 6440-6455.
Wang, Z. and X. Yan (2013). “CD146, a multi-functional molecule beyond adhesion.” Cancer Lett 330(2): 150-162.
Warny, M., Aboudola, S., Robson, S. C., Sevigny, J., Communi, D., Soltoff, S. P. & Kelly, C. P. (2001) P2Y6 nucleotide receptor mediates monocyte interleukin-8 production in response to UDP or lipopolysaccharide. Journal of Biological Chemistry, 276, 26051-26056.
Watanabe, T., Tomioka, N. H., Doshi, M., Watanabe, S., Tsuchiya, M. & Hosoyamada, M. (2013) Macrophage migration inhibitory factor is a possible candidate for the induction of microalbuminuria in diabetic db/db mice. Biological and Pharmaceutical Bulletin, 36, 741-747.
Waters, K. M., Tan, R., Genetos, D. C., Verma, S., Yellowley, C. E. & Karin, N. J. (2007) DNA microarray analysis reveals a role for lysophosphatidic acid in the regulation of anti-inflammatory genes in MC3T3-E1 cells. Bone, 41, 833-841.
Weidle U H, Eggle D, Klostermann S, and Swart G W. ALCAM/CD166: cancer-related issues. Cancer genomics & proteomics. 2010; 7(5):231-43.
Weidle, U. H., D. Eggle, S. Klostermann and G. W. Swart (2010). “ALCAM/CD166: cancer-related issues.” Cancer Genomics Proteomics 7(5): 231-243.
Wensman, H., Kamgari, N., Johansson, A., Grujic, M., Calounova, G., Lundequist, A., Rönnberg, E. & Pejler, G. (2012) Tumor-mast cell interactions: Induction of pro-tumorigenic genes and anti-tumorigenic 4-1BB in MCs in response to Lewis Lung Carcinoma. Molecular immunology, 50, 210-219.
White, J. H., Chiano, M., Wigglesworth, M., Geske, R., Riley, J., White, N., Hall, S., Zhu, G., Maurio, F. & Savage, T. (2008) Identification of a novel asthma susceptibility gene on chromosome 1qter and its functional evaluation. Human molecular genetics, 17, 1890-1903.
Wright, D. H., Ford-Hutchinson, A. W., Chadee, K. & Metters, K. M. (2000) The human prostanoid DP receptor stimulates mucin secretion in LS174T cells. British journal of pharmacology, 131, 1537-1545.
Xiong, X., White, R. E., Xu, L., Yang, L., Sun, X., Zou, B., Pascual, C., Sakurai, T., Giffard, R. G. & Xie, X. S. (2013) Mitigation of murine focal cerebral ischemia by the hypocretin/orexin system is associated with reduced inflammation. Stroke, 44, 764-770.
Yang J, Yan R, Roy A, Xu D, Poisson J, Zhang Y. The I-TASSER Suite: Protein structure and function prediction, Nature Methods, 2015, 12: 7-8
Yang, D., Chen, Q., Gertz, B., He, R., Phulsuksombati, M., Ye, R. D. & Oppenheim, J. J. (2002) Human dendritic cells express functional formyl peptide receptor-like-2 (FPRL2) throughout maturation. J Leukoc Biol, 72, 598-607.
Yang, H.-Y. & Iadarola, M. (2003) Activation of spinal neuropeptide FF and the neuropeptide FF receptor 2 during inflammatory hyperalgesia in rats. Neuroscience, 118, 179-187.
Ye M, Du Y L, Nie Y Q, Zhou Z W, Cao J, and Li Y F. Overexpression of activated leukocute cell adhesion molecule in gastric cancer is associated with advanced stages and poor prognosis and miR-9 deregulation. Molecular medicine reports. 2015; 11(3):2004-12.
Ye, M., Y. L. Du, Y. Q. Nie, Z. W. Zhou, J. Cao and Y. F. Li (2015). “Overexpression of activated leukocute cell adhesion molecule in gastric cancer is associated with advanced stages and poor prognosis and miR-9 deregulation.” Mol Med Rep 11(3): 2004-2012.
Yi, T., Lee, D.-S., Jeon, M.-S., Kwon, S. W. & Song, S. U. (2012) Gene expression profile reveals that STAT2 is involved in the immunosuppressive function of human bone marrow-derived mesenchymal stem cells. Gene, 497, 131-139
Yin, X., Cheng, H., Lin, Y., Fan, X., Cui, Y., Zhou, F., Shen, C., Zuo, X., Zheng, X. & Zhang, W. (2014) Five regulatory genes detected by matching signatures of eQTL and GWAS in psoriasis. Journal of dermatological science, 76, 139-142.
Yokomizo, T., Kato, K., Terawaki, K., Izumi, T. & Shimizu, T. (2000) A Second Leukotriene B4 Receptor, Blt2 A New Therapeutic Target in Inflammation and Immunological Disorders. The Journal of experimental medicine, 192, 421-432.
Yu W, Wang J, Ma L, Tang X, Qiao Y, Pan Q, Yu Y, and Sun F. CD166 plays a pro-carcinogenic role in liver cancer cells via inhibition of FOXO proteins through AKT. Oncology reports. 2014; 32(2):677-83.
Yu, W., J. Wang, L. Ma, X. Tang, Y. Qiao, Q. Pan, Y. Yu and F. Sun (2014). “CD166 plays a pro-carcinogenic role in liver cancer cells via inhibition of FOXO proteins through AKT.” Oncol Rep 32(2): 677-683.
Zen, Q., M. Batchvarova, C. A. Twyman, C. E. Eyler, H. Qiu, L. M. De Castro and M. J. Telen (2004). “B-CAM/LU expression and the role of B-CAM/LU activation in binding of low- and high-density red cells to laminin in sickle cell disease.” Am J Hematol 75(2): 63-72.
Zhang Y. I-TASSER server for protein 3D structure prediction. BMC Bioinformatics, 2008, 9: 40
Zhang, F., Wu, R., Qiang, X., Zhou, M. & Wang, P. (2010a) Antagonism of α2A-adrenoceptor: a novel approach to inhibit inflammatory responses in sepsis. Journal of Molecular Medicine, 88, 289-296.
Zhang, X., Schmudde, I., Laumonnier, Y., Pandey, M., Clark, J., Konig, P., Gerard, N., Gerard, C., Wills-Karp, M. & Kohl, J. (2010b) A critical role for C5L2 in the pathogenesis of experimental allergic asthma. Journal of immunology (Baltimore, Md.: 1950), 185, 6741.
Zhong, H., Shlykov, S. G., Molina, J. G., Sanborn, B. M., Jacobson, M. A., Tilley, S. L. & Blackburn, M. R. (2003) Activation of murine lung mast cells by the adenosine A3 receptor. The Journal of Immunology, 171, 338-345.
Zhou, N., Fan, X., Mukhtar, M., Fang, J., Patel, C. A., DuBois, G. C. & Pomerantz, R. J. (2003) Cell-cell fusion and internalization of the CNS-based, HIV-1 co-receptor, APJ. Virology, 307, 22-36.
Zhu, P., Sun, W., Zhang, C., Song, Z. & Lin, S. (2016) The role of neuropeptide Y in the pathophysiology of atherosclerotic cardiovascular disease. International Journal of Cardiology, 220, 235-241
Zimmerman A W, Nelissen J M, van Emst-de Vries S E, Willems P H, de Lange F, Collard J G, van Leeuwen F N, and Figdor C G. Cytoskeletal restraints regulate homotypic ALCAM-mediated adhesion through PKCalpha independently of Rho-like GTPases. Journal of cell science. 2004; 117(Pt 13):2841-52.
Zimmerman, A. W., J. M. Nelissen, S. E. van Emst-de Vries, P. H. Willems, F. de Lange, J. G. Collard, F. N. van Leeuwen and C. G. Figdor (2004). “Cytoskeletal restraints regulate homotypic ALCAM-mediated adhesion through PKCalpha independently of Rho-like GTPases.” J Cell Sci 117(Pt 13): 2841-2852.
Zimmerman, A. W., Joosten, B., Torensma, R., Parnes, J. R., van Leeuwen, F. N. and Figdor, C. G. (2006). Long-term engagement of CD6 and ALCAM is essential for Tcell proliferation induced by dendritic cells. Blood 107, 3212-3220.
Ziogas, D. C., Gras-Miralles, B., Mustafa, S., Geiger, B. M., Najarian, R. M., Nagel, J. M., Flier, S. N., Popov, Y., Tseng, Y.-H. & Kokkotou, E. (2013) Anti-melanin-concentrating hormone treatment attenuates chronic experimental colitis and fibrosis. American Journal of Physiology-Gastrointestinal and Liver Physiology, 304, G876-G884.

1. A modulator of IgSF CAM activity where such IgSF CAM activity is induced by an activated co-located GPCR;
wherein the modulator is a modulator of IgSF CAM ligand-independent activation of an IgSF CAM, and/or a modulator of IgSF CAM ligand-dependent activation of an IgSF CAM by its cognate ligand;
wherein the activated co-located GPCR is;
i. implicated in inflammation; or
ii. implicated in cell proliferation; or
iii. selected from the group: ADGRA2, ADGRB2, ADGRB3, ADGRF3, ADGRG4, ADGRV1, CELSR1, CELSR2, CELSR3, OX1 receptor, OX2 receptor, PTH1 receptor, PTH2 receptor, AMY1 receptor, AMY2 receptor, AMY3 receptor, AM1 receptor, AM2 receptor, GPR63, GPR75, NMU2 receptor, OPN5, V1B receptor, y6 receptor, 5-HT4 receptor, GPR101, GPR119, GPR135, GPR137, GPR141, GPR149, GPR150, GPR151, GPR152, GPR157, GPR19, GPR25, GPR37, GPR37L1, GPR50, GPR62, LGR5, MRGPRE, MRGPRF, NTS2 receptor, OPN4, OPN4, OR10A7, OR10AG1, OR10Q1, OR10W1, OR12D3, OR13C2, OR13C3, OR13C4, OR13C5, OR13C8, OR13F1, OR13G1, OR1A2, OR1L1, OR1S1, OR1S2, OR2AK2, OR2D2, OR2D3, OR4A15, OR4C11, OR4C12, OR4C13, OR4C15, OR4C16, OR4K13, OR4K14, OR4K15, OR4K17, OR4N5, OR5AC2, OR5AK2, OR5AP2, OR5AR1, OR5AS1, OR5B12, OR5B17, OR5B2, OR5B21, OR5B3, OR5D13, OR5D14, OR5D16, OR5D18, OR5F1, OR51I, OR5J2, OR5K3, OR5L1, OR5L2, OR5M1, OR5M10, OR5M11, OR5M3, OR5M8, OR5M9, OR5R1, OR5T1, OR5T2, OR5T3, OR5W2, OR6C74, OR6K6, OR6M1, OR6Q1, OR6X1, OR8H1, OR8H2, OR8H3, OR8J1, OR8J3, OR8K1, OR8K3, OR8K5, OR8U1, OR8U8, OR9A4, OR9G1, OR9G4, OR9G9, OR9Q2, TAAR3, TPRA1, Y4 receptor, 5-HT1D receptor, 5-HT1E receptor, ADGRB1, AT2 receptor, BB1 receptor, BB3 receptor, CGRP receptor, CRF1 receptor, CRF2 receptor, ETA receptor, ETB receptor, FZD4, FZD5, FZD7, FZD8, FZD9, GABAB receptor, GABAB1, GABAB2, GAL1 receptor, GIP receptor, GLP-1 receptor, GLP-2 receptor, glucagon receptor, GnRH2 receptor, GPER, GPR107, GPR139, GPR156, GPR158, GPR161, GPR171, GPR179, GPR39, GPR45, GPR88, GPRC5A, GPRC5B, GPRC5C, H3 receptor, HCA1 receptor, LPA1 receptor, LPA3 receptor, LPA4 receptor, MC2 receptor, MC4 receptor, mGlu2 receptor, mGlu3 receptor, motilin receptor, MRGPRD, MRGPRX1, MRGPRX3, NK2 receptor, NPFF1 receptor, NPFF2 receptor, NPS receptor, NTS1 receptor, OR1D2, OR2AG1, OT receptor, PAC1 receptor, RXFP1 receptor, secretin receptor, TSH receptor, UT receptor, V1A receptor, V2 receptor, α2A-adrenoceptor, α2B-adrenoceptor, α2C-adrenoceptor, β1-adrenoceptor, β3-adrenoceptor, 5-HT1B receptor, 5-HT1F receptor, 5-HT2B receptor, 5-HT2C receptor, 5-HT5A receptor, 5-HT6 receptor, 5-HT7 receptor, ADGRE4P, ADGRF1, ADGRG1, ADGRG3, ADGRG5, calcitonin receptor-like receptor, CB1 receptor, CB2 receptor, CCK1 receptor, CCK2 receptor, CT receptor, D1 receptor, D2 receptor, D3 receptor, D4 receptor, D5 receptor, FFA1 receptor, FFA3 receptor, FSH receptor, FZD1, FZD2, FZD3, GHRH receptor, GnRH1 receptor, GPBA receptor, GPR1, GPR119, GPR12, GPR142, GPR143, GPR146, GPR148, GPR153, GPR160, GPR162, GPR17, GPR173, GPR174, GPR176, GPR18, GPR182, GPR20, GPR22, GPR26, GPR27, GPR3, GPR33, GPR35, GPR6, GPR61, GPR78, GPR82, GPR83, GPR84, GPR85, GPR87, GPRC5D, GPRC6 receptor, HCA2 receptor, HCA3 receptor, kisspeptin receptor, LGR4, LGR6, LH receptor, LPA2 receptor, LPA6 receptor, M1 receptor, M2 receptor, M3 receptor, M4 receptor, M5 receptor, MAS1L, MC3 receptor, MC5 receptor, MCH2 receptor, mGlu4 receptor, mGlu7 receptor, mGlu8 receptor, MRGPRG, NOP receptor, NPBW1 receptor, NPBW2 receptor, OPN3, OR11H1, OR2A1, OR2A2, OR2A4, OR2A42, OR2A7, OR2B11, OR2B6, OR2C1, OR2C3, OR2J3, OR2L13, OR2T11, OR2T34, OR2W3, OR3A3, OR4D10, OR4M1, OR4Q3, OR51A2, OR51A4, OR51A7, OR51B2, OR51B4, OR51B5, OR51B6, OR51D1, OR51E1, OR51E1, OR51E2, OR51F1, OR51F2, OR51G1, OR51G2, OR51I1, OR51I2, OR51J1, OR51L1, OR51M1, OR51Q1, OR51S1, OR51T1, OR51V1, OR52A1, OR52A4, OR52A5, OR52B2, OR52B4, OR52B6, OR52D1, OR52E2, OR52E4, OR52E5, OR52E6, OR52E8, OR52H1, OR52I1, OR52I2, OR52J3, OR52K1, OR52K2, OR52L1, OR52M1, OR52N1, OR52N2, OR52N4, OR52N5, OR52R1, OR52W1, OR56A1, OR56A3, OR56A4, OR56A5, OR56B1, OR56B4, OR6V1, OR7D2, OR9A2, oxoglutarate receptor, P2RY10, P2RY8, P2Y12 receptor, P2Y4 receptor, PrRP receptor, QRFP receptor, RXFP2 receptor, RXFP4 receptor, sst1 receptor, sst2 receptor, sst3 receptor, sst4 receptor, sst5 receptor, TA1 receptor, TAAR2, TAAR5, TAAR6, TAAR8, TAAR9, TAS1R1, TAS1R2, TAS1R3, TAS2R1, TAS2R10, TAS2R13, TAS2R14, TAS2R16, TAS2R19, TAS2R20, TAS2R3, TAS2R30, TAS2R31, TAS2R38, TAS2R39, TAS2R4, TAS2R40, TAS2R41, TAS2R42, TAS2R43, TAS2R45, TAS2R46, TAS2R5, TAS2R50, TAS2R60, TAS2R7, TAS2R8, TAS2R9, TRH1 receptor, Y1 receptor, Y2 receptor, Y5 receptor, α1A-adrenoceptor, α1B-adrenoceptor, α1D-adrenoceptor, δ receptor, 5-HT1A receptor, 5-HT2A receptor, A1 receptor, A2A receptor, A2B receptor, A3 receptor, ACKR1, ACKR2, ACKR3, ACKR4, ADGRE1, ADGRE2, ADGRE3, ADGRE5, apelin receptor, AT1 receptor, B1 receptor, B2 receptor, BB2 (GRP) receptor, BLT1 receptor, BLT2 receptor, C3a receptor, C5a1 receptor, C5a2 receptor, CaS receptor, CCR1, CCR10, CCR2, CCR3, CCR4, CCR5, CCR6, CCR7, CCR8, CCR9, CCRL2, chemerin receptor, CX3CR1, CXCR1, CXCR2, CXCR3, CXCR4, CXCR5, CXCR6, CysLT1 receptor, CysLT2 receptor, DP1 receptor, DP2 receptor, EP1 receptor, EP2 receptor, EP3 receptor, EP4 receptor, FFA2 receptor, FFA4 receptor, FP receptor, FPR1, FPR2/ALX, FPR2/ALX, FPR3, FZD6, GAL2 receptor, GAL3 receptor, ghrelin receptor, GPR132, GPR15, GPR18, GPR183, GPR21, GPR31, GPR32, GPR34, GPR4, GPR55, GPR55, GPR65, GPR68, H1 receptor, H2 receptor, H4 receptor, IP receptor, LPA5 receptor, MAS1, MC1 receptor, MCH1 receptor, mGlu1 receptor, mGlu5 receptor, MRGPRX2, MT1 receptor, MT2 receptor, NK1 receptor, NK3 receptor, NMU1 receptor, OXE receptor, P2Y1 receptor, P2Y11 receptor, P2Y13 receptor, P2Y14 receptor, P2Y2 receptor, P2Y6 receptor, PAF receptor, PAR1, PAR2, PAR3, PAR4, PKR1, PKR2, S1P1 receptor, S1P2 receptor, S1P3 receptor, S1P4 receptor, S1P5 receptor, succinate receptor, TP receptor, VPAC1 receptor, VPAC2 receptor, XCR1, β2-adrenoceptor, κ receptor, or μ receptor,
and wherein the modulator;
a) consists of the ectodomain of an IgSF CAM; or
b) does not contain the ectodomain of an IgSF CAM; or
c) does not contain an analogue, fragment or derivative of the ectodomain of an IgSF CAM; or
d) does not bind to the ligand-binding domain of an IgSF CAM; or
e) inhibits or facilitates signalling that occurs through the C-terminal cytosolic tail of an IgSF CAM induced by an activated co-located GPCR; or
f) inhibits binding that occurs to the C-terminal cytosolic tail of an IgSF CAM; or
g) inhibits or facilitates the interaction between the IgSF CAM and the GPCR; or
h) inhibits or facilitates the capacity of the GPCR to modulate IgSF CAM-dependent signalling that is dependent upon proximity of an IgSF CAM and the GPCR; or
i) inhibits IgSF CAM ligand-independent activation of IgSF CAM by activated AT₁R; or
j) is a non-functional substitute for the cytosolic tail of RAGE or a part thereof, which is not able to be activated by a co-located GPCR or facilitate downstream RAGE-dependent signalling and inhibits signalling that occurs through the cytosolic tail of an IgSF CAM and IgSF CAM-dependent signalling; or
k) comprises a transmembrane domain of RAGE or a part thereof and a fragment of the RAGE ectodomain; or
l) comprises a transmembrane domain of RAGE or a part thereof and a fragment of the cytosolic tail of RAGE; or
m) comprises a transmembrane domain of RAGE or part thereof and a fragment of the RAGE ectodomain and a fragment of the cytosolic tail of RAGE; or
n) comprises a fragment of the ectodomain of RAGE, which is not greater than 40, not greater than 20, not greater than 10 or not greater than 5 amino acids in length; or
o) does not contain the cytosolic tail of an IgSF CAM; or
p) is an analogue, fragment or derivative of the cytosolic tail of an IgSF CAM; or
q) contains an analogue, fragment or derivative of the transmembrane domain of an IgSF CAM and does not contain the cytosolic tail of an IgSF CAM or a fragment thereof; or
r) contains the entire ectodomain of an IgSF CAM conjugated to an analogue, fragment or derivative of the transmembrane domain of an IgSF CAM; or
s) contains the entire ectodomain of an IgSF CAM conjugated to an analogue, fragment or derivative of the transmembrane domain of an IgSF CAM which is greater than 20, greater than 10, or greater than 5 amino acids in length; or
t) contains a truncated ectodomain of an IgSF CAM; or
u) acts in the presence of a truncated ectodomain of an IgSF CAM; or
v) acts in the absence of the IgSF CAM ligand-binding ectodomain of an IgSF CAM.

2. The modulator of claim 1, wherein the modulator is a modulator of IgSF CAM ligand-independent activation of an IgSF CAM, and is not a modulator of IgSF CAM ligand-dependent activation of an IgSF CAM by its cognate ligand.

3. The modulator of claim 1, wherein the modulator is a modulator of IgSF CAM ligand-independent activation of an IgSF CAM, and is also a modulator of IgSF CAM ligand-dependent activation of an IgSF CAM by its cognate ligand.

4. The modulator of claim 1, wherein the modulator is not a modulator of IgSF CAM ligand-independent activation of an IgSF CAM, and is a modulator of IgSF CAM ligand-dependent activation of an IgSF CAM by its cognate ligand.

5. The modulator of claim 1, wherein;
I. the modulator includes isolated or purified peptides which comprise, consist, or consist essentially of an amino acid sequence represented by Formula I:
Z1MZ2 (I)
wherein:
i. Z1 is absent or Z1 is selected from at least one of a proteinaceous moiety comprising from about 1 to about 50 amino acid residues, or Z1 is a cell membrane penetration molecule or Z1 is a fragment of the RAGE cytosolic tail or an IgSF CAM cytosolic tail;
ii. M is;
A. the amino acid sequence or peptide as set forth in SEQ ID NO: 1; or
B. an analogue, fragment or derivative thereof; or
C. an analogue of the C-terminal cytosolic tail of the ALCAM polypeptide as set forth in SEQ ID NO: 1 that shares at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity or similarity with, or differs at no more than 1, 2, 3, 5, 10, 15 or 20 amino acid residues from the C-terminal cytosolic tail of the ALCAM polypeptide sequence; or
D. comprises any 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 amino acid fragment of the C-terminal cytosolic tail of the ALCAM polypeptide; or
E. is an analogue of the fragment that shares at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity or similarity with, or differs at no more than 1, 2, 3, 5, 10, 15 or 20 amino acid residues from the fragment; or
F. is an analogue, fragment or derivate of SEQ ID NO: 1 that contains at least residues 551-583 of ALCAM; or
G. is a peptide of the formula SEQ ID NO: 2, or an analogue or derivative thereof; or
H. is a peptide of the formula SEQ ID NO: 3, or an analogue or derivative thereof; or
I. is a peptide of the formula SEQ ID NO: 4, or an analogue or derivative thereof; or
J. is a peptide of the formula SEQ ID NO: 5, or an analogue or derivative thereof; or
K. is a peptide of the formula SEQ ID NO: 6, or an analogue or derivative thereof; or
L. is a peptide of the formula SEQ ID NO: 7, or an analogue or derivative thereof; or
M. is a peptide of the formula SEQ ID NO: 8, or an analogue or derivative thereof; or
N. is a peptide of the formula SEQ ID NO: 19, or an analogue or derivative thereof; or
O. is a peptide of the formula SEQ ID NO: 21, or an analogue or derivative thereof; or
P. is a peptide of the formula SEQ ID NO: 22, or an analogue or derivative thereof; or
Q. is a peptide of the formula SEQ ID NO: 23, or an analogue or derivative thereof; or
R. is a peptide of the formula SEQ ID NO: 24, or an analogue or derivative thereof; or
S. is a peptide of the formula SEQ ID NO: 25, or an analogue or derivative thereof; or
T. is a peptide of the formula SEQ ID NO: 26, or an analogue or derivative thereof; or
U. is a peptide of the formula SEQ ID NO: 27, or an analogue or derivative thereof; or
V. is a peptide of the formula SEQ ID NO: 28, or an analogue or derivative thereof; or
W. is a peptide of the formula SEQ ID NO: 29, or an analogue or derivative thereof; or
X. is a peptide of the formula SEQ ID NO: 30, or an analogue or derivative thereof; or
Y. is a peptide of the formula SEQ ID NO: 31, or an analogue or derivative thereof;
and
iii. Z2 is absent or Z2 is a proteinaceous moiety comprising from about 1 to about 50 amino acid residues or Z2 is a cell membrane penetration molecule or Z2 is a fragment of the RAGE cytosolic tail or an IgSF CAM cytosolic tail;
or characterized in that;
II. the modulator is an analogue of the peptide of any one of SEQ ID NOs: 1, 2, 3, 4, 5, 6, 7, 8, 19, 21, 22, 23, 24, 25, 26, 27, 28, 29, 30 or 31, wherein the analogue shares at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity or similarity with, or differs at no more than 1, 2, 3, 5 or even 10 amino acid residues from the peptide of any one of SEQ ID NOs: 1, 2, 3, 4, 5, 6, 7, 8, 19, 21, 22, 23, 24, 25, 26, 27, 28, 29, 30 or 31.

6. The modulator of claim 1, wherein;
i. the modulator is a polypeptide derived from any member of the IgSF CAM superfamily; or
ii. the modulator is a polypeptide derived from ALCAM, BCAM, MCAM, EpCAM or CADM4; or
iii. the modulator is a polypeptide derived from human wild-type RAGE polypeptide, wherein the polypeptide is modified at serine-391, of the C-terminal cytosolic tail of human wild-type RAGE polypeptide; or
iv. the modulator is a polypeptide derived from human wild-type RAGE polypeptide, wherein serine-391 of the C-terminal cytosolic tail of human wild-type RAGE polypeptide is substituted with an amino acid residue selected from the group: glutamine, proline, threonine, leucine, alanine, cysteine, arginine, lysine, aspartate, glutamate, glycine, histidine, methionine, phenylalanine, valine, asparagine, isoleucine, tryptophan or tyrosine.

7. The modulator of claim 1, wherein;
i. the modulator does not modulate the interaction of RAGE and Diaphanous-1; or
ii. the modulator lacks or has an impaired ability to bind Diaphanous-1 relative to human wild-type RAGE; or
iii. the modulator is a peptide characterized in that the peptide lacks the RAGE-Diaphanous-1 binding site R366-Q367; or
iv. the modulator is a peptide having an altered RAGE-Diaphanous-1 binding site R366-Q367; or
v. the modulator is a peptide having an altered RAGE-Diaphanous-1 binding site characterized in that the residues at R366/Q367 are deleted or substituted with other residues in order to impair or abolish this site.

8. The modulator of claim 1, wherein the modulator inhibits activation of the cytosolic tail of an IgSF CAM by activated co-located GPCRs that bind to one or more of the following:
Ras GTPase-activating-like protein (IQGAP1) IgSF CAM-associated proteins, protein kinase C zeta (PKCζ), Dock7, MyD88, TIRAP, IRAK4, ERK1/2, olfactory receptor 2T2, ADP/ATP translocase 2, Protein phosphatase 1G, Intercellular adhesion molecule 1, Protein DJ-1 (PARK7), Calponin-3, Drebrin, Filamin B, Ras-related protein Rab-13, Radixin/Ezrin/Moesin, Proteolipid protein 2, Coronin, S100 A11, Succinyl-CoA ligase [GDP-forming] subunit alpha, Hsc70-interacting protein, Apoptosis Inhibitor 5, neuropilin, cleavage stimulation factor, growth factor receptor-bound protein 2, sec61 beta subunit, or Nck1.

9. The modulator of claim 1, wherein the modulator modulates IgSF CAM transactivation by an activated co-located GPCR, by disrupting the binding of one or more of the following to IgSF CAM and/or to the GPCR;
Ras GTPase-activating-like protein (IQGAP1) IgSF CAM-associated proteins, protein kinase C zeta (PKCζ), Dock7, MyD88, TIRAP, IRAK4, ERK1/2, olfactory receptor 2T2, ADP/ATP translocase 2, Protein phosphatase 1G, Intercellular adhesion molecule 1, Protein DJ-1 (PARK7), Calponin-3, Drebrin, Filamin B, Ras-related protein Rab-13, Radixin/Ezrin/Moesin, Proteolipid protein 2, Coronin, S100 A11, Succinyl-CoA ligase [GDP-forming] subunit alpha, Hsc70-interacting protein, Apoptosis Inhibitor 5, neuropilin, cleavage stimulation factor, growth factor receptor-bound protein 2, sec61 beta subunit, or Nck1.

10. The modulator of claim 1, wherein the modulator modulates IgSF CAM ligand-independent activation of an IgSF CAM by an activated co-located GPCR and/or modulates IgSF CAM ligand-dependent activation of the cytosolic tail of an IgSF CAM, by binding to cytosolic elements of an IgSF CAM and/or elements that complex with an IgSF CAM in the cytosol, to inhibit IgSF CAM ligand-mediated signalling through these elements, such elements including IQGAP-1, PKCζ, Dock7, MyD88, IRAK4, TIRAP, ERK1/2, olfactory receptor 2T2, ADP/ATP translocase 2, Protein phosphatase 1G, Intercellular adhesion molecule 1, Protein DJ-1 (PARK7), Calponin-3, Drebrin, Filamin B, Ras-related protein Rab-13, Radixin/Ezrin/Moesin, Proteolipid protein 2, Coronin, S100 A11, Succinyl-CoA ligase [GDP-forming] subunit alpha, Hsc70-interacting protein, Apoptosis Inhibitor 5, neuropilin, cleavage stimulation factor, growth factor receptor-bound protein 2, sec61 beta subunit, or Nck1.

11. A modulator of RAGE ligand-independent activation of RAGE by an activated co-located GPCR, where the modulator is an analogue, fragment or derivative of an IgSF CAM that modulates transactivation of the cytosolic tail of RAGE triggered by activation of the activated co-located GPCR;
wherein the activated co-located GPCR is;
i. implicated in inflammation; or
ii. implicated in cell proliferation; or
iii. selected from the group: ADGRA2, ADGRB2, ADGRB3, ADGRF3, ADGRG4, ADGRV1, CELSR1, CELSR2, CELSR3, OX1 receptor, OX2 receptor, PTH1 receptor, PTH2 receptor, AMY1 receptor, AMY2 receptor, AMY3 receptor, AM1 receptor, AM2 receptor, GPR63, GPR75, NMU2 receptor, OPN5, V1B receptor, y6 receptor, 5-HT4 receptor, GPR101, GPR119, GPR135, GPR137, GPR141, GPR149, GPR150, GPR151, GPR152, GPR157, GPR19, GPR25, GPR37, GPR37L1, GPR50, GPR62, LGR5, MRGPRE, MRGPRF, NTS2 receptor, OPN4, OPN4, OR10A7, OR10AG1, OR10Q1, OR10W1, OR12D3, OR13C2, OR13C3, OR13C4, OR13C5, OR13C8, OR13F1, OR13G1, OR1A2, OR1L1, OR1S1, OR1S2, OR2AK2, OR2D2, OR2D3, OR4A15, OR4C11, OR4C12, OR4C13, OR4C15, OR4C16, OR4K13, OR4K14, OR4K15, OR4K17, OR4N5, OR5AC2, OR5AK2, OR5AP2, OR5AR1, OR5AS1, OR5B12, OR5B17, OR5B2, OR5B21, OR5B3, OR5D13, OR5D14, OR5D16, OR5D18, OR5F1, OR51I, OR5J2, OR5K3, OR5L1, OR5L2, OR5M1, OR5M10, OR5M11, OR5M3, OR5M8, OR5M9, OR5R1, OR5T1, OR5T2, OR5T3, OR5W2, OR6C74, OR6K6, OR6M1, OR6Q1, OR6X1, OR8H1, OR8H2, OR8H3, OR8J1, OR8J3, OR8K1, OR8K3, OR8K5, OR8U1, OR8U8, OR9A4, OR9G1, OR9G4, OR9G9, OR9Q2, TAAR3, TPRA1, Y4 receptor, 5-HT1D receptor, 5-HT1E receptor, ADGRB1, AT2 receptor, BB1 receptor, BB3 receptor, CGRP receptor, CRF1 receptor, CRF2 receptor, ETA receptor, ETB receptor, FZD4, FZD5, FZD7, FZD8, FZD9, GABAB receptor, GABAB1, GABAB2, GAL1 receptor, GIP receptor, GLP-1 receptor, GLP-2 receptor, glucagon receptor, GnRH2 receptor, GPER, GPR107, GPR139, GPR156, GPR158, GPR161, GPR171, GPR179, GPR39, GPR45, GPR88, GPRC5A, GPRC5B, GPRC5C, H3 receptor, HCA1 receptor, LPA1 receptor, LPA3 receptor, LPA4 receptor, MC2 receptor, MC4 receptor, mGlu2 receptor, mGlu3 receptor, motilin receptor, MRGPRD, MRGPRX1, MRGPRX3, NK2 receptor, NPFF1 receptor, NPFF2 receptor, NPS receptor, NTS1 receptor, OR1D2, OR2AG1, OT receptor, PAC1 receptor, RXFP1 receptor, secretin receptor, TSH receptor, UT receptor, V1A receptor, V2 receptor, α2A-adrenoceptor, α2B-adrenoceptor, α2C-adrenoceptor, β1-adrenoceptor, β3-adrenoceptor, 5-HT1B receptor, 5-HT1F receptor, 5-HT2B receptor, 5-HT2C receptor, 5-HT5A receptor, 5-HT6 receptor, 5-HT7 receptor, ADGRE4P, ADGRF1, ADGRG1, ADGRG3, ADGRG5, calcitonin receptor-like receptor, CB1 receptor, CB2 receptor, CCK1 receptor, CCK2 receptor, CT receptor, D1 receptor, D2 receptor, D3 receptor, D4 receptor, D5 receptor, FFA1 receptor, FFA3 receptor, FSH receptor, FZD1, FZD2, FZD3, GHRH receptor, GnRH1 receptor, GPBA receptor, GPR1, GPR119, GPR12, GPR142, GPR143, GPR146, GPR148, GPR153, GPR160, GPR162, GPR17, GPR173, GPR174, GPR176, GPR18, GPR182, GPR20, GPR22, GPR26, GPR27, GPR3, GPR33, GPR35, GPR6, GPR61, GPR78, GPR82, GPR83, GPR84, GPR85, GPR87, GPRC5D, GPRC6 receptor, HCA2 receptor, HCA3 receptor, kisspeptin receptor, LGR4, LGR6, LH receptor, LPA2 receptor, LPA6 receptor, M1 receptor, M2 receptor, M3 receptor, M4 receptor, M5 receptor, MAS1L, MC3 receptor, MC5 receptor, MCH2 receptor, mGlu4 receptor, mGlu7 receptor, mGlu8 receptor, MRGPRG, NOP receptor, NPBW1 receptor, NPBW2 receptor, OPN3, OR11H1, OR2A1, OR2A2, OR2A4, OR2A42, OR2A7, OR2B11, OR2B6, OR2C1, OR2C3, OR2J3, OR2L13, OR2T11, OR2T34, OR2W3, OR3A3, OR4D10, OR4M1, OR4Q3, OR51A2, OR51A4, OR51A7, OR51B2, OR51B4, OR51B5, OR51B6, OR51D1, OR51E1, OR51E1, OR51E2, OR51F1, OR51F2, OR51G1, OR51G2, OR51I1, OR51I2, OR51J1, OR51L1, OR51M1, OR51Q1, OR51S1, OR51T1, OR51V1, OR52A1, OR52A4, OR52A5, OR52B2, OR52B4, OR52B6, OR52D1, OR52E2, OR52E4, OR52E5, OR52E6, OR52E8, OR52H1, OR52I1, OR52I2, OR52J3, OR52K1, OR52K2, OR52L1, OR52M1, OR52N1, OR52N2, OR52N4, OR52N5, OR52R1, OR52W1, OR56A1, OR56A3, OR56A4, OR56A5, OR56B1, OR56B4, OR6V1, OR7D2, OR9A2, oxoglutarate receptor, P2RY10, P2RY8, P2Y12 receptor, P2Y4 receptor, PrRP receptor, QRFP receptor, RXFP2 receptor, RXFP4 receptor, sst1 receptor, sst2 receptor, sst3 receptor, sst4 receptor, sst5 receptor, TA1 receptor, TAAR2, TAAR5, TAAR6, TAAR8, TAAR9, TAS1R1, TAS1R2, TAS1R3, TAS2R1, TAS2R10, TAS2R13, TAS2R14, TAS2R16, TAS2R19, TAS2R20, TAS2R3, TAS2R30, TAS2R31, TAS2R38, TAS2R39, TAS2R4, TAS2R40, TAS2R41, TAS2R42, TAS2R43, TAS2R45, TAS2R46, TAS2R5, TAS2R50, TAS2R60, TAS2R7, TAS2R8, TAS2R9, TRH1 receptor, Y1 receptor, Y2 receptor, Y5 receptor, α1A-adrenoceptor, α1B-adrenoceptor, α1D-adrenoceptor, δ receptor, 5-HT1A receptor, 5-HT2A receptor, A1 receptor, A2A receptor, A2B receptor, A3 receptor, ACKR1, ACKR2, ACKR3, ACKR4, ADGRE1, ADGRE2, ADGRE3, ADGRE5, apelin receptor, AT1 receptor, B1 receptor, B2 receptor, BB2 (GRP) receptor, BLT1 receptor, BLT2 receptor, C3a receptor, C5a1 receptor, C5a2 receptor, CaS receptor, CCR1, CCR10, CCR2, CCR3, CCR4, CCR5, CCR6, CCR7, CCR8, CCR9, CCRL2, chemerin receptor, CX3CR1, CXCR1, CXCR2, CXCR3, CXCR4, CXCR5, CXCR6, CysLT1 receptor, CysLT2 receptor, DP1 receptor, DP2 receptor, EP1 receptor, EP2 receptor, EP3 receptor, EP4 receptor, FFA2 receptor, FFA4 receptor, FP receptor, FPR1, FPR2/ALX, FPR2/ALX, FPR3, FZD6, GAL2 receptor, GAL3 receptor, ghrelin receptor, GPR132, GPR15, GPR18, GPR183, GPR21, GPR31, GPR32, GPR34, GPR4, GPR55, GPR55, GPR65, GPR68, H1 receptor, H2 receptor, H4 receptor, IP receptor, LPA5 receptor, MAS1, MC1 receptor, MCH1 receptor, mGlu1 receptor, mGlu5 receptor, MRGPRX2, MT1 receptor, MT2 receptor, NK1 receptor, NK3 receptor, NMU1 receptor, OXE receptor, P2Y1 receptor, P2Y11 receptor, P2Y13 receptor, P2Y14 receptor, P2Y2 receptor, P2Y6 receptor, PAF receptor, PAR1, PAR2, PAR3, PAR4, PKR1, PKR2, S1P1 receptor, S1P2 receptor, S1P3 receptor, S1P4 receptor, S1P5 receptor, succinate receptor, TP receptor, VPAC1 receptor, VPAC2 receptor, XCR1, β2-adrenoceptor, κ receptor, or μ receptor,
and wherein the modulator;
a) is an analogue, fragment or derivative of an IgSF CAM that is an activator, an inhibitor, an allosteric modulator, or a non-functional mimic of the cytosolic tail of an IgSF CAM; or
b) mimics the cytosolic tail of an IgSF CAM in the presence of a co-located GPCR, is not able to be activated by it or induce downstream IgSF CAM-dependent signalling, and inhibits signalling that normally occurs through activation of the cytosolic tail of RAGE and RAGE-dependent signalling resulting therefrom; or
c) is an analogue, fragment or derivative of an IgSF CAM that is an activator, an inhibitor, an allosteric modulator, or a non-functional mimic of the transmembrane domain of an IgSF CAM or part thereof; or
d) mimics the transmembrane domain of an IgSF CAM in the presence of a co-located GPCR, is not able to be activated by it or induce downstream IgSF CAM-dependent signalling, and inhibits signalling that normally occurs through activation of the cytosolic tail of RAGE and RAGE-dependent signalling resulting therefrom; or
e) comprises a transmembrane domain of an IgSF CAM or a part thereof and a fragment of an IgSF CAM ectodomain; or
f) comprises a transmembrane domain of an IgSF CAM or a part thereof and a fragment of the cytosolic tail of an IgSF CAM; or
g) comprises a transmembrane domain of an IgSF CAM or part thereof and a fragment of an IgSF CAM ectodomain and a fragment of the cytosolic tail of an IgSF CAM; or
h) contains a fragment of the ectodomain of an IgSF CAM, which is not greater than 40, not greater than 20, not greater than 10 or not greater than 5 amino acids in length; or
i) is an inhibitor of RAGE ligand-independent activation of RAGE; or
j) in addition to being an inhibitor of RAGE ligand-independent activation of RAGE by an activated co-located GPCR, is an inhibitor of the co-located GPCR and/or an inhibitor of the co-located GPCR signalling pathway; or
k) in addition to being an inhibitor of RAGE ligand-independent activation of RAGE by an activated co-located GPCR, is an inhibitor of RAGE ligand-dependent activation of RAGE and/or an inhibitor of constitutively-active RAGE and/or an inhibitor of a RAGE signalling pathway; or
l) where the co-located GPCR is AT₁R, in addition to being an inhibitor of RAGE ligand-independent activation of RAGE, is an AT₁R inhibitor and/or an inhibitor of an AT₁R signalling pathway; or
m) in addition to being an inhibitor of RAGE ligand-independent activation of RAGE by activated angiotensin receptor, preferably activated AT₁R, is an inhibitor of RAGE ligand-dependent activation of RAGE and/or an inhibitor of constitutively-active RAGE and/or an inhibitor of a RAGE signalling pathway; or
n) in addition to being an inhibitor of RAGE ligand-independent activation of RAGE by an activated co-located GPCR, is an inhibitor of the co-located GPCR and/or an inhibitor of the co-located GPCR signalling pathway and an inhibitor of RAGE ligand-dependent activation of RAGE and/or an inhibitor of constitutively-active RAGE and/or an inhibitor of a RAGE signalling pathway; or
o) in addition to being an inhibitor of RAGE ligand-independent activation of RAGE by activated angiotensin receptor, preferably activated AT₁R, is an AT₁R inhibitor and/or an inhibitor of an AT₁R signalling pathway and an inhibitor of RAGE ligand-dependent activation of RAGE and/or an inhibitor of constitutively-active RAGE and/or an inhibitor of a RAGE signalling pathway; or
p) is a non-functional substitute for the cytosolic tail of an IgSF CAM or a part thereof, which is not able to be activated by a co-located GPCR or facilitate downstream IgSF CAM-dependent signalling and inhibits signalling that occurs through the cytosolic tail of RAGE and RAGE-dependent signalling; or
q) is a non-functional substitute for the transmembrane domain of an IgSF CAM or a part thereof, which is not able to be activated by a co-located GPCR or facilitate downstream IgSF CAM-dependent signalling and inhibits signalling that occurs through the cytosolic tail of RAGE and RAGE-dependent signalling.

12. A modulator of RAGE ligand-dependent activation of RAGE by its cognate ligand, wherein the modulator is an analogue, fragment or derivative of an IgSF CAM.

13. The modulator of claim 11, wherein the modulator is also a modulator of RAGE ligand-dependent activation of RAGE by its cognate ligand, in accordance with claim 12 and wherein the modulator is an analogue, fragment or derivative of an IgSF CAM.

14. The modulator of claim 11, wherein;
I. the modulator includes isolated or purified peptides which comprise, consist, or consist essentially of an amino acid sequence represented by Formula I:
Z1MZ2 (I)
wherein:
i. Z1 is absent or Z1 is selected from at least one of a proteinaceous moiety comprising from about 1 to about 50 amino acid residues, or Z1 is a cell membrane penetration molecule or Z1 is a fragment of an IgSF CAM cytosolic tail;
ii. M is;
A. the amino acid sequence or peptide as set forth in SEQ ID NO: 1; or
B. an analogue, fragment or derivative thereof; or
C. an analogue of the C-terminal cytosolic tail of the ALCAM polypeptide as set forth in SEQ ID NO: 1 that shares at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity or similarity with, or differs at no more than 1, 2, 3, 5, 10, 15 or 20 amino acid residues from the C-terminal cytosolic tail of the ALCAM polypeptide sequence; or
D. comprises any 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 amino acid fragment of the C-terminal cytosolic tail of the ALCAM polypeptide; or
E. is an analogue of the fragment that shares at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity or similarity with, or differs at no more than 1, 2, 3, 5, 10, 15 or 20 amino acid residues from the fragment; or
F. is an analogue, fragment or derivate of SEQ ID NO: 1 that contains at least residues 551-583 of ALCAM; or
G. is a peptide of the formula SEQ ID NO: 2, or an analogue or derivative thereof; or
H. is a peptide of the formula SEQ ID NO: 3, or an analogue or derivative thereof; or
I. is a peptide of the formula SEQ ID NO: 4, or an analogue or derivative thereof; or
J. is a peptide of the formula SEQ ID NO: 5, or an analogue or derivative thereof; or
K. is a peptide of the formula SEQ ID NO: 6, or an analogue or derivative thereof;
and
iii. Z2 is absent or Z2 is a proteinaceous moiety comprising from about 1 to about 50 amino acid residues or Z2 is a cell membrane penetration molecule or Z2 is a fragment of an IgSF CAM cytosolic tail;
or characterized in that;
II. the modulator is an analogue of the peptide of any one of SEQ ID NOs: 1, 2, 3, 4, 5 or 6, wherein the analogue shares at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity or similarity with, or differs at no more than 1, 2, 3, 5 or even 10 amino acid residues from the peptide of any one of SEQ ID NOs: 1, 2, 3, 4, 5 or 6.

15. The modulator of claim 11, wherein:
i. the modulator is a polypeptide derived from any member of the IgSF CAM superfamily; or
ii. the modulator is a polypeptide derived from ALCAM, BCAM, MCAM, EpCAM or CADM4.

16. The modulator of claim 11, wherein the modulator it is a modulator of RAGE ligand-independent activation of the cytosolic tail of RAGE by an activated co-located GPCR that binds to Ras GTPase-activating-like protein (IQGAP1) or other RAGE-associated proteins, including protein kinase C zeta (PKCζDock7, MyD88, TIRAP, IRAK4, ERK1/2, olfactory receptor 2T2, ADP/ATP translocase 2, Protein phosphatase 1G, Intercellular adhesion molecule 1, Protein DJ-1 (PARK7), Calponin-3, Drebrin, Filamin B, Ras-related protein Rab-13, Radixin/Ezrin/Moesin, Proteolipid protein 2, Coronin, S100 A11, Succinyl-CoA ligase [GDP-forming] subunit alpha, Hsc70-interacting protein, Apoptosis Inhibitor 5, neuropilin, cleavage stimulation factor, growth factor receptor-bound protein 2, sec61 beta subunit, or Nck1, or disrupts the binding of these elements to RAGE, in order to modulate RAGE transactivation by the activated co-located GPCR, and where the modulator is an analogue, fragment or derivative of an IgSF CAM.

17. The modulator of claim 16, wherein the modulator binds to the cytosolic elements of an activated co-located GPCR, RAGE and/or elements complexed with either, including IQGAP-1, PKCζ, Dock7, MyD88, TIRAP, IRAK4, ERK1/2, olfactory receptor 2T2, ADP/ATP translocase 2, Protein phosphatase 1G, Intercellular adhesion molecule 1, Protein DJ-1 (PARK7), Calponin-3, Drebrin, Filamin B, Ras-related protein Rab-13, Radixin/Ezrin/Moesin, Proteolipid protein 2, Coronin, S100 A11, Succinyl-CoA ligase [GDP-forming] subunit alpha, Hsc70-interacting protein, Apoptosis Inhibitor 5, neuropilin, cleavage stimulation factor, growth factor receptor-bound protein 2, sec61 beta subunit, or Nck1 to modulate RAGE ligand-independent signalling through the cytosolic tail of RAGE, by modulating these signalling elements required for RAGE transactivation by the activated co-located GPCR, and where the modulator is an analogue, fragment or derivative of an IgSF CAM.

18. The modulator of claim 17, wherein the modulator is a modulator of RAGE ligand-independent activation of RAGE by an activated co-located GPCR that also modulates RAGE ligand-dependent activation of the cytosolic tail of RAGE, by binding to cytosolic elements of RAGE and/or elements that complex with RAGE in the cytosol (such as IQGAP-1, PKCζ, Dock7, MyD88, IRAK4, TIRAP, ERK1/2, olfactory receptor 2T2, ADP/ATP translocase 2, Protein phosphatase 1G, Intercellular adhesion molecule 1, Protein DJ-1 (PARK7), Calponin-3, Drebrin, Filamin B, Ras-related protein Rab-13, Radixin/Ezrin/Moesin, Proteolipid protein 2, Coronin, S100 A11, Succinyl-CoA ligase [GDP-forming] subunit alpha, Hsc70-interacting protein, Apoptosis Inhibitor 5, neuropilin, cleavage stimulation factor, growth factor receptor-bound protein 2, sec61 beta subunit, or Nck1) to inhibit RAGE ligand-mediated signalling through these elements, and where the modulator is an analogue, fragment or derivative of an IgSF CAM.

19. The modulator of claim 1, wherein the modulator comprises two or more features selected from the group: a first charged or hydrogen bonding group (A), a second charged or hydrogen bonding group (B), a third charged or hydrogen bonding group (C), and a hydrophobic group (D), wherein the distances between the site points of the features are as follows, within a tolerance of up to ±10 Å, ±5 Å, ±2 Å, or ±1 Å provided the distances between the features is positive in magnitude;

| | A | B | C | D |
|---|---|---|---|---|
| A | | | | |
| B | 10.2 Å | | | |
| C | 13.2 Å | 8.8 Å | | |
| D | 14.6 Å | 5.1 Å | 8 Å | |

20. The modulator of claim 19, wherein the modulator is non-peptidyl.

21-33. (canceled)
